(19) United States
(12) Patent Application Publication
(10) Pub. No.: US 2024/0343800 A1
(43) Pub. Date: Oct. 17, 2024

(54) PROTEINS THAT DECOUPLE T CELL-MEDIATED TUMOR CYTOTOXICITY FROM RELEASE OF PRO-INFLAMMATORY CYTOKINES

(71) Applicant: Merck Patent GmbH, Darmstadt (DE)

(72) Inventors: Brian Rabinovich, Billerica, MA (US); Jessica Dawson, Billerica, MA (US); Xueyuan Zhou, Billerica, MA (US); Stefan Becker, Darmstadt (DE); Yves Fomekong Nanfack, Billerica, MA (US); Christian Schroeter, Darmstadt (DE); Lars Toleikis, Darmstadt (DE); Achim Doerner, Darmstadt (DE); Bjorn Hock, Darmstadt (DE)

(21) Appl. No.: 18/436,931

(22) Filed: Feb. 8, 2024

Related U.S. Application Data
(63) Continuation of application No. PCT/US2022/074686, filed on Aug. 9, 2022.
(60) Provisional application No. 63/231,148, filed on Aug. 9, 2021.

(51) Int. Cl. C07K 16/28; A61P 35/00

(52) U.S. Cl. CPC C07K 16/2809; A61P 35/00; C07K 16/2803; C07K 2317/21; C07K 2317/31; C07K 2317/622; C07K 2317/92

(57) ABSTRACT

This disclosure relates generally to proteins target tumor cell killing comprising: a polypeptide or complex of two or more polypeptides that specifically binds ROR1, and a polypeptide or complex of two or more polypeptides that specifically binds CD3. In some embodiments, the present application provides antibodies that specifically bind ROR1. In some embodiments the application also provides therapeutic methods for using such proteins in the treatment of a cancer.

### CROSS-REFERENCE TO RELATED APPLICATIONS

This application is a continuation of International Patent Application No. PCT/US2022/074686 filed Aug. 9, 2022, which application designates the United States and claims priority to and the benefit of U.S. Provisional Patent Application No. 63/231,148, filed Aug. 9, 2021, the entire disclosures of each of which are incorporated by reference herein for all purposes.

### REFERENCE TO A SEQUENCE LISTING XML

The present application contains a Sequence Listing which has been submitted electronically in XML format. The Sequence Listing XML is hereby incorporated by reference in its entirety. Said XML file, created on Jul. 26, 2022, is named EMD-018WO_SL.xml and is 325,620 bytes in size.

### FIELD OF THE DISCLOSURE

The present disclosure relates generally to bispecific proteins capable of inducing T cell cytotoxicity of tumor cells, and uses of the same for the treatment of cancers.

### BACKGROUND

ROR1 is a cell surface tyrosine kinase predominantly expressed during embryonic development. ROR1 is expressed on both hematological tumor cells including chronic lymphocytic leukemia (CLL) cells and mantle cell leukemia (MCL) cells, and solid tumors including those of the ovary, lung, stomach, pancreas, uterus, breast, and prostate. ROR1 exhibits limited expression on normal adult tissues (for example, adipose tissue, pre-B cells, pancreatic islets) and is therefore considered a relatively tumor-specific tumor-associated antigen (“TAA”). ROR1-specific antibody drug conjugates (ADC) have demonstrated promising efficacy in early clinical trials with manageable adverse event (AE) profiles. However, the low density and heterogeneous nature of cell surface ROR1 expression in solid tumors represents a challenge for many immunotherapeutic modalities including monospecific antibodies that mediate antibody-dependent cytotoxicity and ADCs.

ADCs rely on the magnitude of ADC internalization for efficacy. ROR1 ADCs appear to be highly effective in early clinical trials for ROR1V hematological malignancies including CLL and MCL but this may be attributed at least in part to: (1) greater access of ADC to hematological versus solid tumors that require penetration of the ADC, and (2) a higher frequency and more homogenous expression of ROR1 by hematological versus solid tumors.

CD3 bispecific antibodies (bsAbs) require minimal receptor occupancy to mediate potent functional activity. TAA-CD3 bsAbs activate all CD3⁺ lymphocytes, including αβ T cells, γδ T cells, NK-T cells, regulatory T cells (Tregs), mucosal associated invariant T (MAIT) cells, and their phenotypic subsets, when bridged between CD3 on T cells and the target TAA epitope on tumor cells. In solid tumors, the relative frequency of CD3⁺ lymphocytes, including CD8⁺ cytotoxic T cells, is low relative to tumor cells (generally >10 tumor cells per CD8⁺ T cell), even in highly immunogenic and inflamed tumors such as melanoma. Accordingly, efficacy of CD3 bsAbs is dependent on the capacity of a single CD8⁺ T cell to kill more than one tumor cell, a process termed serial killing.

Crosslinking of the TCR/CD3 complex via this bsAb bridge preferentially activates cytotoxic T cell subsets to mediate killing of TAA-expressing cells (the majority of which are presumed to be tumor cells) and release of pro-inflammatory cytokines capable of shifting the tumor microenvironment (TME) from immunosuppressive to immuno-permissive. In the context of Tregs, CD3 bsAbs can activate Tregs to secrete immunosuppressive cytokines such as TGFβ. This mechanism is an important factor influencing the efficacy of blinatumomab, a CD19-targeting CD3 bsAb for the treatment of hematological malignancies.

However, CD3 bsAbs are linked to potentially lethal AEs including cytokine release syndrome (CRS) and neurotoxicity. Certain bsAbs therapeutic candidates have thus aimed to decouple tumor cytotoxic activity from pro-inflammatory cytokine (e.g., IFNγ, TNFα, IL-2) release (see, for example Zuch de Zafra et al. (2019) Clin Cancer Res.; 25(13):3921-3933; Trinklein et al. (2019) MAbs.; 11(4):639-652; and Hernandez-Hoyos et al. (2016) Mol Cancer Ther.; 15(9):2155-65).

There is a pressing need for novel therapeutics having potent tumor cell-targeted killing activity, minimal off-target toxicity, an expanded therapeutic window for solid tumor and/or hematopoietic tumors, and a manageable AE profile.

### SUMMARY OF THE DISCLOSURE

The present disclosure generally provides bispecific proteins capable of inducing robust T cell cytotoxicity against tumor cells, and uses of the same for the treatment of cancers.

In one aspect, the present disclosure generally provides bispecific proteins capable of inducing and/or that induce robust T cell cytotoxicity against certain TAA expressing cells, and uses of the same for the treatment of cancers. In some embodiments, the present disclosure provides bispecific proteins capable of inducing and/or that induce robust T cell cytotoxicity against ROR1⁺ tumor cells, and uses of the same for the treatment of cancers.

In some embodiments, bispecific proteins of the present disclosure do not induce a significant amount of cytokine release. In some embodiments, bispecific proteins of the present disclosure induce robust T cell cytotoxicity against certain TAA expressing cells but do not induce a significant amount of cytokine release.

In some embodiments, a bispecific protein of the present disclosure, which induces robust T cell cytotoxicity against certain TAA expressing cells but does not induce a significant amount of cytokine release, includes two arms, the first arm comprises a first component, which specifically binds to a TAA, at its N-terminal end, and a second component, which specifically binds to the same TAA, at its C-terminal end, and a second arm comprises a component, which specifically binds to CD3, at its N-terminal end.

In some embodiments, a bispecific protein of the present disclosure, which induces robust T cell cytotoxicity against certain TAA expressing cells but does not induce a significant amount of cytokine release, comprises: (i) a first arm comprising, from N-terminus to C-terminus, a first component comprising a polypeptide or complex of two or more polypeptides that specifically binds a TAA, a first hinge polypeptide, a first polypeptide of a SEED format immunoglobulin Fc domain, and a second component comprising a polypeptide or complex of two or more polypeptides that specifically binds a TAA; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize.

In some embodiments, a protein of the present disclosure comprises: a polypeptide or complex of two or more polypeptides that specifically binds ROR1 connected to an end of a bridging moiety; and a polypeptide or complex of two or more polypeptides that specifically binds CD3 connected to an opposite end of the bridging moiety.

In some embodiments, the polypeptide or complex of two or more polypeptides that specifically binds ROR1 is connected to the C-terminal end of the bridging moiety and the polypeptide or complex of two or more polypeptides that specifically binds CD3 is connected to the N-terminal end of the bridging moiety.

In some embodiments, the protein further comprises a second polypeptide or second complex of two or more polypeptides that specifically binds ROR1 connected to the N-terminal end of the bridging moiety.

In some embodiments, the bridging moiety comprises two polypeptide arms, wherein the polypeptide or complex of two or more polypeptides that specifically binds ROR1 and the second polypeptide or second complex of two or more polypeptides that specifically binds ROR1 are connected to the C-terminal end and the N-terminal end of the same polypeptide arm, respectively, and wherein the polypeptide or complex of two or more polypeptides that specifically binds CD3 is connected to the N-terminal end of the second polypeptide arm.

In some embodiments, a bispecific protein of the present disclosure, which induces robust T cell cytotoxicity against certain TAA expressing tumor cells but does not induce a significant amount of cytokine release, comprises: (i) a first arm comprising, from N-terminus to C-terminus, a first component comprising a polypeptide or complex of two or more polypeptides that specifically binds ROR1, a first hinge polypeptide, a first polypeptide of a SEED format immunoglobulin Fc domain, and a second component comprising a polypeptide or complex of two or more polypeptides that specifically binds ROR1; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize.

In some embodiments, the polypeptide or complex of two or more polypeptides that specifically binds ROR1 is connected to the N-terminal end of the bridging moiety and the polypeptide or complex of two or more polypeptides that specifically binds CD3 is connected to the C-terminal end of the bridging moiety.

In some embodiments, the protein further comprises a second polypeptide or second complex of two or more polypeptides that specifically binds ROR1 connected to the N-terminal end of the bridging moiety.

In some embodiments, the bridging moiety comprises two polypeptide arms, wherein the polypeptide or complex of two or more polypeptides that specifically binds ROR1 and the polypeptide or complex of two or more polypeptides that specifically binds CD3 are connected to the N-terminal end and the C-terminal end of the sample polypeptide arm, respectively, and wherein the second polypeptide or second complex of two or more polypeptides that specifically binds ROR1 is connected to the N-terminal end of the second polypeptide arm.

In some embodiments, the polypeptide or complex of two or more polypeptides that specifically binds ROR1 is selected from the group consisting of: a single-chain variable fragment (scFv), a Fab, a Fab′, a F(ab′)2, a minibody, and a nanobody (VHH). In some embodiments, the polypeptide or complex of two or more polypeptides that specifically binds ROR1 is an scFv.

In some embodiments, the second polypeptide or second complex of two or more polypeptides that specifically binds ROR1 is selected from the group consisting of: an scFv, a Fab, a Fab′, a F(ab′)2, a minibody, and a VHH. In some embodiments, the second polypeptide or second complex of two or more polypeptides that specifically binds ROR1 is an scFv.

In some embodiments, the polypeptide or complex of two or more polypeptides that specifically binds ROR1, or the second polypeptide or second complex of two or more polypeptides that specifically binds ROR1 comprises: (i) a heavy chain complementarity-determining region 1 (VHCDR1) comprising an amino acid sequence of GFTFTSYA (SEQ ID NO: 8) or an amino acid sequence of GFSITTSGVS (SEQ ID NO: 23); (ii) a heavy chain complementarity-determining region 2 (VHCDR2) comprising an amino acid sequence of ISGSGGGT (SEQ ID NO: 9) or an amino acid sequence of IYWDDDK (SEQ ID NO: 24); (iii) a heavy chain complementarity-determining region 3 (VHCDR3) comprising an amino acid sequence of AQGSSIFDY (SEQ ID NO: 10) or an amino acid sequence of AHAPRYTPGGYFDY (SEQ ID NO: 25); (iv) a light chain complementarity-determining region 1 (VLCDR1) comprising an amino acid sequence of QSVSSY (SEQ ID NO: 11) or an amino acid sequence of QSVSSN (SEQ ID NO: 26); (v) a light chain complementarity-determining region 2 (VLCDR2) comprising an amino acid sequence of DAS or GAS; and (vi) a light chain complementarity-determining region 3 (VLCDR3) comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28).

In some embodiments, the polypeptide or complex of two or more polypeptides that specifically binds ROR1 or the second polypeptide or second complex of two or more polypeptides that specifically binds ROR1 comprises: a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14).

In some embodiments, the polypeptide or complex of two or more polypeptides that specifically binds ROR1 or the second polypeptide or second complex of two or more polypeptides that specifically binds ROR1 comprises: a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15).

In some embodiments, the polypeptide or complex of two or more polypeptides that specifically binds ROR1 or the second polypeptide or second complex of two or more polypeptides that specifically binds ROR1 comprises: a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28).

In some embodiments, the polypeptide or complex of two or more polypeptides that specifically binds ROR1 or the second polypeptide or second complex of two or more polypeptides that specifically binds ROR1 comprises, according to the IMGT unique numbering scheme, a VHCDR1, a VHCDR2, a VHCDR3, a VLCDR1, a VLCDR2, and a VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain as listed in the present disclosure.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 29 or SEQ ID NO: 39 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 40.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 29 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 30.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 29 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 31.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 39 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 40.

In some embodiments, the polypeptide or complex of two or more polypeptides that specifically binds ROR1 or the second polypeptide or second complex of two or more polypeptides that specifically binds ROR1 comprises, a heavy chain variable domain and a light chain variable domain each comprising an amino acid sequence corresponding to the heavy chain variable domain and light chain variable domain sequences of a heavy chain variable domain and light chain variable domain disclosed in the present application.

In some embodiments, the polypeptide or complex of two or more polypeptides that specifically binds ROR1 or the second polypeptide or second complex of two or more polypeptides that specifically binds ROR1 comprises a linker polypeptide comprising a (GGGGS)ₙ (SEQ ID NO: 1) sequence, wherein n is 1 to 12.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, or SEQ ID NO: 59.

In some embodiments, polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 42.

In some embodiments, the polypeptide or complex of two or more polypeptides that specifically binds ROR1 or the second polypeptide or second complex of two or more polypeptides that specifically binds ROR1 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 59.

In some embodiments, the polypeptide or complex of two or more polypeptides that specifically binds ROR1 or the second polypeptide or second complex of two or more polypeptides that specifically binds ROR1 comprises, an scFv comprising an amino acid sequence corresponding to an scFv listed in the present disclosure.

In some embodiments, the polypeptide or complex of two or more polypeptides that specifically binds CD3 is selected from the group consisting of: a Fab, a Fab′, a F(ab′)2, an scFv, a minibody, and a VHH. In some embodiments, the polypeptide or complex of two or more polypeptides that specifically binds CD3 is a Fab.

In some embodiments, the polypeptide or complex of two or more polypeptides that specifically binds CD3 comprises: (i) a heavy chain complementarity-determining region 1 (VHCDR1) comprising an amino acid sequence of GFTFNTYA (SEQ ID NO: 61); (ii) a heavy chain complementarity-determining region 2 (VHCDR2) comprising an amino acid sequence of IRSKYNNYAT (SEQ ID NO: 62); (iii) a heavy chain complementarity-determining region 3 (VHCDR3) comprising an amino acid sequence of VRHGNFGX₁X₂YVSWFAY (SEQ ID NO: 67), wherein X₁ is N, A, or E, and X₂ is S or A; (iv) a light chain complementarity-determining region 1 (VLCDR1) comprising an amino acid sequence of TGAVTTSN (SEQ ID NO: 64); (v) a light chain complementarity-determining region 2 (VLCDR2) comprising an amino acid sequence of GT; and (vi) a light chain complementarity-determining region 3 (VLCDR3) comprising an amino acid sequence of ALWYSNLWV (SEQ ID NO: 66).

In some embodiments, the polypeptide or complex of two or more polypeptides that specifically binds CD3 comprises: a VHCDR1 comprising an amino acid sequence of GFTFNTYA (SEQ ID NO: 61); a VHCDR2 comprising an amino acid sequence of IRSKYNNYAT (SEQ ID NO: 62); a VHCDR3 comprising an amino acid sequence of VRHGNFGNSYVSWFAY (SEQ ID NO: 63); a VLCDR1 comprising an amino acid sequence of TGAVTTSN (SEQ ID NO: 64); a VLCDR2 comprising an amino acid sequence of GT; and a VLCDR3 comprising an amino acid sequence of ALWYSNLWV (SEQ ID NO: 66).

In some embodiments, the polypeptide or complex of two or more polypeptides that specifically binds CD3 comprises: a VHCDR1 comprising an amino acid sequence of GFTFNTYA (SEQ ID NO: 61); a VHCDR2 comprising an amino acid sequence of IRSKYNNYAT (SEQ ID NO: 62); a VHCDR3 comprising an amino acid sequence of VRHGNFGASYVSWFAY (SEQ ID NO: 68); a VLCDR1 comprising an amino acid sequence of TGAVTTSN (SEQ ID NO: 64); a VLCDR2 comprising an amino acid sequence of GT; and a VLCDR3 comprising an amino acid sequence of ALWYSNLWV (SEQ ID NO: 66).

In some embodiments, the polypeptide or complex of two or more polypeptides that specifically binds CD3 comprises, according to the IMGT unique numbering scheme, a VHCDR1, a VHCDR2, a VHCDR3, a VLCDR1, a VLCDR2, and a VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in the present disclosure, respectively.

In some embodiments, the polypeptide or complex of two or more polypeptides that specifically binds CD3 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 71, SEQ ID NO: 72, SEQ ID NO: 73, SEQ ID NO: 74, SEQ ID NO: 75, or SEQ ID NO: 80 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 76, SEQ ID NO: 77, SEQ ID NO: 78, SEQ ID NO: 79, or SEQ ID NO: 81.

In some embodiments, the polypeptide or complex of two or more polypeptides that specifically binds CD3 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 72 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 81.

In some embodiments, the polypeptide or complex of two or more polypeptides that specifically binds CD3 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 71 and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 76.

In some embodiments, the polypeptide or complex of two or more polypeptides that specifically binds CD3 comprises, according to the IMGT unique numbering scheme, a heavy chain variable domain and a light chain variable domain each comprising an amino acid sequence corresponding to the heavy chain variable domain and light chain variable domain sequences of a heavy chain variable domain and light chain variable domain listed in the present disclosure, respectively.

In some embodiments, the polypeptide or complex of two or more polypeptides that specifically binds CD3 comprises a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 82 or SEQ ID NO: 83, and a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 84 or SEQ ID NO: 85.

In some embodiments, the polypeptide or complex of two or more polypeptides that specifically binds CD3 comprises, according to the IMGT unique numbering scheme, a heavy chain and a light chain each comprising an amino acid sequence corresponding to the heavy chain and light chain sequences of a heavy chain and light chain listed in the present disclosure, respectively.

In some embodiments, the bridging moiety is functional or non-functional.

In some embodiments, the bridging moiety comprises a strand-exchange engineered domain (SEED) format Fc domain or functional fragment thereof, an immunoglobulin Fc domain or functional fragment thereof, a polypeptide linker, or a polypeptide hinge.

In some embodiments, the SEED format Fc domain comprises one or more than one effector function silencing mutation.

In some embodiments, the SEED format Fc domain comprises a polypeptide comprising a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 86, SEQ ID NO: 117, or SEQ ID NO: 88, and a polypeptide comprising a sequence having at least 90% sequence identity (e.g., at least 91%, at least 92%, at least 93%, at least 94%, at least 95%, at least 96%, at least 97%, at least 98%, at least 99%, or 100%) to SEQ ID NO: 87, SEQ ID NO: 118, or SEQ ID NO: 89.

In some embodiments, the bridging moiety comprises an immunoglobulin Fc domain. In some embodiments, the immunoglobulin Fc domain comprises two polypeptide arms each comprising one or more than one mutation promoting heterodimerization.

In some embodiments, the immunoglobulin Fc domain comprises a first polypeptide arm and a second polypeptide arm, each comprising an amino acid sequence corresponding to the first polypeptide arm and second polypeptide arm sequences of a first polypeptide arm and second polypeptide arm listed in the present disclosure, respectively.

In some embodiments, the bridging moiety further comprises a hinge at its N-terminal end.

In some embodiments, the hinge comprises a polypeptide arm comprising an amino acid sequence comprising of SEQ ID NO: 92.

In some embodiments, the hinge further comprises a second polypeptide arm comprising an amino acid sequence comprising of SEQ ID NO: 90 or SEQ ID NO: 92.

In some embodiments, the hinge comprises a polypeptide arm comprising an amino acid sequence listed in the present disclosure. In some embodiments, the hinge further comprises a second polypeptide arm comprising an amino acid sequence listed in the present disclosure.

In some embodiments, the polypeptide or complex of two or more polypeptides that specifically binds ROR1 is connected to the C-terminal end of the bridging moiety by a linker polypeptide. In some embodiments, the polypeptide or complex of two or more polypeptides that specifically binds CD3 is connected to the C-terminal end of the bridging moiety by a linker polypeptide. In some embodiments, the linker polypeptide comprises a (GGGGS)ₙ (SEQ ID NO: 1) sequence, wherein n is 1 to 12.

In some embodiments, a protein of the present disclosure comprises: (i) a polypeptide or complex of two or more polypeptides that specifically binds ROR1 comprising a heavy chain complementarity-determining region 1 (VHCDR1) comprising an amino acid sequence of GFTFTSYA (SEQ ID NO: 8) or an amino acid sequence of GFSITTSGVS (SEQ ID NO: 23), a heavy chain complementarity-determining region 2 (VHCDR2) comprising an amino acid sequence of ISGSGGGT (SEQ ID NO: 9) or an amino acid sequence of IYWDDDK (SEQ ID NO: 24), a heavy chain complementarity-determining region 3 (VHCDR3) comprising an amino acid sequence of AQGSSIFDY (SEQ ID NO: 10) or an amino acid sequence of AHAPRYTPGGYFDY (SEQ ID NO: 25), a light chain complementarity-determining region 1 (VLCDR1) comprising an amino acid sequence of QSVSSY (SEQ ID NO: 11) or an amino acid sequence of QSVSSN (SEQ ID NO: 26), a light chain complementarity-determining region 2 (VLCDR2) comprising an amino acid sequence of DAS or GAS, and a light chain complementarity-determining region 3 (VLCDR3) comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28); (ii) a polypeptide or complex of two or more polypeptides that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGX₁X₂YVSWFAY (SEQ ID NO: 67), wherein X₁ is N, A, or E, and X₂ is S or A, a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); and (iii) a bridging moiety connecting the polypeptide or complex of two or more polypeptides that specifically binds ROR1 to the polypeptide or complex of two or more polypeptides that specifically binds CD3, wherein the bridging moiety comprises: (a) a polypeptide arm comprising amino acid sequences of SEQ ID NO: 90 and SEQ ID NO: 86 or SEQ ID NO: 92 and SEQ ID NO: 86, and (b) a second polypeptide arm comprising amino acid sequences of SEQ ID NO: 90 and SEQ ID NO: 87, or SEQ ID NO: 92 and SEQ ID NO: 87.

In some embodiments, a protein of the present disclosure comprises a second polypeptide or second complex of two or more polypeptides that specifically binds ROR1 connected to the N-terminal end of the bridging moiety. In some embodiments, the second polypeptide or second complex of two or more polypeptides that specifically binds ROR1 comprises a VHCDR1 comprising an amino acid sequence of GFTFTSYA (SEQ ID NO: 8) or an amino acid sequence of GFSITTSGVS (SEQ ID NO: 23), a VHCDR2 comprising an amino acid sequence of ISGSGGGT (SEQ ID NO: 9) or an amino acid sequence of IYWDDDK (SEQ ID NO: 24), a VHCDR3 comprising an amino acid sequence of AQGSSIFDY (SEQ ID NO: 10) or an amino acid sequence of AHAPRYTPGGYFDY (SEQ ID NO: 25), a VLCDR1 comprising an amino acid sequence of QSVSSY (SEQ ID NO: 11) or an amino acid sequence of QSVSSN (SEQ ID NO: 26), a VLCDR2 comprising an amino acid sequence of DAS or GAS, and a VLCDR3 comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28).

In some embodiments, a protein of the present disclosure comprises: (i) a polypeptide or complex of two or more polypeptides that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15); (ii) a polypeptide or complex of two or more polypeptides that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); (iii) a bridging moiety connecting the N-terminal end of the polypeptide or complex of two or more polypeptides that specifically binds ROR1 to the C-terminal end of the polypeptide or complex of two or more polypeptides that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and (iv) a second polypeptide or second complex of two or more polypeptides that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15).

In some embodiments, a protein of the present disclosure comprises: (i) a polypeptide or complex of two or more polypeptides that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14); (ii) a polypeptide or complex of two or more polypeptides that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); (iii) a bridging moiety connecting the N-terminal end of the polypeptide or complex of two or more polypeptides that specifically binds ROR1 to the C-terminal end of the polypeptide or complex of two or more polypeptides that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and (iv) a second polypeptide or second complex of two or more polypeptides that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14).

In some embodiments, a protein of the present disclosure comprises: (i) a polypeptide or complex of two or more polypeptides that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28); (ii) a polypeptide or complex of two or more polypeptides that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); (iii) a bridging moiety connecting the C-terminal end of the polypeptide or complex of two or more polypeptides that specifically binds ROR1 to the N-terminal end of the polypeptide or complex of two or more polypeptides that specifically binds CD3, wherein the bridging moiety comprises: (a) a polypeptide arm comprising amino acid sequences of SEQ ID NO: 90 and SEQ ID NO: 86 and (b) a second polypeptide arm comprising amino acid sequences of SEQ ID NO: 92 and SEQ ID NO: 87; and (iv) a second polypeptide or second complex of two or more polypeptides that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28).

In some embodiments, a protein of the present disclosure comprises: (i) a polypeptide or complex of two or more polypeptides that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28); (ii) a polypeptide or complex of two or more polypeptides that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); (iii) a bridging moiety connecting the C-terminal end of the polypeptide or complex of two or more polypeptides that specifically binds ROR1 to the N-terminal end of the polypeptide or complex of two or more polypeptides that specifically binds CD3, wherein the bridging moiety comprises: (a) a polypeptide arm comprising amino acid sequences of SEQ ID NO: 92 and SEQ ID NO: 86, and (b) a second polypeptide arm comprising amino acid sequences of SEQ ID NO: 92 and SEQ ID NO: 87; and (iv) a second polypeptide or second complex of two or more polypeptides that specifically binds ROR1 connected to the N-terminal end of the bridging moiety comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28).

In some embodiments, a protein of the present disclosure comprises: (i) a polypeptide or complex of two or more polypeptides that specifically binds ROR1 comprising a heavy chain variable domain (VH) comprising an amino acid sequence of SEQ ID NO: 29 or SEQ ID NO: 39, and a light chain variable domain (VL) comprising an amino acid sequence of SEQ ID NO: 30, SEQ ID NO: 31, SEQ ID NO: 32, SEQ ID NO: 33; SEQ ID NO: 34, SEQ ID NO: 35, SEQ ID NO: 36, SEQ ID NO: 37, SEQ ID NO: 38, or SEQ ID NO: 40; (ii) a polypeptide or complex of two or more polypeptides that specifically binds CD3 comprising a VH comprising an amino acid sequence of SEQ ID NO: 71, SEQ ID NO: 72, SEQ ID NO: 73, SEQ ID NO: 74, SEQ ID NO: 75, or SEQ ID NO: 80, and a VL comprising an amino acid sequence of SEQ ID NO: 76, SEQ ID NO: 77, SEQ ID NO: 78, SEQ ID NO: 79, or SEQ ID NO: 81; and (iii) a bridging moiety connecting the polypeptide or complex of two or more polypeptides that specifically binds ROR1 to the polypeptide or complex of two or more polypeptides that specifically binds CD3, wherein the bridging moiety comprises: (a) a polypeptide arm comprising amino acid sequences of SEQ ID NO: 90 and SEQ ID NO: 86, or SEQ ID NO: 92 and SEQ ID NO: 86, and (b) a second polypeptide arm comprising an amino acid sequence of SEQ ID NO: 90 and SEQ ID NO: 87, or SEQ ID NO: 92 and SEQ ID NO: 87.

In some embodiments, the second polypeptide or second complex of two or more polypeptides that specifically binds ROR1 comprising a heavy chain variable domain (VH) comprising an amino acid sequence of SEQ ID NO: 29 or SEQ ID NO: 39 and a light chain variable domain (VL) comprising an amino acid sequence of SEQ ID NO: 30, SEQ ID NO: 31, SEQ ID NO: 32, SEQ ID NO: 33; SEQ ID NO: 34, SEQ ID NO: 35, SEQ ID NO: 36, SEQ ID NO: 37, SEQ ID NO: 38, or SEQ ID NO: 40.

In some embodiments, a protein of the present invention comprises: (i) a polypeptide or complex of two or more polypeptides that specifically binds ROR1 comprising a VH comprising an amino acid sequence of SEQ ID NO: 29, and a VL comprising an amino acid sequence of SEQ ID NO: 31; (ii) a polypeptide or complex of two or more polypeptides that specifically binds CD3 comprising a VH comprising an amino acid sequence of SEQ ID NO: 71, and a VL comprising an amino acid sequence of SEQ ID NO: 76; (iii) a bridging moiety connecting the N-terminal end of the polypeptide or complex of two or more polypeptides that specifically binds ROR1 to the C-terminal end of the polypeptide or complex of two or more polypeptides that specifically binds CD3, wherein the bridging moiety comprises: (a) two polypeptides at its N-terminal end comprising an amino acid sequence of SEQ ID NO: 92, and (b) a polypeptide comprising an amino acid sequence of SEQ ID NO: 86 and a polypeptide comprising an amino acid sequence of SEQ ID NO: 87 wherein the bridging moiety comprises: (1) a polypeptide arm comprising amino acid sequences of SEQ ID NO: 90 and SEQ ID NO: 86, and (2) a second polypeptide arm comprising an amino acid sequence of SEQ ID NO: 92 and SEQ ID NO: 87; and (iv) a second polypeptide or second complex of two or more polypeptides that specifically binds ROR1 connected to the N-terminal end of the bridging moiety comprising a VH comprising an amino acid sequence of SEQ ID NO: 29, and a VL comprising an amino acid sequence of SEQ ID NO: 31.

In some embodiments, a protein of the present disclosure comprises: (i) a polypeptide or complex of two or more polypeptides that specifically binds ROR1 comprising a VH comprising an amino acid sequence of SEQ ID NO: 29, and a VL comprising an amino acid sequence of SEQ ID NO: 30; (ii) a polypeptide or complex of two or more polypeptides that specifically binds CD3 comprising a VH comprising an amino acid sequence of SEQ ID NO: 71, and a VL comprising an amino acid sequence of SEQ ID NO: 76; (iii) a bridging moiety connecting the N-terminal end of the polypeptide or complex of two or more polypeptides that specifically binds ROR1 to the C-terminal end of the polypeptide or complex of two or more polypeptides that specifically binds CD3, wherein the bridging moiety comprises: (a) a polypeptide arm comprising amino acid sequences of SEQ ID NO: 90 and SEQ ID NO: 86, and (b) a second polypeptide arm comprising an amino acid sequence of SEQ ID NO: 92 and SEQ ID NO: 87; and (iv) a second a polypeptide or complex of two or more polypeptides that specifically binds ROR1 connected to the N-terminal end of the bridging moiety comprising a VH comprising an amino acid sequence of SEQ ID NO: 29, and a VL comprising an amino acid sequence of SEQ ID NO: 30.

In some embodiments, a protein of the present disclosure comprises: (i) an scFv that specifically binds ROR1 comprising an amino acid sequence of SEQ ID NO: 42; (ii) a Fab that specifically binds CD3 comprising a heavy chain comprising an amino acid sequence of SEQ ID NO: 83, and a light chain comprising an amino acid sequence of SEQ ID NO: 85; (iii) a bridging moiety connecting the N-terminal end of the scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and a second scFv that specifically binds ROR1 connected to the N-terminal end of the bridging moiety comprising an amino acid sequence of SEQ ID NO: 42.

In some embodiments, a protein of the present disclosure comprises: (i) an scFv that specifically binds ROR1 comprising an amino acid sequence of SEQ ID NO: 41; (ii) a Fab that specifically binds CD3 comprising a heavy chain comprising an amino acid sequence of SEQ ID NO: 83, and a light chain comprising an amino acid sequence of SEQ ID NO: 85; (iii) a bridging moiety connecting the N-terminal end of the scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and (iv) a second scFv that specifically binds ROR1 connected to the N-terminal end of the bridging moiety comprising an amino acid sequence of SEQ ID NO: 41.

In some embodiments, a protein of the present disclosure comprises a first heavy chain, a second heavy chain, and a light chain, each comprising an amino acid sequence corresponding to a sequence of a first heavy chain, a sequence of a second heavy chain, and a sequence of a light chain listed in the present disclosure, respectively.

In some embodiments, a protein of the present disclosure has a K_{D} for CD3 binding of 10 nM to 50 nM, or 15 nM to 20 nM as measured in a biolayer interferometry assay.

In some embodiments, a protein of the present disclosure has an EC₅₀ of 20 nM to 50 nM, or 25 nM to 35 nM, in a CD3 binding assay using activated T cells.

In some embodiments, a protein of the present disclosure binds to the Ig-like domain of ROR1. In some embodiments, a protein of the present disclosure does not bind to the Frizzled and/or Kringle domain of ROR1.

In some embodiments, a protein of the present disclosure has a K_{D} for binding a TAA of 80 nM to 120 nM, 95 nM to 105 nM, 1 nM to 10 nM, or 3 nM to 5 nM, as measured in a biolayer interferometry assay. In some embodiments, a protein of the present disclosure has a K_{D} for ROR1 binding of 80 nM to 120 nM, 95 nM to 105 nM, 1 nM to 10 nM, or 3 nM to 5 nM, as measured in a biolayer interferometry assay.

In some embodiments, a protein of the present disclosure has an EC₅₀ of 80 nM to 120 nM, 90 nM to 110 nM, 1 nM to 10 nM, or 2 nM to 5 nM in a TAA binding assay using cells expressing about 3.5×10⁴ TAA molecules/cell. In some embodiments, a protein of the present disclosure has an EC₅₀ of 80 nM to 120 nM, 90 nM to 110 nM, 1 nM to 10 nM, or 2 nM to 5 nM in a ROR1 binding assay using cells expressing about 3.5×10⁴ ROR1 molecules/cell.

In some embodiments, a protein of the present disclosure is for use in the treatment of a solid tumor. In some embodiments, a protein of the present disclosure is for use in the treatment of a hematologic tumor.

In some embodiments, a protein of the present disclosure induces T cell lytic granule degranulation. In some embodiments, a protein of the present disclosure has an EC₅₀ of 160 pM to 200 pM, 165 pM to 190 pM, 25 pM to 50 pM, or 30 pM to 40 pM for T cell mediated cytotoxicity of TAA-expressing cells as measured in a T cell/tumor cell co-culture killing assay.

In some embodiments, a protein of the present disclosure has an EC₅₀ of 160 pM to 200 pM, 165 pM to 190 pM, 25 pM to 50 pM, or 30 pM to 40 pM for T cell mediated cytotoxicity of ROR1-expressing tumor cells as measured in a T cell/tumor cell co-culture killing assay.

In some embodiments, a protein of the present disclosure does not induce a significant increase in the production and/or release of pro-inflammatory cytokines by T cells when administered to cultures at concentrations sufficient to induce T cell mediated cytotoxicity.

In some embodiments, a protein of the present disclosure has an EC₅₀ of 1000 pM to 2000 pM, 1400 pM to 1800 pM, 200 pM to 600 pM, or 300 pM to 500 pM as measured in a T cell/tumor cell co-culture IFNγ production assay.

In some embodiments, a protein of the present disclosure has a Cₘₐₓ of 20% to 60%, 35% to 45%, or 45% to 55% compared to a reference T cell-binder as measured in a T cell/tumor cell co-culture IFNγ production assay.

In some embodiments, a protein of the present disclosure does not induce significant levels of TNFα or IL2 release when administered to freshly isolated peripheral mononuclear cell (PBMC) cultures or PBMCs pre-cultured for 48 hours at high density.

In some embodiments, a protein of the present disclosure has a cytokine release to killing decoupling ratio of 1:2 to 1:4, 1:2.2, or 1:2.9 normalized to a reference T cell-binder as measured in a T cell/tumor cell co-culture killing assay and IFNγ production assay.

In some embodiments, a protein of the present disclosure induces a tumor serial killing index of 3 to 5, 3.4 to 3.6, or 3.8 to 4.2 as measured in a T cell/tumor cell co-culture killing assay.

In some embodiments, a protein of the present disclosure is cross-reactive with cynomolgus CD3 but not mouse CD3. In some embodiments, a protein of the present disclosure is cross-reactive with a cynomolgus TAA and mouse TAA. In some embodiments, a protein of the present disclosure is cross-reactive with cynomolgus ROR1 and mouse ROR1.

The present disclosure also provides a formulation comprising a protein of the present invention and a pharmaceutically acceptable carrier.

The present disclosure also provides a nucleic acid encoding a protein of the present invention.

The present disclosure further provides a cell comprising one or more nucleic acids encoding a protein of the present invention.

The present disclosure provides a method of treating a cancer in a patient, comprising administering to the patient a protein or formulation of the present invention. In some embodiments, the cancer is a hematological cancer or a solid tumor cancer. In some embodiments, the cancer is selected from the group consisting of chronic lymphocytic leukemia, mantle cell lymphoma, non-Hodgkin lymphoma, ovarian cancer, lung cancer, bronchial cancer, colon cancer, rectal cancer, melanoma, renal cancer, gastric cancer, pancreatic cancer, prostate cancer, uterine cancer, breast cancer, oral cancer, pharyngeal cancer, hairy cell leukemia, liver cancer, intrahepatic bile duct cancer, and thyroid cancer.

In some embodiments of the present disclosure, an antibody or functional fragment thereof comprising, according to the IMGT unique numbering scheme, a heavy chain variable complementarity-determining region 1 (VHCDR1) comprising an amino acid sequence of GFTFTSYA (SEQ ID NO: 8), a heavy chain variable complementarity-determining region 2 (VHCDR2) comprising an amino acid sequence of ISGSGGGT (SEQ ID NO: 9), a heavy chain variable complementarity-determining region 3 (VHCDR3) comprising an amino acid sequence of AQGSSIFDY (SEQ ID NO: 10), a light chain variable complementarity-determining region 1 (VLCDR1) comprising an amino acid sequence of QSVSSY (SEQ ID NO: 11), a light chain variable complementarity-determining region 2 (VLCDR2) comprising an amino acid sequence of DAS, and a light chain variable complementarity-determining region 3 (VLCDR3) comprising an amino acid sequence of QQRSNWPPFT (SEQ ID NO: 14).

In some embodiments, the antibody comprises a heavy chain variable domain comprising an amino acid sequence at least 90% identical (e.g., at least 91%, at least 92%, at least 93%, at least 94%, at least 95%, at least 96%, at least 97%, at least 98%, at least 99%, or 100%) to SEQ ID NO: 29, and a light chain variable domain comprising an amino acid sequence at least 90% identical (e.g., at least 91%, at least 92%, at least 93%, at least 94%, at least 95%, at least 96%, at least 97%, at least 98%, at least 99%, or 100%) to SEQ ID NO: 30.

In some embodiments, an antibody of the present disclosure comprises, according to the IMGT unique numbering scheme, a heavy chain variable complementarity-determining region 1 (VHCDR1) comprising an amino acid sequence of GFTFTSYA (SEQ ID NO: 8), a heavy chain variable complementarity-determining region 2 (VHCDR2) comprising an amino acid sequence of ISGSGGGT (SEQ ID NO: 9), a heavy chain variable complementarity-determining region 3 (VHCDR3) comprising an amino acid sequence of AQGSSIFDY (SEQ ID NO: 10), a light chain variable complementarity-determining region 1 (VLCDR1) comprising an amino acid sequence of QSVSSY (SEQ ID NO: 11), a light chain variable complementarity-determining region 2 (VLCDR2) comprising an amino acid sequence of DAS, and a light chain variable complementarity-determining region 3 (VLCDR3) comprising an amino acid sequence of QQRSNWPPST (SEQ ID NO: 15). In some embodiments, the antibody comprises a heavy chain variable domain comprising an amino acid sequence at least 90% identical (e.g., at least 91%, at least 92%, at least 93%, at least 94%, at least 95%, at least 96%, at least 97%, at least 98%, at least 99%, or 100%) to SEQ ID NO: 39, and a light chain variable domain comprising an amino acid sequence at least 90% identical (e.g., at least 91%, at least 92%, at least 93%, at least 94%, at least 95%, at least 96%, at least 97%, at least 98%, at least 99%, or 100%) to SEQ ID NO: 40. In some embodiments, the antibody is a human IgG1 antibody

Other embodiments and details of the disclosure are presented herein below.

### BRIEF DESCRIPTION OF THE DRAWINGS

FIGS. 1A and 1B provide schematic illustrations of protein designs of bispecific T cell-engagers (TCEs) comprising a bridging moiety, a first scFv that specifically binds a tumor-associated antigen (“TAA”) (e.g., ROR1), a Fab that specifically binds CD3, and a second scFv that specifically binds ROR1. FIG. 1A is a schematic illustration of an exemplary bispecific TCE in “Format 1A” where the first scFv that specifically binds a TAA (e.g., ROR1) is connected to the C-terminal end of the bridging moiety, the Fab that specifically binds CD3 is connected to the N-terminal end of the bridging moiety, and the second scFv that specifically binds a TAA (e.g., ROR1) is connected to the N-terminal end of the bridging moiety. FIG. 1B is a schematic illustration of an exemplary bispecific TCE in “Format 1B” where the first scFv that specifically binds a TAA (e.g., ROR1) is connected to the N-terminal end of the bridging moiety, the Fab that specifically binds CD3 is connected to the C-terminal end of the bridging moiety, and the second scFv that specifically binds a TAA (e.g., ROR1) is connected to the N-terminal end of the bridging moiety.

FIGS. 2A to 2D are graphs showing the apparent binding affinities of the CD3-binding polypeptide arms of 69A02 and/or 82H02 bispecific TCEs in Format 1A and Format 1B, human IgG1 isotype control (hIgG2), a reference T cell-binder (Ref. T cell-binder), or a reference ROR1 Frizzled domain-binding TCE (Ref. Frz-binding TCE) in Format 1A or Format 1B, for human or cynomolgus CD3 on Jurkat T cells (FIG. 2A), cynomolgus pan-T cells (FIG. 2B), T cells isolated from human CD3 knock-in C57BL6 mice (FIG. 2C), or human CD3 knockout Jurkat T cells (FIG. 2D).

FIGS. 3A to 3D are graphs showing the apparent binding affinities of the ROR1-binding polypeptide arms of 69A02 and/or 82H02 bispecific TCEs in Format 1A and Format 1B, human IgG1 isotype control (Isotype Control), and a reference T cell-binder (Ref. T cell-binder), or a reference ROR1 Frizzled domain-binding TCE (Ref. Frz-binding TCE) in Format 1A or Format 1B for human ROR1 on hROR1⁺ MDA-MB-231 cells (FIG. 3A), hROR1⁻ T47D cells (FIG. 3B), 4T1 cells engineered to overexpress human ROR1 (FIG. 3C), or 4T1 cells engineered to overexpress murine ROR1 (FIG. 3D).

FIGS. 4A and 4B are graphs showing titrations of induction of cytotoxic activity by 69A02 and/or 82H02 bispecific TCEs in Format 1A or Format 1B, a reference T cell-binder (Ref. T cell-binder), or a reference ROR1 Frizzled domain-binding TCE in Format 1A (Ref. Frz-binding TCE 1A) on freshly isolated peripheral blood mononuclear cells (PBMC) co-cultured for 24 hours with hROR1⁺ MDA-MB-231 tumor cells (FIG. 4A), or hROR1⁻ T47D tumor cells (FIG. 4B). FIGS. 4C and 4D are graphs showing titrations of induction of IFNγ release by 69A02 and/or 82H02 bispecific TCEs in Format 1A or Format 1B, a reference T cell-binder (Ref. T cell-binder), or a reference ROR1 Frizzled domain-binding TCE in Format 1A (Ref. Frz-binding TCE 1A) on freshly isolated peripheral blood mononuclear cells (PBMC) co-cultured for 24 hours with hROR1⁺ MDA-MB-231 tumor cells (FIG. 4C), or hROR1⁻ T47D tumor cells (FIG. 4D). FIG. 4E is a bar graph showing the magnitude by which induction of cytotoxic activity is decoupled from induction of proinflammatory cytokine release by 69A02 or 82H02 bispecific TCEs in Format 1A or Format 1B, or a reference ROR1 Frizzled domain-binding TCE in Format 1A (Ref. Frz-binding TCE 1A) normalized to values achieved by a reference T cell-binder (Ref. T cell-binder).

FIG. 5A is a graph showing titrations of induction of cytotoxic activity by 69A02 or 82H02 bispecific TCEs in Format 1A or Format 1B, a reference T cell-binder (Ref. T cell-binder), or a reference ROR1 Frizzled domain-binding TCE in Format 1A (Ref. Frz-binding TCE 1A), on CD8⁺ T cells co-cultured for 24 hours with hROR1⁺ MDA-MB-231 tumor cells. FIG. 5B is a graph showing titrations of induction of IFNγ production by 69A02 or 82H02 bispecific TCEs in Format 1A or Format 1B, a reference T cell-binder (Ref. T cell-binder), or a reference ROR1 Frizzled domain-binding TCE in Format 1A (Ref. Frz-binding TCE 1A), on CD8⁺ T cells co-cultured for 24 hours with hROR1⁺ MDA-MB-231 tumor cells. FIG. 5C and FIG. 5D are graphs showing titrations of induction of IFNγ production by 69A02 bispecific TCEs in Format 1A or Format 1B, or a reference ROR1 Frizzled domain-binding TCE (Ref. Frz-binding TCE) in Format 1A or Format 1B, on CD8⁺ T cells co-cultured for 24 hours with MCF-7 tumor cells and T47D cells, respectively.

FIG. 6A is a bar graph showing the serial killing frequency of hROR1⁺ NCI-H1975 tumor cells by tumor infiltrating lymphocytes (TILs) expanded from triple negative breast cancer (TNBC) biopsies, in the presence of 69A02 or 82H02 bispecific TCEs in Format 1A or Format 1B, a reference T cell-binder (Ref. T cell-binder), or a reference ROR1 Frizzled domain-binding TCE (Ref. Frz-binding TCE) in Format 1A or Format 1B. FIG. 6B is a bar graph showing the serial killing frequency of hROR1⁻ T47D tumor cells by TILs expanded from TNBC biopsies, in the presence of 69A02 or 82H02 bispecific TCEs in Format 1A or Format 1B, a reference T cell-binder (Ref. T cell-binder), or a reference ROR1 Frizzled domain-binding TCE (Ref. Frz-binding TCE) in Format 1A or Format 1B.

FIG. 7A is a graph showing titrations of induction of cytotoxic activity by the 82H02 bispecific TCE in Format 1A (82H02 TCE 1A), or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1⁻ MCF-7 tumor cells. FIG. 7B is a graph showing titrations of induction of IFNγ release by the 82H02 bispecific TCE in Format 1A (82H02 TCE 1A), or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1⁻ MCF-7 tumor cells. FIG. 7C is a graph showing titrations of induction of cytotoxic activity by the 82H02 bispecific TCE in Format 1B (82H02 TCE 1B), or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1-MCF-7 tumor cells. FIG. 7D is a graph showing titrations of induction of IFNγ release by the 82H02 bispecific TCE in Format 1B (82H02 TCE 1B), or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1-MCF-7 tumor cells. FIG. 7E is a graph showing titrations of induction of cytotoxic activity by the reference ROR1 Frizzled domain-binding TCE (Ref. Frz-binding TCE) in Format 1A, or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1⁻ MCF-7 tumor cells. FIG. 7F is a graph showing titrations of induction of IFNγ release by the reference ROR1 Frizzled domain-binding TCE (Ref. Frz-binding TCE) in Format 1A, or a reference T cell-binder (Ref. T cell-binder), on freshly isolated peripheral blood mononuclear cells (PBMC) co-cultured for 24 hours with hROR1⁺ Du-145, A549, NCI-H1975, MDA-MB-231, NCCIT tumor cells, or hROR1⁻ MCF-7 tumor cells.

FIGS. 8A and 8B are graphs showing titrations of induction of cytotoxic activity by the 82H02 bispecific TCE in Format 1A or Format 1B, a reference T cell-binder (Ref. T cell-binder), or a reference ROR1 Frizzled domain-binding TCE (Ref. Frz-binding TCE) in Format 1A or Format 1B, on human CD8⁺ T cells (FIG. 8A) or cynomolgus CD8⁺ T cells (FIG. 8B) co-cultured for 24 hours with hROR1⁺ MDA-MB-231 tumor cells. FIG. 8C and FIG. 8D are graphs showing titrations of induction of IFNγ release by the 82H02 bispecific TCE in Format 1A or Format 1B, a reference T cell-binder (Ref. T cell-binder), or a reference ROR1 Frizzled domain-binding TCE (Ref. Frz-binding TCE) in Format 1A or Format 1B, on human CD8⁺ T cells (FIG. 8C) or cynomolgus CD8⁺ T cells (FIG. 8D) co-cultured for 24 hours with hROR1⁺ MDA-MB-231 tumor cells.

FIGS. 9A and 9C are graphs showing titrations of induction of cytotoxic activity by the 82H02 bispecific TCE in Format 1A or Format 1B, a reference T cell-binder (Ref. T cell-binder), or a reference ROR1 Frizzled domain-binding TCE (Ref. Frz-binding TCE) in Format 1A or Format 1B, on freshly isolated splenocytes from human CD3ε knock-in mice co-cultured for 24 hours with human ROR1⁺ 4T1 tumor cells (FIG. 9A) or murine ROR1⁺ 4T1 tumor cells (FIG. 9C). FIGS. 9B and 9D are graphs showing titrations of induction of IFNγ release by the 82H02 bispecific TCE in Format 1A or Format 1B, a reference T cell-binder (Ref. T cell-binder), or a reference ROR1 Frizzled domain-binding TCE (Ref. Frz-binding TCE) in Format 1A or Format 1B, on freshly isolated splenocytes from human CD3ε knock-in mice co-cultured for 24 hours with human ROR1⁺ 4T1 tumor cells (FIG. 9B) or murine ROR1⁺ 4T1 tumor cells (FIG. 9D). FIGS. 9E and 9F are bar graphs showing the magnitudes by which induction of cytotoxic activity against hROR-1⁺ 4T1 tumor cells (FIG. 9E) or mROR1⁺ 4T1 tumor cells (FIG. 9F) are decoupled from induction of proinflammatory cytokine release by the 82H02 bispecific TCE in Format 1A or Format 1B, or a reference ROR1 Frizzled domain-binding TCE (Ref. Frz-binding TCE) in Format 1A or Format 1B, normalized to values achieved by a reference T cell-binder (Ref. T cell-binder).

FIGS. 10A to 10D are graphs showing titrations of induction of IFNγ (FIG. 10A), TNFα (FIG. 10B), IL-10 (FIG. 10C), or IL-2 (FIG. 10D) release by the 82H02 bispecific TCE in Format 1A or Format 1B an OKT3 α-CD3 positive control (OKT3), a hIgG1 isotype control (hIgG1), a reference T cell-binder (Ref. T cell-binder), or a reference ROR1 Frizzled domain-binding TCE (Ref. Frz-binding TCE) in Format 1A or Format 1B, on freshly isolated PBMCs from healthy human donors cultured at a cell density of 1.0×10⁷ cells/ml.

FIGS. 11A to 11D are graphs showing titrations of induction of IFNγ (FIG. 11A), TNFα (FIG. 11B), IL-10 (FIG. 11C), or IL-2 (FIG. 11D) release by the 82H02 bispecific TCE in Format 1A or Format 1B, an OKT3 α-CD3 positive control (OKT3), a hIgG1 isotype control (hIgG1), a reference T cell-binder (Ref. T cell-binder), or a reference ROR1 Frizzled domain-binding TCE (Ref. Frz-binding TCE) in Format 1A or Format 1B, on PBMCs from healthy human donors pre-cultured for 48 hours at a cell density of 1×10⁷ cells/ml.

FIGS. 12A and 12B are graphs showing the apparent binding affinities of the CD3-binding polypeptide arms of four sequence optimized 82H02 bispecific TCEs in Format 1A (82H02 TCE #1, 82H02 TCE #2, 82H02 TCE #3, 82H02 TCE #4), human IgG1 isotype control (hIgG1), or a reference T cell-binder (Ref. T cell-binder), for human CD8⁺ cells (FIG. 12A) or cynomolgus Pan-T cells (FIG. 12B).

FIGS. 13A to 13D are graphs showing the apparent binding affinities of the ROR1-binding polypeptide arms of sequence optimized 82H02 bispecific TCEs in Format 1A (FIG. 13A; 82H02 #1, 82H02 #2, FIG. 13B; 82H02 #3, 82H02 #4), human IgG1 isotype control (hIgG1), or a reference T cell-binder (Ref. T cell-binder), for MDA-MB-231 tumor cells (FIG. 13A and FIG. 13B) or mROR1⁺ 4T1 tumor cells (FIG. 13C and FIG. 13D).

FIG. 14A is a graph showing titrations of induction of cytotoxic activity by four sequence optimized 82H02 bispecific TCEs in Format 1A (82H02 TCE #1, 82H02 TCE #2, 82H02 TCE #3, 82H02 TCE #4), or a reference T cell-binder (Ref. T cell-binder), on freshly isolated peripheral blood mononuclear cells (PBMC) co-cultured for 24 hours with hROR1⁺ MDA-MB-231 tumor cells. FIG. 14B is a graph showing titrations of induction of IFNγ release by four sequence optimized 82H02 bispecific TCEs in Format 1A (82H02 TCE #1, 82H02 TCE #2, 82H02 TCE #3, 82H02 TCE #4), or a reference T cell-binder (Ref. T cell-binder), on freshly isolated peripheral blood mononuclear cells (PBMC) co-cultured for 24 hours with hROR1⁺ MDA-MB-231 tumor cells. FIG. 14C is a bar graph showing the magnitude by which induction of cytotoxic activity is decoupled from induction of proinflammatory cytokine release by four sequence optimized 82H02 bispecific TCEs in Format 1A (82H02 TCE #1, 82H02 TCE #2, 82H02 TCE #3, 82H02 TCE #4), normalized to values achieved by a reference T cell-binder (Ref. T cell-binder).

FIG. 15 is a bar graph showing the serial killing frequency of hROR1⁺ NCI-H1975 tumor cells by tumor infiltrating lymphocytes (TILs) expanded from triple negative breast cancer (TNBC) biopsies in the presence of four sequence optimized 82H02 bispecific TCEs in Format 1A (82H02 TCE #1, 82H02 TCE #2, 82H02 TCE #3, 82H02 TCE #4), or a reference T cell-binder (Ref. T cell-binder).

FIGS. 16A to 16F are graphs showing titrations of induction of cytotoxic activity by four sequence optimized 82H02 bispecific TCEs in Format 1A (82H02 TCE #1, 82H02 TCE #2, 82H02 TCE #3, 82H02 TCE #4), or a reference T cell-binder (Ref. T cell-binder), on freshly isolated peripheral blood mononuclear cells (PBMC) co-cultured for 24 hours with MDA-MB-231 tumor cells having cell surface densities of hROR1 expression of approximately 7×10⁶ (FIG. 16A), 1.3×10⁶ (FIG. 16B), 6×10⁵ (FIG. 16C), 4×10⁵ (FIG. 16D), 2×10⁵ (FIG. 16E) molecules/cell, or hROR1 knockout cells (FIG. 16F).

FIGS. 17A to 17F are graphs showing titrations of induction of IFNγ release by four sequence optimized 82H02 bispecific TCEs in Format 1A (82H02 TCE #1, 82H02 TCE #2, 82H02 TCE #3, 82H02 TCE #4), or a reference T cell-binder (Ref. T cell-binder), on freshly isolated peripheral blood mononuclear cells (PBMC) co-cultured for 24 hours with MDA-MB-231 tumor cells having cell surface densities of hROR1 expression of approximately 7×10⁶ (FIG. 17A), 1.3×10⁶ (FIG. 17B), 6×10⁵ (FIG. 17C), 4×10⁵ (FIG. 17D), 2×10⁵ (FIG. 17E) molecules/cell, or hROR1 knockout cells (FIG. 17F).

FIG. 18 is bar graph showing the magnitudes by which induction of cytotoxic activity against MDA-MB-231 tumor cells having cell surface densities of hROR1 expression of approximately 2×10⁵, 4×10⁵, 6×10⁵, 1.3×10⁶, or 7×10⁶ molecules/cell, are decoupled from induction of proinflammatory cytokine release by four sequence optimized 82H02 bispecific TCEs in Format 1A (82H02 TCE #1, 82H02 TCE #2, 82H02 TCE #3, 82H02 TCE #4), normalized to values achieved by a reference T cell-binder (Ref. T cell-binder).

FIG. 19 is a graph showing mean fluorescence intensities (MFI) of NCCIT cell cultures pre-treated with ROR1-binding clones of the present disclosure in scFv-Fc format (82H02 scFv-Fc and 69A02 scFv-Fc), a reference T cell-binder in scFv format (Ref. T cell-binder scFv-Fc), or a reference Frizzled-binding clone in scFv-Fc format (Ref. Frz-binding scFv-Fc) then incubated with 82H02 scFv-Fc labeled with Alexa Fluor 647-conjugated Zenon fragments in a competitive binding assay.

### DETAILED DESCRIPTION

The present application provides bispecific proteins that bind a TAA (e.g., ROR1) on the surface of tumor cells and CD3 on the surface of T cells, and are capable of inducing potent T cell cytotoxic activity against ROR1⁺ tumor cells. This anti-tumor cytotoxicity inducing activity is decoupled from its ability to induce proinflammatory cytokine production. In some embodiments, the application also provides therapeutic methods for using such proteins for the treatment of cancers. Various aspects of the bispecific proteins described in the present application are set forth below in sections; however, aspects of the proteins described in one particular section are not to be limited to any particular section. Also provided in the present application are antibodies that specifically bind the Ig-like domain of ROR1.

### Definitions

To facilitate an understanding of the present application, a number of terms and phrases are defined below.

The terms “a” and “an” as used herein mean “one or more” and include the plural unless the context is inappropriate.

As used herein, the term “antigen-binding site” refers to the part of the immunoglobulin (Ig) molecule that participates in antigen binding. In human antibodies, the antigen-binding site is formed by amino acid residues of the N-terminal variable (“V”) domains of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus, the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of an antigen to which the antigen-binding site specifically binds, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface (for example. Fab, Fab′, F(ab′)₂, or in a recombinant polypeptide such as an scFv, using a peptide linker to connect the heavy chain variable domain to the light chain variable domain in a single polypeptide, a minibody, or a nanobody (VHH).

As used herein, the term “functional fragment thereof” refers to a portion of a protein or polypeptide that maintains the ability to perform a biological function of the whole protein or polypeptide. For example, a functional fragment of a polypeptide or protein of the present application maintains its ability to bind its cognate binding partner or ligand.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a bispecific protein or antibody of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, “significantly” or “significant” refers to a change or alteration in a measurable parameter to a statistically significant degree as determined in accordance with an appropriate statistically relevant test. For example, in some embodiments, a change or alteration is significant if it is statistically significant in accordance with, e.g., a Student's t-test, chi-square, or Mann Whitney test.

As used herein, ROR1 or ROR-1 refers to the protein of SEQ ID NO: 268 and related isoforms and orthologs.

ROR1 Amino Acid Sequence:
(SEQ ID NO: 268)
MHRPRRRGTRPPLLALLAALLLAARGAAAQETELSVSAELVPTSSWN
ISSELNKDSYLTLDEPMNNITTSLGQTAELHCKVSGNPPPTIRWFKND
APVVQEPRRLSFRSTIYGSRLRIRNLDTTDTGYFQCVATNGKEVVSST
GVLFVKFGPPPTASPGYSDEYEEDGFCQPYRGIACARFIGNRTVYMES
LHMQGEIENQITAAFTMIGTSSHLSDKCSQFAIPSLCHYAFPYCDETSS
VPKPRDLCRDECEILENVLCQTEYIFARSNPMILMRLKLPNCEDLPQPE
SPEAANCIRIGIPMADPINKNHKCYNSTGVDYRGTVSVTKSGRQCQP
WNSQYPHTHTFTALRFPELNGGHSYCRNPGNQKEAPWCFTLDENFKS
DLCDIPACDSKDSKEKNKMEILYILVPSVAIPLAIALLFFFICVCRNNQK
SSSAPVQRQPKHVRGQNVEMSMLNAYKPKSKAKELPLSAVRFMEEL
GECAFGKIYKGHLYLPGMDHAQLVAIKTLKDYNNPQQWTEFQQEAS
LMAELHHPNIVCLLGAVTQEQPVCMLFEYINQGDLHEFLIMRSPHSD
VGCSSDEDGTVKSSLDHGDFLHIAIQIAAGMEYLSSHFFVHKDLAARN
ILIGEQLHVKISDLGLSREIYSADYYRVQSKSLLPIRWMPPEAIMYGKF
SSDSDIWSFGVVLWEIFSFGLQPYYGFSNQEVIEMVRKRQLLPCSEDC
PPRMYSLMTECWNEIPSRRPRFKDIHVRLRSWEGLSSHTSSTTPSGGN
ATTQTTSLSASPVSNLSNPRYPNYMFPSQGITPQGQIAGFIGPPIPQNQR
FIPINGYPIPPGYAAFPAAHYQPTGPPRVIQHCPPPKSRSPSSASGSTST
GHVTSLPSSGSNQEANIPLLPHMSIPNHPGGMGITVFGNKSQKPYKIDSK
QASLLGDANIHGHTESMISAEL

As used herein, CD3 refers to the protein comprising the CM3ε (SEQ ID NO: 269), CD3γ (SEQ ID NO: 270), CD38 (SEQ ID NO: 271), and CD3ξ (SEQ ID NO: 96) polypeptides, which together with the T-cell receptor α/β and γ/δ heterodimers form the T-cell receptor-CD3 complex.

CD3ϵ Sequence:
(SEQ ID NO: 269)
MQSGTHWRVLGLCLLSVGVWGQDGNEEMGGITQTPYKVSISGTTVIL
TCPQYPGSEILWQHNDKNIGGDEDDKNIGSDEDHLSLKEFSELEQSGY
YVCYPRGSKPEDANFYLYLRARVCENCMEMDVMSVATIVIVDICITG
GLLLLVYYWSKNRKAKAKPVTRGAGAGGRQRGQNKERPPPVPNPD
YEPIRKGQRDLYSGLNQRRI
CD3γ Sequence:
(SEQ ID NO: 270)
MEQGKGLAVLILAIILLQGTLAQSIKGNHLVKVYDYQEDGSVLLTCD
AEAKNITWFKDGKMIGFLTEDKKKWNLGSNAKDPRGMYQCKGSQN
KSKPLQVYYRMCQNCIELNAATISGFLFAEIVSIFVLAVGVYFIAGQD
GVRQSRASDKQTLLPNDQLYQPLKDREDDQYSHLQGNQLRRN
CD3δ Sequence:
(SEQ ID NO: 271)
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCV
ELDPATVAGIIVTDVIATLLLALGVFCFAGHETGRLSGAADTQALLRN
DQVYQPLRDRDDAQYSHLGGNWARNK
CD3ξ Sequence:
(SEQ ID NO: 96)
MKWKALFTAAILQAQLPITEAQSFGLLDPKLCYLLDGILFIYGVILTAL
FLRVKFSRSADAPAYQQGQNQLYNELNLGRREEYDVLDKRRGRDPE
MGGKPQRRKNPQEGLYNELQKDKMAEAYSEIGMKGERRRGKGHDG
LYQGLSTATKDTYDALHMQALPPR

For sequences presented herein with signal peptides, it is understood that mature versions of the proteins are expressed without the signal peptide.

Throughout the description, where a protein is described as having, including, or comprising specific components, or where processes and methods are described as having, including, or comprising specific steps, it is contemplated that, additionally, there are proteins described in the present application that consist essentially of or consist of the recited components, and that there are processes and methods according to the present application that consist essentially of or consist of the recited processing steps.

### I. Proteins

The present application provides bispecific proteins that specifically bind to a TAA.

In some embodiments, bispecific proteins of the present disclosure induce robust T cell cytotoxicity against certain TAA expressing cells but do not induce a significant amount of cytokine release.

In some embodiments, a bispecific protein of the present disclosure, which induces robust T cell cytotoxicity against certain TAA expressing cells but does not induce a significant amount of cytokine release, includes two arms, the first arm comprises a first component, which specifically binds to a TAA, at its N-terminal end, and a second component, which specifically binds to the same TAA, at its C-terminal end, and a second arm comprises a component, which specifically binds to CD3, at its N-terminal end.

For example, in some embodiments, bispecific proteins of the present application specifically bind to the Ig-like domain of ROR1 expressed on cancer cells in a bivalent manner. As compared to high affinity and/or monovalent antibodies, proteins of the present application have a greater capacity to distinguish between target tumor cells and off-target non-tumor cells based on ROR1 surface density. Furthermore, by bridging ROR1 on the surface of target tumor cells and CD3 on the surface of T cells, proteins of the present application activate the cytotoxic activity of CD8⁺ T cells and induces T cell serial killing of the ROR1-expressing tumor cells. This cytotoxic activity is decoupled from induction of T cell cytokine release.

The bispecific proteins of the present disclosure include two arms, each arm including at least one component (e.g., a polypeptide or complex of two or more polypeptides), wherein a first arm comprises a first component which specifically binds to ROR1 at its N-terminal or C-terminal end. In some embodiments, a second arm comprises a component which specifically binds to CD3 at its N-terminal or C-terminal end, and the first arm further comprises a second component which specifically binds to ROR1 at an end that is opposite to the first component. In other embodiments, a second arm comprises a component which specifically binds to CD3 at its N-terminal or C-terminal end, and further comprises a second component which specifically binds to ROR1 at an end of the second arm that is opposite to the component which specifically binds CD3. In some embodiments, the first and second components that specifically bind to ROR1 can comprise a single polypeptide. In some embodiments, the component that specifically binds to CD3 can comprise a complex of two or more polypeptides. In some embodiments, the complex of two or more polypeptides of the component that specifically binds to CD3 may be connected by one or more than one covalent linkage (e.g., a disulfide bond) and/or one or more than one non-covalent interaction (e.g., an ionic or hydrophobic interaction). Further description of exemplary bispecific proteins is provided below.

A bispecific protein of the present disclosure includes a first component and second component that are polypeptides or complexes of two or more polypeptides that specifically bind ROR1. In some embodiments, the first component and second component can be, but are not limited to, one or more antigen-binding sites, for example, single-chain variable fragments (scFvs), antibodies, Fab, Fab′, F(ab′)₂, minibodies, or nanobodies (VHHs).

A bispecific protein of the present disclosure includes a component that is a polypeptide or complex of two or more polypeptides that specifically binds CD3. In some embodiments, the component can be, but is not limited to, one or more antigen-binding site, for example, a Fab, an antibody, a Fab′, a F(ab′)₂, a single-chain variable fragment (scFv), a minibody, or a nanobody (VHH).

In some embodiments, a bispecific protein of the present disclosure additionally comprises a bridging moiety, which can include, but is not limited to: a strand-exchange engineered domain (SEED) format immunoglobulin Fc domain or functional fragment thereof, an immunoglobulin Fc domain or functional fragment thereof, polypeptide linkers, or polypeptide hinges. In some embodiments, the bridging moiety comprises one or more than one mutation that promotes heterodimerization of the two arms of the protein. In some embodiments, the bridging moiety is a SEED format immunoglobulin Fc domain comprising a first and second polypeptide. In some embodiments, a bispecific protein of the present disclosure further comprises a hinge polypeptide immediately N-terminal to each of the first and second polypeptide of the SEED format immunoglobulin Fc domain.

In some embodiments, a bispecific protein of the present disclosure further comprises a linker polypeptide, which connects the first component or second component that specifically binds ROR1, or the component that specifically binds CD3 to the N-terminal or C-terminal end of the bridging moiety.

In some embodiments, a bispecific protein of the present disclosure, which induces robust T cell cytotoxicity against certain TAA expressing cells but does not induce a significant amount of cytokine release, comprises: (i) a first arm comprising, from N-terminus to C-terminus, a first component comprising a polypeptide or complex of two or more polypeptides that specifically binds a TAA, a first hinge polypeptide, a first polypeptide of a SEED format immunoglobulin Fc domain, and a second component comprising a polypeptide or complex of two or more polypeptides that specifically binds a TAA; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize.

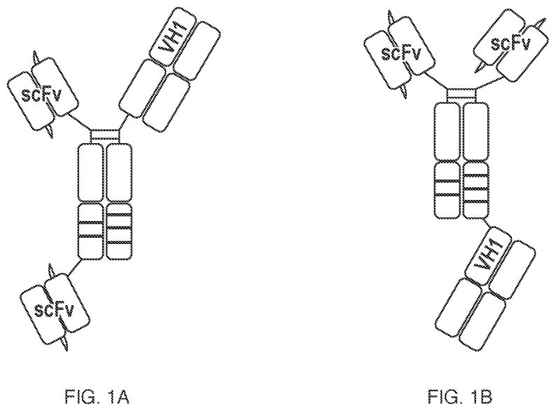

The bispecific proteins described herein can take various formats. For example, one bispecific protein format comprises: (i) a first arm comprising, from N-terminus to C-terminus, a first component comprising a polypeptide or complex of two or more polypeptides that specifically binds ROR1, a first hinge polypeptide, a first polypeptide of a SEED format immunoglobulin Fc domain, and a second component comprising a polypeptide or complex of two or more polypeptides that specifically binds ROR1; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize. In this format, the first and second components that specifically bind ROR1 are antigen-binding sites (e.g., scFvs, antibodies, Fab, Fab′, F(ab′)₂, minibodies, or VHHs) and the component that specifically binds CD3 is an antigen-binding site (e.g., Fab, scFv, antibody, Fab′, F(ab′)₂, minibody, or VHH). In some embodiments, the first and second components that specifically bind ROR1 are scFvs and the component that specifically binds CD3 is a Fab (FIG. 1A). In some embodiments, the component that specifically binds CD3 comprises a Fab, wherein a partial hinge (for example, a polypeptide having the sequence of SEQ ID NO: 133) is connected to the C-terminus of the Fab heavy chain (HC) CH1 domain and forms a disulfide bond with the Fab light chain (LC). Bispecific proteins of this format may also optionally comprise a linker polypeptide connecting the C-terminus of the first polypeptide of the SEED format immunoglobulin Fc domain to the N-terminus of the second component that specifically binds ROR1.

Another bispecific protein format comprises: (i) a first arm comprising, from N-terminus to C-terminus, a first component comprising a polypeptide or complex of two or more polypeptides that specifically binds ROR1, a first hinge polypeptide, a first polypeptide of a SEED format immunoglobulin Fc domain, and a component comprising a polypeptide or complex of two or more polypeptides that specifically binds CD3; and (ii) a second arm comprising, from N-terminus to C-terminus, a second component comprising a polypeptide or complex of two or more polypeptides that specifically binds ROR1, a second hinge polypeptide, and a second polypeptide of the SEED format immunoglobulin Fc domain, wherein the first and second polypeptides of the SEED format immunoglobulin Fc domain dimerize. In this format, the first and second components that specifically bind ROR1 are antigen-binding sites (e.g., scFvs, antibodies, Fab, Fab′, F(ab′)₂, minibodies, or VHHs) and the component that specifically binds CD3 is an antigen-binding site (e.g., Fab, scFv, antibody, Fab′, F(ab′)₂, minibody, or VHH). In some embodiments, the first and second components that specifically bind ROR1 are scFvs and the component that specifically binds CD3 is a Fab (FIG. 1B). In some embodiments, the component that specifically binds CD3 comprises a Fab, wherein a partial hinge (for example, a polypeptide having the sequence of SEQ ID NO: 133) is connected to the C-terminus of the Fab heavy chain (HC) CH1 domain and forms a disulfide bond with the Fab light chain (LC). Bispecific proteins of this format may also optionally comprise a linker polypeptide connecting the C-terminus of the first polypeptide of the SEED format immunoglobulin Fc domain to the N-terminus of the component that specifically binds CD3.

In another format, a bispecific protein comprises: (i) a first arm comprising, from N-terminus to C-terminus, a first component comprising a polypeptide or complex of two or more polypeptides that specifically binds ROR1, a first hinge polypeptide, a first polypeptide of an immunoglobulin Fc domain, and a second component comprising a polypeptide or complex of two or more polypeptides that specifically binds ROR1; and (ii) a second arm comprising, from N-terminus to C-terminus, a polypeptide or complex of two or more polypeptides that specifically binds CD3, a second hinge polypeptide, and a second polypeptide of an immunoglobulin Fc domain, wherein the first and second polypeptides of the immunoglobulin Fc domain each comprise one or more than one mutation that promotes heterodimerization of the first and second arms. In this format, the first and second components that specifically bind ROR1 are antigen-binding sites (e.g., scFvs, antibodies, Fab, Fab′, F(ab′)₂, minibodies, or VHHs) and the component that specifically binds CD3 is an antigen-binding site (e.g., Fab, scFv, antibody, Fab′, F(ab′)₂, minibody, or VHH). In some embodiments, the first and second components that specifically bind ROR1 are scFvs and the component that specifically binds CD3 is a Fab (FIG. 1A). In some embodiments, the component that specifically binds CD3 comprises a Fab, wherein a partial hinge (for example, a polypeptide having the sequence of SEQ ID NO: 133) is connected to the C-terminus of the Fab heavy chain (HC) CH1 domain and forms a disulfide bond with the Fab light chain (LC). Bispecific proteins of this format may also optionally comprise a linker polypeptide connecting the C-terminus of the first polypeptide of the immunoglobulin Fc domain to the N-terminus of the second component that specifically binds ROR1.

In yet another format, a bispecific protein comprises: (i) a first arm comprising, from N-terminus to C-terminus, a first component comprising a polypeptide or complex of two or more polypeptides that specifically binds ROR1, a first hinge polypeptide, a first polypeptide of an immunoglobulin Fc domain, and a component comprising a polypeptide or complex of two or more polypeptides that specifically binds CD3; and (ii) a second arm comprising, from N-terminus to C-terminus, a second component comprising a polypeptide or complex of two or more polypeptides that specifically binds ROR1, a second hinge polypeptide, and a second polypeptide of the immunoglobulin Fc domain, wherein the first and second polypeptides of the immunoglobulin Fc domain each comprise one or more than one mutation that promotes heterodimerization of the first and second arms. In this format, the first and second components that specifically bind ROR1 are antigen-binding sites (e.g., scFvs, antibodies, Fab, Fab′, F(ab′)₂, minibodies, or VHHs) and the component that specifically binds CD3 is an antigen-binding site (e.g., Fab, scFv, antibody, Fab′, F(ab′)₂, minibody, or VHH). In some embodiments, the first and second components that specifically bind ROR1 are scFvs and the component that specifically binds CD3 is a Fab (FIG. 1B). In some embodiments, the component that specifically binds CD3 comprises a Fab, wherein a partial hinge (for example, a polypeptide having the sequence of SEQ ID NO: 133) is connected to the C-terminus of the Fab heavy chain (HC) CH1 domain and forms a disulfide bond with the Fab light chain (LC). Bispecific proteins of this format may also optionally comprise a linker polypeptide connecting the C-terminus of the first polypeptide of the immunoglobulin Fc domain to the N-terminus of the component that specifically binds CD3.

Individual components of the proteins are described in more detail below.

I.A Polypeptides or Complexes of Two or More Polypeptides that Specifically Bind ROR1

Polypeptides or complexes of two or more polypeptides that specifically bind ROR1 on the surface of tumor cells function to target bispecific proteins of the present disclosure to hematological tumor cells, for example chronic lymphocytic leukemia (CLL) cells and mantle cell leukemia (MCL) cells, as well as solid tumor cells, for example ovarian, lung, gastric, prostate, uterine, and breast tumor cells. Although ROR1 is expressed at relatively low density on the cell surface, it is considered to be a relatively specific TAA. Therefore, ROR1-binding polypeptides or complexes of two or more polypeptides of the present disclosure have limited binding to normal, non-cancerous, adult tissues. In some embodiments, ROR1-binding polypeptides or complexes of two or more polypeptides of the present disclosure have relatively lower binding affinity as compared to ROR1-binding high affinity and/or monovalent antibodies known in the art. In some embodiments, bispecific proteins of the present disclosure comprise two lower affinity ROR1-binding polypeptides or complexes of two or more polypeptides, thereby conferring greater capacity to distinguish between cells having different densities of surface ROR1 expression.

As used herein, a polypeptide or complex of two or more polypeptides that specifically binds ROR1 is a polypeptide or complex of two or more polypeptides that binds to a protein having a sequence of SEQ ID NO: 268 and related isoforms and orthologs. In some embodiments, ROR1-binding polypeptides or complexes of two or more polypeptides specifically bind one or more than one epitope on the extracellular domain of ROR1. In some embodiments, ROR1-binding polypeptides or complexes of two or more polypeptides specifically bind the distal Ig-like domain of ROR1 (SEQ ID NO: 99). In some embodiments, ROR1-binding polypeptides or complexes of two or more polypeptides of the present disclosure do not bind to the Frizzled or Kringle domain of ROR1 (SEQ ID NO: 102 and SEQ ID NO: 105, respectively).

ROR1 Ig-like Domain:
(SEQ ID NO: 99)
PTSSWNISSELNKDSYLTLDEPMNNITTSLGQTAELHCKVSGNPPPTIR
WFKNDAPVVQEPRRLSFRSTIYGSRLRIRNLDTTDTGYFQCVATNGKEV
VSSTGVLF
ROR1 Frizzled Domain:
(SEQ ID NO: 102)
EEDGFCQPYRGIACARFIGNRTVYMESLHMQGEIENQITAAFTMIGTSS
HLSDKCSQFAIPSLCHYAFPYCDETSSVPKPRDLCRDECEILENVLCQT
EYIFARSNPMILMRLKLPNCEDLPQPESPEAANCIRI
ROR1 Kringle Domain:
(SEQ ID NO: 105)
KCYNSTGVDYRGTVSVTKSGRQCQPWNSQYPHTHTFTALRFPELNGGHS
YCRNPGNQKEAPWCFTLDENFKSDLCDIPAC

In some embodiments, a polypeptide or complex of two or more polypeptides that specifically binds ROR1 includes, but is not limited to, a single-chain variable fragment (scFv), an antibody, a Fab, a Fab′, a F(ab′)₂, a minibody, or a nanobody (VHH). For example, in some embodiments, bispecific proteins of the present disclosure comprise two scFv polypeptides that each specifically bind to the Ig-like domain of ROR1.

In some embodiments, a polypeptide or complex of two or more polypeptides that specifically binds ROR1 comprises a heavy chain variable domain (VH) and a light chain variable domain (VL). TABLE 1 lists VH and VL complementarity-determining regions (CDRs) that, in combination, can specifically bind to ROR1. In some embodiments, a polypeptide or complex of two or more polypeptides that specifically binds ROR1 comprises VHCDR1, VHCDR2, VHCDR3, VLCDR1, VLCDR2, and VLCDR3 sequences selected from the VHCDR1, VHCDR2, VHCDR3, VLCDR1, VLCDR2, and VLCDR3 sequences listed in TABLE 1, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art.

Unless indicated otherwise, the CDR sequences provided in TABLE 1 are determined under the IMGT unique numbering scheme.

TABLE 1
VHCDR and VLCDR sequences for polypeptides or
complexes of two or more polypeptides that
specifically bind ROR1

| | |
|---|---|
| Heavy Chain | Light Chain |

CDR1

| | |
|---|---|
| GFTFTSYA | QSVSSY |
| (SEQ ID NO: 8) | (SEQ ID NO: 11) |
| GFSITTSGVS | QSVSSN |
| (SEQ ID NO: 23) | (SEQ ID NO: 26) |
| GFTFSSYA | PDINSY |
| (SEQ ID NO: 127) | (SEQ ID NO: 130) |
| GYTGTSYT | QDIGSS |
| (SEQ ID NO: 179) | (SEQ ID NO: 182) |
| GYTFTEYT | SSVSY |
| (SEQ ID NO: 173) | (SEQ ID NO: 176) |
| GYTFSDYE | QNVDAA |
| (SEQ ID NO: 143) | (SEQ ID NO: 146) |
| GFSLTSYG | SNVSY |
| (SEQ ID NO: 151) | (SEQ ID NO: 154) |
| GFAFTGYN | QGINSY |
| (SEQ ID NO: 172) | (SEQ ID NO: 264) |
| GYNFTNYW | QDINNY |
| (SEQ ID NO: 159) | (SEQ ID NO: 162) |
| GFTSSYA | QDINSY |
| (SEQ ID NO: 248) | (SEQ ID NO: 170) |

CDR2

| | |
|---|---|
| ISGSGGGT | DAS |
| (SEQ ID NO: 9) | |
| IYWDDDK | GAS |
| (SEQ ID NO: 24) | |
| ISRGGTT | RAN |
| (SEQ ID NO: 128) | |
| INPSSGYT | ATS |
| (SEQ ID NO: 180) | |
| INPNNGGT | LTS |
| (SEQ ID NO: 174) | |
| IDPETGGT | SAS |
| (SEQ ID NO: 144) | |
| IWAGGFT | EIS |
| (SEQ ID NO: 152) | |
| IDPYYGGS | RGN |
| (SEQ ID NO: 262) | |
| IYPGSGST | YTS |
| (SEQ ID NO: 160) | |
| ISTGASA | RAN |
| (SEQ ID NO: 168) | |

CDR3

| | |
|---|---|
| AQGSSIFDY | QQRSNWPPXT |
| (SEQ ID NO: 10) | wherein X is F, S, C, R, |
| | Q, I, L Y, or V |
| | (SEQ ID NO: 13) |
| | QQRSNWPPFT |
| | (SEQ ID NO: 14) |
| | QQRSNWPPST |
| | (SEQ ID NO: 15) |
| | QQRSNWPPCT |
| | (SEQ ID NO: 16) |
| | QQRSNWPPRT |
| | (SEQ ID NO: 17) |
| | QQRSNWPPQT |
| | (SEQ ID NO: 18) |
| | QQRSNWPPIT |
| | (SEQ ID NO: 19) |
| | QQRSNWPPLT |
| | (SEQ ID NO: 20) |
| | QQRSNWPPYT |
| | (SEQ ID NO: 21) |
| | QQRSNWPPVT |
| | (SEQ ID NO: 22) |
| AHAPRYTPGGYFDY | QQYKNWPPA |
| (SEQ ID NO: 25) | (SEQ ID NO: 28) |
| YDYDGYYAMDY | LQYDEFPYT |
| (SEQ ID NO: 129) | (SEQ ID NO: 132) |
| ARRVLWLRRGDY | LQYASSP |
| (SEQ ID NO: 181) | (SEQ ID NO: 184) |
| ALQGFAY | QQWSSNPYT |
| (SEQ ID NO: 175) | (SEQ ID NO: 178) |
| TGYYDYDSFTY | QQYDIYPYT |
| (SEQ ID NO: 145) | (SEQ ID NO: 148) |
| ARRGSSYSMDY | QQWNYPLIT |
| (SEQ ID NO: 153) | (SEQ ID NO: 156) |
| ARSPGGDYAMDY | LQYDEFPYT |
| (SEQ ID NO: 263) | (SEQ ID NO: 132) |
| ARDGNYYAMDY | QQGNTLPPYT |
| (SEQ ID NO: 161) | (SEQ ID NO: 164) |
| ARITTSTWYFDV | LQYDEFPYT |
| (SEQ ID NO: 169) | (SEQ ID NO: 132) |

In some embodiments, a polypeptide or complex of two or more polypeptides that specifically binds ROR1 comprises: (i) a VHCDR1 comprising an amino acid sequence of SEQ ID NO: 8 or SEQ ID NO: 23; (ii) a VHCDR2 comprising an amino acid sequence of SEQ ID NO: 9 or SEQ ID NO: 24; (iii) a VHCDR3 comprising an amino acid sequence of SEQ ID NO: 10 or SEQ ID NO: 25; (iv) a VLCDR1 comprising an amino acid sequence of SEQ ID NO: 11 or SEQ ID NO: 26; (v) a VLCDR2 comprising an amino acid sequence DAS or GAS; and (vi) a VLCDR3 comprising an amino acid sequence of SEQ ID NO: 13 or SEQ ID NO: 28. For example, in some embodiments, a polypeptide or complex of two or more polypeptides that specifically binds ROR1 comprises a VLCDR3 comprising an amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, or SEQ ID NO: 28.

In some embodiments, a polypeptide or complex of two or more polypeptides that specifically binds ROR1 comprises: (i) a VHCDR1 comprising an amino acid sequence of SEQ ID NO: 8; (ii) a VHCDR2 comprising an amino acid sequence of SEQ ID NO: 9; (iii) a VHCDR3 comprising an amino acid sequence of SEQ ID NO: 10; (iv) a VLCDR1 comprising an amino acid sequence of SEQ ID NO: 11; (v) a VLCDR2 comprising an amino acid sequence of DAS; and (vi) a VLCDR3 comprising an amino acid sequence of SEQ ID NO: 14.

In some embodiments, a polypeptide or complex of two or more polypeptides that specifically binds ROR1 comprises: (i) a VHCDR1 comprising an amino acid sequence of SEQ ID NO: 8; (ii) a VHCDR2 comprising an amino acid sequence of SEQ ID NO: 9; (iii) a VHCDR3 comprising an amino acid sequence of SEQ ID NO: 10; (iv) a VLCDR1 comprising an amino acid sequence of SEQ ID NO: 11; (v) a VLCDR2 comprising an amino acid sequence of DAS; and (vi) a VLCDR3 comprising an amino acid sequence of SEQ ID NO: 15.

In some embodiments, a polypeptide or complex of two or more polypeptides that specifically binds ROR1 comprises: (i) a VHCDR1 comprising an amino acid sequence of SEQ ID NO: 23; (ii) a VHCDR2 comprising an amino acid sequence of SEQ ID NO: 24; (iii) a VHCDR3 comprising an amino acid sequence of SEQ ID NO: 25; (iv) a VLCDR1 comprising an amino acid sequence of SEQ ID NO: 26; (v) a VLCDR2 comprising an amino acid sequence of GAS; and (vi) a VLCDR3 comprising an amino acid sequence of SEQ ID NO: 28.

TABLE 2 lists amino acid sequences of exemplary VH and VL domains that, in combination, can specifically bind to ROR1. In some embodiments, polypeptides or complexes of two or more polypeptides of the present disclosure comprise VH and VL having at least 90% sequence identity (e.g., at least 91%, at least 92%, at least 93%, at least 94%, at least 95%, at least 96%, at least 97%, at least 98%, at least 99%, or 100%) to VH domain and VL sequences listed in TABLE 2. In certain embodiments, the VHCDR1, VHCDR2, and VHCDR3 and the VLCDR1, VLCDR2, and VLCDR3 sequences are determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences disclosed in TABLE 2.

Unless indicated otherwise, the CDR sequences provided in TABLE 2 are determined under the IMGT unique numbering scheme.

TABLE 2
VH and VL sequences for polypeptides or complexes of two or more
polypeptides that specifically bind ROR1

| Clone | Amino Acid Sequence | |
|---|---|---|
| 82H02 | Heavy Chain Variable Domain | Light Chain Variable Domain |
| | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPCTFGQGTRLEIK |
| | QGTLVTVSS | (SEQ ID NO: 32) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPCT |
| | (SEQ ID NO: 9) | (SEQ ID NO: 16) |
| | CDR3-AQGSSIFDY | |
| | (SEQ ID NO: 10) | |
| 82H02 | Heavy Chain Variable Domain | Light Chain Variable Domain |
| v1.06 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPFTFGQGTRLEIK |
| | QGTLVTVSS | (SEQ ID NO: 30) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPFT |
| | (SEQ ID NO: 9) | (SEQ ID NO: 14) |
| | CDR3-AQGSSIFDY | |
| | (SEQ ID NO: 10) | |
| 82H02 | Heavy Chain Variable Domain | Light Chain Variable Domain |
| v1.07 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPSTFGQGTRLEIK |
| | QGTLVTVSS | (SEQ ID NO: 31) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPST |
| | (SEQ ID NO: 9) | (SEQ ID NO: 15) |
| | CDR3-AQGSSIFDY | |
| | (SEQ ID NO: 10) | |
| 82H02 | Heavy Chain Variable Domain | Light Chain Variable Domain |
| v1.02 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPRTFGQGTRLEIK |
| | QGTLVTVSS | (SEQ ID NO: 33) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPRT |
| | (SEQ ID NO: 9) | (SEQ ID NO: 17) |
| | CDR3-AQGSSIFDY | |
| | (SEQ ID NO: 10) | |
| 82H02 | Heavy Chain Variable Domain | Light Chain Variable Domain |
| v1.03 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPQTFGQGTRLEIK |
| | QGTLVTVSS | (SEQ ID NO: 34) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPQT |
| | (SEQ ID NO: 9) | (SEQ ID NO: 18) |
| | CDR3-AQGSSIFDY | |
| | (SEQ ID NO: 10) | |
| 82H02 | Heavy Chain Variable Domain | Light Chain Variable Domain |
| v1.04 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPITFGQGTRLEIK |
| | QGTLVTVSS | (SEQ ID NO: 35) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPIT |
| | (SEQ ID NO: 9) | |
| | CDR3-AQGSSIFDY | |
| | (SEQ ID NO: 10) | |
| 82H02 | Heavy Chain Variable Domain | Light Chain Variable Domain |
| v1.05 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPLTFGQGTRLEIK |
| | QGTLVTVSS | (SEQ ID NO: 36) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPLT |
| | (SEQ ID NO: 9) | (SEQ ID NO: 20) |
| | CDR3-AQGSSIFDY | |
| | (SEQ ID NO: 10) | |
| 82H02 | Heavy Chain Variable Domain | Light Chain Variable Domain |
| v1.08 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPYTFGQGTRLEIK |
| | QGTLVTVSS | (SEQ ID NO: 37) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPYT |
| | (SEQ ID NO: 9) | (SEQ ID NO: 21) |
| | CDR3-AQGSSIFDY | |
| | (SEQ ID NO: 10) | |
| 82H02 | Heavy Chain Variable Domain | Light Chain Variable Domain |
| v1.09 | (VH) | (VL) |
| | EVQLLESGGGLVQPGGSLRLSC | EIVLTQSPATLSLSPGERATLSCR |
| | AASGFTFTSYAMSWVRQAPGK | ASQSVSSYLAWYQQKPGQAPRL |
| | GLEWVSTISGSGGGTYYTDSV | LIYDASNRATGIPARFSGSGSGTD |
| | KGRFTISRDNSKNTLYLQMNSL | FTLTISSLEPEDFAVYYCQQRSN |
| | RAEDTAVYYCAQGSSIFDYWG | WPPVTFGQGTRLEIK |
| | QGTLVTVSS | (SEQ ID NO: 38) |
| | (SEQ ID NO: 29) | CDR1-QSVSSY |
| | CDR1-GFTFTSYA | (SEQ ID NO: 11) |
| | (SEQ ID NO: 8) | CDR2-DAS |
| | CDR2-ISGSGGGT | CDR3-QQRSNWPPVT |
| | (SEQ ID NO: 9) | (SEQ ID NO: 22) |
| | CDR3-AQGSSIFDY | |
| | (SEQ ID NO: 10) | |
| 69A02 | Heavy Chain Variable Domain | Light Chain Variable Domain |
| | (VH) | (VL) |
| | QVTLKESGPTLVKPTQTLTLTC | ETTLTQSPATLSVSPGETATLSCR |
| | SFSGFSITTSGVSVGWIRQPPGK | ASQSVSSNLAWYQQKPGQAPRL |
| | ALEWLALIYWDDDKRYSPSLK | LIYGASTRATGIPATFSGSGSGTE |
| | SRLTITRDTSKNQVVLTMTNM | FTLTISSLQSEDFAIYYCQQYKN |
| | DPVDTATYYCAHAPRYTPGGY | WPPAFGGGTELEIK |
| | FDYWGQGTLVTVSS | (SEQ ID NO: 40) |
| | (SEQ ID NO: 39) | CDR1-QSVSSN |
| | CDR1-GFSITTSGVS | (SEQ ID NO: 26) |
| | (SEQ ID NO: 23) | CDR2-GAS |
| | CDR2-IYWDDDK | CDR3-QQYKNWPPA |
| | (SEQ ID NO: 24) | (SEQ ID NO: 28) |
| | CDR3-AHAPRYTPGGYFDY | |
| | (SEQ ID NO: 25) | |
| U.S. | Heavy Chain Variable Domain | Light Chain Variable Domain |
| 9,150,647, | (VH) | (VL) |
| U.S. | EVKLQQSGPELVKPGASVKISC | EIEITQTPALMSASPGEKVTMTCS |
| 10,544,201 | KTSGYTFTEYTMHWVKQSHG | ASSSVSYMYWYQQKPRSSPKPW |
| 2A4 | KSLEWIGGINPNNGGTSYNQKF | IYLTSNLASGVPARFSGSGSGTSY |
| | KGKATLTVDKSSSTAYMELRS | SLTISSMEAEDAATYYCQQWSSN |
| | LTSEDSAVYYCALQGFAYWGQ | PYTFGGGTRLELK |
| | GTPLTVSS | (SEQ ID NO: 124) |
| | (SEQ ID NO: 123) | CDR1-SSVSY |
| | CDR1-GYTFTEYT | (SEQ ID NO: 176) |
| | (SEQ ID NO: 173) | CDR2-LTS |
| | CDR2-INPNNGGT | CDR3-QQWSSNPYT |
| | (SEQ ID NO: 174) | (SEQ ID NO: 178) |
| | CDR3-ALQGFAY | |
| | (SEQ ID NO: 175) | |
| US2013/ | Heavy Chain Variable Domain | Light Chain Variable Domain |
| 0101607, | (VH) | (VL) |
| U.S. | EVKLVESGGGLVKPGGSLKLS | DIKMTQSPSSMYASLGERVTITC |
| 10,544,201 | CAASGFTFSSYAMSWVRQIPEK | KASPDINSYLSWFQQKPGKSPKT |
| 4A5 | RLEWVASISRGGTTYYPDSVK | LIYRANRLVDGVPSRFSGGGSGQ |
| | GRFTISRDNVRNILYLQMSSLR | DYSLTINSLEYEDMGIYYCLQYD |
| | SEDTAMYYCGRYDYDGYYAM | EFPYTFGGGTKLEMK |
| | DYWGQGTSVSS | (SEQ ID NO: 126) |
| | (SEQ ID NO: 125) | CDR1-PDINSY |
| | CDR1-GFTFSSYA | (SEQ ID NO: 130) |
| | (SEQ ID NO: 127) | CDR2-RAN |
| | CDR2-ISRGGTT | CDR3-LQYDEFPYT |
| | (SEQ ID NO: 128) | (SEQ ID NO: 132) |
| | CDR3-YDYDGYYAMDY | |
| | (SEQ ID NO: 129) | |
| U.S. | QVQLQQSGAELVRPGASVTLS | DIVMTQSQKIMSTTVGDRVSITC |
| 10,544,201 | CKASGYTFSDYEMHWVIQTPV | KASQNVDAAVAWYQQKPGQSP |
| 2A2 | HGLEWIGAIDPETGGTAYNQKF | KLLIYSASNRYTGVPDRFTGSGS |
| | KGKAILTADKSSSTAYMELRSL | GTDFTLTISNMQSEDLADYFCQQ |
| | TSEDSAVYYCTGYYDYDSFTY | YDIYPYTFGGGTKLEIK |
| | WGQGTLVTVSA | (SEQ ID NO: 142) |
| | (SEQ ID NO: 141) | CDR1-QNVDAA |
| | CDR1-GYTFSDYE | (SEQ ID NO: 146) |
| | (SEQ ID NO: 143) | CDR2-SAS |
| | CDR2-IDPETGGT | CDR3-QQYDIYPYT |
| | (SEQ ID NO: 144) | (SEQ ID NO: 148) |
| | CDR3-TGYYDYDSFTY | |
| | (SEQ ID NO: 145) | |
| U.S. | QVQLKESGPGLVAPSQTLSITC | EIVLSQSPAITAASLGQKVTITCS |
| 10,544,201 | TVSGFSLTSYGVHWVRQPPGK | ASSNVSYIHWYQQRSGTSPRPWI |
| D10 | GLEWLGVIWAGGFTNYNSALK | YEISKLASGVPVRFSGSGSGTSYS |
| | SRLSISKDNSKSQVLLKMTSLQ | LTISSMEAEDAAIYYCQQWNYPL |
| | TDDTAMYYCARRGSSYSMDY | ITFGSGTKLEIQ |
| | WGQGTSVTVSS | (SEQ ID NO: 150) |
| | (SEQ ID NO: 149) | CDR1-SNVSY |
| | CDR1-GFSLTSYG | (SEQ ID NO: 154) |
| | (SEQ ID NO: 151) | CDR2-EIS |
| | CDR2-IWAGGFT | CDR3-QQWNYPLIT |
| | (SEQ ID NO: 152) | (SEQ ID NO: 156) |
| | CDR3-ARRGSSYSMDY | |
| | (SEQ ID NO: 153) | |
| U.S. | EVQLQQSGPELEKPGASVKISC | DIKMTQSPSSMYASVGERVTITC |
| 10,544,201 | KASGFAFTGYNMNWVKQTNG | KASQGINSYSGWFQQKPGKSPKT |
| G6 | KSLEWIGSIDPYYGGSTYNQKF | LIYRGNRLVDGVPSRFSGSGSGQ |
| | KDKATLTVDKSSSTAYMQLKS | DYSLTISSLEYEDMGIYYCLQYD |
| | LTSDDSAVYYCARSPGGDYAM | EFPYTFGGGTKLEIK |
| | DYWGQGTSVTVSS | (SEQ ID NO: 171) |
| | (SEQ ID NO: 167) | CDR1-QGINSY |
| | CDR1-GFAFTGYN | (SEQ ID NO: 264) |
| | (SEQ ID NO: 172) | CDR2-RGN |
| | CDR2-IDPYYGGS | CDR3-LQYDEFPYT |
| | (SEQ ID NO: 262) | (SEQ ID NO: 132) |
| | CDR3-ARSPGGDYAMDY | |
| | (SEQ ID NO: 263) | |
| U.S. | QVQLQQPGAELVKPGTSVKLS | DIQMTQTTSSLSASLGDRVTITCR |
| 10,544,201 | CKASGYNFTNYWINWVKLRPG | ASQDINNYLNWYQQKPDGTVKL |
| G3 | QGLEWIGEIYPGSGSTNYNEKF | LIYYTSALHSGVPSRFSGSGSGTD |
| | KSKATLTADTSSSTAYMQLSSL | YSLTISNLEQEDIATYFCQQGNTL |
| | ASEDSALYYCARDGNYYAMD | PPYTFGGGTKLEIK |
| | YWGQGTSVTVSS | (SEQ ID NO: 158) |
| | (SEQ ID NO: 157) | CDR1-QDINNY |
| | CDR1-GYNFTNYW | (SEQ ID NO: 162) |
| | (SEQ ID NO: 159) | CDR2-YTS |
| | CDR2-IYPGSGST | CDR3-QQGNTLPPYT |
| | (SEQ ID NO: 160) | (SEQ ID NO: 164) |
| | CDR3-ARDGNYYAMDY | |
| | (SEQ ID NO: 161) | |
| U.S. | EVKLVESGGGLVKPGGSLKLS | DIKMTQSPSSMYASLGERVTITC |
| 10,544,201 | CAASGFTFSSYAMSWVRQTPE | KASQDINSYLSWFQQKPGKSPKT |
| H10 | KRLEWVASISTGASAYFPDSVK | LIYRANRLVDGVPSRFSGSGSGQ |
| | GRFTISRDNARNILYLQMSSLR | DYSLTISSLEYEDMGIYYCLQYD |
| | SEDTAMYYCARITTSTWYFDV | EFPYTFGGGTKLEIK |
| | WGAGTTVTVSS | (SEQ ID NO: 166) |
| | (SEQ ID NO: 165) | CDR1-QDINSY |
| | CDR1-GFTFSSYA | (SEQ ID NO: 170) |
| | (SEQ ID NO: 127) | CDR2-RAN |
| | CDR2-ISTGASA | CDR3-LQYDEFPYT |
| | (SEQ ID NO: 168) | (SEQ ID NO: 132) |
| | CDR3-ARITTSTWYFDV | |
| | (SEQ ID NO: 169) | |

In some embodiments, a polypeptide or complex of two or more polypeptides that specifically binds ROR1 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 29 or SEQ ID NO: 39; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 40.

In some embodiments, a polypeptide or complex of two or more polypeptides that specifically binds ROR1 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 29; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 30.

In some embodiments, a polypeptide or complex of two or more polypeptides that specifically binds ROR1 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 29; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 31.

In some embodiments, a polypeptide or complex of two or more polypeptides that specifically binds ROR1 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 39; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 40.

In some embodiments, a polypeptide or complex of two or more polypeptides that specifically binds ROR1 is in an scFv format. In some embodiments, a ROR1-binding scFv of the present disclosure comprises a linker polypeptide that connects a VH domain and VL domain. In some embodiments, a linker polypeptide comprises the sequence (GGGGS)ₙ (SEQ ID NO: 1), wherein n is 1 to 12. For example, in some embodiments, a ROR-1 binding scFv comprises, from N-terminus to C-terminus, a VL domain, a linker polypeptide, and a VH domain. In other embodiments, a ROR-1 binding scFv comprises, from N-terminus to C-terminus, a VH domain, a linker polypeptide, and a VL domain.

TABLE 3 lists amino acid sequences of exemplary ROR1-binding scFvs. In some embodiments, bispecific proteins of the present disclosure comprise two scFvs comprising scFv sequences at least 90% identical (e.g., at least 91%, at least 92%, at least 93%, at least 94%, at least 95%, at least 96%, at least 97%, at least 98%, at least 99%, or 1⁰⁰%) to an scFv sequence listed in TABLE 3.

TABLE 3
Exemplary ROR1-binding scFv amino acid sequences

| Clone | Amino Acid Sequence |
|---|---|
| 82H02 | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPCT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 43) |
| 82H02 | EIVLTQSPATLSLSPGERATLSCRASQSVSSYLAWYQQKPGQAPRLLIY |
| V1.06(1) | DASNRATGIPARFSGSGSGTDFTLTISSLEPEDFAVYYCQQRSNWPPFTF |
| | GQGTRLEIKGGGGSGGGGSGGGGSGGGGSEVQLLESGGGLVQPGGSL |
| | RLSCAASGFTFTSYAMSWVRQAPGKGLEWVSTISGSGGGTYYTDSVK |
| | GRFTISRDNSKNTLYLQMNSLRAEDTAVYYCAQGSSIFDYWGQGTLV |
| | TVSS |
| | (SEQ ID NO: 41) |
| 82H02 | EIVLTQSPATLSLSPGERATLSCRASQSVSSYLAWYQQKPGQAPRLLIY |
| V1.07(1) | DASNRATGIPARFSGSGSGTDFTLTISSLEPEDFAVYYCQQRSNWPPSTF |
| | GQGTRLEIKGGGGSGGGGSGGGGSGGGGSEVQLLESGGGLVQPGGSL |
| | RLSCAASGFTFTSYAMSWVRQAPGKGLEWVSTISGSGGGTYYTDSVK |
| | GRFTISRDNSKNTLYLQMNSLRAEDTAVYYCAQGSSIFDYWGQGTLV |
| | TVSS |
| | (SEQ ID NO: 42) |
| 82H02 | EIVLTQSPATLSLSPGERATLSCRASQSVSSYLAWYQQKPGQAPRLLIY |
| V1.02(1) | DASNRATGIPARFSGSGSGTDFTLTISSLEPEDFAVYYCQQRSNWPPRT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 44) |
| 82H02 | EIVLTQSPATLSLSPGERATLSCRASQSVSSYLAWYQQKPGQAPRLLIY |
| V1.03(1) | DASNRATGIPARFSGSGSGTDFTLTISSLEPEDFAVYYCQQRSNWPPQT |
| | FGQGTRLEIKGGGGSGGGGSGGGGGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 45) |
| 82H02 | EIVLTQSPATLSLSPGERATLSCRASQSVSSYLAWYQQKPGQAPRLLIY |
| V1.04(1) | DASNRATGIPARFSGSGSGTDFTLTISSLEPEDFAVYYCQQRSNWPPITF |
| | GQGTRLEIKGGGGSGGGGSGGGGSGGGGSEVQLLESGGGLVQPGGSL |
| | RLSCAASGFTFTSYAMSWVRQAPGKGLEWVSTISGSGGGTYYTDSVK |
| | GRFTISRDNSKNTLYLQMNSLRAEDTAVYYCAQGSSIFDYWGQGTLV |
| | TVSS |
| | (SEQ ID NO: 46) |
| 82H02 | EIVLTQSPATLSLSPGERATLSCRASQSVSSYLAWYQQKPGQAPRLLIY |
| V1.05(1) | DASNRATGIPARFSGSGSGTDFTLTISSLEPEDFAVYYCQQRSNWPPLT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 47) |
| 82H02 | EIVLTQSPATLSLSPGERATLSCRASQSVSSYLAWYQQKPGQAPRLLIY |
| V1.08(1) | DASNRATGIPARFSGSGSGTDFTLTISSLEPEDFAVYYCQQRSNWPPYT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 48) |
| 82H02 | EIVLTQSPATLSLSPGERATLSCRASQSVSSYLAWYQQKPGQAPRLLIY |
| V1.09(1) | DASNRATGIPARFSGSGSGTDFTLTISSLEPEDFAVYYCQQRSNWPPVT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSS |
| | (SEQ ID NO: 49) |
| 82H02(2) | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPCTFGQGTR |
| | LEIK |
| | (SEQ ID NO: 52) |
| 82H02 | EVQLLESGGGLVQPGGSLRLSCAASGFTFTSYAMSWVRQAPGKGLEW |
| V1.06(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPFTFGQGTRL |
| | EIK |
| | (SEQ ID NO: 50) |
| 82H02 | EVQLLESGGGLVQPGGSLRLSCAASGFTFTSYAMSWVRQAPGKGLEW |
| V1.07(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPSTFGQGTRL |
| | EIK |
| | (SEQ ID NO: 51) |
| 82H02 | EVQLLESGGGLVQPGGSLRLSCAASGFTFTSYAMSWVRQAPGKGLEW |
| V1.02(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPRTFGQGTR |
| | LEIK |
| | (SEQ ID NO: 53) |
| 82H02 | EVQLLESGGGLVQPGGSLRLSCAASGFTFTSYAMSWVRQAPGKGLEW |
| V1.03(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPQTFGQGTR |
| | LEIK |
| | (SEQ ID NO: 54) |
| 82H02 | EVQLLESGGGLVQPGGSLRLSCAASGFTFTSYAMSWVRQAPGKGLEW |
| V1.04(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPITFGQGTRL |
| | EIK |
| | (SEQ ID NO: 55) |
| 82H02 | EVQLLESGGGLVQPGGSLRLSCAASGFTFTSYAMSWVRQAPGKGLEW |
| V1.05(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPLTFGQGTRL |
| | EIK |
| | (SEQ ID NO: 56) |
| 82H02 | EVQLLESGGGLVQPGGSLRLSCAASGFTFTSYAMSWVRQAPGKGLEW |
| V1.08(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPYTFGQGTR |
| | LEIK |
| | (SEQ ID NO: 57) |
| 82H02 | EVQLLESGGGLVQPGGSLRLSCAASGFTFTSYAMSWVRQAPGKGLEW |
| V1.09(2) | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQS |
| | PATLSLSPGERATLSCRASQSVSSYLAWYQQKPGQAPRLLIYDASNRA |
| | TGIPARFSGSGSGTDFTLTISSLEPEDFAVYYCQQRSNWPPVTFGQGTR |
| | LEIK |
| | (SEQ ID NO: 58) |
| 69A02 | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAFG |
| | GGTELEIKGGGGSGGGGSGGGGSGGGGSQVTLKESGPTLVKPTQTLTL |
| | TCSFSGFSITTSGVSVGWIRQPPGKALEWLALIYWDDDKRYSPSLKSRL |
| | TITRDTSKNQVVLTMTNMDPVDTATYYCAHAPRYTPGGYFDYWGQG |
| | TLVTVSS |
| | (SEQ ID NO: 59) |
| 69A02(2) | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAFG |
| | GGTELEIK |
| | (SEQ ID NO: 60) |
| U.S. | EIEITQTPALMSASPGEKVTMTCSASSSVSYMYWYQQKPRSSPKPWIY |
| 9150647 | LTSNLASGVPARFSGSGSGTSYSLTISSMEAEDAATYYCQQWSSNPYTF |
| | GGGTRLELKGGGGSGGGGSGGGGSGGGGSEVKLQQSGPELVKPGASV |
| 2A4 | KISCKTSGYTFTEYTMHWVKQSHGKSLEWIGGINPNNGGTSYNQKFK |
| | GKATLTVDKSSSTAYMELRSLTSEDSAVYYCALQGFAYWGQGTPLTV |
| | SS |
| | (SEQ ID NO: 135) |
| U.S. | DIKMTQSPSSMYASLGERVTITCKASPDINSYLSWFQQKPGKSPKTLIY |
| 2013/0101607 | RANRLVDGVPSRFSGGGSGQDYSLTINSLEYEDMGIYYCLQYDEFPYT |
| 4A5 | FGGGTKLEMKGGGGSGGGGSGGGGSGGGGSEVKLQQSGPELVKPGA |
| | SVKISCKTSGYTFTEYTMHWVKQSHGKSLEWIGGINPNNGGTSYNQKF |
| | KGKATLTVDKSSSTAYMELRSLTSEDSAVYYCALQGFAYWGQGTPLT |
| | VSS |
| | (SEQ ID NO: 136) |
| U.S. | EVKLQQSGPELVKPGASVKISCKTSGYTFTEYTMHWVKQSHGKSLEWI |
| 9150647 | GGINPNNGGTSYNQKFKGKATLTVDKSSSTAYMELRSLTSEDSAVYYC |
| 2A4(2) | ALQGFAYWGQGTPLTVSSGGGGSGGGGSGGGGSGGGGSEIEITQTPAL |
| | MSASPGEKVTMTCSASSSVSYMYWYQQKPRSSPKPWIYLTSNLASGV |
| | PARFSGSGSGTSYSLTISSMEAEDAATYYCQQWSSNPYTFGGGTRLEL |
| | K |
| | (SEQ ID NO: 139) |
| U.S. | EVKLQQSGPELVKPGASVKISCKTSGYTFTEYTMHWVKQSHGKSLEWI |
| 2013/0101607 | GGINPNNGGTSYNQKFKGKATLTVDKSSSTAYMELRSLTSEDSAVYYC |
| 4A5(2) | ALQGFAYWGQGTPLTVSSGGGGSGGGGSGGGGSGGGGSDIKMTQSPS |
| | SMYASLGERVTITCKASPDINSYLSWFQQKPGKSPKTLIYRANRLVDG |
| | VPSRFSGGGSGQDYSLTINSLEYEDMGIYYCLQYDEFPYTFGGGTKLE |
| | MK |
| | (SEQ ID NO: 140) |
| U.S. 10,544,201 | DIVMTQSQKIMSTTVGDRVSITCKASQNVDAAVAWYQQKPGQSPKLLI |
| 2A2 | YSASNRYTGVPDRFTGSGSGTDFTLTISNMQSEDLADYFCQQYDIYPY |
| | TFGGGTKLEIKGGGGSGGGGSGGGGSGGGGSQVQLQQSGAELVRPGA |
| | SVTLSCKASGYTFSDYEMHWVIQTPVHGLEWIGAIDPETGGTAYNQKF |
| | KGKAILTADKSSSTAYMELRSLTSEDSAVYYCTGYYDYDSFTYWGQG |
| | TLVTVSA |
| | (SEQ ID NO: 185) |
| U.S. 10,544,201 | EIVLSQSPAITAASLGQKVTITCSASSNVSYIHWYQQRSGTSPRPWIYEIS |
| D10 | KLASGVPVRFSGSGSGTSYSLTISSMEAEDAAIYYCQQWNYPLITFGSG |
| | TKLEIQGGGGSGGGGSGGGGSGGGGSQVQLKESGPGLVAPSQTLSITC |
| | TVSGFSLTSYGVHWVRQPPGKGLEWLGVIWAGGFTNYNSALKSRLSIS |
| | KDNSKSQVLLKMTSLQTDDTAMYYCARRGSSYSMDYWGQGTSVTVS |
| | S |
| | (SEQ ID NO: 186) |
| U.S. 10,544,201 | DIKMTQSPSSMYASVGERVTITCKASQGINSYSGWFQQKPGKSPKTLIY |
| G6 | RGNRLVDGVPSRFSGSGSGQDYSLTISSLEYEDMGIYYCLQYDEFPYTF |
| | GGGTKLEIKGGGGSGGGGSGGGGSGGGGSEVQLQQSGPELEKPGASV |
| | KISCKASGFAFTGYNMNWVKQTNGKSLEWIGSIDPYYGGSTYNQKFK |
| | DKATLTVDKSSSTAYMQLKSLTSDDSAVYYCARSPGGDYAMDYWGQ |
| | GTSVTVSS |
| | (SEQ ID NO: 266) |
| U.S. 10,544,201 | DIQMTQTTSSLSASLGDRVTITCRASQDINNYLNWYQQKPDGTVKLLI |
| G3 | YYTSALHSGVPSRFSGSGSGTDYSLTISNLEQEDIATYFCQQGNTLPPY |
| | TFGGGTKLEIK |
| | GGGGSGGGGSGGGGSGGGGSQVQLQQPGAELVKPGTSVKLSCKASG |
| | YNFTNYWINWVKLRPGQGLEWIGEIYPGSGSTNYNEKFKSKATLTADT |
| | SSSTAYMQLSSLASEDSALYYCARDGNYYAMDYWGQGTSVTVSS |
| | (SEQ ID NO: 187) |
| U.S. 10,544,201 | DIKMTQSPSSMYASLGERVTITCKASQDINSYLSWFQQKPGKSPKTLIY |
| H10 | RANRLVDGVPSRFSGSGSGQDYSLTISSLEYEDMGIYYCLQYDEFPYTF |
| | GGGTKLEIK |
| | GGGGSGGGGSGGGGSGGGGSEVKLVESGGGLVKPGGSLKLSCAASGF |
| | TFSSYAMSWVRQTPEKRLEWVASISTGASAYFPDSVKGRFTISRDNAR |
| | NILYLQMSSLRSEDTAMYYCARITTSTWYFDVWGAGTTVTVSS |
| | (SEQ ID NO: 188) |
| U.S. 10,544,201 | QVQLQQSGAEL VRPGASVTLSCKASGYTFSDYEMHWVIQTPVHGLEW |
| 2A2(2) | IGAIDPETGGTAYNQKFKGKAILTADKSSSTAYMELRSLTSEDSAVYYC |
| | TGYYDYDSFTYWGQGTLVTVSAGGGGSGGGGSGGGGSGGGGSDIVM |
| | TQSQKIMSTTVGDRVSITCKASQNVDAAVAWYQQKPGQSPKLLIYSAS |
| | NRYTGVPDRFTGSGSGTDFTLTISNMQSEDLADYFCQQYDIYPYTFGG |
| | GTKLEIK |
| | (SEQ ID NO: 189) |
| U.S. 10,544,201 | QVQLKESGPGLVAPSQTLSITCTVSGFSLTSYGVHWVRQPPGKGLEWL |
| D10(2) | GVIWAGGFTNYNSALKSRLSISKDNSKSQVLLKMTSLQTDDTAMYYC |
| | ARRGSSYSMDYWGQGTSVTVSSGGGGSGGGGSGGGGSGGGGSEIVLS |
| | QSPAITAASLGQKVTITCSASSNVSYIHWYQQRSGTSPRPWIYEISKLAS |
| | GVPVRFSGSGSGTSYSLTISSMEAEDAAIYYCQQWNYPLITFGSGTKLEI |
| | Q |
| | (SEQ ID NO: 190) |
| U.S. 10,544,201 | EVQLQQSGPELEKPGASVKISCKASGFAFTGYNMNWVKQTNGKSLEW |
| G6(2) | IGSIDPYYGGSTYNQKFKDKATLTVDKSSSTAYMQLKSLTSDDSAVYY |
| | CARSPGGDYAMDYWGQGTSVTVSSGGGGSGGGGSGGGGSGGGGSDI |
| | KMTQSPSSMYASVGERVTITCKASQGINSYSGWFQQKPGKSPKTLIYR |
| | GNRLVDGVPSRFSGSGSGQDYSLTISSLEYEDMGIYYCLQYDEFPYTFG |
| | GGTKLEIK |
| | (SEQ ID NO: 267) |
| U.S. 10,544,201 | QVQLQQPGAELVKPGTSVKLSCKASGYNFTNYWINWVKLRPGQGLE |
| G3(2) | WIGEIYPGSGSTNYNEKFKSKATLTADTSSSTAYMQLSSLASEDSALYY |
| | CARDGNYYAMDYWGQGTSVTVSSGGGGSGGGGSGGGGSGGGGS |
| | DIQMTQTTSSLSASLGDRVTITCRASQDINNYLNWYQQKPDGTVKLLI |
| | YYTSALHSGVPSRFSGSGSGTDYSLTISNLEQEDIATYFCQQGNTLPPY |
| | TFGGGTKLEIK |
| | (SEQ ID NO: 191) |
| U.S. 10,544,201 | EVKLVESGGGLVKPGGSLKLSCAASGFTFSSYAMSWVRQTPEKRLEW |
| H10(2) | VASISTGASAYFPDSVKGRFTISRDNARNILYLQMSSLRSEDTAMYYCA |
| | RITTSTWYFDVWGAGTTVTVSSGGGGSGGGGSGGGGSGGGGS |
| | DIKMTQSPSSMYASLGERVTITCKASQDINSYLSWFQQKPGKSPKTLIY |
| | RANRLVDGVPSRFSGSGSGQDYSLTISSLEYEDMGIYYCLQYDEFPYTF |
| | GGGTKLEIK |
| | (SEQ ID NO: 192) |

In some embodiments, bispecific proteins of the present disclosure comprise an scFv that specifically binds ROR1 comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, or SEQ ID NO: 59.

In some embodiments, bispecific proteins of the present disclosure comprise two scFvs that specifically bind ROR1, each comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, or SEQ ID NO: 59.

In some embodiments, bispecific proteins of the present disclosure comprise an scFv that specifically binds ROR1 comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41.

In some embodiments, bispecific proteins of the present disclosure comprise two scFvs that specifically bind ROR1, each comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 41.

In some embodiments, bispecific proteins of the present disclosure comprise an scFv that specifically binds ROR1 comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 42.

In some embodiments, bispecific proteins of the present disclosure comprise two scFvs that specifically bind ROR1, each comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 42.

In some embodiments, bispecific proteins of the present disclosure comprise an scFv that specifically binds ROR1 comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 59.

In some embodiments, bispecific proteins of the present disclosure comprise two scFvs that specifically bind ROR1, each comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 59.

I.B Polypeptides or Complexes of Two or More Polypeptides that Specifically Bind CD3

Bispecific proteins of the present disclosure comprise a polypeptide or complex of two or more polypeptides that specifically bind CD3 on the surface of T cells. In some embodiments, polypeptides or complexes of two or more polypeptides specifically bind the CD3ε chain of the CD3/T cell receptor complex, which is broadly expressed on all mature T lymphocytes, for example, as αβ cells, γδ T cells, NK-T cells, mucosal-associated invariant T (MAIT) cells, and their phenotypic subsets. In some embodiments, binding of the CD3ε chain induces activation of the T cell when bridged to ROR1.

As used herein, a polypeptide or complex of two or more polypeptides that specifically binds CD3, is a polypeptide or complex of two or more polypeptides that specifically binds CD3ε (SEQ ID NO: 269).

In some embodiments, a polypeptide or complex of two or more polypeptides that specifically binds CD3 includes, but is not limited to: a Fab, an antibody, a Fab′, a F(ab′)₂, a single-chain variable fragment (scFv), a minibody, or a nanobody (VHH).

In some embodiments, a polypeptide or complex of two or more polypeptides that specifically binds ROR1 includes, but is not limited to, a single-chain variable fragment (scFv), an antibody, a Fab, a Fab′, a F(ab′)₂, a minibody, or a nanobody (VHH). For example, in some embodiments, bispecific proteins of the present disclosure comprise two scFv polypeptides that each specifically bind to the Ig-like domain of ROR1.

In some embodiments, a polypeptide or complex of two or more polypeptides that specifically binds CD3 comprises a VH domain and a VL domain. TABLE 4 lists VH and VL CDRs that, in combination, can specifically bind to CD3. In some embodiments, a polypeptide or complex of two or more polypeptides that specifically binds CD3 comprises VHCDR1, VHCDR2, VHCDR3, VLCDR1, VLCDR2, and VLCDR3 sequences selected from the VHCDR1, VHCDR2, VHCDR3, VLCDR1, VLCDR2, and VLCDR3 sequences listed in TABLE 4, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art.

Unless indicated otherwise, the CDR sequences provided in TABLE 4 are determined under the IMGT unique numbering scheme.

TABLE 4
VHCDR and VLCDR sequences for polypeptides or
complexes of two or more polypeptides that
specifically bind CD3

| | |
|---|---|
| Heavy Chain | Light Chain |

CDR1

| | |
|---|---|
| GFTFNTYA | TGAVTTSN |
| (SEQ ID NO: 61) | (SEQ ID NO: 64) |

CDR2

| | |
|---|---|
| IRSKYNNYAT | GT |
| (SEQ ID NO: 62) | |

CDR3

| | |
|---|---|
| VRHGNFGX₁X₂YVSWFAY | ALWYSNLWV |
| wherein X₁ is N, A, or E, and | (SEQ ID NO: 66) |
| X₂ is S or A | |
| (SEQ ID NO: 67) | |
| VRHGNFGNSYVSWFAY | |
| (SEQ ID NO: 63) | |
| VRHGNFGASYVSWFAY | |
| (SEQ ID NO: 68) | |
| VRHGNFGESYVSWFAY | |
| (SEQ ID NO: 69) | |
| VRHGNFGNAYVSFAY | |

(SEQ ID NO: 70)

In some embodiments, a polypeptide or complex of two or more polypeptides that specifically binds CD3 comprises: (i) a VHCDR1 comprising an amino acid sequence of SEQ ID NO: 61; (ii) a VHCDR2 comprising an amino acid sequence of SEQ ID NO: 62; (iii) a VHCDR3 comprising an amino acid sequence of SEQ ID NO: 67; (iv) a VLCDR1 comprising an amino acid sequence of SEQ ID NO: 64; (v) a VLCDR2 comprising an amino acid sequence of GT; and (vi) a VLCDR3 comprising an amino acid sequence of SEQ ID NO: 66. For example, in some embodiments, a polypeptide or complex of two or more polypeptides that specifically binds CD3 comprises a VHCDR3 comprising an amino acid sequence of SEQ ID NO: 63, SEQ ID NO: 68, SEQ ID NO: 69, or SEQ ID NO: 70.

In certain embodiments, a polypeptide or complex of two or more polypeptides that specifically binds CD3 comprises: (i) a VHCDR1 comprising an amino acid sequence of SEQ ID NO: 61; (ii) a VHCDR2 comprising an amino acid sequence of SEQ ID NO: 62; (iii) a VHCDR3 comprising an amino acid sequence of SEQ ID NO: 63; (iv) a VLCDR1 comprising an amino acid sequence of SEQ ID NO: 64; (v) a VLCDR2 comprising an amino acid sequence of GT; and (vi) a VLCDR3 comprising an amino acid sequence of SEQ ID NO: 66.

In some embodiments, a polypeptide or complex of two or more polypeptides that specifically binds CD3 comprises: (i) a VHCDR1 comprising an amino acid sequence of SEQ ID NO: 61; (ii) a VHCDR2 comprising an amino acid sequence of SEQ ID NO: 62; (iii) a VHCDR3 comprising an amino acid sequence of SEQ ID NO: 68; (iv) a VLCDR1 comprising an amino acid sequence of SEQ ID NO: 64; (v) a VLCDR2 comprising an amino acid sequence of GT; and (vi) a VLCDR3 comprising an amino acid sequence of SEQ ID NO: 66.

TABLE 5 lists amino acid sequences of exemplary VH and VL domains that, in combination, can specifically bind to CD3. In some embodiments, polypeptides or complexes of two or more polypeptides of the present disclosure comprise VH and VL having at least 90% sequence identity (e.g., at least 91%, at least 92%, at least 93%, at least 94%, at least 95%, at least 96%, at least 97%, at least 98%, at least 99%, or 100%) to VH domain and VL sequences listed in TABLE 5. In certain embodiments, the VHCDR1, VHCDR2, and VHCDR3 and the VLCDR1, VLCDR2, and VLCDR3 sequences are determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences disclosed in in Format 1A or Format 1B.

Unless indicated otherwise, the CDR sequences provided in TABLE 5 are determined under the IMGT unique numbering scheme.

TABLE 5
VH and VL sequences for polypeptides or complexes of two or more
polypeptides that specifically bind CD3

| Clone | Amino Acid Sequence | |
|---|---|---|
| IF3.5 | Heavy Chain Variable Domain (VH) | Light Chain Variable Domain (VL) |
| | EVQLVESGGGLVQPGGSLRLSCA | QAVVTQEPSLTVSPGGTVTLTCRS |
| | ASGFTFNTYAMNWVRQAPGKGL | STGAVTTSNYANWVQQKPGQAP |
| | EWVARIRSKYNNYATYYADSVK | RGLIGGTNKRAPGTPARFSGSLLG |
| | DRFTISRDDSKNTLYLQMNSLRA | GKAALTLSGVQPEDEAEYYCAL |
| | EDTAVYYCVRHGNFGNSYVSWF | WYSNLWVFGGGTKLTVL |
| | AYWGQGTMVTVSS | (SEQ ID NO: 81) |
| | (SEQ ID NO: 80) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGNSYVSWFAY | |
| | (SEQ ID NO: 63) | |
| IF3.5 v1.01 | Heavy Chain Variable Domain (VH) | Light Chain Variable Domain (VL) |
| | EVQLVESGGGLVQPGGSLKLSCA | QTVVTQEPSLTVSPGGTVTLTCAS |
| | ASGFTFNTYAMNWVRQASGKGL | STGAVTTSNYANWVQQKPGQAP |
| | EWVGRIRSKYNNYATYYADSVK | RALIGGTNKRAPGTPARFSGSLLG |
| | GRFTISRDDSKNTLYLQMNSLKT | GKAALTLSGVQPEDEAEYYCAL |
| | EDTAVYYCVRHGNFGNSYVSWF | WYSNLWVFGGGTKLTVL |
| | AYWGQGTMVTVSS | (SEQ ID NO: 76) |
| | (SEQ ID NO: 71) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGNSYVSWFAY | |
| | (SEQ ID NO: 63) | |
| IF3.5 v1.03 | Heavy Chain Variable Domain (VH) | Light Chain Variable Domain (VL) |
| | EVQLVESGGGLVQPGGSLKLSCA | QTVVTQEPSLTVSPGGTVTLTCAS |
| | ASGFTFNTYAMNWVRQASGKGL | STGAVTTSNYANWVQQKPGQAP |
| | EWVGRIRSKYNNYATYYADSVK | RALIYGTNKRAPGTPARFSGSLLG |
| | GRFTISRDDSKNTLYLQMNSLKT | GKAALTLSGVQPEDEAEYYCAL |
| | EDTAVYYCVRHGNFGASYVSWF | WYSNLWVFGGGTKLTVL |
| | AYWGQGTMVTVSS | (SEQ ID NO: 78) |
| | (SEQ ID NO: 72) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGASYVSWFAY | |
| | (SEQ ID NO: 68) | |
| IF3.5 v1.02 | Heavy Chain Variable Domain (VH) | Light Chain Variable Domain (VL) |
| | EVQLVESGGGLVQPGGSLKLSCA | QTVVTQEPSLTVSPGGTVTLTCAS |
| | ASGFTFNTYAMNWVRQASGKGL | STGAVTTSNYANWFQQKPGQAP |
| | EWVGRIRSKYNNYATYYADSVK | RALIGGTNKRAPGTPARFSGSLLG |
| | GRFTISRDDSKNTAYLQMNSLKT | GKAALTLSGVQPEDEAEYYCAL |
| | EDTAVYYCVRHGNFGNSYVSWF | WYSNLWVFGGGTKLTVL |
| | AYWGQGTMVTVSS | (SEQ ID NO: 77) |
| | (SEQ ID NO: 73) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGNSYVSWFAY | |
| | (SEQ ID NO: 63) | |
| IF3.5 v1.04 | Heavy Chain Variable Domain (VH) | Light Chain Variable Domain (VL) |
| | EVQLVESGGGLVQPGGSLKLSCA | QTVVTQEPSLTVSPGGTVTLTCAS |
| | ASGFTFNTYAMNWVRQASGKGL | STGAVTTSNYANWFQQKPGQAP |
| | EWVGRIRSKYNNYATYYADSVK | RALIYGTNKRAPGTPARFSGSLLG |
| | GRFTISRDDSKNTLYLQMNSLKT | GKAALTLSGVQPEDEAEYYCAL |
| | EDTAVYYCVRHGNFGESYVSWF | WYSNLWVFGGGTKLTVL |
| | AYWGQGTMVTVSS | (SEQ ID NO: 79) |
| | (SEQ ID NO: 74) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGESYVSWFAY | |
| | (SEQ ID NO: 69) | |
| IF3.5 v1.05 | Heavy Chain Variable Domain (VH) | Light Chain Variable Domain (VL) |
| | EVQLVESGGGLVQPGGSLKLSCA | QTVVTQEPSLTVSPGGTVTLTCAS |
| | ASGFTFNTYAMNWVRQASGKGL | STGAVTTSNYANWVQQKPGQAP |
| | EWVGRIRSKYNNYATYYADSVK | RALIGGTNKRAPGTPARFSGSLLG |
| | GRFTISRDDSKNTAYLQMNSLKT | GKAALTLSGVQPEDEAEYYCAL |
| | EDTAVYYCVRHGNFGNAYVSFA | WYSNLWVFGGGTKLTVL |
| | YWGQGTMVTVSS | (SEQ ID NO: 76) |
| | (SEQ ID NO: 75) | CDR1-TGAVTTSN |
| | CDR1-GFTFNTYA | (SEQ ID NO: 64) |
| | (SEQ ID NO: 61) | CDR2-GT |
| | CDR2-IRSKYNNYAT | CDR3-ALWYSNLWV |
| | (SEQ ID NO: 62) | (SEQ ID NO: 66) |
| | CDR3-VRHGNFGNAYVSFAY | |
| | (SEQ ID NO: 70) | |

In some embodiments, a polypeptide or complex of two or more polypeptides that specifically binds CD3 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 71, SEQ ID NO: 72, SEQ ID NO: 73, SEQ ID NO: 74, SEQ ID NO: 75 or SEQ ID NO: 80; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 76, SEQ ID NO: 77, SEQ ID NO: 78, SEQ ID NO: 79, or SEQ ID NO: 81.

In some embodiments, a polypeptide or complex of two or more polypeptides that specifically binds CD3 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 72; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 81.

In some embodiments, a polypeptide or complex of two or more polypeptides that specifically binds CD3 comprises: (i) a VH having at least 90% sequence identity (e.g., at least 91%, at least 92%, at least 93%, at least 94%, at least 95%, at least 96%, at least 97%, at least 98%, at least 99%, or 100%) to SEQ ID NO: 71; and (ii) a VL having at least 90% sequence identity (e.g., at least 91%, at least 92%, at least 93%, at least 94%, at least 95%, at least 96%, at least 97%, at least 98%, at least 99%, or 100%) to SEQ ID NO: 76.

In some embodiments, a polypeptide or complex of two or more polypeptides that specifically binds CD3 is in a Fab format. TABLE 6 lists polypeptide sequences of heavy chains (HC) and light chains (LC) that, in combination, can specifically bind to CD3. In some embodiments, the heavy chain and the light chains are arranged in Fab format having HC and LC sequences at least 90% identical (e.g., at least 91%, at least 92%, at least 93%, at least 94%, at least 95%, at least 96%, at least 97%, at least 98%, at least 99%, or 100%) to HC and LC

TABLE 6
Heavy chain and light chain sequences for antigen binding sites in Fab
format that specifically bind CD3

| | |
|---|---|
| Clone | Amino Acid Sequence |

Heavy Chain

| | |
|---|---|
| 1F3.5 | EVQLVESGGGLVQPGGSLRLSCAASGFTFNTYAMNWVRQAPGKGLE |
| | WVARIRSKYNNYATYYADSVKDRFTISRDDSKNTLYLQMNSLRAEDT |
| | AVYYCVRHGNFGNSYVSWFAYWGQGTMVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKV |
| | (SEQ ID NO: 93) |
| 1F3.5_VH1.01 | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGNSYVSWFAYWGQGTMVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKV |
| | (SEQ ID NO: 83) |
| 1F3.5_VH1.03 | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGASYVSWFAYWGQGTMVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKV |
| | (SEQ ID NO: 82) |
| 1F3.5_VH1.02 | EVQLVESGGGLVQPGGSLKLSCAASGFTFNTYAMNWVRQASGKGLE |
| | WVGRIRSKYNNYATYYADSVKGRFTISRDDSKNTAYLQMNSLKTEDT |
| | AVYYCVRHGNFGNSYVSWFAYWGQGTMVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKV |
| | (SEQ ID NO: 108) |
| 1F3.5_VH1.04 | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGESYVSWFAYWGQGTMVTVSSASTKGPSVFPLAPSS |
| | KSTSGGTAALGCLVKDYFPEPVTVSWNSGALTSGVHTFPAVLQSSGLY |
| | SLSSVVTVPSSSLGTQTYICNVNHKPSNTKVDKKV |
| | (SEQ ID NO: 111) |
| 1F3.5_VH1.05 | EVQLVESGGGLVQPGGSLKLSCAASGFTFNTYAMNWVRQASGKGLE |
| | WVGRIRSKYNNYATYYADSVKGRFTISRDDSKNTAYLQMNSLKTEDT |
| | AVYYCVRHGNFGNAYVSFAYWGQGTMVTVSSASTKGPSVFPLAPSSK |
| | STSGGTAALGCLVKDYFPEPVTVSWNSGALTSGVHTFPAVLQSSGLYS |
| | LSSVVTVPSSSLGTQTYICNVNHKPSNTKVDKKV |
| | (SEQ ID NO: 114) |

Light Chain

| | |
|---|---|
| 1F3.5 | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWYS |
| | NLWVFGGGTKLTVL |
| | GQPKAAPSVTLFPPSSEELQANKATLVCLISDFYPGAVTVAWKADSSP |
| | VKAGVETTTPSKQSNNKYAASSYLSLTPEQWKSHRSYSCQVTHEGSTV |
| | EKTVAPTECS |
| | (SEQ ID NO: 84) |
| 1F3.5_VL1.01 | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWYS |
| | NLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSH |
| | RSYSCQVTHEGSTVEKTVAPTECS |
| | (SEQ ID NO: 85) |
| 1F3.5_VL1.03 | QTVVTQEPSLTVSPGGTVTLTCASSTGAVTTSNYANWVQQKPGQAPR |
| | ALIYGTNKRAPGTPARFSGSLLGGKAALTLSGVQPEDEAEYYCALWYS |
| | NLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSH |
| | RSYSCQVTHEGSTVEKTVAPTECS |
| | (SEQ ID NO: 119) |
| 1F3.5_VL1.02 | QTVVTQEPSLTVSPGGTVTLTCASSTGAVTTSNYANWFQQKPGQAPR |
| | ALIGGTNKRAPGTPARFSGSLLGGKAALTLSGVQPEDEAEYYCALWYS |
| | NLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSH |
| | RSYSCQVTHEGSTVEKTVAPTECS |
| | (SEQ ID NO: 120) |
| 1F3.5_VL1.04 | QTVVTQEPSLTVSPGGTVTLTCASSTGAVTTSNYANWFQQKPGQAPR |
| | ALIYGTNKRAPGTPARFSGSLLGGKAALTLSGVQPEDEAEYYCALWYS |
| | NLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSH |
| | RSYSCQVTHEGSTVEKTVAPTECS |
| | (SEQ ID NO: 121) |
| 1F3.5_VL1.05 | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWYS |
| | NLWVFGGGTKLTVLGQPKAAPSVTLFPPSSEELQANKATLVCLISDFYP |
| | GAVTVAWKADSSPVKAGVETTTPSKQSNNKYAASSYLSLTPEQWKSH |
| | RSYSCQVTHEGSTVEKTVAPTECS |
| | (SEQ ID NO: 122) |

In some embodiments, bispecific proteins of the present disclosure comprise a Fab that specifically binds CD3 comprising a heavy chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 82 or SEQ ID NO: 83, and a light chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 84 or SEQ ID NO: 85.

In certain embodiments, bispecific proteins of the present disclosure comprise a Fab that specifically binds CD3 comprising a heavy chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 82, and a light chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 84.

In certain embodiments, bispecific proteins of the present disclosure comprise a Fab that specifically binds CD3 comprising a heavy chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 83, and a light chain comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 85.

In some embodiments, a partial hinge (for example, a polypeptide having the sequence of EPKSC (SEQ ID NO: 133)) is connected to the C-terminus of a CH1 domain of a Fab heavy chain (HC) that specifically binds CD3 and forms a disulfide bond with the Fab light chain (LC) that specifically binds CD3.

### I.C Bridging Moieties

In some embodiments, bispecific proteins of the present disclosure further comprise a bridging moiety. In certain embodiments, the bridging moiety may be non-functional, i.e. merely serves as a structural connection and/or appendage and does not perform a biological function or have a biological purpose. In other embodiments, the bridging moiety is functional and has a biological function in the context of the protein.

In some embodiments, the N-terminus of the bridging moiety is connected to the C-terminus of a polypeptide or complex of two or more polypeptides that specifically binds CD3 and the C-terminus of the bridging moiety is connected to the N-terminus of a polypeptide or complex of two or more polypeptides that specifically binds ROR1. In some embodiments, the N-terminus of the bridging moiety is further connected to the C-terminus of a second polypeptide or second complex of two or more polypeptides that specifically binds ROR1. In some embodiments, the bridging moiety comprises two polypeptide arms, wherein the N-terminus of a first polypeptide arm is connected to the C-terminus of a polypeptide or complex of two or more polypeptides that specifically binds CD3, the C-terminus of a second polypeptide arm is connected to the N-terminus of a polypeptide or complex of two or more polypeptides that specifically binds ROR1, and the N-terminus of the second polypeptide arm is connected to the C-terminus of a second polypeptide or second complex of two or more polypeptides that specifically binds ROR1.

In some embodiments, the N-terminus of the bridging moiety is connected to the C-terminus of a polypeptide or complex of two or more polypeptides that specifically binds ROR1 and the C-terminus of the bridging moiety is connected to the N-terminus of a polypeptide or complex of two or more polypeptides that specifically binds CD3. In some embodiments, the N-terminus of the bridging moiety is further connected to the C-terminus of a second polypeptide or second complex of two or more polypeptides that specifically binds ROR1. In some embodiments, the bridging moiety comprises two polypeptide arms, wherein the N-terminus of a first polypeptide arm is connected to the C-terminus of a polypeptide or complex of two or more polypeptides that specifically binds ROR1, the C-terminus of the first polypeptide arm is connected to the N-terminus of a polypeptide or complex of two or more polypeptides that specifically binds CD3, and the N-terminus of a second polypeptide arm is connected to the C-terminus of a second polypeptide or second complex of two or more polypeptides that specifically binds ROR1.

In some embodiments, the bridging moiety comprises, but is not limited to, a strand-exchange engineered domain (SEED) format Fc domain or functional fragment thereof, an immunoglobulin Fc domain or functional fragment thereof, a polypeptide linker, or a polypeptide hinge.

### SEED Format Fc Domain

In certain embodiments, the bridging moiety comprises a SEED format Fc domain or functional fragment thereof comprising two asymmetric, complementary polypeptide arms, designated AG and GA, each comprising alternating sequences of IgA and IgG CH3 domains. In some embodiments, the AG and GA polypeptide arms of the SEED format Fc domain comprise alternating sequences of human IgA and IgG CH3 domains. In some embodiments, a SEED format Fc domain promotes AG/GA heterodimerization and disfavors AG and GA homodimers.

Within an Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273), numbered according to the EU index as in Kabat. Based on the known domains, mutations can be selected to enhance or reduce the binding affinity to CD16, such as by using phage-displayed libraries or yeast surface-displayed cDNA libraries, or can be designed based on the known three-dimensional structure of the interaction. Accordingly, in certain embodiments, a SEED format Fc domain or functional portion thereof comprises a hinge polypeptide and a CH2 domain.

In some embodiments, the SEED format AG polypeptide arm and GA polypeptide arm each comprise one or more mutation(s) to reduce binding to an Fcγ receptor (e.g., FcγRI, FcγRIIA, FcγRIIB, FcγRIIIA, or FcγRIIIB) or a complement component (e.g., C1q) in the first and/or second polypeptide arms of the SEED format Fc domain. Such mutations are useful for reducing effector functions. For example, a bispecific protein of the present disclosure includes LALA (L234A and L235A) mutations, LALAPA (L234A, L235A, and P329A) mutations, LALAPG (L234A, L235A, and P329G) mutations, or LALEGAASPS (L234A, L235E, G237A, A330S, and P331S) mutations. In some embodiments, the terminal lysine residue of a SEED format Fc domain is mutated (K447A) or deleted (K447A). In some embodiments, amino acids at any one or more of positions 322, 330, 331, 355, and 358 may be mutated. The positions of the amino acid substitutions are all numbered according to the EU index as in Kabat, unless otherwise stated.

TABLE 7 lists polypeptide sequences of SEED format AG and GA polypeptide arms that heterodimerize with each other at their respective CH3 domains. In some embodiments, bispecific proteins of the present disclosure comprise: (i) an AG polypeptide arm having an amino acid sequence at least 90% identical (e.g., at least 91%, at least 92%, at least 93%, at least 94%, at least 95%, at least 96%, at least 97%, at least 98%, at least 99%, or 100%) to an AG polypeptide arm sequence listed in TABLE 7; and (ii) a GA polypeptide arm having an amino acid sequence at least 90% identical (e.g., at least 91%, at least 92%, at least 93%, at least 94%, at least 95%, at least 96%, at least 97%, at least 98%, at least 99%, or 100%) to an GA polypeptide arm sequence listed in TABLE 7.

TABLE 7
Exemplary SEED format Fc domain sequences

| | AG Arm | GA Arm |
|---|---|---|
| Parental | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPFRPEVH | APIEKTISKAKGQPREPQVYTLPPP |
| | LLPPSREEMTKNQVSLTCLARGF | SEELALNELVTLTCLVKGFYPSDI |
| | YPKDIAVEWESNGQPENNYKTTP | AVEWLQGSQELPREKYLTWAPV |
| | SRQEPSQGTTTFAVTSKLTVDKS | LDSDGSFFLYSILRVAAEDWKKG |
| | RWQQGNVFSCSVMHEALHNHY | DTFSCSVMHEALHNHYTQKSLDR |
| | TQKTISLSPGK | SPGK |
| | (SEQ ID NO: 88) | (SEQ ID NO: 89) |
| 82H02 | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALG |
| | KALGAPIEKTISKAKGQPFRPEVH | APIEKTISKAKGQPREPQVYTLPPP |
| | LLPPSREEMTKNQVSLTCLARGF | SEELALNELVTLTCLVKGFYPSDI |
| | YPKDIAVEWESNGQPENNYKTTP | AVEWLQGSQELPREKYLTWAPV |
| | SRQEPSQGTTTFAVTSKLTVDKS | LDSDGSFFLYSILRVAAEDWKKG |
| | RWQQGNVFSCSVMHEALHNHY | DTFSCSVMHEALHNHYTQKSLDR |
| | TQKTISLSPGK | SPGK |
| | (SEQ ID NO: 86) | (SEQ ID NO: 87) |
| 69A02 | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCAVSN | VLHQDWLNGKEYKCAVSNKALA |
| | KALAAPIEKTISKAKGQPFRPEVH | APIEKTISKAKGQPREPQVYTLPPP |
| | LLPPSREEMTKNQVSLTCLARGF | SEELALNELVTLTCLVKGFYPSDI |
| | YPKDIAVEWESNGQPENNYKTTP | AVEWLQGSQELPREKYLTWAPV |
| | SRQEPSQGTTTFAVTSKLTVDKS | LDSDGSFFLYSILRVAAEDWKKG |
| | RWQQGNVFSCSVMHEALHNHY | DTFSCSVMHEALHNRFTQKSLDR |
| | TQKTISLSPGK | SPGK |
| | (SEQ ID NO: 117) | (SEQ ID NO: 118) |
| Parental | GQPFRPEVHLLPPSREEMTKNQV | GQPREPQVYTLPPPSEELALNELV |
| (CH3) | SLTCLARGFYPKDIAVEWESNGQ | TLTCLVKGFYPSDIAVEWLQGSQ |
| | PENNYKTTPSRQEPSQGTTTFAV | ELPREKYLTWAPVLDSDGSFFLYS |
| | TSKLTVDKSRWQQGNVFSCSVM | ILRVAAEDWKKGDTFSCSVMHEA |
| | HEALHNHYTQKTISLSPGK | LHNHYTQKSLDRSPGK |
| | (SEQ ID NO: 296) | (SEQ ID NO: 297) |
| 82H02 | GQPFRPEVHLLPPSREEMTKNQV | GQPREPQVYTLPPPSEELALNELV |
| (CH3) | SLTCLARGFYPKDIAVEWESNGQ | TLTCLVKGFYPSDIAVEWLQGSQ |
| | PENNYKTTPSRQEPSQGTTTFAV | ELPREKYLTWAPVLDSDGSFFLYS |
| | TSKLTVDKSRWQQGNVFSCSVM | ILRVAAEDWKKGDTFSCSVMHEA |
| | HEALHNHYTQKTISLSPGK | LHNHYTQKSLDRSPGK |
| | (SEQ ID NO: 298) | (SEQ ID NO: 299) |
| 69A02 | GQPFRPEVHLLPPSREEMTKNQV | GQPREPQVYTLPPPSEELALNELV |
| (CH3) | SLTCLARGFYPKDIAVEWESNGQ | TLTCLVKGFYPSDIAVEWLQGSQ |
| | PENNYKTTPSRQEPSQGTTTFAV | ELPREKYLTWAPVLDSDGSFFLYS |
| | TSKLTVDKSRWQQGNVFSCSVM | ILRVAAEDWKKGDTFSCSVMHEA |
| | HEALHNHYTQKTISLSPGK | LHNRFTQKSLDRSPGK |
| | (SEQ ID NO: 300) | (SEQ ID NO: 301) |

In some embodiments, bispecific proteins of the present disclosure comprise a bridging moiety comprising a SEED format AG polypeptide arm comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 86, SEQ ID NO: 117, or SEQ ID NO: 88, and a SEED format GA polypeptide arm comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 87, SEQ ID NO: 118, or SEQ ID NO: 89.

In certain embodiments, bispecific proteins of the present disclosure comprise a bridging moiety comprising a SEED format AG polypeptide arm comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 86, and a SEED format GA polypeptide arm comprising an amino acid sequence having at least 90% sequence identity (e.g., at least 91%, at least 92%, at least 93%, at least 94%, at least 95%, at least 96%, at least 97%, at least 98%, at least 99%, or 100%) to SEQ ID NO: 87.

### IrG Fc Heterodimers

In certain embodiments, the bridging moiety comprises an immunoglobulin Fc domain. For example, in some embodiments, the bridging moiety comprises an IgG, IgM, IgA, IgD, or IgE Fc domain. In certain embodiments, the bridging moiety comprises an IgG1, IgG2, IgG3, or IgG4 Fc domain. In certain embodiments, the bridging moiety comprises an IgG1 Fc domain. In certain embodiments, the bridging moiety comprises a human IgG1 Fc domain. In certain embodiments, an immunoglobulin Fc domain bridging moiety comprises a first polypeptide arm of an immunoglobulin Fc domain and a second polypeptide arm of an immunoglobulin Fc domain which dimerize with each other.

In some embodiments, a bispecific protein of the present disclosure includes, a first immunoglobulin Fc domain polypeptide arm and a second immunoglobulin Fc domain polypeptide arm, which are both IgG1 Fc domain polypeptides comprising one or more mutation(s) that promote heterodimerization with each other. In some embodiments, the one or more mutation(s) that promote heterodimerization are within the CH3 of the IgG1 Fc domain. For example, immunoglobulin Fc domain can be engineered for heterodimerization using, but not limited to, a “knobs-into-holes” format, duobody format, azymetric format, charge pair format, crossMab format, electrostatic steering format, or HA-TF format. For example, if the Fc domain is derived from the Fc of a human IgG1, the Fc domain can comprise an amino acid sequence at least 90% identical (e.g., at least 91%, at least 92%, at least 93%, at least 94%, at least 95%, at least 96%, at least 97%, at least 98%, at least 99%, or 100%) to amino acids 234-332 of a human IgG1 antibody, and differ at one or more position(s) selected from the group consisting of E345, Q347, Y349, T350, L351, S354, E356, E357, K360, S364, T366, L368, K370, K392, T394, D399, F405, Y407, and K409 according to EU numbering.

In certain embodiments, an immunoglobulin Fc domain or functional portion thereof comprises a hinge polypeptide and a CH2 domain.

In certain embodiments, the bridging moiety comprises a human IgG1 Fc domain comprising one or more mutation(s) to reduce binding to an Fcγ receptor (e.g., FcγRI, FcγRIIA, FcγRIIB, FcγRIIIA, or FcγRIIIB) or a complement component (e.g., C1q) in the first and/or second polypeptide arms of the human IgG1 Fc domain. Such mutations are useful for reducing effector functions. For example, a bispecific protein of the present disclosure includes LALA (L234A and L235A) mutations, LALAPA (L234A, L235A, and P329A) mutations, LALAPG (L234A, L235A, and P329G) mutations, or LALEGAASPS (L234A, L235E, G237A, A330S, and P331S) mutations. In some embodiments, the terminal lysine residue of human IgG1 Fc domain is mutated (K447A) or deleted (K447A). In some embodiments, amino acids at any one or more of positions 322, 330, 331, 355, and 358 may be mutated. The positions of the amino acid substitutions are all numbered according to the EU index as in Kabat, unless otherwise stated.

TABLE 8 lists polypeptide sequences of immunoglobulin Fc domain polypeptide arms that heterodimerize with each other. In some embodiments, bispecific proteins of the present disclosure comprise: (i) a first polypeptide arm of an immunoglobulin Fc domain having an amino acid sequence at least 90% identical (e.g., at least 91%, at least 92%, at least 93%, at least 94%, at least 95%, at least 96%, at least 97%, at least 98%, at least 99%, or 100%) to a first polypeptide arm sequence listed in TABLE 8; and (ii) a second polypeptide arm of an immunoglobulin Fc domain having an amino acid sequence at least 90% identical (e.g., at least 91%, at least 92%, at least 93%, at least 94%, at least 95%, at least 96%, at least 97%, at least 98%, at least 99%, or 100%) to a second polypeptide arm sequence listed in TABLE 8.

TABLE 8
Exemplary human immunoglobulin Fc domain heterodimerization
sequences

| | First Polypeptide Arm | Second Polypeptide Arm |
|---|---|---|
| Knob-in-hole | PSVFLFPPKPKDTLMISRTPEVTC | PSVFLFPPKPKDTLMISRTPEVTCV |
| (1) | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTKNQVSLWCLVKGF | RDELTKNQVSLSCAVKGFYPSDIA |
| | YPSDIAVEWESNGQPENNYKTTP | VEWESNGQPENNYKTTPPVLDSD |
| | PVLDSDGSFFLYSKLTVDKSRWQ | GSFFLVSKLTVDKSRWQQGNVFS |
| | QGNVFSCSVMHEALHNHYTQKS | CSVMHEALHNHYTQKSLSLSPGK |
| | LSLSPGK | (SEQ ID NO: 195) |
| | (SEQ ID NO: 194) | |
| Knob-in-hole | PSVFLFPPKPKDTLMISRTPEVTC | PSVFLFPPKPKDTLMISRTPEVTCV |
| (2) | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVCTLPPS |
| | TLPPCRDELTKNQVSLWCLVKGF | RDELTKNQVSLSCAVKGFYPSDIA |
| | YPSDIAVEWESNGQPENNYKTTP | VEWESNGQPENNYKTTPPVLDSD |
| | PVLDSDGSFFLYSKLTVDKSRWQ | GSFFLVSKLTVDKSRWQQGNVFS |
| | QGNVFSCSVMHEALHNHYTQKS | CSVMHEALHNHYTQKSLSLSPGK |
| | LSLSPGK | (SEQ ID NO: 197) |
| | (SEQ ID NO: 196) | |
| HA-TF (1) | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVTTLPPS |
| | TLPPSRDELTKNQVHLTCLVKGF | RDELTKNQVSLTCLVKGFYPSDIA |
| | YPSDIAVEWESNGQPENNYKTTP | VEWESNGQPENNYKTFPPVLDSD |
| | PVLDSDGSFALYSKLTVDKSRW | GSFFLYSKLTVDKSRWQQGNVFS |
| | QQGNVFSCSVMHEALHNHYTQK | CSVMHEALHNHYTQKSLSLSPGK |
| | SLSLSPGK | (SEQ ID NO: 199) |
| | (SEQ ID NO: 198) | |
| XmAb | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTKNQVSLTCDVSGF | RDQLTKNQVKLTCLVKGFYPSDI |
| | YPSDIAVEWESNGQPENNYKTTP | AVEWESNGQPENNYKTTPPVLDS |
| | PVLDSDGSFFLYSKLTVDKSRWQ | DGSFFLYSKLTVDKSRWQQGNVF |
| | QGNVFSCSVMHEALHNHYTQKS | SCSVMHEALHNHYTQKSLSLSPG |
| | LSLSPGK | K |
| | (SEQ ID NO: 200) | (SEQ ID NO: 201) |
| ZW1 | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYVLPP |
| | VYPPSRDELTKNQVSLTCLVKGF | SRDELTKNQVSLLCLVKGFYPSDI |
| | YPSDIAVEWESNGQPENNYKTTP | AVEWESNGQPENNYLTWPPVLDS |
| | PVLDSDGSFALVSKLTVDKSRW | DGSFFLYSKLTVDKSRWQQGNVF |
| | QQGNVFSCSVMHEALHNHYTQK | SCSVMHEALHNHYTQKSLSLSPG |
| | SLSLSPGK | K |
| | (SEQ ID NO: 202) | (SEQ ID NO: 203) |
| 7.8.60 | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPRRPRVYTLPPS |
| | TLPPSRDELTDNQVSLTCLVKGF | RDELTKNQVSLVCLVKGFYPSDI |
| | YPSDIAVEWESNGQPENNYKTTP | AVEWESNGQPENNYKTTPPVLDS |
| | PVLMSDGSFFLASKLTVDKSRW | DGSFFLYSVLTVDKSRWQQGNVF |
| | QQGNVFSCSVMHEALHNHYTQK | SCSVMHEALHNHYTQKSLSLSPG |
| | SLSLSPGK | K |
| | (SEQ ID NO: 204) | (SEQ ID NO: 205) |
| Electrostatic | PSVFLFPPKPKDTLMISRTPEVTC | PSVFLFPPKPKDTLMISRTPEVTCV |
| Steering | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTKNQVSLTCLVKGF | RDELTKNQVSLTCLVKGFYPSDIA |
| | YPSDIAVEWESNGQPENNYKTTP | VEWESNGQPENNYKTTPPVLKSD |
| | PVLDSDGSFFLYSNLTVDKSRWQ | GSFFLYSKLTVDKSRWQQGNVFS |
| | QGNVFSCSVMHEALHNHYTQKS | CSVMHEALHNHYTQKSLSLSPGK |
| | LSLSPGK | (SEQ ID NO: 207) |
| | (SEQ ID NO: 206) | |
| DD-KK | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTKNQVSLTCLVKGF | RDKLTKNQVSLTCLVKGFYPSDI |
| | YPSDIAVEWESNGQPENNYDTTP | AVEWESNGQPENNYKTTPPVLKS |
| | PVLDSDGSFFLYSDLTVDKSRWQ | DGSFFLYSKLTVDKSRWQQGNVF |
| | QGNVFSCSVMHEALHNHYTQKS | SCSVMHEALHNHYTQKSLSLSPG |
| | LSLSPGK | K |
| | (SEQ ID NO: 208) | (SEQ ID NO: 209) |
| EW-RVT | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPRVYTLPPS |
| | TLPPSRDELTENQVSLTCLVKGF | RDELTKNQVSLTCLVKGFYPSDIA |
| | YPSDIAVEWESNGQPENNYKTTP | VEWESNGQPENNYKTTPPVLVSD |
| | PVLDSDGSFFLYSWLTVDKSRW | GSFTLYSKLTVDKSRWQQGNVFS |
| | QQGNVFSCSVMHEALHNHYTQK | CSVMHEALHNHYTQKSLSLSPGK |
| | SLSLSPGK (SEQ ID NO: 210) | (SEQ ID NO: 211) |
| EW-RVT (2) | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVC | APIEKTISKAKGQPREPRVYTLPPC |
| | TLPPSRDELTENQVSLTCLVKGF | RDELTKNQVSLTCLVKGFYPSDIA |
| | YPSDIAVEWESNGQPENNYKTTP | VEWESNGQPENNYKTTPPVLVSD |
| | PVLDSDGSFFLYSWLTVDKSRW | GSFTLYSKLTVDKSRWQQGNVFS |
| | QQGNVFSCSVMHEALHNHYTQK | CSVMHEALHNHYTQKSLSLSPGK |
| | SLSLSPGK | (SEQ ID NO: 213) |
| | (SEQ ID NO: 212) | |
| A107 | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTENQVSLTCLVKGF | RDNLTKNQVSLTCLVKGFYPSDI |
| | YPSDIAVEWESNGQPENNYKTTP | AVEWESNGQPENNYKTTPPVLVS |
| | PVLDSDGSFFLYSWLTVDKSRW | DGSFTLYSKLTVDKSRWQQGNVF |
| | QQGNVFSCSVMHEALHNHYTQK | SCSVMHEALHNHYTQKSLSLSPG |
| | SLSLSPGK | K |
| | (SEQ ID NO: 214) | (SEQ ID NO: 215) |
| Duobody | PSVFLFPPKPKDTLMISRTPEVTC | PSVFLFPPKPKDTLMISRTPEVTCV |
| | VVVDVSHEDPEVKFNWYVDGV | VVDVSHEDPEVKFNWYVDGVEV |
| | EVHNAKTKPREEQYNSTYRVVS | HNAKTKPREEQYNSTYRVVSVLT |
| | VLTVLHQDWLNGKEYKCKVSN | VLHQDWLNGKEYKCKVSNKALP |
| | KALPAPIEKTISKAKGQPREPQVY | APIEKTISKAKGQPREPQVYTLPPS |
| | TLPPSRDELTKNQVSLTCLVKGF | RDELTKNQVSLTCLVKGFYPSDIA |
| | YPSDIAVEWESNGQPENNYKTTP | VEWESNGQPENNYKTTPPVLDSD |
| | PVLDSDGSFFLYSRLTVDKSRWQ | GSFLLYSKLTVDKSRWQQGNVFS |
| | QGNVFSCSVMHEALHNHYTQKS | CSVMHEALHNHYTQKSLSLSPGK |
| | LSLSPGK | (SEQ ID NO: 217) |
| | (SEQ ID NO: 216) | |
| Knob-in-hole | GQPREPQVYTLPPSRDELTKNQV | GQPREPQVYTLPPSRDELTKNQVS |
| (1) | SLWCLVKGFYPSDIAVEWESNG | LSCAVKGFYPSDIAVEWESNGQP |
| CH3 | QPENNYKTTPPVLDSDGSFFLYS | ENNYKTTPPVLDSDGSFFLVSKLT |
| | KLTVDKSRWQQGNVFSCSVMHE | VDKSRWQQGNVFSCSVMHEALH |
| | ALHNHYTQKSLSLSPGK | NHYTQKSLSLSPGK |
| | (SEQ ID NO: 272) | (SEQ ID NO: 273) |
| Knob-in-hole | GQPREPQVYTLPPCRDELTKNQV | GQPREPQVCTLPPSRDELTKNQVS |
| (2) | SLWCLVKGFYPSDIAVEWESNG | LSCAVKGFYPSDIAVEWESNGQP |
| CH3 | QPENNYKTTPPVLDSDGSFFLYS | ENNYKTTPPVLDSDGSFFLVSKLT |
| | KLTVDKSRWQQGNVFSCSVMHE | VDKSRWQQGNVFSCSVMHEALH |
| | ALHNHYTQKSLSLSPGK | NHYTQKSLSLSPGK |
| | (SEQ ID NO: 274) | (SEQ ID NO: 275) |
| HA-TF (1) | GQPREPQVYTLPPSRDELTKNQV | GQPREPQVTTLPPSRDELTKNQVS |
| CH3 | HLTCLVKGFYPSDIAVEWESNGQ | LTCLVKGFYPSDIAVEWESNGQP |
| | PENNYKTTPPVLDSDGSFALYSK | ENNYKTFPPVLDSDGSFFLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 276) | (SEQ ID NO: 277) |
| XmAb | GQPREPQVYTLPPSRDELTKNQV | GQPREPQVYTLPPSRDQLTKNQV |
| CH3 | SLTCDVSGFYPSDIAVEWESNGQ | KLTCLVKGFYPSDIAVEWESNGQ |
| | PENNYKTTPPVLDSDGSFFLYSK | PENNYKTTPPVLDSDGSFFLYSKL |
| | LTVDKSRWQQGNVFSCSVMHEA | TVDKSRWQQGNVFSCSVMHEAL |
| | LHNHYTQKSLSLSPGK | HNHYTQKSLSLSPGK |
| | (SEQ ID NO: 278) | (SEQ ID NO: 279) |
| ZW1 | GQPREPQVYVYPPSRDELTKNQV | GQPREPQVYVLPPSRDELTKNQV |
| CH3 | SLTCLVKGFYPSDIAVEWESNGQ | SLLCLVKGFYPSDIAVEWESNGQP |
| | PENNYKTTPPVLDSDGSFALVSK | ENNYLTWPPVLDSDGSFFLYSKL |
| | LTVDKSRWQQGNVFSCSVMHEA | TVDKSRWQQGNVFSCSVMHEAL |
| | LHNHYTQKSLSLSPGK | HNHYTQKSLSLSPGK |
| | (SEQ ID NO: 280) | (SEQ ID NO: 281) |
| 7.8.60 | GQPREPQVYTLPPSRDELTDNQV | GQPRRPRVYTLPPSRDELTKNQVS |
| CH3 | SLTCLVKGFYPSDIAVEWESNGQ | LVCLVKGFYPSDIAVEWESNGQP |
| | PENNYKTTPPVLMSDGSFFLASK | ENNYKTTPPVLDSDGSFFLYSVLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 282) | (SEQ ID NO: 283) |
| Electrostatic | GQPREPQVYTLPPSRDELTKNQV | GQPREPQVYTLPPSRDELTKNQVS |
| Steering | SLTCLVKGFYPSDIAVEWESNGQ | LTCLVKGFYPSDIAVEWESNGQP |
| CH3 | PENNYKTTPPVLDSDGSFFLYSN | ENNYKTTPPVLKSDGSFFLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 284) | (SEQ ID NO: 285) |
| DD-KK | GQPREPQVYTLPPSRDELTKNQV | GQPREPQVYTLPPSRDKLTKNQV |
| CH3 | SLTCLVKGFYPSDIAVEWESNGQ | SLTCLVKGFYPSDIAVEWESNGQP |
| | PENNYDTTPPVLDSDGSFFLYSD | ENNYKTTPPVLKSDGSFFLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 286) | (SEQ ID NO: 287) |
| EW-RVT | GQPREPQVYTLPPSRDELTENQV | GQPREPRVYTLPPSRDELTKNQVS |
| CH3 | SLTCLVKGFYPSDIAVEWESNGQ | LTCLVKGFYPSDIAVEWESNGQP |
| | PENNYKTTPPVLDSDGSFFLYSW | ENNYKTTPPVLVSDGSFTLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 288) | (SEQ ID NO: 289) |
| EW-RVT (2) | GQPREPQVCTLPPSRDELTENQV | GQPREPRVYTLPPCRDELTKNQV |
| CH3 | SLTCLVKGFYPSDIAVEWESNGQ | SLTCLVKGFYPSDIAVEWESNGQP |
| | PENNYKTTPPVLDSDGSFFLYSW | ENNYKTTPPVLVSDGSFTLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 290) | (SEQ ID NO: 291) |
| A107 | GQPREPQVYTLPPSRDELTENQV | GQPREPQVYTLPPSRDNLTKNQV |
| CH3 | SLTCLVKGFYPSDIAVEWESNGQ | SLTCLVKGFYPSDIAVEWESNGQP |
| | PENNYKTTPPVLDSDGSFFLYSW | ENNYKTTPPVLVSDGSFTLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 292) | (SEQ ID NO: 293) |
| Duobody | GQPREPQVYTLPPSRDELTKNQV | GQPREPQVYTLPPSRDELTKNQVS |
| CH3 | SLTCLVKGFYPSDIAVEWESNGQ | LTCLVKGFYPSDIAVEWESNGQP |
| | PENNYKTTPPVLDSDGSFFLYSR | ENNYKTTPPVLDSDGSFLLYSKLT |
| | LTVDKSRWQQGNVFSCSVMHEA | VDKSRWQQGNVFSCSVMHEALH |
| | LHNHYTQKSLSLSPGK | NHYTQKSLSLSPGK |
| | (SEQ ID NO: 294) | (SEQ ID NO: 295) |

### Hinge Polypeptide

In some embodiments, the bridging moiety comprises at least a portion of a hinge polypeptide arm. The hinge polypeptide arm can be derived from an immunoglobulin heavy chain, e.g., IgG1, IgG2, IgG3, IgG4, or other classes. Preferably, the hinge polypeptide arm is derived from human IgG1, IgG2, IgG3, or IgG4. More preferably, the hinge polypeptide arm is derived from a human IgG1 heavy chain.

In some embodiments, at least a portion of a hinge polypeptide arm is connected to the N-terminus of the heavy chain constant domain 2 (CH2) of an AG polypeptide arm and GA polypeptide arm of a SEED format Fc domain, or a first polypeptide arm and second polypeptide arm of a human immunoglobulin Fc domain.

In some embodiments, a hinge polypeptide arm connects the C-terminus of a polypeptide or complex of two or more polypeptides that specifically binds CD3 to the N-terminus of the CH2 domain of a SEED format Fc domain AG or GA polypeptide arm, or the N-terminus of the CH2 domain of an immunoglobulin Fc domain first polypeptide arm or second polypeptide arm. In some embodiments, a hinge polypeptide arm connects the C-terminus of a polypeptide or complex of two or more polypeptides that specifically binds ROR1 to the N-terminus of the CH2 domain of a SEED format Fc domain AG or GA polypeptide arm, or the N-terminus of the CH2 domain of an immunoglobulin Fc domain first polypeptide arm or second polypeptide arm.

In some embodiments, a hinge polypeptide arm connects the C-terminus of a polypeptide or complex of two or more polypeptides that specifically binds ROR1 to the N-terminus of the CH2 domain of a SEED format Fc domain AG or GA polypeptide arm, or the N-terminus of the CH2 domain of an immunoglobulin Fc domain first polypeptide arm or second polypeptide arm. In some embodiments, a hinge polypeptide arm connects the C-terminus of a second polypeptide or second complex of two or more polypeptides that specifically binds ROR1 to the N-terminus of the CH2 domain of a SEED format Fc domain AG or GA polypeptide arm, or the N-terminus of the CH2 domain of an immunoglobulin Fc domain first polypeptide arm or second polypeptide arm.

In some embodiments, amino acids at any one or more of positions C220, E233, L234 or L235 may be mutated in a hinge polypeptide arm of human IgG1. The positions of the amino acid substitutions are all numbered according to the EU index as in Kabat, unless otherwise stated.

TABLE 9 lists exemplary hinge polypeptide arm sequences. In some embodiments, a bridging moiety of the present disclosure comprises a hinge polypeptide arm comprising an amino acid sequence at least 90% (e.g., at least 91%, at least 92%, at least 93%, at least 94%, at least 95%, at least 96%, at least 97%, at least 98%, at least 99%, or 100%) identical to a hinge polypeptide arm sequence listed in TABLE 9.

TABLE 9
Exemplary hinge polypeptide arm sequences

| Hinge | Amino Acid Sequence |
|---|---|
| Parental | EPKSCDKTHTCPPCPAPELLGG |
| | (SEQ ID NO: 91) |
| Parental C220S | EPKSSDKTHTCPPCPAPELLGG |
| | (SEQ ID NO: 193) |
| 82H02 (LALA) | EPKSCDKTHTCPPCPAPEAAGG |
| | (SEQ ID NO: 90) |
| 82H02 (LALA)(2) | EPKSSDKTHTCPPCPAPEAAGG |
| | (SEQ ID NO: 92) |
| 69A02 (LALA) | EPKSCDKTHTCPPCPAPPVAG |
| | (SEQ ID NO: 115) |
| 69A02 (LALA)(2) | EPKSSDKTHTCPPCPAPPVAG |
| | (SEQ ID NO: 116) |

In certain embodiments, a bridging moiety of the present invention comprises: (i) a hinge polypeptide arm comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 92; and a second hinge polypeptide arm comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 90.

In certain embodiments, a bridging moiety of the present invention comprises: (i) a hinge polypeptide arm comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 92; and a second hinge polypeptide arm comprising an amino acid at least 90% (e.g., at least 91%, at least 92%, at least 93%, at least 94%, at least 95%, at least 96%, at least 97%, at least 98%, at least 99%, or 100%) identical to SEQ ID NO: 92.

### I.D Linkers

In some embodiments, the N-terminus of a polypeptide or complex of two or more polypeptides that specifically binds ROR1 is connected to the C-terminus of a bridging moiety via a linker polypeptide. In other embodiments, the N-terminus of a polypeptide or complex of two or more polypeptides that specifically binds CD3 is connected to the C-terminus of a bridging moiety via a linker polypeptide.

Regarding the amino acid composition of the linker polypeptide, linker polypeptide sequences are selected with properties that confer flexibility, and for minimal interference with the structure and function of the other domains and/or polypeptides of the proteins described in the present application. Linker polypeptide sequences are also selected to be resistant to proteolytic cleavage. For example, glycine and serine residues generally provide protease resistance.

In certain embodiments, bispecific proteins described herein comprise a (GlyGlyGlyGlySer)₄ ((G₄S)₄) linker (SEQ ID NO: 249). The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, bispecific proteins of the present invention comprise one or more polypeptide linker comprising or consisting of an amino acid sequence listed in TABLE 10.

TABLE 10
Exemplary Linker Polypeptides

| SEQ ID | Amino Acid Sequence |
|---|---|
| SEQ ID NO: 1 | (GGGGS)ₙ, wherein n is 1 to 12 |
| SEQ ID NO: 2 | GSGSGSGSGSGSGSGSGSGS |
| SEQ ID NO: 3 | GGSGGSGGSGGSGGSGGSGGSGGSGGSGGS |
| SEQ ID NO: 4 | GGGSGGGSGGGSGGGSGGGSGGGSGGGSGGGSGGG |
| | SGGGS |
| SEQ ID NO: 5 | GGSGGGSGGGSGGGSGGGSGGGSGGGSGGGSGGGS |
| | GGGSG |
| SEQ ID NO: 6 | GGSGGGGSGGGGSGGGGSGGGGSGGGGSGGGGSGG |
| | GGSGGGGSGGGGSGG |
| SEQ ID NO: 7 | GGSGGGGSGGGGSGGGGSGGGGSGGGGSGGGGSGG |
| | GGSGGGGSGGGGSGGGGSGGGGSGGGGSGGGGSGG |
| | GGSGGGGSGGGGSGGGGSGGGGSGGGGSGG |

### I.E Exemplary Bispecific Proteins

Listed below are examples of bispecific proteins of the present invention comprising a polypeptide or complex of two or more polypeptides that specifically binds a TAA (e.g., ROR1) and a polypeptide or complex of two or more polypeptides that specifically binds CD3.

In one aspect, a bispecific protein of the present disclosure, which induces robust T cell cytotoxicity against certain TAA expressing cells but does not induce a significant amount of cytokine release, includes two arms, the first arm comprises a first component, which specifically binds to a TAA, at its N-terminal end, and a second component, which specifically binds to the same TAA, at its C-terminal end, and a second arm comprises a component, which specifically binds to CD3, at its N-terminal end.

In some embodiments, a bispecific protein of the present disclosure, which induces robust T cell cytotoxicity against certain TAA expressing cells but does not induce a significant amount of cytokine release, comprises: (i) a first arm comprising, from N-terminus to C-terminus, a first component comprising a polypeptide or complex of two or more polypeptides that specifically binds a TAA, a first hinge polypeptide, a first polypeptide of a SEED format immunoglobulin Fc domain, and a second component comprising a polypeptide or complex of two or more polypeptides that specifically binds a TAA; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize.

In some embodiments, a bispecific protein of the present disclosure, which induces robust T cell cytotoxicity against certain TAA expressing tumor cells but does not induce a significant amount of cytokine release, comprises: (i) a first arm comprising, from N-terminus to C-terminus, a first component comprising a polypeptide or complex of two or more polypeptides that specifically binds ROR1, a first hinge polypeptide, a first polypeptide of a SEED format immunoglobulin Fc domain, and a second component comprising a polypeptide or complex of two or more polypeptides that specifically binds ROR1; and (ii) a second arm comprising, from N-terminus to C-terminus, a polypeptide or complex of two or more polypeptides that specifically binds CD3, a second hinge polypeptide, and a second polypeptide of the SEED format immunoglobulin Fc domain, wherein the first and second polypeptides of the SEED format immunoglobulin Fc domain dimerize.

In some embodiments, the polypeptide or complex of two or more polypeptides that specifically binds ROR1 can comprise an antigen binding site (e.g., a single-chain variable fragment (scFv); an antibody, a Fab; a Fab′, a F(ab′)2, a minibody; or a nanobody (VHH)). For example, antigen binding sites that specifically bind ROR1 can comprise an scFv comprising the VHCDR1, VHCDR2, VHCDR3, VLCDR1, VLCDR2, and VLCDR3 sequences selected from any of the VHCDR1, VHCDR2, VHCDR3, VLCDR1, VLCDR2, and VLCDR3 sequences listed in TABLE 1, the VH and VL sequences listed in TABLE 2, and/or the scFv sequences listed in TABLE 3.

As described above, bispecific proteins of the present invention may comprise a bridging moiety. In some embodiments, the polypeptide or complex of two or more polypeptides that specifically binds ROR1 is connected at its N-terminus to the C-terminus of a bridging moiety and the polypeptide or complex of two or more polypeptides that specifically binds CD3 is connected at its C-terminus to the N-terminus of the bridging moiety. In some embodiments, a second polypeptide or second complex of two or more polypeptides that specifically binds ROR1 is connected at its C-terminus to the N-terminus of the bridging moiety.

In other embodiments, the polypeptide or complex of two or more polypeptides that specifically binds ROR1 is connected at its C-terminus to the N-terminus of a bridging moiety and the polypeptide or complex of two or more polypeptides that specifically binds CD3 is connected at its N-terminus to the C-terminus of the bridging moiety. In some embodiments, a second polypeptides or second complex of two or more polypeptides that specifically binds ROR1 is connected at its C-terminus to the N-terminus of the bridging moiety.

As described above, the bridging moiety can be a strand-exchange engineered domain (SEED) format Fc domain or functional fragment thereof, an immunoglobulin Fc domain or functional fragment thereof, a polypeptide linker, or a polypeptide hinge. For example, the bridging moiety can comprise any AG and GA pair of SEED format Fc domain polypeptide arms listed in TABLE 7. In other embodiments, the bridging moiety can comprise an immunoglobulin Fc domain comprising any first polypeptide arm and second polypeptide arm pair listed in TABLE 8. The bridging moiety can also comprise a hinge polypeptide at its N-terminus optionally comprising one or more mutation such as a sequence selected from any one of the sequences listed in TABLE 9.

As also described above, the polypeptide or complex of two or more polypeptides that specifically binds ROR1, or the polypeptide or complex of two or more polypeptides that specifically binds CD3, can be connected to the C-terminus of the bridging moiety via a linker polypeptide. For example, the linker polypeptide can comprise a polypeptide comprising a sequence selected from any one of the sequences listed in TABLE 10.

Proteins of the present invention can comprise a first heavy chain, a second heavy chain, and a light chain. For example, a first heavy chain of the present invention can comprise, from N-terminus to C-terminus: (i) a heavy chain variable (VH) domain and a heavy chain constant domain 1 (CH1) of a Fab that specifically binds CD3; and (ii) a bridging moiety comprising at least a portion of a hinge polypeptide at its N-terminus and an AG or GA polypeptide arm of a SEED format Fc domain. A second heavy chain of the present invention can comprise, from N-terminus to C-terminus: (i) an scFv that specifically binds ROR1; (ii) a bridging moiety comprising at least a portion of a hinge polypeptide at its N-terminus and a GA or AG polypeptide arm of a SEED format Fc domain; (iii) a linker polypeptide; and (iv) a second scFv that specifically binds ROR1. A light chain of the present invention can comprise, from N-terminus to C-terminus: (i) a light chain variable (VL) domain and a light chain constant (CL) domain of a Fab that specifically binds CD3.

In other embodiments, a first heavy chain of the present invention can comprise, from N-terminus to C-terminus: (i) a VH domain and CH1 domain of a Fab that specifically binds CD3; and (ii) a bridging moiety comprising at least a portion of a hinge polypeptide at its N-terminus and a first or second polypeptide arm of an immunoglobulin Fc domain comprising one or more than one mutation that promotes heterodimerization. A second heavy chain of the present invention can comprise, from N-terminus to C-terminus: (i) an scFv that specifically binds ROR1; (ii) a bridging moiety comprising at least a portion of a hinge polypeptide at its N-terminus and a second or first polypeptide arm of an immunoglobulin Fc domain comprising one or more than one mutation that promotes heterodimerization; (iii) a linker polypeptide; and (iv) a second scFv that specifically binds ROR1. A light chain of the present invention can comprise, from N-terminus to C-terminus: (i) a VL domain and a CL domain of a Fab that specifically binds CD3.

In some embodiments, a first heavy chain of the present invention can comprise, from N-terminus to C-terminus: (i) an scFv that specifically binds ROR1; (ii) a bridging moiety comprising at least a portion of a hinge polypeptide at its N-terminus and an AG or GA polypeptide arm of a SEED format Fc domain; (iii) a linker polypeptide; and (iv) a VH domain and CH1 domain of a Fab that specifically binds CD3. A second heavy chain of the present invention can comprise, from N-terminus to C-terminus: (i) an scFv that specifically binds ROR1; (ii) a bridging moiety comprising at least a portion of a hinge polypeptide at its N-terminus and a GA or AG polypeptide arm of a SEED format Fc domain. A light chain of the present invention can comprise, from N-terminus to C-terminus: (i) a VL domain and a CL domain of a Fab that specifically binds CD3. The first heavy chain can additionally comprise at the C-terminus of the CH1 domain, a partial IgG1 hinge sequence (for example, a polypeptide comprising the amino acid sequence of SEQ ID NO: 133) comprising a cysteine residue capable of forming a disulfide bond with a cysteine residue in a CL.

In other embodiments, a first heavy chain of the present invention can comprise, from N-terminus to C-terminus: (i) an scFv that specifically binds ROR1; (ii) a bridging moiety comprising at least a portion of a hinge polypeptide at its N-terminus and a first or second polypeptide arm of an immunoglobulin Fc domain comprising one or more than one mutation that promotes heterodimerization; (iii) a linker polypeptide; and (iv) a VH domain and CH1 domain of a Fab that specifically binds CD3. A second heavy chain of the present invention can comprise, from N-terminus to C-terminus: (i) an scFv that specifically binds ROR1; and (ii) a bridging moiety comprising at least a portion of a hinge polypeptide at its N-terminus and a second or first polypeptide arm of an immunoglobulin Fc domain comprising one or more than one mutation that promotes heterodimerization. A light chain of the present invention can comprise, from N-terminus to C-terminus: (i) a VL domain and a CL domain of a Fab that specifically binds CD3. The first heavy chain can additionally comprise at the C-terminus of the CH1 domain, a partial IgG1 hinge sequence (for example, a polypeptide comprising the amino acid sequence of SEQ ID NO: 133) comprising a cysteine residue capable of forming a disulfide bond with a cysteine residue in a CL.

TABLE 11 lists exemplary first heavy chain, second heavy chain, and light chain sequences of bispecific proteins of the present invention. In combination, a first heavy chain and light chain, or a second heavy chain and light chain as listed in TABLE 11 associate, for example linked by a disulfide bond, and can specifically bind to CD3. As contemplated in bispecific proteins of the present invention, a hinge polypeptide and/or CH3 domain of an AG polypeptide arm of a SEED format Fc domain can facilitate dimerization with a hinge polypeptide and/or CH3 domain of a GA polypeptide arm of a SEED format Fc domain resulting in a bispecific protein comprising a first and second heavy chain and a light chain. As contemplated in other embodiments, a hinge polypeptide and/or CH2 domain of a first polypeptide arm of an immunoglobulin Fc domain comprising one or more than one mutation can facilitate heterodimerization with a hinge polypeptide and/or CH2 domain of a second polypeptide arm of an immunoglobulin Fc domain comprising one or more than one mutation resulting in a bispecific protein comprising a first and second heavy chain and a light chain.

TABLE 11
Exemplary First Heavy Chain, Second Heavy Chain and Light Chain
Sequences

| | Amino Acid Sequence |
|---|---|
| 82H02 | First Heavy Chain: |
| VL1.07x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYK |
| | CKVSNKALGAPIEKTISKAKGQPFRPEVHLLPPSREEMTKNQVSLTCL |
| | ARGFYPKDIAVEWESNGQPENNYKTTPSRQEPSQGTTTFAVTSKLTVD |
| | KSRWQQGNVFSCSVMHEALHNHYTQKTISLSPGK |
| | (SEQ ID NO: 94) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLP |
| | PPSEELALNELVTLTCLVKGFYPSDIAVEWLQGSQELPREKYLTWAPV |
| | LDSDGSFFLYSILRVAAEDWKKGDTFSCSVMHEALHNHYTQKSLDRS |
| | PGKGGGGSGGGGSGGGGSGGGGSEIVLTQSPATLSLSPGERATLSCRA |
| | SQSVSSYLAWYQQKPGQAPRLLIYDASNRATGIPARFSGSGSGTDFTL |
| | TISSLEPEDFAVYYCQQRSNWPPSTFGQGTRLEIKGGGGSGGGGSGGG |
| | GSGGGGSEVQLLESGGGLVQPGGSLRLSCAASGFTFTSYAMSWVRQA |
| | PGKGLEWVSTISGSGGGTYYTDSVKGRFTISRDNSKNTLYLQMNSLR |
| | AEDTAVYYCAQGSSIFDYWGQGTLVTVSS |
| | (SEQ ID NO: 95) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYK |
| | CKVSNKALGAPIEKTISKAKGQPFRPEVHLLPPSREEMTKNQVSLTCL |
| | ARGFYPKDIAVEWESNGQPENNYKTTPSRQEPSQGTTTFAVTSKLTVD |
| | KSRWQQGNVFSCSVMHEALHNHYTQKTISLSPGK |
| | (SEQ ID NO: 97) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLP |
| | PPSEELALNELVTLTCLVKGFYPSDIAVEWLQGSQELPREKYLTWAPV |
| | LDSDGSFFLYSILRVAAEDWKKGDTFSCSVMHEALHNHYTQKSLDRS |
| | PGKGGGGSGGGGSGGGGSGGGGSEIVLTQSPATLSLSPGERATLSCRA |
| | SQSVSSYLAWYQQKPGQAPRLLIYDASNRATGIPARFSGSGSGTDFTL |
| | TISSLEPEDFAVYYCQQRSNWPPSTFGQGTRLEIKGGGGSGGGGSGGG |
| | GSGGGGSEVQLLESGGGLVQPGGSLRLSCAASGFTFTSYAMSWVRQA |
| | PGKGLEWVSTISGSGGGTYYTDSVKGRFTISRDNSKNTLYLQMNSLR |
| | AEDTAVYYCAQGSSIFDYWGQGTLVTVSS |
| | (SEQ ID NO: 98) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02 | First Heavy Chain: |
| VL1.06x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYK |
| | CKVSNKALGAPIEKTISKAKGQPFRPEVHLLPPSREEMTKNQVSLTCL |
| | ARGFYPKDIAVEWESNGQPENNYKTTPSRQEPSQGTTTFAVTSKLTVD |
| | KSRWQQGNVFSCSVMHEALHNHYTQKTISLSPGK |
| | (SEQ ID NO: 100) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPFT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLP |
| | PPSEELALNELVTLTCLVKGFYPSDIAVEWLQGSQELPREKYLTWAPV |
| | LDSDGSFFLYSILRVAAEDWKKGDTFSCSVMHEALHNHYTQKSLDRS |
| | PGKGGGGSGGGGSGGGGSGGGGSEIVLTQSPATLSLSPGERATLSCRA |
| | SQSVSSYLAWYQQKPGQAPRLLIYDASNRATGIPARFSGSGSGTDFTL |
| | TISSLEPEDFAVYYCQQRSNWPPFTFGQGTRLEIKGGGGSGGGGSGGG |
| | GSGGGGSEVQLLESGGGLVQPGGSLRLSCAASGFTFTSYAMSWVRQA |
| | PGKGLEWVSTISGSGGGTYYTDSVKGRFTISRDNSKNTLYLQMNSLR |
| | AEDTAVYYCAQGSSIFDYWGQGTLVTVSS |
| | (SEQ ID NO: 101) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.06x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYK |
| | CKVSNKALGAPIEKTISKAKGQPFRPEVHLLPPSREEMTKNQVSLTCL |
| | ARGFYPKDIAVEWESNGQPENNYKTTPSRQEPSQGTTTFAVTSKLTVD |
| | KSRWQQGNVFSCSVMHEALHNHYTQKTISLSPGK |
| | (SEQ ID NO: 103) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPFT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLP |
| | PPSEELALNELVTLTCLVKGFYPSDIAVEWLQGSQELPREKYLTWAPV |
| | LDSDGSFFLYSILRVAAEDWKKGDTFSCSVMHEALHNHYTQKSLDRS |
| | PGKGGGGSGGGGSGGGGSGGGGSEIVLTQSPATLSLSPGERATLSCRA |
| | SQSVSSYLAWYQQKPGQAPRLLIYDASNRATGIPARFSGSGSGTDFTL |
| | TISSLEPEDFAVYYCQQRSNWPPFTFGQGTRLEIKGGGGSGGGGSGGG |
| | GSGGGGSEVQLLESGGGLVQPGGSLRLSCAASGFTFTSYAMSWVRQA |
| | PGKGLEWVSTISGSGGGTYYTDSVKGRFTISRDNSKNTLYLQMNSLR |
| | AEDTAVYYCAQGSSIFDYWGQGTLVTVSS |
| | (SEQ ID NO: 104) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 69A02x1F3.5: | First Heavy Chain: |
| | EVQLVESGGGLVQPGGSLRLSCAASGFTFNTYAMNWVRQAPGKGLE |
| | WVARIRSKYNNYATYYADSVKDRFTISRDDSKNTLYLQMNSLRAEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPPVAGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | AVSNKALAAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 106) |
| | Second Heavy Chain: |
| | ETTLTQSPATLSVSPGETATLSCRASQSVSSNLAWYQQKPGQAPRLLI |
| | YGASTRATGIPATFSGSGSGTEFTLTISSLQSEDFAIYYCQQYKNWPPA |
| | FGGGTELEIKGGGGSGGGGSGGGGSGGGGSQVTLKESGPTLVKPTQT |
| | LTLTCSFSGFSITTSGVSVGWIRQPPGKALEWLALIYWDDDKRYSPSL |
| | KSRLTITRDTSKNQVVLTMTNMDPVDTATYYCAHAPRYTPGGYFDY |
| | WGQGTLVTVSSEPKSSDKTHTCPPCPAPPVAGPSVFLFPPKPKDTLMIS |
| | RTPEVTCVVVDVSHEDPEVKFNWYVDGVEVHNAKTKPREEQYNSTY |
| | RVVSVLTVLHQDWLNGKEYKCAVSNKALAAPIEKTISKAKGQPREPQ |
| | VYTLPPPSEELALNELVTLTCLVKGFYPSDIAVEWLQGSQELPREKYL |
| | TWAPVLDSDGSFFLYSILRVAAEDWKKGDTFSCSVMHEALHNRFTQK |
| | SLDRSPGKGGGGSGGGGSGGGGSGGGGSETTLTQSPATLSVSPGETAT |
| | LSCRASQSVSSNLAWYQQKPGQAPRLLIYGASTRATGIPATFSGSGSG |
| | TEFTLTISSLQSEDFAIYYCQQYKNWPPAFGGGTELEIKGGGGSGGGG |
| | SGGGGSGGGGSQVTLKESGPTLVKPTQTLTLTCSFSGFSITTSGVSVG |
| | WIRQPPGKALEWLALIYWDDDKRYSPSLKSRLTITRDTSKNQVVLTM |
| | TNMDPVDTATYYCAHAPRYTPGGYFDYWGQGTLVTVSS |
| | (SEQ ID NO: 107) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 1A(2) | First Heavy Chain: |
| | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKF |
| | NWYVDGVEVHNAKTKPREEQYNSTYRVVSVLTVLHQDWLNGKEYK |
| | CKVSNKALGAPIEKTISKAKGQPFRPEVHLLPPSREEMTKNQVSLTCL |
| | ARGFYPKDIAVEWESNGQPENNYKTTPSRQEPSQGTTTFAVTSKLTVD |
| | KSRWQQGNVFSCSVMHEALHNHYTQKTISLSPGK |
| | (SEQ ID NO: 218) |
| | Second Heavy Chain: |
| | ETTLTQSPATLSVSPGETATLSCRASQSVSSNLAWYQQKPGQAPRLLI |
| | YGASTRATGIPATFSGSGSGTEFTLTISSLQSEDFAIYYCQQYKNWPPA |
| | FGGGTELEIKGGGGSGGGGSGGGGSGGGGSQVTLKESGPTLVKPTQT |
| | LTLTCSFSGFSITTSGVSVGWIRQPPGKALEWLALIYWDDDKRYSPSL |
| | KSRLTITRDTSKNQVVLTMTNMDPVDTATYYCAHAPRYTPGGYFDY |
| | WGQGTLVTVSSEPKSSDKTHTCPPCPAPEAAGGPSVFLFPPKPKDTLM |
| | ISRTPEVTCVVVDVSHEDPEVKFNWYVDGVEVHNAKTKPREEQYNST |
| | YRVVSVLTVLHQDWLNGKEYKCKVSNKALGAPIEKTISKAKGQPREP |
| | QVYTLPPPSEELALNELVTLTCLVKGFYPSDIAVEWLQGSQELPREKY |
| | LTWAPVLDSDGSFFLYSILRVAAEDWKKGDTFSCSVMHEALHNHYTQ |
| | KSLDRSPGKGGGGSGGGGSGGGGSGGGGSETTLTQSPATLSVSPGET |
| | ATLSCRASQSVSSNLAWYQQKPGQAPRLLIYGASTRATGIPATFSGSG |
| | SGTEFTLTISSLQSEDFAIYYCQQYKNWPPAFGGGTELEIKGGGGSGG |
| | GGSGGGGSGGGGSQVTLKESGPTLVKPTQTLTLTCSFSGFSITTSGVSV |
| | GWIRQPPGKALEWLALIYWDDDKRYSPSLKSRLTITRDTSKNQVVLT |
| | MTNMDPVDTATYYCAHAPRYTPGGYFDYWGQGTLVTVSS |
| | (SEQ ID NO: 219) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02- | First Heavy Chain: |
| VL1.07x1F3.5 | EIVLTQSPATLSLSPGERATLSCRASQSVSSYLAWYQQKPGQAPRLLIY |
| (1B) | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPFRPEVHLLPP |
| | SREEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEVQLVESGGGLVQPGGSLRLSCAA |
| | SGFTFNTYAMNWVRQAPGKGLEWVARIRSKYNNYATYYADSVKDR |
| | FTISRDDSKNTLYLQMNSLRAEDTAVYYCVRHGNFGNSYVSWFAYW |
| | GQGTMVTVSSASTKGPSVFPLAPSSKSTSGGTAALGCLVKDYFPEPVT |
| | VSWNSGALTSGVHTFPAVLQSSGLYSLSSVVTVPSSSLGTQTYICNVN |
| | HKPSNTKVDKKVEPKSC |
| | (SEQ ID NO: 109) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEV |
| | TCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLP |
| | PPSEELALNELVTLTCLVKGFYPSDIAVEWLQGSQELPREKYLTWAPV |
| | LDSDGSFFLYSILRVAAEDWKKGDTFSCSVMHEALHNHYTQKSLDRS |
| | PGK |
| | (SEQ ID NO: 110) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 1B_69A02x1F3.5 | First Heavy Chain: |
| (1B) | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPPVAGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCAVSNKALAAPIEKTISKAKGQPFRPEVHL |
| | LPPSREEMTKNQVSLTCLARGFYPKDIAVEWESNGQPENNYKTTPSRQ |
| | EPSQGTTTFAVTSKLTVDKSRWQQGNVFSCSVMHEALHNHYTQKTIS |
| | LSPGKGGGGSGGGGSGGGGSGGGGSEVQLVESGGGLVQPGGSLRLSC |
| | AASGFTFNTYAMNWVRQAPGKGLEWVARIRSKYNNYATYYADSVK |
| | DRFTISRDDSKNTLYLQMNSLRAEDTAVYYCVRHGNFGNSYVSWFA |
| | YWGQGTMVTVSSASTKGPSVFPLAPSSKSTSGGTAALGCLVKDYFPEP |
| | VTVSWNSGALTSGVHTFPAVLQSSGLYSLSSVVTVPSSSLGTQTYICN |
| | VNHKPSNTKVDKKVEPKSS |
| | (SEQ ID NO: 112) |
| | Second Heavy Chain: |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPPVAGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCAVSNKALAAPIEKTISKAKGQPREPQVYT |
| | LPPPSEELALNELVTLTCLVKGFYPSDIAVEWLQGSQELPREKYLTWA |
| | PVLDSDGSFFLYSILRVAAEDWKKGDTFSCSVMHEALHNRFTQKSLD |
| | RSPGK |
| | (SEQ ID NO: 113) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 (1B)(2) | First Heavy Chain: |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPFRPEV |
| | HLLPPSREEMTKNQVSLTCLARGFYPKDIAVEWESNGQPENNYKTTPS |
| | RQEPSQGTTTFAVTSKLTVDKSRWQQGNVFSCSVMHEALHNHYTQK |
| | TISLSPGKGGGGSGGGGSGGGGSGGGGSEVQLVESGGGLVQPGGSLR |
| | LSCAASGFTFNTYAMNWVRQAPGKGLEWVARIRSKYNNYATYYADS |
| | VKDRFTISRDDSKNTLYLQMNSLRAEDTAVYYCVRHGNFGNSYVSW |
| | FAYWGQGTMVTVSSASTKGPSVFPLAPSSKSTSGGTAALGCLVKDYF |
| | PEPVTVSWNSGALTSGVHTFPAVLQSSGLYSLSSVVTVPSSSLGTQTYI |
| | CNVNHKPSNTKVDKKVEPKSC |
| | (SEQ ID NO: 220) |
| | Second Heavy Chain: |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPREPQV |
| | YTLPPPSEELALNELVTLTCLVKGFYPSDIAVEWLQGSQELPREKYLT |
| | WAPVLDSDGSFFLYSILRVAAEDWKKGDTFSCSVMHEALHNHYTQKS |
| | LDRSPGK |
| | (SEQ ID NO: 221) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| VH-VL scFv | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 94) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGS |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPREPQV |
| | YTLPPPSEELALNELVTLTCLVKGFYPSDIAVEWLQGSQELPREKYLT |
| | WAPVLDSDGSFFLYSILRVAAEDWKKGDTFSCSVMHEALHNHYTQKS |
| | LDRSPGKGGGGSGGGGSGGGGSGGGGSEVQLLESGGGLVQPGGSLRL |
| | SCAASGFTFTSYAMSWVRQAPGKGLEWVSTISGSGGGTYYTDSVKGR |
| | FTISRDNSKNTLYLQMNSLRAEDTAVYYCAQGSSIFDYWGQGTLVTV |
| | SSGGGGSGGGGSGGGGSGGGGSEIVLTQSPATLSLSPGERATLSCRAS |
| | QSVSSYLAWYQQKPGQAPRLLIYDASNRATGIPARFSGSGSGTDFTLTI |
| | SSLEPEDFAVYYCQQRSNWPPSTFGQGTRLEIK |
| | (SEQ ID NO: 222) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| VH-VL scFv | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 97) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GKEVQLLESGGGLVQPGGSLRLSCAASGFTFTSYAMSWVRQAPGKGL |
| | EWVSTISGSGGGTYYTDSVKGRFTISRDNSKNTLYLQMNSLRAEDTA |
| | VYYCAQGSSIFDYWGQGTLVTVSSGGGGSGGGGSGGGGSGGGGSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPSTFGQGTRLEIK |
| | (SEQ ID NO: 223) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02 | First Heavy Chain: |
| VL1.06x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| VH-VL scFv | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 100) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPFTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GKGGGGSGGGGSGGGGSGGGGSEVQLLESGGGLVQPGGSLRLSCAA |
| | SGFTFTSYAMSWVRQAPGKGLEWVSTISGSGGGTYYTDSVKGRFTISR |
| | DNSKNTLYLQMNSLRAEDTAVYYCAQGSSIFDYWGQGTLVTVSSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPFTFGQGTRLEIK |
| | (SEQ ID NO: 224) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.06x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| VH-VL scFv | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 103) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPFTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GKGGGGSGGGGSGGGGSGGGGSEVQLLESGGGLVQPGGSLRLSCAA |
| | SGFTFTSYAMSWVRQAPGKGLEWVSTISGSGGGTYYTDSVKGRFTISR |
| | DNSKNTLYLQMNSLRAEDTAVYYCAQGSSIFDYWGQGTLVTVSSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPFTFGQGTRLEIK |
| | (SEQ ID NO: 225) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 69A02x1F3.5: | First Heavy Chain: |
| VH-VL scFv | EVQLVESGGGLVQPGGSLRLSCAASGFTFNTYAMNWVRQAPGKGLE |
| | WVARIRSKYNNYATYYADSVKDRFTISRDDSKNTLYLQMNSLRAEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPPVAGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | AVSNKALAAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 106) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPPVAGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCAVSNKALAAPIEKTISKAKGQPREPQVYT |
| | LPPPSEELALNELVTLTCLVKGFYPSDIAVEWLQGSQELPREKYLTWA |
| | PVLDSDGSFFLYSILRVAAEDWKKGDTFSCSVMHEALHNRFTQKSLD |
| | RSPGKQVTLKESGPTLVKPTQTLTLTCSFSGFSITTSGVSVGWIRQPPG |
| | KALEWLALIYWDDDKRYSPSLKSRLTITRDTSKNQVVLTMTNMDPVD |
| | TATYYCAHAPRYTPGGYFDYWGQGTLVTVSSGGGGSGGGGSGGGGS |
| | GGGGSGGGGSGGGGSGGGGSGGGGSETTLTQSPATLSVSPGETATLS |
| | CRASQSVSSNLAWYQQKPGQAPRLLIYGASTRATGIPATFSGSGSGTE |
| | FTLTISSLQSEDFAIYYCQQYKNWPPAFGGGTELEIK |
| | (SEQ ID NO: 226) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 1A(2) | First Heavy Chain: |
| VH-VL scFv | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPFRPEVHLLPPSREEMTKNQVSLTCLA |
| | RGFYPKDIAVEWESNGQPENNYKTTPSRQEPSQGTTTFAVTSKLTVDK |
| | SRWQQGNVFSCSVMHEALHNHYTQKTISLSPGK |
| | (SEQ ID NO: 218) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPREPQVY |
| | TLPPPSEELALNELVTLTCLVKGFYPSDIAVEWLQGSQELPREKYLTW |
| | APVLDSDGSFFLYSILRVAAEDWKKGDTFSCSVMHEALHNHYTQKSL |
| | DRSPGKGGGGSGGGGSGGGGSGGGGSQVTLKESGPTLVKPTQTLTLT |
| | CSFSGFSITTSGVSVGWIRQPPGKALEWLALIYWDDDKRYSPSLKSRLT |
| | ITRDTSKNQVVLTMTNMDPVDTATYYCAHAPRYTPGGYFDYWGQGT |
| | LVTVSSGGGGSGGGGSGGGGSGGGGSETTLTQSPATLSVSPGETATLS |
| | CRASQSVSSNLAWYQQKPGQAPRLLIYGASTRATGIPATFSGSGSGTE |
| | FTLTISSLQSEDFAIYYCQQYKNWPPAFGGGTELEIK |
| | (SEQ ID NO: 227) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02- | First Heavy Chain: |
| VL1.07x1F3.5 | EVQLLESGGGLVQPGGSLRLSCAASGFTFTSYAMSWVRQAPGKGLEW |
| (1B) | VSTISGSGGGTYYTDSVKGRFTISRDNSKNTLYLQMNSLRAEDTAVYY |
| VH-VL scFv | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEVQLVESGGGLVQPGGSLRLSCAA |
| | SGFTFNTYAMNWVRQAPGKGLEWVARIRSKYNNYATYYADSVKDRF |
| | TISRDDSKNTLYLQMNSLRAEDTAVYYCVRHGNFGNSYVSWFAYWG |
| | QGTMVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNH |
| | KPSNTKVDKKVEPKSC |
| | (SEQ ID NO: 228) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GK |
| | (SEQ ID NO: 229) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 1B_69A02x1F3.5 | First Heavy Chain: |
| (1B) | QVTLKESGPTLVKPTQTLTLTCSFSGFSITTSGVSVGWIRQPPGKALEW |
| VH-VL scFv | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPPVAGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCAVSNKALAAPIEKTISKAKGQPFRPEVHL |
| | LPPSREEMTKNQVSLTCLARGFYPKDIAVEWESNGQPENNYKTTPSRQ |
| | EPSQGTTTFAVTSKLTVDKSRWQQGNVFSCSVMHEALHNHYTQKTIS |
| | LSPGKGGGGSGGGGSGGGGSGGGGSEVQLVESGGGLVQPGGSLRLSC |
| | AASGFTFNTYAMNWVRQAPGKGLEWVARIRSKYNNYATYYADSVK |
| | DRFTISRDDSKNTLYLQMNSLRAEDTAVYYCVRHGNFGNSYVSWFA |
| | YWGQGTMVTVSSASTKGPSVFPLAPSSKSTSGGTAALGCLVKDYFPEP |
| | VTVSWNSGALTSGVHTFPAVLQSSGLYSLSSVVTVPSSSLGTQTYICN |
| | VNHKPSNTKVDKKVEPKSS |
| | (SEQ ID NO: 230) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPPVAGPSVFLFPPKPKDTLMISRTP |
| | EVTCVVVDVSHEDPEVKFNWYVDGVEVHNAKTKPREEQYNSTYRVV |
| | SVLTVLHQDWLNGKEYKCAVSNKALAAPIEKTISKAKGQPREPQVYT |
| | LPPPSEELALNELVTLTCLVKGFYPSDIAVEWLQGSQELPREKYLTWA |
| | PVLDSDGSFFLYSILRVAAEDWKKGDTFSCSVMHEALHNRFTQKSLD |
| | RSPGK |
| | (SEQ ID NO: 231) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 (1B)(2) | First Heavy Chain: |
| VH-VL scFv | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPFRPEVH |
| | LLPPSREEMTKNQVSLTCLARGFYPKDIAVEWESNGQPENNYKTTPSR |
| | QEPSQGTTTFAVTSKLTVDKSRWQQGNVFSCSVMHEALHNHYTQKTI |
| | SLSPGKGGGGSGGGGSGGGGSGGGGSEVQLVESGGGLVQPGGSLRLS |
| | CAASGFTFNTYAMNWVRQAPGKGLEWVARIRSKYNNYATYYADSV |
| | KDRFTISRDDSKNTLYLQMNSLRAEDTAVYYCVRHGNFGNSYVSWF |
| | AYWGQGTMVTVSSASTKGPSVFPLAPSSKSTSGGTAALGCLVKDYFP |
| | EPVTVSWNSGALTSGVHTFPAVLQSSGLYSLSSVVTVPSSSLGTQTYIC |
| | NVNHKPSNTKVDKKVEPKSC |
| | (SEQ ID NO: 232) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPREPQVY |
| | TLPPPSEELALNELVTLTCLVKGFYPSDIAVEWLQGSQELPREKYLTW |
| | APVLDSDGSFFLYSILRVAAEDWKKGDTFSCSVMHEALHNHYTQKSL |
| | DRSPGK |
| | (SEQ ID NO: 233) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| GA-AG | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 234) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEIVLTQSPATLSLSPGERATLSCRAS |
| | QSVSSYLAWYQQKPGQAPRLLIYDASNRATGIPARFSGSGSGTDFTLTI |
| | SSLEPEDFAVYYCQQRSNWPPSTFGQGTRLEIKGGGGSGGGGSGGGG |
| | SGGGGSEVQLLESGGGLVQPGGSLRLSCAASGFTFTSYAMSWVRQAP |
| | GKGLEWVSTISGSGGGTYYTDSVKGRFTISRDNSKNTLYLQMNSLRA |
| | EDTAVYYCAQGSSIFDYWGQGTLVTVSS |
| | (SEQ ID NO: 235) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| GA-AG | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 236) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEIVLTQSPATLSLSPGERATLSCRAS |
| | QSVSSYLAWYQQKPGQAPRLLIYDASNRATGIPARFSGSGSGTDFTLTI |
| | SSLEPEDFAVYYCQQRSNWPPSTFGQGTRLEIKGGGGSGGGGSGGGG |
| | SGGGGSEVQLLESGGGLVQPGGSLRLSCAASGFTFTSYAMSWVRQAP |
| | GKGLEWVSTISGSGGGTYYTDSVKGRFTISRDNSKNTLYLQMNSLRA |
| | EDTAVYYCAQGSSIFDYWGQGTLVTVSS |
| | (SEQ ID NO: 237) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02 | First Heavy Chain: |
| VL1.06x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| GA-AG | AVYYCVRHGNFGASYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 238) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPFT |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEIVLTQSPATLSLSPGERATLSCRAS |
| | QSVSSYLAWYQQKPGQAPRLLIYDASNRATGIPARFSGSGSGTDFTLTI |
| | SSLEPEDFAVYYCQQRSNWPPFTFGQGTRLEIKGGGGSGGGGSGGGG |
| | SGGGGSEVQLLESGGGLVQPGGSLRLSCAASGFTFTSYAMSWVRQAP |
| | GKGLEWVSTISGSGGGTYYTDSVKGRFTISRDNSKNTLYLQMNSLRA |
| | EDTAVYYCAQGSSIFDYWGQGTLVTVSS |
| | (SEQ ID NO: 239) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.06x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| GA-AG | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 240) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPFT |
| | FGQGTRLEIKGGGGGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEIVLTQSPATLSLSPGERATLSCRAS |
| | QSVSSYLAWYQQKPGQAPRLLIYDASNRATGIPARFSGSGSGTDFTLTI |
| | SSLEPEDFAVYYCQQRSNWPPFTFGQGTRLEIKGGGGSGGGGSGGGG |
| | SGGGGSEVQLLESGGGLVQPGGSLRLSCAASGFTFTSYAMSWVRQAP |
| | GKGLEWVSTISGSGGGTYYTDSVKGRFTISRDNSKNTLYLQMNSLRA |
| | EDTAVYYCAQGSSIFDYWGQGTLVTVSS |
| | (SEQ ID NO: 241) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 69A02 1A(2) | First Heavy Chain: |
| GA-AG | EVQLVESGGGLVQPGGSLKLSCAASGFTFNTYAMNWVRQASGKGLE |
| | WVGRIRSKYNNYATYYADSVKGRFTISRDDSKNTLYLQMNSLKTEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 242) |
| | Second Heavy Chain: |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPFRPEV |
| | HLLPPSREEMTKNQVSLTCLARGFYPKDIAVEWESNGQPENNYKTTPS |
| | RQEPSQGTTTFAVTSKLTVDKSRWQQGNVFSCSVMHEALHNHYTQK |
| | TISLSPGKGGGGSGGGGSGGGGSGGGGSETTLTQSPATLSVSPGETAT |
| | LSCRASQSVSSNLAWYQQKPGQAPRLLIYGASTRATGIPATFSGSGSG |
| | TEFTLTISSLQSEDFAIYYCQQYKNWPPAFGGGTELEIKGGGGSGGGGS |
| | GGGGSGGGGSQVTLKESGPTLVKPTQTLTLTCSFSGFSITTSGVSVGWI |
| | RQPPGKALEWLALIYWDDDKRYSPSLKSRLTITRDTSKNQVVLTMTN |
| | MDPVDTATYYCAHAPRYTPGGYFDYWGQGTLVTVSS |
| | (SEQ ID NO: 243) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02- | First Heavy Chain: |
| VL1.07x1F3.5 | EIVLTQSPATLSLSPGERATLSCRASQSVSSYLAWYQQKPGQAPRLLIY |
| (1B) | DASNRATGIPARFSGSGSGTDFTLTISSLEPEDFAVYYCQQRSNWPPST |
| GA-AG | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GKGGGGSGGGGSGGGGSGGGGSEVQLVESGGGLVQPGGSLRLSCAA |
| | SGFTFNTYAMNWVRQAPGKGLEWVARIRSKYNNYATYYADSVKDRF |
| | TISRDDSKNTLYLQMNSLRAEDTAVYYCVRHGNFGNSYVSWFAYWG |
| | QGTMVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNH |
| | KPSNTKVDKKVEPKSC |
| | (SEQ ID NO: 244) |
| | Second Heavy Chain: |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKGGGGSGGGGSGGGGSGGGGSEVQLLESGGGLVQPGGS |
| | LRLSCAASGFTFTSYAMSWVRQAPGKGLEWVSTISGSGGGTYYTDSV |
| | KGRFTISRDNSKNTLYLQMNSLRAEDTAVYYCAQGSSIFDYWGQGTL |
| | VTVSSEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GK |
| | (SEQ ID NO: 245) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 (1B)(2) | First Heavy Chain: |
| GA-AG | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPREPQV |
| | YTLPPPSEELALNELVTLTCLVKGFYPSDIAVEWLQGSQELPREKYLT |
| | WAPVLDSDGSFFLYSILRVAAEDWKKGDTFSCSVMHEALHNHYTQKS |
| | LDRSPGKGGGGSGGGGSGGGGSGGGGSEVQLVESGGGLVQPGGSLR |
| | LSCAASGFTFNTYAMNWVRQAPGKGLEWVARIRSKYNNYATYYADS |
| | VKDRFTISRDDSKNTLYLQMNSLRAEDTAVYYCVRHGNFGNSYVSW |
| | FAYWGQGTMVTVSSASTKGPSVFPLAPSSKSTSGGTAALGCLVKDYF |
| | PEPVTVSWNSGALTSGVHTFPAVLQSSGLYSLSSVVTVPSSSLGTQTYI |
| | CNVNHKPSNTKVDKKVEPKSC |
| | (SEQ ID NO: 246) |
| | Second Heavy Chain: |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKGGGGSGGGGSGGGGSGGGGSQVTLKESGPTLVKPTQTL |
| | TLTCSFSGFSITTSGVSVGWIRQPPGKALEWLALIYWDDDKRYSPSLKS |
| | RLTITRDTSKNQVVLTMTNMDPVDTATYYCAHAPRYTPGGYFDYWG |
| | QGTLVTVSSEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPFRPEV |
| | HLLPPSREEMTKNQVSLTCLARGFYPKDIAVEWESNGQPENNYKTTPS |
| | RQEPSQGTTTFAVTSKLTVDKSRWQQGNVFSCSVMHEALHNHYTQK |
| | TISLSPGK |
| | (SEQ ID NO: 247) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| VH-VL scFv | AVYYCVRHGNFGASYVSWFAYWGQGTMVTVSSASTKGPSVFPLAPS |
| GA-AG | SKSTSGGTAALGCLVKDYFPEPVTVSWNSGALTSGVHTFPAVLQSSGL |
| | YSLSSVVTVPSSSLGTQTYICNVNHKPSNTKVDKKEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 306) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGS |
| | EIVLTQSPATLSLSPGERATLSCRASQSVSSYLAWYQQKPGQAPRLLIY |
| | DASNRATGIPARFSGSGSGTDFTLTISSLEPEDFAVYYCQQRSNWPPST |
| | FGQGTRLEIKEPKSSDKTHTCPPCPAPEAAGGPSVFLFPPKPKDTLMISR |
| | TPEVTCVVVDVSHEDPEVKFNWYVDGVEVHNAKTKPREEQYNSTYR |
| | VVSVLTVLHQDWLNGKEYKCKVSNKALGAPIEKTISKAKGQPFRPEV |
| | HLLPPSREEMTKNQVSLTCLARGFYPKDIAVEWESNGQPENNYKTTPS |
| | RQEPSQGTTTFAVTSKLTVDKSRWQQGNVFSCSVMHEALHNHYTQK |
| | TISLSPGKGGGGSGGGGSGGGGSGGGGSEVQLLESGGGLVQPGGSLR |
| | LSCAASGFTFTSYAMSWVRQAPGKGLEWVSTISGSGGGTYYTDSVKG |
| | RFTISRDNSKNTLYLQMNSLRAEDTAVYYCAQGSSIFDYWGQGTLVT |
| | VSSGGGGSGGGGSGGGGSGGGGSEIVLTQSPATLSLSPGERATLSCRA |
| | SQSVSSYLAWYQQKPGQAPRLLIYDASNRATGIPARFSGSGSGTDFTL |
| | TISSLEPEDFAVYYCQQRSNWPPSTFGQGTRLEIK |
| | (SEQ ID NO: 307) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.07x1F3.5- | EVQLVESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| VH-VL scFv | SKSTSGGTAALGCLVKDYFPEPVTVSWNSGALTSGVHTFPAVLQSSGL |
| GA-AG | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 250) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKEVQLLESGGGLVQPGGSLRLSCAASGFTFTSYAMSWVRQAPGKGL |
| | EWVSTISGSGGGTYYTDSVKGRFTISRDNSKNTLYLQMNSLRAEDTA |
| | VYYCAQGSSIFDYWGQGTLVTVSSGGGGSGGGGSGGGGSGGGGSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPSTFGQGTRLEIK |
| | (SEQ ID NO: 251) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02 | First Heavy Chain: |
| VL1.06x1F3.5- | EVQLVESGGGLVQPGGSLKLSCAASGFTFNTYAMNWVRQASGKGLE |
| VH1.03 | WVGRIRSKYNNYATYYADSVKGRFTISRDDSKNTLYLQMNSLKTEDT |
| VH-VL scFv | AVYYCVRHGNFGASYVSWFAYWGQGTMVTVSSASTKGPSVFPLAPS |
| GA-AG | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 252) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPFTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEVQLLESGGGLVQPGGSLRLSCAA |
| | SGFTFTSYAMSWVRQAPGKGLEWVSTISGSGGGTYYTDSVKGRFTISR |
| | DNSKNTLYLQMNSLRAEDTAVYYCAQGSSIFDYWGQGTLVTVSSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPFTFGQGTRLEIK |
| | (SEQ ID NO: 253) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 82H02 | First Heavy Chain: |
| VL1.06x1F3.5- | EVQL VESGGGLVQPGGSLKLSCAASGFTFNTYAMNWVRQASGKGLE |
| VH1.01- | WVGRIRSKYNNYATYYADSVKGRFTISRDDSKNTLYLQMNSLKTEDT |
| VL1.01 | AVYYCVRHGNFGNSYVSWFAYWGQGTMVTVSSASTKGPSVFPLAPS |
| VH-VL scFv | SKSTSGGTAALGCLVKDYFPEPVTVSWNSGALTSGVHTFPAVLQSSGL |
| GA-AG | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 254) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPFTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GKGGGGSGGGGSGGGGSGGGGSEVQLLESGGGLVQPGGSLRLSCAA |
| | SGFTFTSYAMSWVRQAPGKGLEWVSTISGSGGGTYYTDSVKGRFTISR |
| | DNSKNTLYLQMNSLRAEDTAVYYCAQGSSIFDYWGQGTLVTVSSGG |
| | GGSGGGGSGGGGSGGGGSEIVLTQSPATLSLSPGERATLSCRASQSVSS |
| | YLAWYQQKPGQAPRLLIYDASNRATGIPARFSGSGSGTDFTLTISSLEP |
| | EDFAVYYCQQRSNWPPFTFGQGTRLEIK |
| | (SEQ ID NO: 255) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 69A02 1A(2) | First Heavy Chain: |
| VH-VL scFv | EVQLVESGGGLVQPGGSLKLSCAASGFTFNTYAMNWVRQASGKGLE |
| GA-AG | WVGRIRSKYNNYATYYADSVKGRFTISRDDSKNTLYLQMNSLKTEDT |
| | AVYYCVRHGNFGNSYVSWFAYWGQGTMVTVSSASTKGPSVFPLAPS |
| | SKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKC |
| | KVSNKALGAPIEKTISKAKGQPREPQVYTLPPPSEELALNELVTLTCLV |
| | KGFYPSDIAVEWLQGSQELPREKYLTWAPVLDSDGSFFLYSILRVAAE |
| | DWKKGDTFSCSVMHEALHNHYTQKSLDRSPGK |
| | (SEQ ID NO: 256) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPFRPEVH |
| | LLPPSREEMTKNQVSLTCLARGFYPKDIAVEWESNGQPENNYKTTPSR |
| | QEPSQGTTTFAVTSKLTVDKSRWQQGNVFSCSVMHEALHNHYTQKTI |
| | SLSPGKGGGGSGGGGSGGGGSGGGGSQVTLKESGPTLVKPTQTLTLT |
| | CSFSGFSITTSGVSVGWIRQPPGKALEWLALIYWDDDKRYSPSLKSRLT |
| | ITRDTSKNQVVLTMTNMDPVDTATYYCAHAPRYTPGGYFDYWGQGT |
| | LVTVSSGGGGSGGGGSGGGGSGGGGSETTLTQSPATLSVSPGETATLS |
| | CRASQSVSSNLAWYQQKPGQAPRLLIYGASTRATGIPATFSGSGSGTE |
| | FTLTISSLQSEDFAIYYCQQYKNWPPAFGGGTELEIK |
| | (SEQ ID NO: 257) |
| | Light Chain: |
| | QTVVTQEPSLTVSPGGTVTLTCASSTGAVTTSNYANWVQQKPGQAPR |
| | ALIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 85) |
| 82H02- | First Heavy Chain: |
| VL1.07x1F3.5 | EVQLLESGGGLVQPGGSLRLSCAASGFTFTSYAMSWVRQAPGKGLEW |
| (1B) | VSTISGSGGGTYYTDSVKGRFTISRDNSKNTLYLQMNSLRAEDTAVYY |
| VH-VL scFv | CAQGSSIFDYWGQGTLVTVSSGGGGSGGGGSGGGGSGGGGSEIVLTQ |
| GA-AG | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPREPQVYTLPP |
| | PSEELALNELVTLTCLVKGFYPSDIAVEWLQGSQELPREKYLTWAPVL |
| | DSDGSFFLYSILRVAAEDWKKGDTFSCSVMHEALHNHYTQKSLDRSP |
| | GKGGGGSGGGGSGGGGSGGGGSEVQLVESGGGLVQPGGSLRLSCAA |
| | SGFTFNTYAMNWVRQAPGKGLEWVARIRSKYNNYATYYADSVKDRF |
| | TISRDDSKNTLYLQMNSLRAEDTAVYYCVRHGNFGNSYVSWFAYWG |
| | QGTMVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNH |
| | KPSNTKVDKKVEPKSC |
| | (SEQ ID NO: 258) |
| | Second Heavy Chain: |
| | EVQLLESGGGLVQPGGSLRLSCAASGFTFTSYAMSWVRQAPGKGLEW |
| | VSTISGSGGGTYYTDSVKGRFTISRDNSKNTLYLQMNSLRAEDTAVYY |
| | CAQGSSIFDYWGQGTLVTVSSGGGGSGGGGSGGGGSGGGGSEIVLTQ |
| | SPATLSLSPGERATLSCRASQSVSSYLAWYQQKPGQAPRLLIYDASNR |
| | ATGIPARFSGSGSGTDFTLTISSLEPEDFAVYYCQQRSNWPPSTFGQGT |
| | RLEIKEPKSSDKTHTCPPCPAPEAAGGPSVFLFPPKPKDTLMISRTPEVT |
| | CVVVDVSHEDPEVKFNWYVDGVEVHNAKTKPREEQYNSTYRVVSVL |
| | TVLHQDWLNGKEYKCKVSNKALGAPIEKTISKAKGQPFRPEVHLLPPS |
| | REEMTKNQVSLTCLARGFYPKDIAVEWESNGQPENNYKTTPSRQEPS |
| | QGTTTFAVTSKLTVDKSRWQQGNVFSCSVMHEALHNHYTQKTISLSP |
| | GK |
| | (SEQ ID NO: 259) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |
| 69A02 (1B)(2) | First Heavy Chain: |
| VH-VL scFv | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPREPQVY |
| | TLPPPSEELALNELVTLTCLVKGFYPSDIAVEWLQGSQELPREKYLTW |
| | APVLDSDGSFFLYSILRVAAEDWKKGDTFSCSVMHEALHNHYTQKSL |
| | DRSPGKGGGGSGGGGSGGGGSGGGGSEVQLVESGGGLVQPGGSLRLS |
| | CAASGFTFNTYAMNWVRQAPGKGLEWVARIRSKYNNYATYYADSV |
| | KDRFTISRDDSKNTLYLQMNSLRAEDTAVYYCVRHGNFGNSYVSWF |
| | AYWGQGTMVTVSSASTKGPSVFPLAPSSKSTSGGTAALGCLVKDYFP |
| | EPVTVSWNSGALTSGVHTFPAVLQSSGLYSLSSVVTVPSSSLGTQTYIC |
| | NVNHKPSNTKVDKKVEPKSC |
| | (SEQ ID NO: 260) |
| | Second Heavy Chain: |
| | QVTLKESGPTLVKPTQTLTLTCSFSGFSITTSGVSVGWIRQPPGKALEW |
| | LALIYWDDDKRYSPSLKSRLTITRDTSKNQVVLTMTNMDPVDTATYY |
| | CAHAPRYTPGGYFDYWGQGTLVTVSSGGGGSGGGGSGGGGSGGGGS |
| | ETTLTQSPATLSVSPGETATLSCRASQSVSSNLAWYQQKPGQAPRLLIY |
| | GASTRATGIPATFSGSGSGTEFTLTISSLQSEDFAIYYCQQYKNWPPAF |
| | GGGTELEIKEPKSSDKTHTCPPCPAPEAAGGPSVFLFPPKPKDTLMISRT |
| | PEVTCVVVDVSHEDPEVKFNWYVDGVEVHNAKTKPREEQYNSTYRV |
| | VSVLTVLHQDWLNGKEYKCKVSNKALGAPIEKTISKAKGQPFRPEVH |
| | LLPPSREEMTKNQVSLTCLARGFYPKDIAVEWESNGQPENNYKTTPSR |
| | QEPSQGTTTFAVTSKLTVDKSRWQQGNVFSCSVMHEALHNHYTQKTI |
| | SLSPGK |
| | (SEQ ID NO: 261) |
| | Light Chain: |
| | QAVVTQEPSLTVSPGGTVTLTCRSSTGAVTTSNYANWVQQKPGQAPR |
| | GLIGGTNKRAPGTPARFSGSLLGGKAALTLSGVQPEDEAEYYCALWY |
| | SNLWVFGGGTKLTVLGQPKAAPSVTLFPPSSEELQANKATLVCLISDF |
| | YPGAVTVAWKADSSPVKAGVETTTPSKQSNNKYAASSYLSLTPEQW |
| | KSHRSYSCQVTHEGSTVEKTVAPTECS |
| | (SEQ ID NO: 84) |

Also provided herein is a use of a first or second heavy chain comprising a sequence selected from any of the first or second heavy chain amino acid sequences listed in TABLE 11, respectively, in the preparation of a bispecific protein wherein the first heavy chain or second heavy chain is associated: (i) with a light chain selected from any of the light chain amino acid sequences listed in TABLE 11 (e.g., via a disulfide bond); and (ii) a second heavy chain, or first heavy chain comprising a sequence selected from any of the second or first heavy chain amino acid sequences listed in TABLE 11. Additionally provided herein is a use of a nucleic acid that encodes a first or second heavy chain comprising a sequence selected from any of the first or second heavy chain amino acid sequences listed in TABLE 11, respectively, in the preparation of a bispecific protein wherein the first heavy chain or second heavy chain is associated: (i) with a light chain selected from any of the light chain amino acid sequences listed in TABLE 11 (e.g., via a disulfide bond); and (ii) a second heavy chain, or first heavy chain comprising a sequence selected from any of the second or first heavy chain amino acid sequences listed in TABLE 11. Nucleic acids encoding the bispecific proteins disclosed herein may be codon optimized for optimal expression using standard bioinformatic methods. Cells comprising one or more nucleic acid encoding a bispecific protein of the present invention are also contemplated and can be produced by a standard transfection or transduction method (e.g., electroporation, calcium chloride transfection, lipofection, lentiviral deliver, or adeno-associated virus delivery).

### I.F Characteristics of the Proteins

In some embodiments, a bispecific protein of the present disclosure, which induces robust T cell cytotoxicity against certain TAA expressing cells but does not induce a significant amount of cytokine release, includes two arms, the first arm comprises a first component, which specifically binds to a TAA, at its N-terminal end, and a second component, which specifically binds to the same TAA, at its C-terminal end, and a second arm comprises a component, which specifically binds to CD3, at its N-terminal end. In some embodiments, the present disclosure provides bispecific proteins that are capable of specifically binding or binds to ROR1 and CD3.

In some embodiments, bispecific proteins of the present disclosure specifically bind the Ig-like domain of ROR1. In some embodiments, bispecific proteins of the present disclosure do not bind the Frizzled and/or Kringle domain of ROR1. In some embodiments, bispecific proteins of the present invention specifically bind the CD3ε (SEQ ID NO: 269), CD3γ (SEQ ID NO: 270), CD3δ (SEQ ID NO: 271), or CD3ξ (SEQ ID NO: 96) polypeptide of CD3. For example, in certain embodiments, bispecific proteins of the present invention specifically bind the CD3ε polypeptide of CD3.

In some embodiments, bispecific proteins of the present invention bind to both a TAA and CD3 with high affinity as measured by any one of a variety of assays known in the field (for example, binding assays as described in EXAMPLE 2). In some embodiments, bispecific proteins of the present invention bind to both ROR1 and CD3 with high affinity as measured by any one of a variety of assays known in the field (for example, binding assays as described in EXAMPLE 2). In some embodiments, bispecific proteins of the present invention have a K_{D} for ROR1 binding of 1 nM to 10 nM, for example, 2 nM to 8 nM, 3 nM to 5 nM, or 4 nM to 6 nM, as measured in a biolayer interferometry assay. For example, in some embodiments, bispecific proteins of the present invention have a K_{D} for ROR1 binding of 3 nM to 5 nM (e.g., 3.0 nM, 3.2 nM, 3.4 nM, 3.6 nM, 3.8 nM, 4.0 nM, 4.2 nM, 4.4 nM, 4.6 nM, 4.8 nM, or 5.0 nM) as measured in a biolayer interferometry assay. In some embodiments, bispecific proteins of the present invention have a K_{D} for CD3 binding of 1 nM to 10 nM (e.g., 1 nM, 2 nM, 3 nM, 4 nM, 5 nM, 6 nM, 7 nM, 8 nM, 9 nM, or 10 nM) as measured in a biolayer interferometry assay. For example, in some embodiments, bispecific proteins of the present invention have a K_{D} for CD3 binding of 4.0 nM to 6.0 nM (e.g., 4.0 nM, 4.2 nM, 4.4 nM, 4.6 nM, 4.8 nM, 5.0 nM, 5.2 nM, 5.4 nM, 5.6 nM, 5.8 nM, or 6.0 nM) as measured in a biolayer interferometry assay.

In some embodiments, bispecific proteins of the present invention bind to a TAA with low affinity and CD3 with high affinity as measured by cell binding assays (for example, cell binding assays as described in EXAMPLE 2). In some embodiments, bispecific proteins of the present invention bind to ROR1 with low affinity and CD3 with high affinity as measured by cell binding assays (for example, cell binding assays as described in EXAMPLE 2). In some embodiments, bispecific proteins of the present invention have a K_{D} for ROR1 binding of 75 nM to 150 nM, for example, 80 nM to 120 nM, 85 nM to 115 nM, 90 nM to 110 nM, or 95 to 105 nM, as measured in a biolayer interferometry assay. In some embodiments, bispecific proteins of the present invention have a K_{D} for CD3 binding of 1 nM to 10 nM (e.g., 1 nM, 2 nM, 3 nM, 4 nM, 5 nM, 6 nM, 7 nM, 8 nM, 9 nM, or 10 nM) as measured in a biolayer interferometry assay. For example, in some embodiments, bispecific proteins of the present invention have a K_{D} for CD3 binding of 4.0 nM to 6.0 nM (e.g., 4.0 nM, 4.2 nM, 4.4 nM, 4.6 nM, 4.8 nM, 5.0 nM, 5.2 nM, 5.4 nM, 5.6 nM, 5.8 nM, or 6.0 nM) as measured in a biolayer interferometry assay.

In some embodiments, bispecific proteins of the present invention bind to a TAA with high affinity and CD3 with low affinity as measured by cell binding assays (for example, cell binding assays as described in EXAMPLE 2). In some embodiments, bispecific proteins of the present invention bind to ROR1 with high affinity and CD3 with low affinity as measured by cell binding assays (for example, cell binding assays as described in EXAMPLE 2). In some embodiments, bispecific proteins of the present invention have a K_{D} for ROR1 binding of 1 nM to 10 nM, for example, 2 nM to 8 nM, 3 nM to 5 nM, or 4 nM to 6 nM, as measured in a biolayer interferometry assay. For example, in some embodiments, bispecific proteins of the present invention have a K_{D} for ROR1 binding of 3 nM to 5 nM (e.g., 3.0 nM, 3.2 nM, 3.4 nM, 3.6 nM, 3.8 nM, 4.0 nM, 4.2 nM, 4.4 nM, 4.6 nM, 4.8 nM, or 5.0 nM) as measured in a biolayer interferometry assay. In some embodiments, bispecific proteins of the present invention have a K_{D} for CD3 binding of 10 nM to 100 nM, for example, 10 nM to 80 nM, 10 nM to 60 nM, 10 nM to 50 nM, 10 nM to 40 nM, 20 nM to 60 nM, 30 nM to 50 nM, 35 nM to 45 nM, 15 nM to 80 nM, 15 nM to 60 nM, 15 nM to 40 nM, or 15 nM to 20 nM as measured in a biolayer interferometry assay.

In some embodiments, bispecific proteins of the present invention bind to a TAA with low affinity and CD3 with low affinity as measured by cell binding assays (for example, cell binding assay as described in EXAMPLE 2). In some embodiments, bispecific proteins of the present invention bind to ROR1 with low affinity and CD3 with low affinity as measured by cell binding assays (for example, cell binding assay as described in EXAMPLE 2). In some embodiments, bispecific proteins of the present invention have a K_{D} for ROR1 binding of 75 nM to 150 nM, for example, 80 nM to 120 nM, 85 nM to 115 nM, 90 nM to 110 nM, or 95 to 105 nM, as measured in a biolayer interferometry assay. In some embodiments, bispecific proteins of the present invention have a K_{D} for CD3 binding of 10 nM to 100 nM, for example, 10 nM to 80 nM, 10 nM to 60 nM, 10 nM to 50 nM, 10 nM to 40 nM, 20 nM to 60 nM, 30 nM to 50 nM, 35 nM to 45 nM, 15 nM to 80 nM, 15 nM to 60 nM, 15 nM to 40 nM, or 15 nM to 20 as measured in a biolayer interferometry assay.

In some embodiments, bispecific proteins of the present invention bind to both a TAA and CD3 with high affinity as measured in flow cytometry-based binding assays (for example, flow cytometry-based binding assays as described in EXAMPLE 2). In some embodiments, bispecific proteins of the present invention bind to both ROR1 and CD3 with high affinity as measured in flow cytometry-based binding assays (for example, flow cytometry-based binding assays as described in EXAMPLE 2). In some embodiments, bispecific proteins of the present invention have an EC₅₀ for ROR1 binding of 0.1 nM to 10 nM, for example, 0.1 nM to 1 nM, 0.2 nM to 8 nM, 0.4 nM to 6 nM, 0.6 nM to 4 nM, 0.8 nM to 2 nM, 0.1 nM to 1 nM, 0.2 nM to 1 nM, 0.4 nM to 1 nM, 0.6 nM to 1 nM, 0.8 nM to 1 nM, 1 nM to 10 nM, 2 nM to 10 nM, 6 nM to 10 nM, 8 nM to 10 nM, 2 nM to 4 nM, 2 nM to 5 nM, 2 nM to 6 nM, 2 nM to 8 nM, or 2 nM to 10 nM as measured in a flow cytometry-based ROR1 binding assay. For example, in some embodiments bispecific proteins of the present invention have an EC₅₀ for ROR1 binding of 0.1 nM to 1 nM (e.g., 0.1 nM, 0.2 nM, 0.3 nM, 0.4 nM, 0.5 nM, 0.6 nM, 0.7 nM, 0.8 nM, 0.9 nM, or 1 nM) as measured in a flow cytometry-based ROR1 binding assay using cells expressing about 3.5×10⁴ ROR1 molecules/cell. In some embodiments bispecific proteins of the present invention have an EC₅₀ for ROR1 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based ROR1 binding assay using cells expressing about 3.5×10⁴ ROR1 molecules/cell. In some embodiments, bispecific proteins of the present invention have an EC₅₀ for ROR1 binding of 2 nM to 5 nM (e.g., 2 nM, 3 nM, 4 nM, or 5 nM) as measured in a flow cytometry-based ROR1 binding assay using cells expressing about 3.5×10⁴ ROR1 molecules/cell. In some embodiments, bispecific proteins of the present invention have an EC₅₀ for CD3 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based CD3 binding assay using activated T cells (for example, a CD3 binding assay as described in EXAMPLE 2). For example, in some embodiments, bispecific proteins of the present invention have an EC₅₀ for CD3 binding of 2 nM to 4 nM (e.g., 2.0 nM, 2.2 nM, 2.4 nM, 2.6 nM, 2.8 nM, 3.0 nM, 3.2 nM, 3.4 nM, 3.6 nM, 3.8 nM, or 4.0 nM) as measured in a flow cytometry-based CD3 binding assay using activated T cells.

In some embodiments, bispecific proteins of the present invention bind to a TAA with high affinity and CD3 with low affinity as measured in flow cytometry-based binding assays (for example, flow cytometry-based binding assays as described in EXAMPLE 2). In some embodiments, bispecific proteins of the present invention bind to ROR1 with high affinity and CD3 with low affinity as measured in flow cytometry-based binding assays (for example, flow cytometry-based binding assays as described in EXAMPLE 2). In some embodiments, bispecific proteins of the present invention have an EC₅₀ for ROR1 binding of 0.1 nM to 10 nM, for example, 0.1 nM to 1 nM, 0.2 nM to 8 nM, 0.4 nM to 6 nM, 0.6 nM to 4 nM, 0.8 nM to 2 nM, 0.1 nM to 1 nM, 0.2 nM to 1 nM, 0.4 nM to 1 nM, 0.6 nM to 1 nM, 0.8 nM to 1 nM, 1 nM to 10 nM, 2 nM to 10 nM, 6 nM to 10 nM, 8 nM to 10 nM, 2 nM to 4 nM, 2 nM to 5 nM, 2 nM to 6 nM, 2 nM to 8 nM, or 2 nM to 10 nM as measured in a flow cytometry-based ROR1 binding assay. For example, in some embodiments bispecific proteins of the present invention have an EC₅₀ for ROR1 binding of 0.1 nM to 1 nM (e.g., 0.1 nM, 0.2 nM, 0.3 nM, 0.4 nM, 0.5 nM, 0.6 nM, 0.7 nM, 0.8 nM, 0.9 nM, or 1 nM) as measured in a flow cytometry-based ROR1 binding assay using cells expressing about 3.5×10⁴ ROR1 molecules/cell. In some embodiments bispecific proteins of the present invention have an EC₅₀ for ROR1 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based ROR1 binding assay using cells expressing about 3.5×10⁴ ROR1 molecules/cell. In some embodiments, bispecific proteins of the present invention have an EC₅₀ for ROR1 binding of 2 nM to 5 nM (e.g., 2 nM, 3 nM, 4 nM, or 5 nM) as measured in a flow cytometry-based ROR1 binding assay using cells expressing about 3.5×10⁴ ROR1 molecules/cell. In some embodiments, bispecific proteins of the present invention have an EC₅₀ for CD3 binding of 10 nM to 100 nM, 10 nM to 90 nM, 10 nM to 80 nM, 10 nM to 70 nM, 10 nM to 60 nM, 10 nM to 50 nM, 10 nM to 40 nM, 10 nM to 30 nM, 10 nM to 20 nM, 20 nM to 100 nM, 20 nM to 90 nM, 20 nM to 80 nM, 20 nM to 70 nM, 20 nM to 60 nM, 20 nM to 50 nM, 25 nM to 75 nM, 25 nM to 65 nM, 25 nM to 55 nM, 25 nM to 45 nM, or 25 nM to 35 nM as measured in a flow cytometry-based CD3 binding assay using activated T cells.

In some embodiments, bispecific proteins of the present invention bind to a TAA with low affinity and CD3 with high affinity as measured in flow cytometry-based binding assays (for example, flow cytometry-based binding assays as described in EXAMPLE 2). In some embodiments, bispecific proteins of the present invention bind to ROR1 with low affinity and CD3 with high affinity as measured in flow cytometry-based binding assays (for example, flow cytometry-based binding assays as described in EXAMPLE 2). In some embodiments, bispecific proteins of the present invention have an EC₅₀ for ROR1 binding of 50 nM to 220 nM, 60 nM to 220 nM, 70 nM to 220 nM, 80 nM to 220 nM, 90 nM to 220 nM, 100 nM to 220 nM, 110 nM to 220 nM, 120 nM to 220 nM, 130 nM to 220 nM, 140 nM to 220 nM, 150 nM to 220 nM, 175 nM to 220 nM, 200 nM to 220 nM, 50 nM to 200 nM, 60 nM to 200 nM, 70 nM to 200 nM, 80 nM to 200 nM, 90 nM to 200 nM, 100 nM to 200 nM, 110 nM to 200 nM, 120 nM to 200 nM, 130 nM to 200 nM, 140 nM to 200 nM, 150 nM to 200 nM, 50 nM to 180 nM, 60 nM to 180 nM, 70 nM to 180 nM, 80 nM to 180 nM, 90 nM to 180 nM, 100 nM to 180 nM, 110 nM to 180 nM, 120 nM to 180 nM, 130 nM to 180 nM, 140 nM to 180 nM, 150 nM to 180 nM, 50 nM to 160 nM, 60 nM to 160 nM, 70 nM to 160 nM, 80 nM to 160 nM, 90 nM to 160 nM, 100 nM to 160 nM, 110 nM to 160 nM, 120 nM to 160 nM, 130 nM to 160 nM, 140 nM to 160 nM, 150 nM to 160 nM, 50 nM to 140 nM, 60 nM to 140 nM, 70 nM to 140 nM, 80 nM to 140 nM, 90 nM to 140 nM, 100 nM to 140 nM, 110 nM to 140 nM, 120 nM to 140 nM, 130 nM to 140 nM, 50 nM to 120 nM, 60 nM to 120 nM, 70 nM to 120 nM, 80 nM to 120 nM, 90 nM to 120 nM, 100 nM to 120 nM, 110 nM to 120 nM, 50 nM to 110 nM, 60 nM to 110 nM, 70 nM to 110 nM, 80 nM to 110 nM, 90 nM to 110 nM, 100 nM to 110 nM, 50 nM to 100 nM, 60 nM to 100 nM, 70 nM to 100 nM, 80 nM to 100 nM, or 90 nM to 100 nM as measured in a flow cytometry-based ROR1 binding assay. For example, in some embodiments, bispecific proteins of the present invention have an EC₅₀ for ROR1 binding of 80 nM to 120 nM or 90 nM to 110 nM as measured in a flow cytometry-based ROR1 binding assay using cells expressing about 3.5×10⁴ ROR1 molecules/cell. In some embodiments bispecific proteins of the present invention have an EC₅₀ for ROR1 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based ROR1 binding assay using cells expressing about 3.5×10⁴ ROR1 molecules/cell. In some embodiments, bispecific proteins of the present invention have an EC₅₀ for ROR1 binding of 2 nM to 5 nM (e.g., 2 nM, 3 nM, 4 nM, or 5 nM) as measured in a flow cytometry-based ROR1 binding assay using cells expressing about 3.5×10⁴ ROR1 molecules/cell. In some embodiments, bispecific proteins of the present invention have an EC₅₀ for CD3 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based CD3 binding assay using activated T cells (for example, a CD3 binding assay as described in EXAMPLE 2). For example, in some embodiments, bispecific proteins of the present invention have an EC₅₀ for CD3 binding of 2 nM to 4 nM (e.g., 2.0 nM, 2.2 nM, 2.4 nM, 2.6 nM, 2.8 nM, 3.0 nM, 3.2 nM, 3.4 nM, 3.6 nM, 3.8 nM, or 4.0 nM) as measured in a flow cytometry-based CD3 binding assay using activated T cells.

In some embodiments, bispecific proteins of the present invention bind to a TAA with low affinity and CD3 with high affinity as measured in flow cytometry-based binding assays (for example, flow cytometry-based binding assays as described in EXAMPLE 2). In some embodiments, bispecific proteins of the present invention bind to ROR1 with low affinity and CD3 with high affinity as measured in flow cytometry-based binding assays (for example, flow cytometry-based binding assays as described in EXAMPLE 2). In some embodiments, bispecific proteins of the present invention have an EC₅₀ for ROR1 binding of 50 nM to 220 nM, 60 nM to 220 nM, 70 nM to 220 nM, 80 nM to 220 nM, 90 nM to 220 nM, 100 nM to 220 nM, 110 nM to 220 nM, 120 nM to 220 nM, 130 nM to 220 nM, 140 nM to 220 nM, 150 nM to 220 nM, 175 nM to 220 nM, 200 nM to 220 nM, 50 nM to 200 nM, 60 nM to 200 nM, 70 nM to 200 nM, 80 nM to 200 nM, 90 nM to 200 nM, 100 nM to 200 nM, 110 nM to 200 nM, 120 nM to 200 nM, 130 nM to 200 nM, 140 nM to 200 nM, 150 nM to 200 nM, 50 nM to 180 nM, 60 nM to 180 nM, 70 nM to 180 nM, 80 nM to 180 nM, 90 nM to 180 nM, 100 nM to 180 nM, 110 nM to 180 nM, 120 nM to 180 nM, 130 nM to 180 nM, 140 nM to 180 nM, 150 nM to 180 nM, 50 nM to 160 nM, 60 nM to 160 nM, 70 nM to 160 nM, 80 nM to 160 nM, 90 nM to 160 nM, 100 nM to 160 nM, 110 nM to 160 nM, 120 nM to 160 nM, 130 nM to 160 nM, 140 nM to 160 nM, 150 nM to 160 nM, 50 nM to 140 nM, 60 nM to 140 nM, 70 nM to 140 nM, 80 nM to 140 nM, 90 nM to 140 nM, 100 nM to 140 nM, 110 nM to 140 nM, 120 nM to 140 nM, 130 nM to 140 nM, 50 nM to 120 nM, 60 nM to 120 nM, 70 nM to 120 nM, 80 nM to 120 nM, 90 nM to 120 nM, 100 nM to 120 nM, 110 nM to 120 nM, 50 nM to 110 nM, 60 nM to 110 nM, 70 nM to 110 nM, 80 nM to 110 nM, 90 nM to 110 nM, 100 nM to 110 nM, 50 nM to 100 nM, 60 nM to 100 nM, 70 nM to 100 nM, 80 nM to 100 nM, or 90 nM to 100 nM as measured in a flow cytometry-based ROR1 binding assay. For example, in some embodiments, bispecific proteins of the present invention have an EC₅₀ for ROR1 binding of 80 nM to 120 nM or 90 nM to 110 nM as measured in a flow cytometry-based ROR1 binding assay using cells expressing about 3.5×10⁴ ROR1 molecules/cell. In some embodiments bispecific proteins of the present invention have an EC₅₀ for ROR1 binding of 1 nM to 10 nM (e.g., 1 nM, 2 nM, 3 nM, 4 nM, 5 nM, 6 nM, 7 nM, 8 nM, 9 nM, or 10 nM) as measured in a flow cytometry-based ROR1 binding assay using cells expressing about 3.5×10⁴ ROR1 molecules/cell. In some embodiments, bispecific proteins of the present invention have an EC₅₀ for ROR1 binding of 2 nM to 5 nM (e.g., 2 nM, 3 nM, 4 nM, or 5 nM) as measured in a flow cytometry-based ROR1 binding assay using cells expressing about 3.5×10⁴ ROR1 molecules/cell. In some embodiments, bispecific proteins of the present invention have an EC₅₀ for CD3 binding of 10 nM to 100 nM, 10 nM to 90 nM, 10 nM to 80 nM, 10 nM to 70 nM, 10 nM to 60 nM, 10 nM to 50 nM, 10 nM to 40 nM, 10 nM to 30 nM, 10 nM to 20 nM, 20 nM to 100 nM, 20 nM to 90 nM, 20 nM to 80 nM, 20 nM to 70 nM, 20 nM to 60 nM, 20 nM to 50 nM, 25 nM to 75 nM, 25 nM to 65 nM, 25 nM to 55 nM, 25 nM to 45 nM, or 25 nM to 35 nM as measured in a flow cytometry-based CD3 binding assay using activated T cells.

Activation of T cells, e.g., cytotoxic CD8⁺ T cells, by binding of the CD3/TCR complex to its cognate peptide ligand presented on the major histocompatibility complex (MHC) molecules of target cells, induces degranulation of preformed lytic granules and release of cytotoxic enzymes into the immune synapse formed between the T cell and the target cell. In some embodiments, bispecific proteins of the present disclosure promote T cell lytic granule degranulation.

In some embodiments, bispecific proteins of the present disclosure induce T cell mediated cytotoxicity of TAA-expressing tumor cells. In some embodiments, bispecific proteins of the present disclosure induce T cell mediated cytotoxicity of ROR1-expressing tumor cells. For example, in some embodiments, a bispecific protein of the present disclosure has an EC₅₀ for T cell mediated cytotoxicity of ROR1-expressing tumor cells of 150 pM to 250 pM, 155 pM to 250 pM, 160 pM to 250 pM, 165 pM to 250 pM, 170 pM to 250 pM, 175 pM to 250 pM, 150 pM to 225 pM, 155 pM to 225 pM, 160 pM to 225 pM, 165 pM to 225 pM, 170 pM to 225 pM, 175 pM to 225 pM, 150 pM to 200 pM, 155 pM to 200 pM, 160 pM to 200 pM, 165 pM to 200 pM, 170 pM to 200 pM, 175 pM to 200 pM, 150 pM to 190 pM, 155 pM to 190 pM, 160 pM to 190 pM, 165 pM to 190 pM, 170 pM to 190 pM, or 175 pM to 190 pM, as measured in a T cell/tumor cell co-culture killing assay as described in EXAMPLE 3.

In other embodiments, a bispecific protein of the present disclosure has an EC₅₀ for T cell mediated cytotoxicity of ROR1-expressing tumor cells of 10 pM to 100 pM, 15 pM to 100 pM, 20 pM to 100 pM, 25 pM to 100 pM, 30 pM to 100 pM, 35 pM to 100 pM, 40 pM to 100 pM, 50 pM to 100 pM, 10 pM to 75 pM, 15 pM to 75 pM, 20 pM to 75 pM, 25 pM to 75 pM, 30 pM to 75 pM, 35 pM to 75 pM, 40 pM to 75 pM, 50 pM to 75 pM, 10 pM to 50 pM, 15 pM to 50 pM, 20 pM to 50 pM, 25 pM to 50 pM, 30 pM to 50 pM, 35 pM to 50 pM, 40 pM to 50 pM, 10 pM to 40 pM, 15 pM to 40 pM, 20 pM to 40 pM, 25 pM to 40 pM, 30 pM to 40 pM, or 35 pM to 40 pM, as measured in a T cell/tumor cell co-culture killing assay as described in EXAMPLE 3.

Activation of T cells, by binding of the CD3/TCR complex to its cognate peptide ligand presented on the major histocompatibility complex (MHC) molecules of target cells, can induce the release of proinflammatory cytokines such as, but not limited to, IFNγ, TNFα, and IL2. In some embodiments, bispecific proteins of the present disclosure do not induce the release of significant levels of proinflammatory cytokines from activated T cells as compared to a reference control molecule. For example, in some embodiments, bispecific proteins of the present disclosure have an EC₅₀ for IFNγ release of 1000 pM to 2000 pM, 1200 pM to 2000 pM, 1400 pM to 2000 pM, 1600 pM to 2000 pM, 1800 pM to 2000 pM, 1000 pM to 1800 pM, 1200 pM to 1800 pM, 1400 pM to 1800 pM, or 1600 pM to 1800 pM, as measured in a T cell/tumor cell co-culture IFNγ production assay as described in EXAMPLE 3.

In some embodiments, bispecific proteins of the present disclosure do not induce the release of significant levels of proinflammatory cytokines from activated T cells as compared to a reference control molecule. For example, in some embodiments, bispecific proteins of the present disclosure have an EC₅₀ for IFNγ release of 1000 pM to 2000 pM, 1200 pM to 2000 pM, 1400 pM to 2000 pM, 1600 pM to 2000 pM, 1800 pM to 2000 pM, 1000 pM to 1800 pM, 1200 pM to 1800 pM, 1400 pM to 1800 pM, or 1600 pM to 1800 pM, as measured in a T cell/tumor cell co-culture IFNγ production assay as described in EXAMPLE 3.

In other embodiments, bispecific proteins of the present disclosure have an EC₅₀ for IFNγ release of 100 pM to 1000 pM, 200 pM to 1000 pM, 300 pM to 1000 pM, 400 pM to 1000 pM, 500 pM to 1000 pM, 600 pM to 1000 pM, 700 pM to 1000 pM, 800 pM to 1000 pM, 900 pM to 1000 pM, 100 pM to 800 pM, 200 pM to 100 pM, 300 pM to 800 pM, 400 pM to 800 pM, 500 pM to 800 pM, 600 pM to 800 pM, 700 pM to 800 pM, 100 pM to 600 pM, 200 pM to 100 pM, 300 pM to 600 pM, 400 pM to 600 pM, 500 pM to 600 pM, 100 pM to 500 pM, 200 pM to 500 pM, 300 pM to 500 pM, or 400 pM to 500 pM, as measured in a T cell/tumor cell co-culture IFNγ production assay as described in EXAMPLE 3.

As used herein, “maximal concentration” or “Cₘₐₓ” refers to the maximal concentration of a cytokine that can be released from a cell. In some embodiments, bispecific proteins of the present disclosure can induce a Cₘₐₓ of 10% to 80%, 20% to 60%, 35% to 45%, 45% to 55%, or 35% to 75% compared to a reference T cell-binder as measured in a T cell/tumor cell co-culture IFNγ production assay. For example, a reference T cell-binder can include, but is not limited to, a bispecific protein comprising four polypeptides having amino acid sequences of SEQ ID NO: 302, SEQ ID NO: 303, SEQ ID NO: 304, and SEQ ID NO: 305.

Reference T Cell Binder Polypeptide 1:
(SEQ ID NO: 302)
MGWSCIILFLVATATGVHSELVLTQSPSVSAALGSPAKITCTLSSAHKT
DTIDWYQQLQGEAPRYLMQVQSDGSYTKRPGVPDRFSGSSSGADRY
LIIPSVQADDEADYYCGADYIGGYVFGGGTQLTVLGQPKAAPSVTLFP
PSSEELQANKATLVCLISDFYPGAVTVAWKADSSPVKAGVETTTPSK
QSNNKYAASSYLSLTPEQWKSHKSYSCQVTHEGSTVEKTVAPTECS
Reference T Cell Binder Polypeptide 2:
(SEQ ID NO: 303)
QEQLVESGGRLVTPGGSLTLSCKASGFDFSAYYMSWVRQAPGKGLE
WIATIYPSSGKTYYATWVNGRFTISSDNAQNTVDLQMNSLTAADRAT
YFCARDSYADDGALFNIWGPGTLVTISSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDGGGGSGGGGSE
VQLLESGGGLVQPGGSLRLSCAASGFTFSTYAMNWVRQAPGKGLEW
VSRIRSKYNNYATYYADSVKGRFTISRDDSKNTLYLQMNSLRAEDTA
VYYCVRHGNFGNSYVSWFAYWGQGTLVTVSSASVAAPSVFIFPPSDE
QLKSGTASVVCLLNNFYPREAKVQWKVDNALQSGNSQESVTEQDSK
DSTYSLSSTLTLSKADYEKHKVYACEVTHQGLSSPVTKSFNRGECDK
THTCPPCPAPEAAGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALGAPIEKTISKAKGQPREPQVYTLPPCRDELTKNQ
VSLWCLVKGFYPSDIAVEWESNGQPENNYKTTPPVLDSDGSFFLYSK
LTVDKSRWQQGNVFSCSVMHEALHNHYTQKSLSLSPGK
Reference T Cell Binder Polypeptide 3:
(SEQ ID NO: 304)
QEQLVESGGRLVTPGGSLTLSCKASGFDFSAYYMSWVRQAPGKGLE
WIATIYPSSGKTYYATWVNGRFTISSDNAQNTVDLQMNSLTAADRAT
YFCARDSYADDGALFNIWGPGTLVTISSASTKGPSVFPLAPSSKSTSGG
TAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVD
GVEVHNAKTKPREEQYNSTYRVVSVLTVLHQDWLNGKEYKCKVSN
KALGAPIEKTISKAKGQPREPQVCTLPPSRDELTKNQVSLSCAVKGFY
PSDIA VEWESNGQPENNYKTTPPVLDSDGSFFLVSKLTVDKSRWQQG
NVFSCSVMHEALHNHYTQKSLSLSPGK
Reference T Cell Binder Polypeptide 4:
(SEQ ID NO: 305)
QAVVTQEPSLTVSPGGTVTLTCGSSTGAVTTSNYANWVQEKPGQAFR
GLIGGTNKRAPGTPARFSGSLLGGKAALTLSGAQPEDEAEYYCALWY
SNLWVFGGGTKLTVLSSASTKGPSVFPLAPSSKSTSGGTAALGCLVKD
YFPEPVTVSWNSGALTSGVHTFPAVLQSSGLYSLSSVVTVPSSSLGTQ
TYICNVNHKPSNTKVDKKVEPKSC

In some embodiments, bispecific proteins of the present disclosure do not induce significant levels of TNFα release when administered to freshly isolated PBMC cultures as compared to an anti-CD3 reference antibody. In some embodiments, bispecific proteins of the present disclosure do not induce significant levels of TNFα release when administered to PBMCs cultured for 48 hours at high density (e.g., 1.0×10⁷ cells/mL) as compared to an anti-CD3 reference antibody.

In some embodiments, bispecific proteins of the present disclosure do not induce significant levels of IL2 release when administered to freshly isolated PBMC cultures as compared to an anti-CD3 reference antibody. In some embodiments, bispecific proteins of the present disclosure do not induce significant levels of IL2 release when administered to PBMCs cultured for 48 hours at high density (e.g., 1.0×10⁷ cells/mL) as compared to an anti-CD3 reference antibody.

As used herein, a “cytokine release to killing decoupling ratio” for a given bispecific protein refers to the ratio between the area under the curve (AUC) for induction of IFNγ release (as measured in a T cell/tumor cell co-culture IFNγ production assay as described in EXAMPLE 3) and the AUC for cytotoxic killing (as measured in a T cell/tumor cell co-culture killing assay as described in EXAMPLE 3). In some embodiments, bispecific proteins of the present disclosure have a cytokine release to killing decoupling ratio of 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, or 1:5, normalized to a reference T cell-binder as measured in a T cell/tumor cell co-culture killing assay and IFNγ production assay. For example, in some embodiments, bispecific proteins of the present disclosure have a cytokine release to killing decoupling ratio of 1:2 to 1:4 normalized to a reference T cell-binder as measured in a T cell/tumor cell co-culture killing assay and IFNγ production assay. In certain embodiments, bispecific proteins of the present disclosure have a cytokine release to killing decoupling ratio of 1:2.2 normalized to a reference T cell-binder as measured in a T cell/tumor cell co-culture killing assay and IFNγ production assay. In certain embodiments, bispecific proteins of the present disclosure have a cytokine release to killing decoupling ratio of 1:2.9 normalized to a reference T cell-binder as measured in a T cell/tumor cell co-culture killing assay and IFNγ production assay.

As used herein, the term “serial killing index” or “tumor serial killing index” refers to the average number of tumor cells killed by a single CD8⁺ T cell. In some embodiments, bispecific proteins of the present disclosure induce a tumor serial killing index of 2 to 6. For example, T cells treated with bispecific proteins of the present disclosure have a serial killing index of 2.2 to 5.5, 2.4 to 5.5, 2.6 to 5.5, 2.8 to 5.5, 3.0 to 5.5, 3.2 to 5.5, 3.4 to 5.5, 3.6 to 5.5, 3.8 to 5.5, 4.0 to 5.5, 2.2 to 5.0, 2.4 to 5.0, 2.6 to 5.0, 2.8 to 5.0, 3.0 to 5.0, 3.2 to 5.0, 3.4 to 5.0, 3.6 to 5.0, 3.8 to 5.0, 4.0 to 5.0, 2.2 to 4.8, 2.4 to 4.8, 2.6 to 4.8, 2.8 to 4.8, 3.0 to 4.8, 3.2 to 4.8, 3.4 to 4.8, 3.6 to 4.8, 3.8 to 4.8, 4.0 to 4.8, 2.2 to 4.6, 2.4 to 4.6, 2.6 to 4.6, 2.8 to 4.6, 3.0 to 4.6, 3.2 to 4.6, 3.4 to 4.6, 3.6 to 4.6, 3.8 to 4.6, 4.0 to 4.6, 2.2 to 4.4, 2.4 to 4.4, 2.6 to 4.4, 2.8 to 4.4, 3.0 to 4.4, 3.2 to 4.4, 3.4 to 4.4, 3.6 to 4.4, 3.8 to 4.4, 4.0 to 4.4, 2.2 to 4.2, 2.4 to 4.2, 2.6 to 4.2, 2.8 to 4.2, 3.0 to 4.2, 3.2 to 4.2, 3.4 to 4.2, 3.6 to 4.2, 3.8 to 4.2, 4.0 to 4.2, 2.2 to 4.0, 2.4 to 4.0, 2.6 to 4.0, 2.8 to 4.0, 3.0 to 4.0, 3.2 to 4.0, 3.4 to 4.0, 3.6 to 4.0, 3.8 to 4.0, 2.2 to 3.8, 2.4 to 3.8, 2.6 to 3.8, 2.8 to 3.8, 3.0 to 3.8, 3.2 to 3.8, 3.4 to 3.8, 3.6 to 3.8, 2.2 to 3.6, 2.4 to 3.6, 2.6 to 3.6, 2.8 to 3.6, 3.0 to 3.6, 3.2 to 3.6, 3.4 to 3.6, 2.2 to 3.4, 2.4 to 3.4, 2.6 to 3.4, 2.8 to 3.4, 3.0 to 3.4, or 3.2 to 3.4 as measured in a T cell/tumor cell co-culture killing assay. In certain embodiments, bispecific proteins of the present disclosure induce a tumor serial killing index of 3.4 to 3.6. In certain embodiments, bispecific proteins of the present disclosure induce a tumor serial killing index of 3.8 to 4.2.

In some embodiments, bispecific proteins of the present disclosure are cross-reactive with cynomolgus CD3 but not with mouse CD3. In some embodiments, bispecific proteins of the present disclosure are cross-reactive with cynomolgus ROR1 and mouse ROR1.

### II Antibodies

Also provided herein are antibodies that specifically bind ROR1. In some embodiments, an antibody that specifically binds ROR1 comprises a heavy chain variable domain (VH) and light chain variable domain (VL) selected from the sequences listed in TABLE 2.

In some embodiments, the heavy chain variable domain and the light chain variable domain of antibodies described herein comprise VH and VL CDR sequences selected from the VHCDR and VLCDR sequences listed in TABLE 1.

Unless indicated otherwise, the CDR sequences provided in TABLE 1 are determined under the IMGT unique numbering scheme

In some embodiments, antibodies of the present invention may be IgG, IgM, IgA, IgD, or IgE. In certain embodiments, antibodies described herein are IgG1, IgG1, IgG3, or IgG4. In certain embodiments, the antibodies of the present invention are human IgG1 antibodies.

In certain embodiments, antibodies that specifically bind to ROR1 comprise a VHCDR3 sequence selected from the sequence of SEQ ID NO: 10 or SEQ ID NO: 25. In certain embodiments, antibodies that specifically bind to ROR1 comprise a VLCDR3 sequence selected from the consensus sequence of SEQ ID NO: 13 or the sequence of SEQ ID NO: 28.

In some embodiments, antibodies that specifically binds to ROR1 comprise: a heavy chain variable complementarity-determining region 1 (VHCDR1) comprising an amino acid sequence of SEQ ID NO: 8 or SEQ ID NO: 23; a heavy chain variable complementarity-determining region 2 (VHCDR2) comprising an amino acid sequence of SEQ ID NO: 9, or SEQ ID NO: 24; and a heavy chain variable complementarity-determining region 3 (VHCDR3) comprising an amino acid sequence of: SEQ ID NO: 10 or SEQ ID NO: 25.

In some embodiments, antibodies that specifically bind to ROR1 comprise: a light chain variable complementarity-determining region 1 (VLCDR1) comprising an amino acid sequence of SEQ ID NO: 11, or SEQ ID NO: 26; a light chain variable complementarity-determining region 2 (VLCDR2) comprising an amino acid sequence DAS or GAS; and a light chain variable complementarity-determining region 3 (VLCDR3) comprising an amino acid sequence of SEQ ID NO: 14, SEQ ID NO: 15, or SEQ ID NO: 28.

In some embodiments, antibodies that specifically bind to ROR1 comprise: a VHCDR1 comprising an amino acid sequence of SEQ ID NO: 8; a VHCDR2 comprising an amino acid sequence of SEQ ID NO: 9; a VHCDR3 comprising an amino acid sequence of SEQ ID NO: 10; a VLCDR1 comprising an amino acid sequence of SEQ ID NO: 11; a VLCDR2 comprising an amino acid sequence of DAS; and a VLCDR3 comprising an amino acid sequence of SEQ ID NO: 14.

In some embodiments, antibodies that specifically bind ROR1 comprise: a VHCDR1 comprising an amino acid sequence of SEQ ID NO: 8; a VHCDR2 comprising an amino acid sequence of SEQ ID NO: 9; a VHCDR3 comprising an amino acid sequence of SEQ ID NO: 10; a VLCDR1 comprising an amino acid sequence of SEQ ID NO: 11; a VLCDR2 comprising an amino acid sequence of DAS; and a VLCDR3 comprising an amino acid sequence of SEQ ID NO: 15.

In some embodiments, antibodies that specifically bind ROR1 comprise: a VHCDR1 comprising an amino acid sequence of SEQ ID NO: 23; a VHCDR2 comprising an amino acid sequence of SEQ ID NO: 24; a VHCDR3 comprising an amino acid sequence of SEQ ID NO: 25; a VLCDR1 comprising an amino acid sequence of SEQ ID NO: 26; a VLCDR2 comprising an amino acid sequence of GAS; and a VLCDR3 comprising an amino acid sequence of SEQ ID NO: 28.

In some embodiments, antibodies that specifically bind to ROR1 comprise, according to the IMGT unique numbering scheme, VHCDR1, VHCDR2, VHCDR3, VLCDR1, VLCDR2, and VLCDR3 each comprising an amino acid sequence corresponding to the VHCDR1, VHCDR2, VHCDR3, VLCDR1, VLCDR2, and VLCDR3 sequences of a VHCDR and VLCDR consensus sequences of TABLE 1, respectively.

In certain embodiments, the antibody that specifically binds ROR1 comprises an antibody heavy chain variable domain (VH) that comprises an amino acid sequence at least 90% (e.g., at least 91%, at least 92%, at least 93%, at least 94%, at least 95%, at least 96%, at least 97%, at least 98%, at least 99%, or 100%) identical to the VH of an antibody disclosed in TABLE 2, and an antibody light chain variable domain (VL) that comprises an amino acid sequence at least 90% (e.g., at least 91%, at least 92%, at least 93%, at least 94%, at least 95%, at least 96%, at least 97%, at least 98%, at least 99%, or 100%) identical to the VL of the same antibody disclosed in TABLE 2. In certain embodiments, the antibody comprises the heavy chain CDR1, CDR2, and CDR3 and the light chain CDR1, CDR2, and CDR3, determined under IMGT unique numbering scheme, Kabat (see Kabat et al., (1991) Sequences of Proteins of Immunological Interest, NIH Publication No. 91-3242, Bethesda), Chothia (see, e.g., Chothia C & Lesk A M, (1987), J. Mol. Biol. 196: 901-917), MacCallum (see MacCallum R M et al., (1996) J. Mol. Biol. 262: 732-745), or any other CDR determination method known in the art, of the VH and VL sequences disclosed in TABLE 1.

In certain embodiments, the antibody that specifically binds ROR1 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 30.

In certain embodiments, the antibody that specifically binds ROR1 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 29, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 31.

In certain embodiments, the antibody that specifically binds ROR1 comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 39, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 40.

### III Therapeutic Applications

The present disclosure also provides pharmaceutical formulations that contain a therapeutically effective amount of a protein disclosed herein. The pharmaceutical formulation comprises one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

The present application provides methods for treating a cancer using a protein described herein and/or a pharmaceutical formulation described herein. The methods may be used to treat a variety of cancers including, but not limited to, chronic lymphocytic leukemia, mantle cell lymphoma, non-Hodgkin lymphoma, ovarian cancer, lung cancer, bronchial cancer, colon cancer, rectal cancer, melanoma, renal cancer, gastric cancer, pancreatic cancer, prostate cancer, uterine cancer, breast cancer, oral cancer, pharyngeal cancer, hairy cell leukemia, liver cancer, intrahepatic bile duct cancer, and thyroid cancer

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Reagent Preparation

This example describes the reagents, and screening assays that were used to generate the data of the present disclosure.

### 1.1 Luciferase Reporter Target Cells

Target cells were engineered to stably express firefly luciferase and eGFP via lentiviral transduction. Loss of luciferase activity upon addition of D-luciferin to a sample was used as an indication of cell killing.

### 1.2 Screening and Identification of ROR1-Binding Clones

Clone 69A02 was obtained from a naïve human scFv phage library panning on recombinant ROR1 protein, using standard techniques well known to persons of ordinary skill in the art. Counterselection was performed on His-tagged human ROR2 extracellular domain (ECD) and an irrelevant His-tagged negative target. Positive selection was carried out on His-tagged human ROR1 ECD ROR1 binders. Primary screening for human ROR1 vs. human ROR2 was followed by secondary screening for specific cellular binding to recombinantly overexpressed human ROR1. Subsequent ELISA domain binning applying coated human Fc-Ig (huFc-Ig) (SEQ ID NO: 134), human Fc-Ig-Frizzled (huFc-Ig-frz) (SEQ ID NO: 137), or human Fc-Frizzled-Kringle (huFc-frz-KNG) (SEQ ID NO: 138) fusion proteins revealed specificity of 69A02 towards the Ig-like domain of human ROR1. An additional sandwich ELISA-based Ig-binder binning (coating of binder A, pre-incubation binder B+human ROR1 ECD-His, detection of non-overlapping binding via anti-His-horseradish peroxidase (HRP)) indicated a unique epitope that did not overlap with further internal hit candidates.

Clone 82H02 was identified by applying a randomly paired Fab yeast surface display library derived from transgenic rats producing human variable antibody domains immunized with human ROR1 ECD-His protein. 82H02 was selected by gating on mouse and human ROR1 and in subsequent sorting rounds further sorted for huFc-Ig (SEQ ID NO: 134) but not huFc-frz-KNG (SEQ ID NO: 138) binding. Flow cytometry using recombinant cell lines confirmed specificity to human ROR1 without binding human ROR2. This was further confirmed using a standard ELISA assay.

huFc-Ig:
(SEQ ID NO: 134)
MKLPVRLLVLMFWIPASLSEPKSSDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGKQETELSVSAELVPTSSWNISSELNKDSYLTLDEPMNNITTSLG
QTAELHCKVSGNPPPTIRWFKNDAPVVQEPRRLSFRSTIYGSRLRIRNL
DTTDTGYFQCVATNGKEVVSSTGVLFVKFGPPPTASPGYSDEY
huFc-frz:
(SEQ ID NO: 137)
MKLPVRLLVLMFWIPASLSEPKSSDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGKQETELSVSAELVPTSSWNISSELNKDSYLTLDEPMNNITTSLG
QTAELHCKVSGNPPPTIRWFKNDAPVVQEPRRLSFRSTIYGSRLRIRNL
DTTDTGYFQCVATNGKEVVSSTGVLFVKFGPPPTASPGYSDEYEEDGFC
QPYRGIACARFIGNRTVYMESLHMQGEIENQITAAFTMIGTSSHLSDKC
SQFAIPSLCHYAFPYCDETSSVPKPRDLCRDECEILENVLCQTEYIFAR
SNPMILMRLKLPNCEDLPQPESPEAANCIRIGIPMADPINKNH
huFc-frz-KNG:
(SEQ ID NO: 138)
MKLPVRLLVLMFWIPASLSEPKSSDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGKVKFGPPPTASPGYSDEYEEDGFCQPYRGIACARFIGNRTVYME
SLHMQGEIENQITAAFTMIGTSSHLSDKCSQFAIPSLCHYAFPYCDETS
SVPKPRDLCRDECEILENVLCQTEYIFARSNPMILMRLKLPNCEDLPQP
ESPEAANCIRIGIPMADPINKNHKCYNSTGVDYRGTVSVTKSGRQCQPW
NSQYPHTHTFTALRFPELNGGHSYCRNPGNQKEAPWCFTLDENFKSDLC
DIPACDSKDSKEKNKMEILY

### Example 2: Cell Binding Assays

This example describes cell binding assays that were used to determine the binding affinities of bispecific TCEs of the present disclosure.

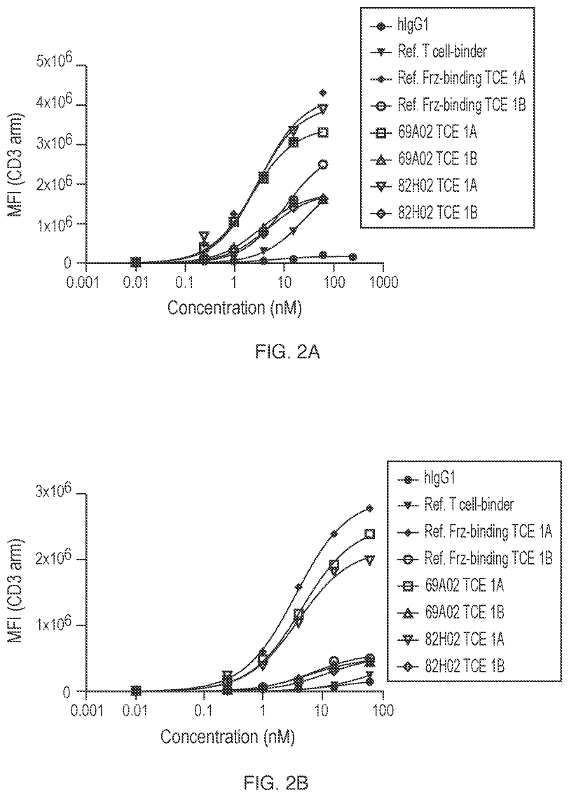

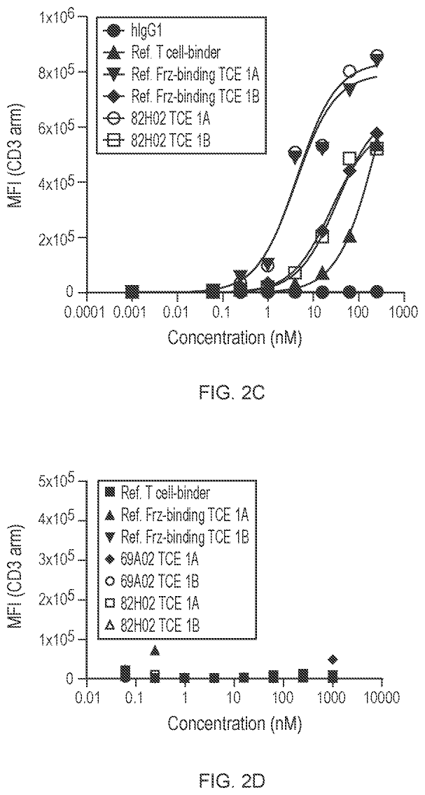

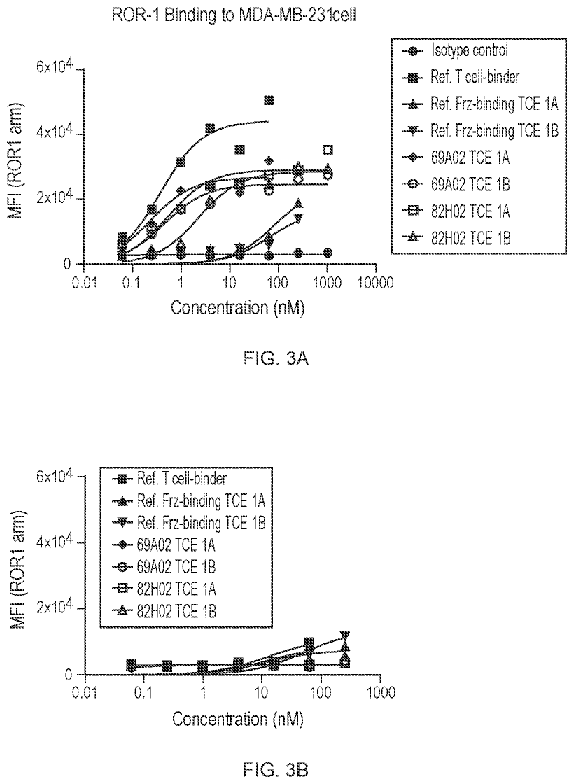

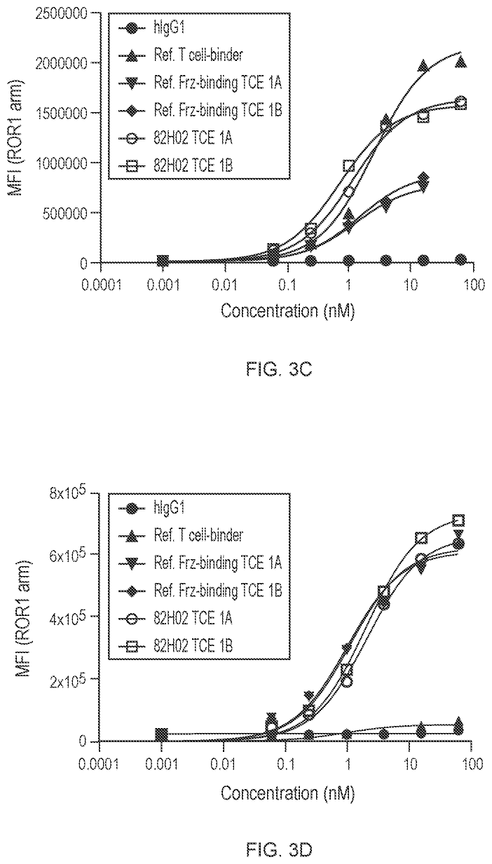

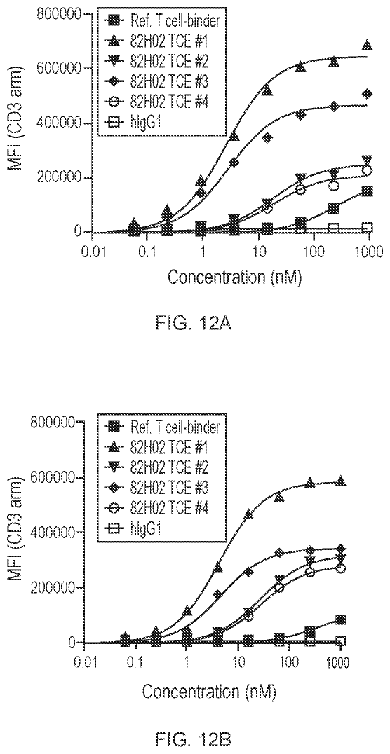

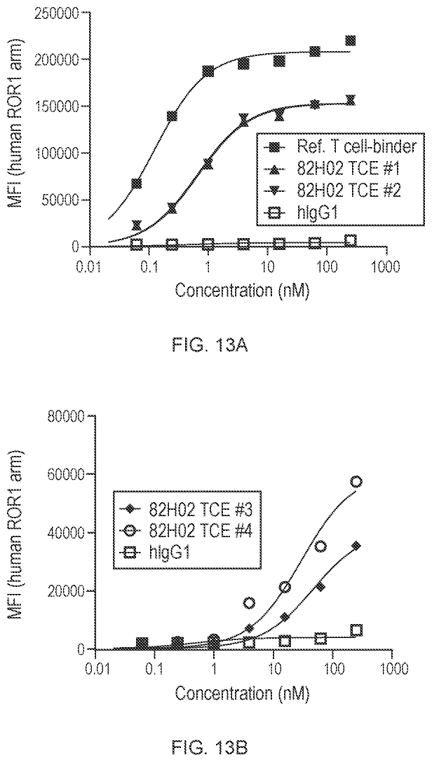

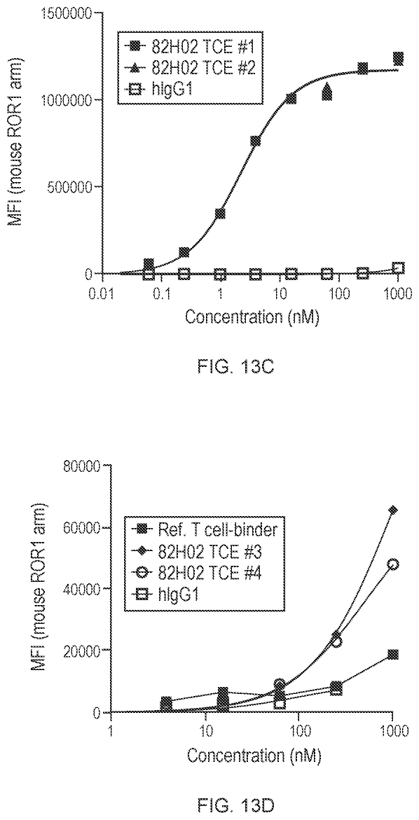

Target cells (e.g., human Jurkat-T cells (FIG. 2A); cynomolgus pan-T cells (FIG. 2B); C57BL6 human CD3ε knock-in mouse T cells (FIG. 2C); CD3ε knock-out human Jurkat-T cells (FIG. 2D); MDA-MB-231 cells (FIG. 3A); T-47D cells (FIG. 3B); human ROR1 expressing mouse 4T1 cells (FIG. 3C); mouse ROR1 expressing mouse 4T1 cells (FIG. 3D); human CD8⁺ T cells (FIG. 12A); cynomolgus pan-T cells (FIG. 12B); MDA-MB-231 cells (FIG. 13A and FIG. 13B); mouse ROR1 expressing mouse 4T1 cells (FIG. 13C and FIG. 13D) were added to V-bottom 96-well plates at 300,000 cells per well. After washing twice with 200 μL of phosphate buffered saline (PBS) by centrifugation at 500×g for 5 minutes at room temperature, the cells were resuspended in 100 μL of Live/Dead fixable green working buffer (ThermoFisher Scientific, Cat No: L23101; one vial of Live/Dead fixable green powder was dissolved into 50 μL of DMSO and 1 μL of dissolved Live/Dead fixable green was added to 1 mL of PBS to prepare the working buffer). After 15-minutes of incubation at room temperature, the cells were washed twice with 200 μL of PBS by centrifugation at 500×g for 5 minutes at room temperature and resuspended in 50 mL of 10% goat serum in BD staining buffer (BD Biosciences, Cat No: 554657). Next, the cells were incubated at room temperature for 20 minutes and 50 μL of a bispecific reference T cell-binder (Ref. T cell-binder)(comprising polypeptides having amino acid sequences of SEQ ID NO: 302, SEQ ID NO: 303, SEQ ID NO: 304, SEQ ID NO: 305), reference Frizzled-binding TCE (Ref Frz-binding TCE 1A or Ref Frz-binding TCE 1B), 69A02 TCE (69A02 TCE 1A or 69A02 TCE 1B) or 82H02 TCE (82H02 TCE 1A or 82H02 1B) (serial dilutions in BD staining buffer from 250 nM to 10 pM, as indicated in each figure) were added to the cells without washing. The plates were then covered with foil to avoid direct light and incubated for 60 minutes at 4° C. The cells were then washed twice with 200 μL of PBS by centrifugation at 500×g for 5 minutes at room temperature and resuspended in 100 μL of 3 μg/mL goat anti-human IgG-Alexa Fluor 647 antibody (Jackson ImmunoResearch Lab, Cat No: 109-606-003) in BD staining buffer. After incubating for 30 minutes at 4° C., the cells were washed twice with 200 μL of PBS by centrifugation at 500×g for 5 minutes at room temperature and resuspended into 100 μL of fixation buffer (BioLegend, Cat No: 420801). The samples were then analyzed on a flow cytometer for binding signal using FlowJo software. ROR and CD3 binding EC₅₀s (concentration vs mean fluorescence intensity (MFI)) for reference molecules and test bispecific TCEs were obtained using 4-parameter non-linear regression curve fitting in GraphPad Prism.

### 2.1 CD3 Binding Affinity

The apparent binding affinities for human or cynomolgus CD3 associated with a reference T cell-binder (Ref. T cell-binder), reference ROR Frizzled domain-binding TCE in Format 1A (Ref. Frz-binding TCE 1A), reference ROR Frizzled domain-binding TCE in Format 1B (Ref. Frz-binding TCE 1B), bispecific TCE 69A02 in Format 1A (69A02 TCE 1A), bispecific TCE 69A02 in Format 1B (69A02 TCE 1B), bispecific TCE 82H02 in Format 1A (82H02 TCE 1A), and bispecific TCE 82H02 in Format 1B (82H02 TCE 1B), on Jurkat T cells, cynomolgus pan-T cells, T cells isolated from C57BL/6 mice expressing a chimeric CD3ε consisting of the ectodomain of human CD3 and the transmembrane and cytosolic domains of mouse CD3, or human CD3 knockout Jurkat T cells are shown in FIG. 2A (and summarized in TABLE 12), FIG. 2B (and summarized in TABLE 12), FIG. 2C (and summarized in TABLE 12), and FIG. 2D (and summarized in TABLE 12), respectively.

TABLE 12
Apparent CD3 Binding Affinities

| | | | T cells from | |
|---|---|---|---|---|
| | | | huCD3 | huCD3 |
| | | Cynomol- | knock-in | knockout |
| | Jurkat | gus pan | C57BL/6 | Jurkat |
| | T cells | T cells | mice | T cells |
| | EC₅₀ (nM) | EC₅₀ (nM) | EC₅₀ (nM) | EC₅₀ (nM) |
| Control | No binding | No binding | No binding | Not Tested |
| Ref. T cell- | 157.3 | 164.28 | 258.8 | No binding |
| binder | | | | |
| Ref. Frz- | 9.1 | 10.47 | 4.06 | No binding |
| binding TCE | | | | |
| 1A | | | | |
| Ref. Frz- | 37.6 | 34.92 | 33.84 | No binding |
| binding TCE | | | | |
| 1B | | | | |
| 69A02 TCE 1A | 10.4 | 13.362 | Not Tested | No binding |
| 69A02 TCE 1B | 35.86 | 17.64 | Not Tested | No binding |
| 82H02 TCE 1A | 8.6 | 11.73 | 4.34 | No binding |
| 82H02 TCE 1B | 67.3 | 34.92 | 24.5 | No binding |

As shown in TABLE 12, the EC₅₀s indicate that the apparent binding affinities of 69A02 TCE 1A, 69A02 TCE 1B, 82H02 TCE 1A, and 82H02 TCE 1B for human, cynomolgus, and human/mouse chimeric CD3 were less than 3 fold different from each other. The apparent binding affinities for CD3 of 69A02 and 82H02 TCEs were approximately 4-fold lower in Format 1B as compared to Format 1A. Reference T cell-binder had an approximately 15-fold lower apparent binding affinity to CD3 as compared to bispecific TCEs 69A02 and 82H02 in Format 1A and approximately 4-fold lower apparent binding affinity to CD3 as compared to bispecific TCEs 69A02 and 82H02 in Format 1B. To address specificity for CD3, binding of bispecific TCEs 69A02 and 82H02 in Format 1A and Format 1B to Jurkat T cells devoid of CD3 was assessed (FIG. 2D). No non-specific binding was observed.

The apparent binding affinities for human and cynomolgus CD3 associated with the bispecific TCEs incorporating sequence optimized variants of 82H02 (82H02 TCE #1 having high ROR1 binding affinity and high CD3 affinity), 82H02 TCE #2 (having high ROR1 binding affinity and low CD3 binding affinity), 82H02 TCE #3 (having low ROR1 binding affinity and high CD3 binding affinity), and 82H02 TCE #4 (having low ROR1 binding affinity and low CD3 binding affinity) and reference T cell-binder (Ref. T cell-binder) were determined by flow cytometry using human CD8⁺ T cells (FIG. 12A and summarized in TABLE 13) and pan T cells from cynomolgus macaque (FIG. 12B and summarized in TABLE 13).

TABLE 13
Apparent CD3 Binding Affinities of Sequence Optimized Variants

| | Human CD8⁺ T Cells | Cynomolgus Pan T cells |
|---|---|---|
| | EC₅₀ (nM) | EC₅₀ (nM) |
| Anti-HEL Control | No binding | No binding |
| Ref. T cell-binder | 266.3 | 358.2 |
| 82H02 TCE #1 | 2.8 | 4.1 |
| 82H02 TCE #2 | 19.8 | 26.2 |
| 82H02 TCE #3 | 3.0 | 4.4 |
| 82H02 TCE #4 | 20.4 | 27.7 |

As shown in TABLE 13, reference T cell-binder had the weakest affinity for both human and cynomolgus CD3 at about 250 and 350 nM, respectively. These values were approximately 100-fold lower affinity than sequence optimized variants 82H02 TCE #1 and 82H02 TCE #3, and approximately 10 to 15-fold lower affinity than the sequence optimized variants 82H02 TCE #2 and 82H02 TCE #4. The difference between apparent CD3 binding affinity to human and cynomolgus CD3 for each sequence optimized 82H02 TCE variant was less than 2-fold. Interestingly, the maximum number of CD3 molecules bound by 82H02 TCE #1 (Cₘₐₓ) was between 30% (human) and 40% higher (cynomolgus) than the Cₘₐₓ observed for 82H02 TCE #3. The mechanism of action for this observation is unclear but may be caused by a difference in conformation between the two bispecific TCEs that results in easier access of 82H02 TCE #1 to CD3 than 82H02 TCE #3.

### 2.2 ROR1 Binding Affinity

The apparent binding affinities for human ROR1 associated with a reference T cell-binder (Ref. T cell-binder), reference ROR1 Frizzled domain-binding TCE in Format 1A (Ref. Frz-binding TCE 1A), reference ROR1 Frizzled domain-binding TCE in Format 1B (Ref. Frz-binding TCE 1B), bispecific TCE 69A02 in Format 1A (69A02 TCE 1A), bispecific TCE 69A02 in Format 1B (69A02 TCE 1B), bispecific TCE 82H02 in Format 1A (82H02 TCE 1A), and bispecific TCE 82H02 in Format 1B (82H02 TCE 1B), on MDA-MB-231 cells, T47D cells, 4T1 cells engineered to overexpress human ROR1, or 4T1 cells engineered to overexpress murine ROR1 are shown in FIG. 3A (and summarized in TABLE 14), FIG. 3B (and summarized in TABLE 14), FIG. 3C (and summarized in TABLE 14), and FIG. 3D (and summarized in TABLE 14), respectively.

TABLE 14
Apparent ROR1 Binding Affinities

| | MDA-MB- | T-47D | 4T1 | 4T1 |
|---|---|---|---|---|
| | 231 Cells | Cells | huROR1 | muROR1 |
| | EC₅₀ (nM) | EC₅₀ (nM) | EC₅₀ (nM) | EC₅₀ (nM) |
| Control | No binding | No binding | No binding | No binding |
| Ref. T cell- | 0.37 | Minimal | 2.48 | No binding |
| binder | | | | |
| Ref. Frz- | 81.63 | Minimal | 1.27 | 1.12 |
| binding TCE | | | | |
| 1A | | | | |
| Ref. Frz- | 63.72 | Minimal | 1.45 | 1.01 |
| binding TCE | | | | |
| 1B | | | | |
| 69A02 TCE 1A | 0.23 | No binding | Not Tested | Not Tested |
| 69A02 TCE 1B | 0.43 | No binding | Not Tested | Not Tested |
| 82H02 TCE 1A | 0.52 | No binding | 1.14 | 2.08 |
| 82H02 TCE 1B | 2.23 | No binding | 0.7 | 2.01 |

As shown in TABLE 14, the apparent binding affinities of reference T cell-binder, 69A02 TCE and 82H02 TCE were greater than 30-fold higher than reference ROR1 Frizzled domain-binding TCE using wild type MDA-MB-231 cells, which express low levels of ROR1. Minimal non-specific binding to ROR1⁻ T47D cells was observed with reference T cell binder and reference ROR1 Frizzled domain-binding TCE at high concentrations. No non-specific binding was observed with 69A02 or 82H02 TCEs

To determine the apparent binding affinities of bispecific 82H02 in Format 1A and Format 1B for mouse ROR1, 4T1 cells were engineered using the PiggyBAC transposon to overexpress mouse ROR-1 (FIG. 3D) and compared to cells engineered to express human ROR1 (FIG. 3C). The apparent affinities for human and mouse ROR1 were less than 3-fold different between bispecific TCE 82H02 in Format 1A, bispecific TCE 82H02 in Format 1B, and reference ROR1 Frizzled domain-binding TCE. Purified human IgG was used as a negative control. Reference T cell-binder did not bind mouse ROR1.

The apparent ROR1 binding affinity for reference ROR1 Frizzled domain-binding TCE was greater than 30-fold higher in 4T1 cells engineered to express human ROR1 (FIG. 3C) as compared to MDA-MB-231 cells (FIG. 3A). This increased binding affinity may be because the density of ROR1 expression on 4T1-hROR1⁺ cells is greater than 10-fold higher than expression on MDA-MB-231 cells. Further, the maximum number of hROR1 molecules bound by reference ROR1 Frizzled domain-binding TCE in Formats 1A and 1B was approximately 2.5 fold less than bispecific TCE 82H02 in Formats 1A and 1B. In contrast, for mouse ROR1, the maximum number of muROR1 molecules bound by reference ROR1 Frizzled domain-binding TCE and bispecific TCE 82H02 in Formats 1A and 1B was about the same. The reason for this difference in binding to human versus mouse ROR1 may be due to (1) access to the human versus mouse ROR1 Frizzled domain for which reference ROR1 Frizzled domain-binding TCE is specific and/or (2) a difference in density between human and mouse ROR1 expression on 4T1 cells.

The apparent binding affinities for human and mouse ROR1 associated with the bispecific TCEs incorporating sequence optimized variants of 82H02 (82H02 TCE #1, 82H02 TCE #2, 82H02 TCE #3, 82H02 TCE #4) and reference T cell-binder (Ref. T cell-binder) were determined by flow cytometry using ROR1⁺ MDA-MB-231 cells (FIG. 13A and summarized in TABLE 15) and 4T1 cells engineered to overexpress murine ROR1 (FIG. 13B and summarized in TABLE 15).

TABLE 15
Apparent ROR1 Binding Affinities
of Sequence Optimized Variants

| | ROR1⁺ MDA-MB-231 | 4T1 muROR1 |
|---|---|---|
| | EC₅₀ (nM) | EC₅₀ (nM) |
| Anti-HEL Control | No binding | No binding |
| Ref. T cell-binder | 0.14 | No binding |
| 82H02 TCE #1 | 0.81 | 2.19 |
| 82H02 TCE #2 | 0.72 | 2.24 |
| 82H02 TCE #3 | 211.18 | 1037 |
| 82H02 TCE #4 | 76.48 | 496 |

As shown in TABLE 15, both sequence optimized variants 82H02 TCE #1 and 82H02 TCE #2 had an approximately 3-fold difference in apparent ROR1 binding affinity between human and mouse ROR1. For sequence optimized variants 82H02 TCE #3 and 82H02 TCE #4, the difference in apparent ROR1 binding affinity between human and mouse was approximately 5-fold and 6.5-fold, respectively. The difference in apparent ROR1 binding affinity between bispecific TCEs having high ROR1 binding affinities (82H02 TCE #1 and 82H02 TCE #2) and bispecific TCEs having lowROR1 binding affinities (82H02 TCE #3 and 82H02 TCE #4) ranged between 100 to 250-fold for human ROR1 and 250 to 500-fold for mouse ROR1. Interestingly, although the apparent ROR1 binding affinities of bispecific TCEs having high ROR1 binding affinity were not appreciably impacted by high or low CD3 binding affinities, the difference in ROR1 binding affinity in 82H02 TCE #3 (having low 82H02 binding affinity and high CD3 binding affinity) versus 82H02 TCE #4 (having low 82H02 binding affinity and low CD3 binding affinity), was about 3-fold and 2-fold higher for human ROR1 and mouse ROR1, respectively. This result may be due to the maximum number of ROR1 molecules bound by 82H02 TCE #3 and 82H02 TCE #4, relative to 82H02 TCE #1 and 82H02 TCE #2, which made it difficult to calculate accurate EC₅₀ binding values for 82H02 TCE #3 and 82H02 TCE #4.

### Example 3: Target-Dependent Cytotoxicity (TDCC) and Cytokine Release Assays (TDCR)

This example describes the induction of target tumor cell killing and cytokine release by CD8⁺ T cells in the presence or absence of bispecific TCEs of the present disclosure.

### 3.1 TDCC and TDCR Assays Using Human CD8⁺ T Cells

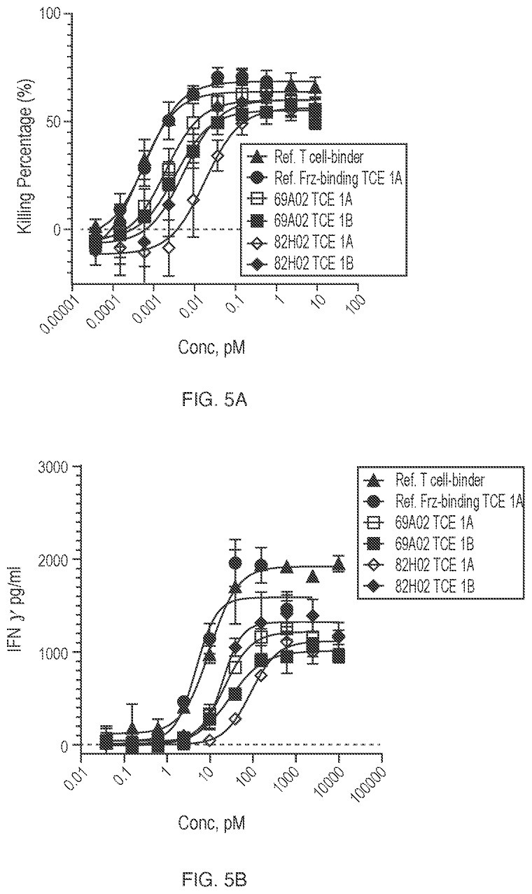

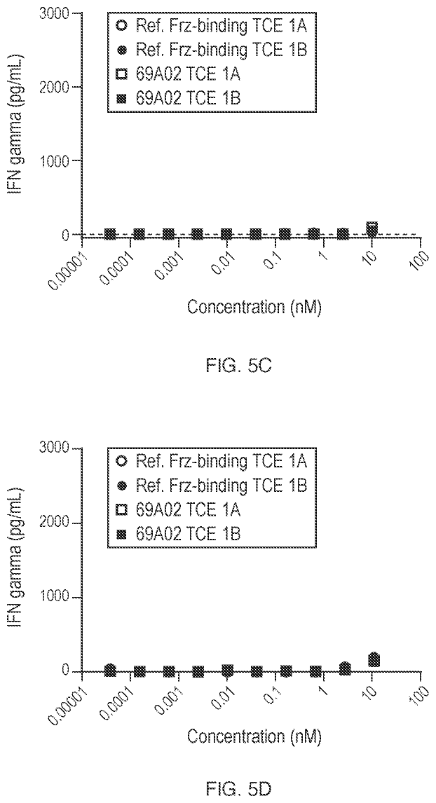

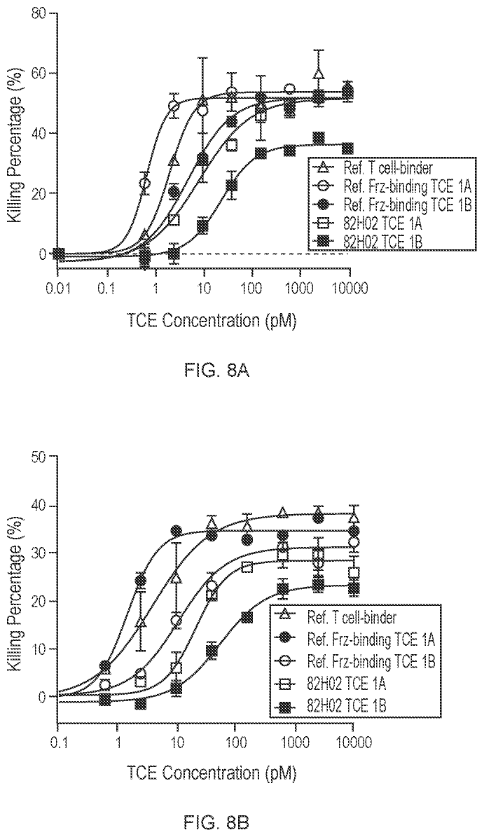

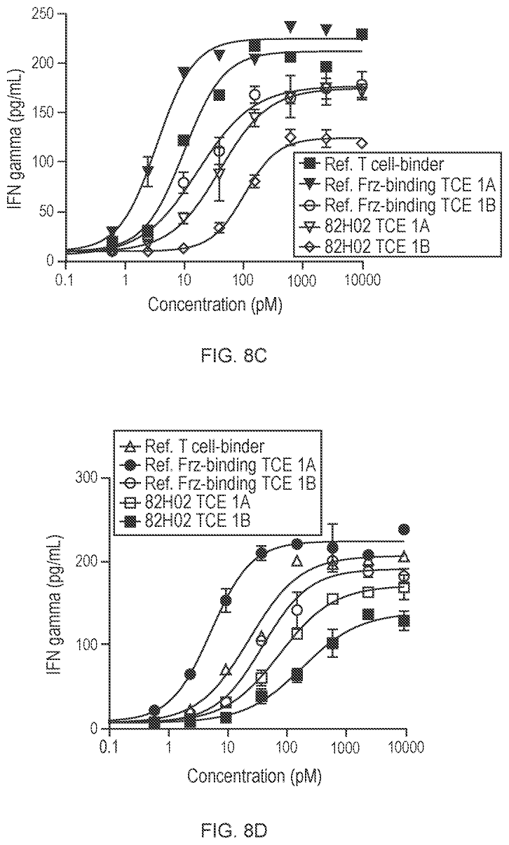

24 hours before TDCC and TDCR experiments, target cells (MDA-MB-231 cells (FIG. 5A and FIG. 5B); MCF-7 cells (FIG. 5C); T-47D cells (FIG. 5D); MDA-MB-231 cells (FIG. 8A and FIG. 8C)) were cultured in antibiotic-free media. 100 μL of target cell suspension was dispensed into each well of a black walled, clear flat-bottom plate (5000 cells per well) and incubated for 4 hours. The viability of human CD8⁺ T cells (HemaCare Bioresearch (Cat No. PB08NC)) cultured for 48 hours in 250 IU/mL IL2 was determined via trypan blue exclusion and the viable cell density was adjusted to 500,000 cells/mL in AIM-V medium (Gibco, Cat No: 12055-091) supplemented with 250 U/mL IL2. Test bispecific TCEs, reference T cell-binders, and reference Frizzled-binding TCEs were serially diluted in AIM-V media. At the end of the 4 hour incubation of target cells, culture medium was removed and 50 μL of media control was added to designated wells or 50 μl of CD8⁺ T cell suspension were added to each designated well to make an effector CD8⁺ T cell:target cell (E:T) ratio of 5:1. 50 μL of media control, test bispecific TCE, reference T cell-binder, or reference Frizzled-binding TCE dilution were added to designated wells and the plates were incubated for 24 hours at 37° C., 5% CO₂.

After the 24 hour incubation, plates were centrifuged for 30 seconds at 1000 RPM and 50 μl of supernatant was transferred to a black-walled, clear flat-bottom plate and 50 μL of ONE-Glo Luciferase Assay solution (Promega, Cat No: E6120) was added to each well and incubated for 2 minutes at room temperature. Bioluminescence of the samples in the plates was measured using an Envision plate reader (or equivalent). The killing percentage of target cells in a sample was calculated by treating the bioluminescence of samples comprising target cells co-cultured with CD8⁺ T cells and media control as 100% killing of target cells, while the bioluminescence of samples comprising target cells and the bioluminescence of samples comprising target cells in the absence of CD8⁺ T cells as 0% killing of target cells. The killing EC₅₀ (concentration vs killing percentage) was obtained using 4-parameter non-linear regression curve fitting in GraphPad Prism.

After the 24 hour incubation, 50 μL of supernatant was also transferred to wells of a V-bottom storage plate for IFNγ release detection. IFNγ release was measured using a human IFNγ AlphaLISA Detection Kit (PerkinElmer, Cat No: AL217F). In brief, 5 μL of samples or standards were added to a non-clear 384-well flat bottom assay plate. AlphaLISA anti-IFNγ acceptor beads (1 volume) were mixed with biotinylated anti-IFNγ antibody (1 volume) in 198 volumes of prepared 1× assay buffer. 20 μL of mixed acceptor bead/antibody was added to the samples and standards and incubated for 60 minutes at room temperature in the dark. Next, 1 volume of streptavidin donor beads were diluted in 61.5 volumes of 1× assay buffer and 25 μL of diluted streptavidin donor beads was added to wells containing the samples and standards and incubated for 30 minutes at room temperature in the dark. After 30 minutes incubation, the plate was read using an Envision plate reader. Conversion to the concentrations of IFNγ was calculated as per the manufacturer's specifications. EC₅₀ of IFNγ release (concentration vs IFNγ) was obtained using 4-parameter non-linear regression curve fitting in GraphPad Prism.

As shown in FIG. 5A (and summarized in TABLE 16), reference T cell-binder and reference ROR1 Frizzled domain-binding TCE in Format 1A were the most potent molecules tested in their ability to induce CD8⁺ T cell cytotoxicity when co-cultured with MDA-MB-231 cells and were less than 2-fold different from each other. Potency was defined as the EC₅₀ for target cell killing. The EC₅₀s for bispecific TCE 69A02 in Format 1A and bispecific TCE 82H02 in Format 1A were both approximately 20 pM and within 2 to 3 fold difference of the EC₅₀s for bispecific TCE 69A02 in Format 1B and bispecific TCE 82H02 in Format 1B, respectively.

The magnitude of killing of the reference T cell-binder, reference ROR1 Frizzled domain-binding TCE in Format 1A, bispecific TCE 69A02 in Format 1A, and bispecific TCE 82H02 in Format 1A were not appreciably different, whereas bispecific TCE 69A02 in Format 1B and bispecific TCE 82H02 in Format 1B, had slightly reduced potency with EC₅₀s ranging from about 35 to 60 pm.

TABLE 16
Target-dependent Cytotoxicity of Human CD8⁺ T Cells

| | CD8⁺ T cell: ROR1⁺ MDA- |
|---|---|
| | MB-231 |
| | EC₅₀ (pM) |
| Ref. T cell- | 6.9 |
| binder | |
| Ref. Frz- | 4.7 |
| binding TCE | |
| 1A | |
| Ref. Frz- | Not Tested |
| binding TCE | |
| 1B | |
| 69A02 TCE 1A | 20.2 |
| 69A02 TCE 1B | 35.6 |
| 82H02 TCE 1A | 19 |
| 82H02 TCE 1B | 58.1 |

As shown in FIG. 5B (and summarized in TABLE 17), reference T cell-binder and reference ROR1 Frizzled domain-binding TCE were the most potent molecules tested in their ability to induce CD8⁺ T cell IFNγ release when co-cultured with MDA-MB-231 cells and were approximately 2-fold different from each other. Potency was defined as the EC₅₀ for IFNγ release.

Bispecific TCE 69A02 in Format 1A and bispecific TCE 82H02 in Format 1A were approximately 2 to 3-fold less potent than reference T cell-binder and induced approximately 30% less maximum IFNγ release indicating a decoupling from T cell cytotoxic activity induction. Bispecific TCE 69A02 in Format 1B and bispecific TCE 82H02 in Format 1B were approximately 3 to 9-fold less potent than reference T cell-binder and induced approximately 40% less maximum IFNγ release

As shown in FIGS. 5C and 5D (and summarized in TABLE 17), neither reference ROR1 Frizzled domain-binding TCE in Format 1A or Format 1B, or bispecific TCE 69A02 in Format 1A or Format 1B induced detectable levels of IFNγ release indicating that IFNγ release was ROR1-dependent.

TABLE 17
Target-dependent Cytokine Release of Human CD8⁺ T Cells

| | | CD8⁺ T cell: | | | | CD8⁺ T |
|---|---|---|---|---|---|---|
| | CD8⁺ T cell: | ROR1⁺ | | CD8⁺ T cell: | CD8⁺ T | cell: |
| | ROR1⁺ | MDA-MB- | CD8⁺ T cell: | ROR1⁻ | cell: | ROR1⁻ |
| | MDA-MB- | 231 | ROR1⁻ | MCF7 | ROR1⁻ | T47D |
| | 231 | Max. IFNγ | MCF7 | Max. IFNγ | T47D | Max. IFNγ |
| | EC₅₀ (pM) | (pg/mL) | EC₅₀ (pM) | (pg/mL) | EC₅₀ (pM) | (pg/mL) |
| Ref. T cell- | 9.9 | 1814 | Not Tested | Not Tested | Not Tested | Not Tested |
| binder | | | | | | |
| Ref. Frz- | 4.7 | 1128 | No Activity | No Activity | No Activity | No Activity |
| binding TCE | | | | | | |
| 1A | | | | | | |
| Ref. Frz- | Not Tested | Not Tested | No Activity | No Activity | No Activity | No Activity |
| binding TCE | | | | | | |
| 1B | | | | | | |
| 69A02 TCE | 21.5 | 1203 | No Activity | No Activity | No Activity | No Activity |
| 1A | | | | | | |
| 69A02 TCE | 28.9 | 1099 | No Activity | No Activity | No Activity | No Activity |
| 1B | | | | | | |
| 82H02 TCE | 19.8 | 1271 | Not Tested | Not Tested | Not Tested | Not Tested |
| 1A | | | | | | |
| 82H02 TCE | 88.9 | 1094 | Not Tested | Not Tested | Not Tested | Not Tested |
| 1B | | | | | | |

### 3.2 TDCC and TDCR Assays Using Cynomolgus CD8⁺ T Cells

To assess whether cynomolgus monkeys are a relevant species with which to study the bispecific TCEs of the present disclosure, we compared the functional activity of human and cynomolgus CD8⁺ T cells in the in vitro target-dependent cytotoxicity and IFNγ release assays.

TDCC assays using cynomolgus CD8⁺ T cells (IQ Biosciences, Cat No: IQB-Mn1-CD8T2) were performed using methods analogous to TDCC assays using human CD8⁺ T cells as described in Example 3.1 above.

TDCR assays using cynomolgus CD8⁺ T cells were performed using a cynomolgus IFNγ AlphaLISA Detection Kit (PerkinElmer, Cat No: AL561C) having a manufacturer's protocol analogous to that of the human IFNγ AlphaLISA Detection Kit as described in EXAMPLE 3.1 above.

As shown in FIG. 8A and FIG. 8B (and summarized in TABLE 18), the rank order of molecules tested in their ability to induce CD8⁺ T cell cytotoxicity when co-cultured with MDA-MB-231 tumor cells was the same for both human and cynomolgus CD8⁺ T cells; namely, reference ROR1 Frizzled domain-binding TCE in Format 1A>reference T cell-binder>reference ROR1 Frizzled domain-binding TCE in Format 1B>bispecific TCE 82H02 in Format 1A>bispecific TCE 82H02 in Format 1B. For each molecule tested, there was an approximate 2 fold difference in potency of induction of cytotoxic activity between treated human CD8⁺ T cells and treated cynomolgus CD8⁺ T cells.

TABLE 18
Target-dependent Cytotoxicity of
Human and Cynomolgus CD8⁺ T Cells

| | | Cynomolgus |
|---|---|---|
| | Human CD8⁺ T | CD8⁺ T cell: |
| | cell: ROR1⁺ | ROR1⁺ MDA- |
| | MDA-MB-231 | MB-231 |
| | EC₅₀ (pM) | EC₅₀ (pM) |
| Ref. T cell- | 2.5 | 3.858 |
| binder | | |
| Ref. Frz- | 0.7 | 1.446 |
| binding TCE | | |
| 1A | | |
| Ref. Frz- | 4.8 | 10.61 |
| binding TCE | | |
| 1B | | |
| 69A02 TCE 1A | Not Tested | Not Tested |
| 69A02 TCE 1B | Not Tested | Not Tested |
| 82H02 TCE 1A | 8.3 | 21.08 |
| 82H02 TCE 1B | 23.7 | 54.81 |

As shown in FIG. 8C and FIG. 8D (and summarized in TABLE 19), the rank order of molecules tested in their ability to induce IFNγ release from CD8⁺ T cells co-cultured with MDA-MB-231 tumor cells was the same for both human and cynomolgus CD8⁺ T cells; namely, reference ROR1 Frizzled domain-binding TCE in Format 1A>reference T cell-binder>reference ROR1 Frizzled domain-binding TCE in Format 1B>bispecific TCE 82H02 in Format 1A>bispecific TCE 82H02 in Format 1B. For each molecule tested, there was an approximate 2 fold difference in potency of induction of IFNγ release between treated human CD8⁺ T cells and treated cynomolgus CD8⁺ T cells.

The magnitude of ROR1-dependent tumor cell cytotoxicity and IFNγ release was similar between human and cynomolgus CD8⁺ T cells. This supports the use of cynomolgus monkeys for non-clinical studies of bispecific TCEs of the present disclosure.

TABLE 19
Target-dependent Cytokine Release of Human and Cynomolgus CD8⁺ T Cells

| | | Human CD8⁺ | | Cynomolgus |
|---|---|---|---|---|
| | Human CD8⁺ | T cell: ROR1⁺ | Cynomolgus | CD8⁺ T cell: |
| | T cell: ROR1⁺ | MDA-MB-231 | CD8⁺ T cell: | ROR1⁺ MDA- |
| | MDA-MB-231 | Cells | ROR1⁺ MDA- | MB-231 Cells |
| | Cells | Max. IFNγ | MB-231 Cells | Max. IFNγ |
| | EC₅₀ (pM) | (pg/mL) | EC₅₀ (pM) | (pg/mL) |
| Ref. T cell- | 12.8 | 201.2 | 26.7 | 211.5 |
| binder | | | | |
| Ref. Frz- | 5.7 | 249.6 | 10.2 | 249.5 |
| binding TCE | | | | |
| 1A | | | | |
| Ref. Frz- | 17.2 | 177.1 | 41.2 | 190.8 |
| binding TCE | | | | |
| 1B | | | | |
| 69A02 TCE 1A | Not Tested | Not Tested | Not Tested | Not Tested |
| 69A02 TCE 1B | Not Tested | Not Tested | Not Tested | Not Tested |
| 82H02 TCE 1A | 40.0 | 174.8 | 79.7 | 170.9 |
| 82H02 TCE 1B | 104.4 | 124.6 | 191 | 138.6 |

### 3.3 TDCC and TDCR Assays Using Human PBMCs

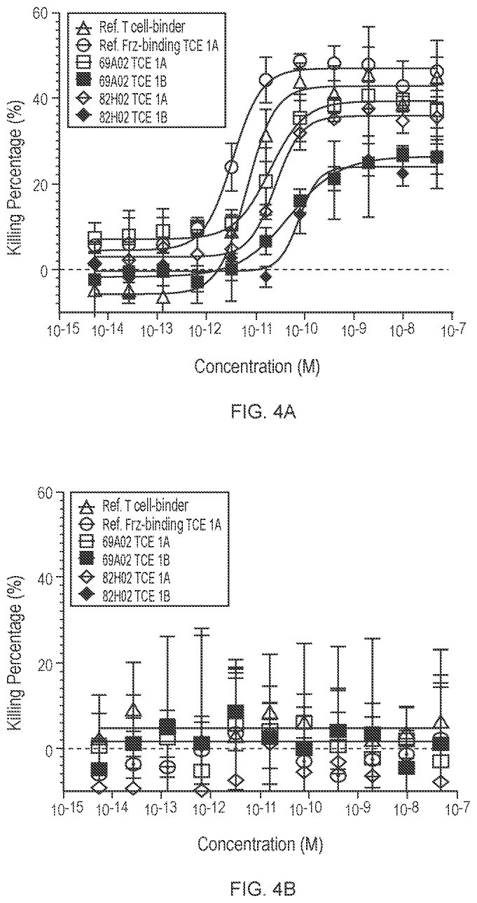

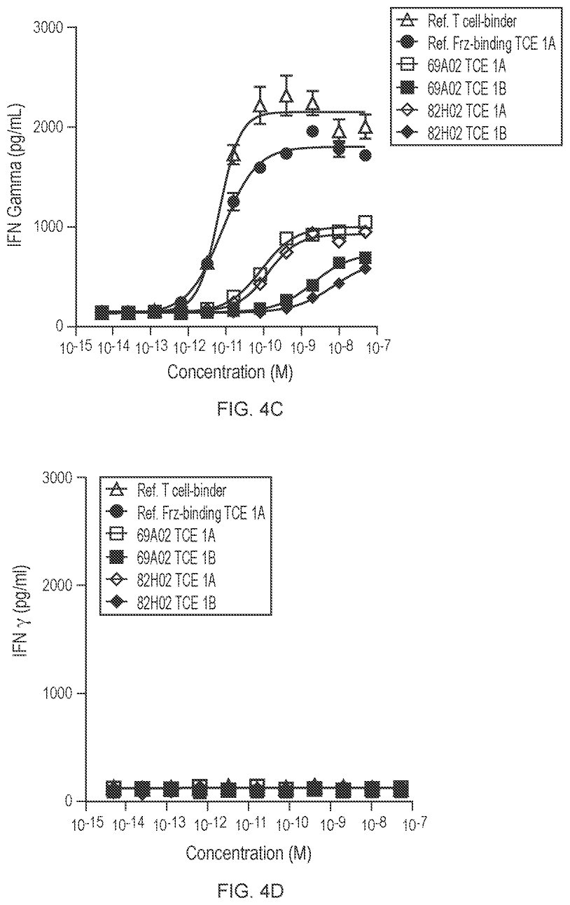

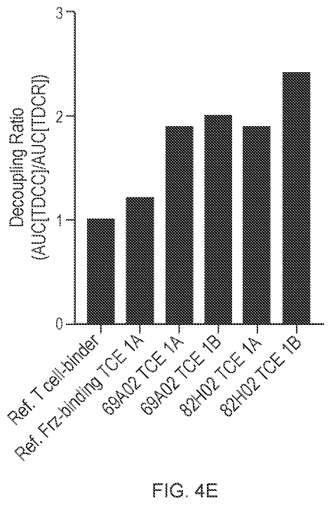

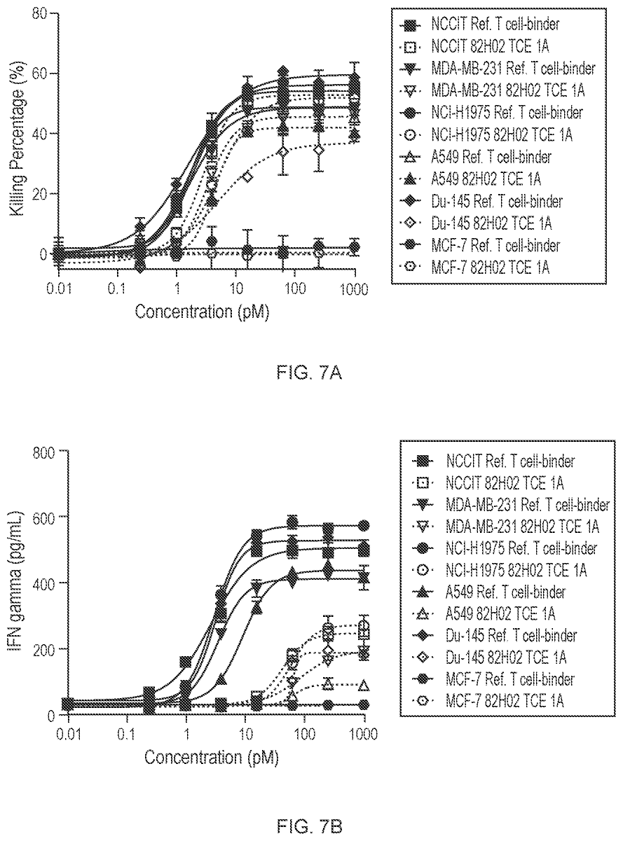

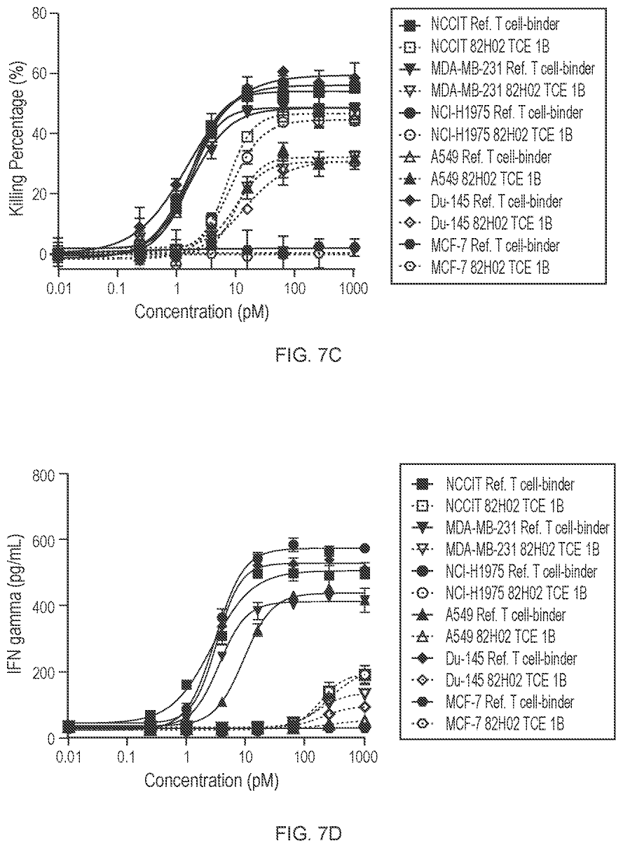

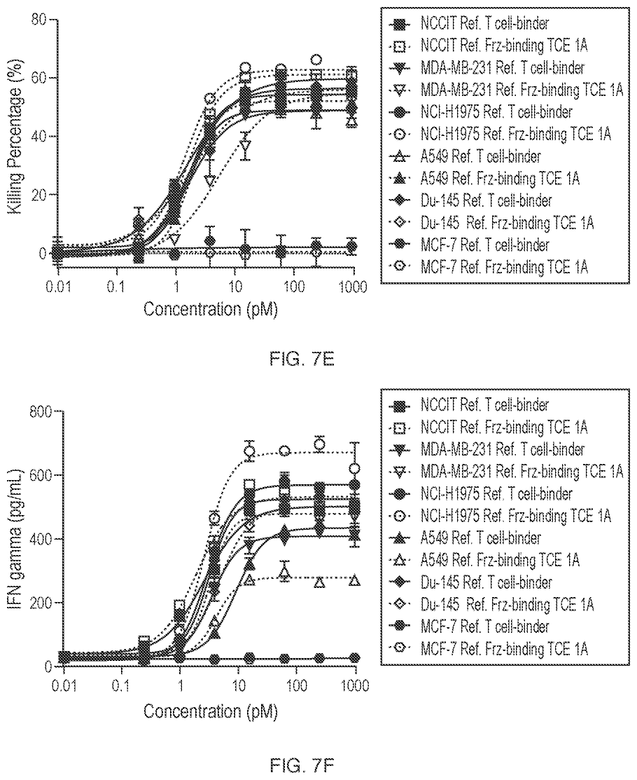

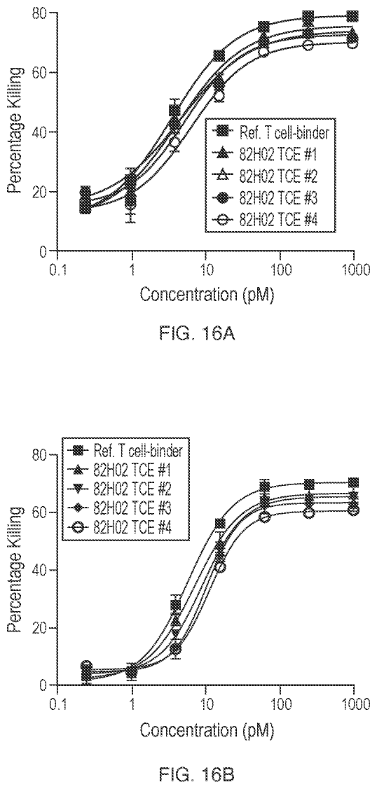

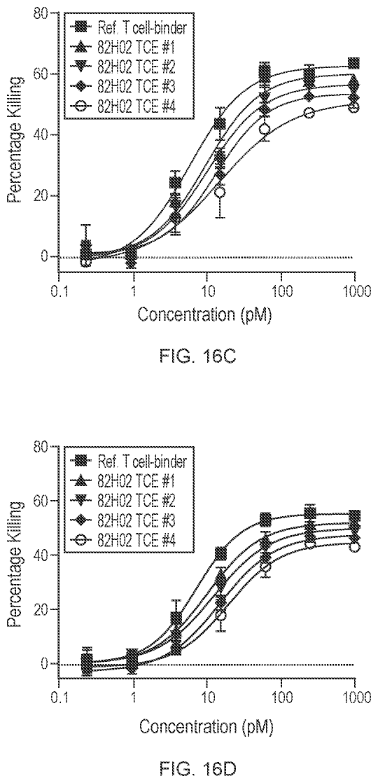

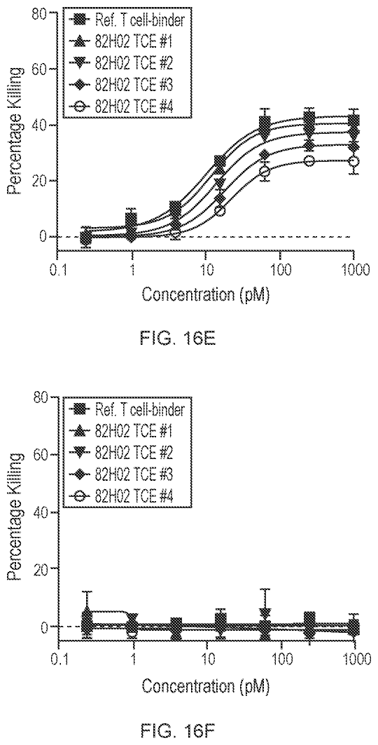

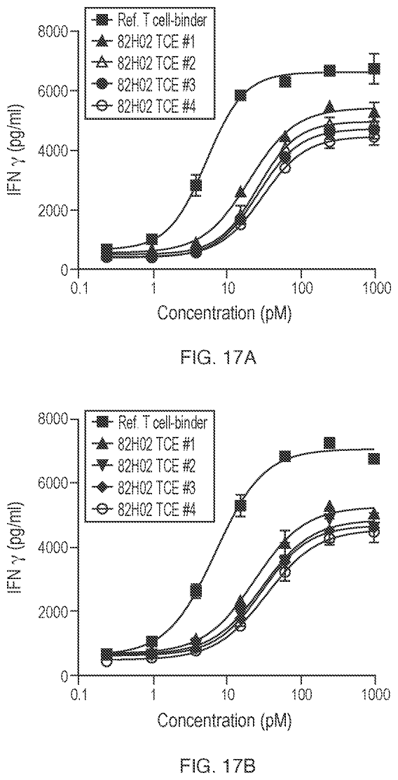

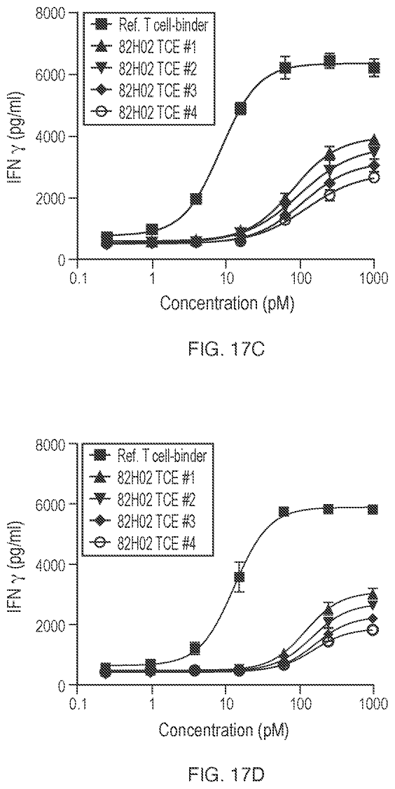

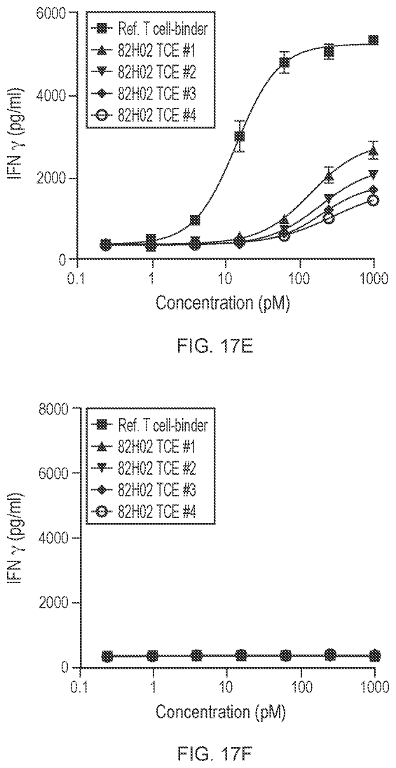

TDCC assays using PBMCs isolated from human whole blood and target cells (MDA-MB-231 cells (FIG. 4A, FIG. 4C, and FIG. 14); T-47D cells (FIG. 4B and FIG. 4D); Du-145 cells, A549 cells, NCI-H1975 cells, MDA-MB-231 cells, NCCIT cells and MCF-7 cells (FIG. 7); and MDA-MB-231 cells with different expression levels of ROR1 (FIG. 16 and FIG. 17)), were performed using methods analogous to TDCC assays using human CD8⁺ T cells as described in EXAMPLE 3.1 above.

Specifically, PBMCs were isolated from whole blood using Ficoll-Paque Plus medium (GE Healthcare, Cat No: 17144002) according to the manufacturer's instructions and resuspended in RPMI1640 media supplemented with 10% FBS and 1% penicillin/streptomycin at a cell density of 3 million cells/mL.

TDCR assays using PBMCs were performed using a human IFNγ AlphaLISA Detection Kit (PerkinElmer, Cat No: AL217F) as described in EXAMPLE 3.1 above.

To identify bispecific TCEs that induce robust TDCC of target tumor cells and low levels of TDCR from PBMCs, a decoupling ratio was calculated and defined as the quotient of the area under the curve (AUC) of a TDCC assay killing percentage vs concentration plot and the AUC of a TDCR assay IFNγ release vs concentration plot.

As shown in FIG. 4A (and summarized in TABLE 20), reference T cell-binder and reference ROR1 Frizzled domain-binding TCE in Format 1A were the most potent molecules tested in their ability to induce CD8⁺ T cell cytotoxicity when co-cultured with MDA-MB-231 cells and were less than 2-fold different from each other. Potency was defined as the EC₅₀ for target cell killing.

The EC₅₀s for bispecific TCE 69A02 in Format 1A and bispecific TCE 82H02 in Format 1A were approximately 5-fold less potent than reference T cell binder (˜19 to 25 pM). EC₅₀s for bispecific TCE 69A02 in Format 1B and bispecific TCE 82H02 in Format 1B, were 4-fold less potent than bispecific TCE 69A02 in Format 1A and bispecific TCE 82H02 in Format 1A, respectively (˜48 to 77 pM).

The magnitude of killing of the reference T cell-binder, reference ROR1 Frizzled domain-binding TCE in Format 1A, bispecific TCE 69A02 in Format 1A, and bispecific TCE 82H02 in Format 1A were not appreciably different. As shown in FIG. 4B (and summarized in TABLE 20), no cytotoxicity was measurably detected in co-cultures of PBMCs with ROR1⁻ T47D target tumor cells, indicating that the observed cytotoxicity was ROR1-dependent.

TABLE 20
Target-dependent Cytotoxicity of Human PBMCs

| | PBMCs: | PBMCs: |
|---|---|---|
| | ROR1⁺ MDA- | ROR1⁻ T47D |
| | MB-231 cells | cells |
| | EC₅₀ (pM) | EC₅₀ (pM) |
| Ref. T cell- | 6.4 | No activity |
| binder | | |
| Ref. Frz- | 3.4 | No activity |
| binding TCE | | |
| 1A | | |
| Ref. Frz- | Not Tested | Not Tested |
| binding TCE | | |
| 1B | | |
| 69A02 TCE 1A | 19.4 | No activity |
| 69A02 TCE 1B | 48.0 | No activity |
| 82H02 TCE 1A | 24.8 | No activity |
| 82H02 TCE 1B | 76.6 | No activity |

As shown in FIG. 4C (and summarized in TABLE 21), reference T cell-binder and reference ROR1 Frizzled domain-binding TCE in Format 1A were the most potent molecules tested in their ability to induce PBMC IFNγ release when co-cultured with MDA-MB-231 cells and were less than 2-fold different from each other. Potency was defined as the EC₅₀ for IFNγ release.

Bispecific TCE 69A02 in Format 1A and bispecific TCE 82H02 in Format 1A were approximately 10-fold less potent than reference T cell-binder. Bispecific TCE 69A02 in Format 1B and bispecific TCE 82H02 in Format 1B were approximately 300 to 1000-fold less potent than reference T cell-binder and induced approximately 60 to 70% less maximum IFNγ release.

As shown in FIG. 4D (and summarized in TABLE 21), neither reference T cell-binder, reference ROR1 Frizzled domain-binding TCE in Format 1A, bispecific TCE 69A02 in Format 1A or Format 1B, or bispecific TCE 82H02 in Format 1A or Format 1B induced detectable levels of IFNγ release indicating that IFNγ release was ROR1-dependent.

While reference T cell-binder and reference ROR1 Frizzled domain-binding TCE in Format 1A were highly potent at the induction of PBMC cytotoxic killing of tumor cells and IFNγ release, bispecific TCEs 69A02 and 82H02 in Formats 1A and 1B were more potent inducers of cytotoxic killing than IFNγ release.

TABLE 21
Target-dependent Cytokine Release of Human PBMCs

| | | PBMCs: | | PBMCs: |
|---|---|---|---|---|
| | PBMCs: | ROR1⁺ MDA- | PBMCs: | ROR1⁻ T47D |
| | ROR1⁺ MDA- | MB-231 Cells | ROR1⁻ T47D | Cells |
| | MB-231 Cells | Max. IFNγ | Cells | Max. IFNγ |
| | EC₅₀ (pM) | (pg/mL) | EC₅₀ (pM) | (pg/mL) |
| Ref. T cell- | 6.6 | 1806 | No activity | No activity |
| binder | | | | |
| Ref. Frz- | 8 | 1324 | No activity | No activity |
| binding TCE | | | | |
| 1A | | | | |
| Ref. Frz- | Not Tested | Not Tested | Not Tested | Not Tested |
| binding TCE | | | | |
| 1B | | | | |
| 69A02 TCE 1A | 87.6 | 1084.1 | No activity | No activity |
| 69A02 TCE 1B | 2090 | 707.4 | No activity | No activity |
| 82H02 TCE 1A | 130 | 996.7 | No activity | No activity |
| 82H02 TCE 1B | 7120 | 600.3 | No activity | No activity |

To quantify the magnitude by which each test bispecific TCE decoupled cytotoxicity from proinflammatory cytokine release, a “decoupling ratio” was calculated as the quotient between the area under the curve (AUC) for target killing and the AUC for IFNγ release for a given test or reference molecule.

AUC values were normalized to the values calculated for the reference T cell-binder, for which the EC₅₀s (potency) of target cell killing and IFNγ release were both about 6 pM (i.e. a decoupling ratio of 1, meaning that cytotoxicity was not decoupled from IFNγ release). As shown in FIG. 4E, bispecific TCE 69A02 in Formats 1A and 1B, and bispecific TCE 82H02 in Formats 1A and 1B had decoupling ratios significantly greater than reference T cell binder (i.e. greater than 1).

Bispecific TCE 69A02 in Formats 1A and 1B, and bispecific TCE 82H02 in Format 1A had comparable decoupling ratios ranging from approximately 1.8 to 2. Bispecific TCE 82H02 in Format 1B had the highest decoupling ratio.

The observed decoupling of cytotoxicity from proinflammatory cytokine release in PBMCs was more pronounced than the decoupling observed in pure CD8⁺ T cell populations (EXAMPLE 3.1, above), particularly for bispecific TCE 82H02 in Format 1B. These results suggest that the presence of CD4⁺ helper T cells may play a role in the mechanism of action by which bispecific TCEs of the present disclosure maintain cytotoxicity while having reduced IFNγ release.

To determine whether decoupling of cytotoxicity from proinflammatory cytokine release mediated by bispecific TCEs of the present disclosure was a generalized characteristic across an array of ROR1⁺ tumor cell lines, ROR1 dependent target cell killing and ROR1-dependent IFNγ release was assessed across multiple tumor cell lines having various surface expression levels of ROR1. Average cell surface densities of ROR1 on cell lines used in this example are shown in TABLE 22.

TABLE 22
ROR1 Cell Surface Densities of Various Cell Lines

| Cell Line | ROR1 density (molecules per cell) |
|---|---|
| MCF-7 | 0 |
| Du-145 | 13336 |
| A549 | 36334 |
| NCI-H1975 | 38486 |
| MDA-MB-231 | 46660 |
| NCCIT | 94426 |

As shown in FIGS. 7A to 7D, and as was previously observed with MDA-MB-231 targets, decoupling of cytotoxicity from proinflammatory cytokine release was observed with all ROR-1⁺ tumor cell lines when co-cultured with PBMCs in the presence of bispecific TCE 82H02 in Formats 1A (FIG. 7A and FIG. 7B) and 1B (FIG. 7C and FIG. 7D). Decoupling was not observed in either reference T cell-binder or reference ROR1 Frizzled domain-binding TCE in Format 1A (FIG. 7E and FIG. 7F). No target cell killing or IFNγ release was observed in PBMC co-cultures with ROR1⁻ MCF-7 cells.

Overall, both target cell killing and proinflammatory cytokine release were observed to be ROR1 dependent. Further, given that the observed decoupling of cytotoxicity from cytokine release by bispecific TCE 82H02 in Formats 1A and 1B was observed for all ROR1⁺ cell lines tested, it appears that this phenomenon is not limited to MDA-MB-231 but is more generally applicable to all ROR1⁺ cell lines.

As shown in FIG. 14A (and summarized in TABLE 23), reference T cell-binder was 2 to 4-fold and 15-fold more potent in inducing cytotoxic tumor cell killing when PBMCs were co-cultured with MDA-MB-231 cells as compared to sequence optimized bispecific TCEs 82H02 TCE #1 and 82H02 TCE #2; and bispecific TCEs 82H02 TCE #3 and 82H02 TCE #4, respectively. The decreased potencies of bispecific TCEs 82H02 TCE #1 and 82H02 TCE #2 (high-affinity ROR1 binding) compared to bispecific TCEs 82H02 TCE #3 and 82H02 TCE #4 (low-affinity ROR1 binding), was between 4 to 6-fold. The decreased potencies of bispecific TCEs 82H02 TCE #1 and 82H02 TCE #3 (high-affinity CD3 binding) compared to bispecific TCEs 82H02 TCE #2 and 82H02 TCE #4 (low-affinity CD3 binding), was less than 2-fold. These results suggested that the major difference in potency for inducing cytotoxic killing activity between bispecific TCEs of the present disclosure is primarily driven by the apparent affinity for ROR1 and not CD3.

As shown in FIG. 14B (and summarized in TABLE 24), reference T cell-binder was about 20 to 25-fold and 40 to 50-fold more potent in inducing IFNγ release when PBMCs were co-cultured with MDA-MB-231 cells as compared to sequence optimized bispecific TCEs 82H02 TCE #1 and 82H02 TCE #2; and bispecific TCEs 82H02 TCE #3, and 82H02 TCE #4, respectively. The decreased potencies of bispecific TCEs 82H02 TCE #1 and 82H02 TCE #2 (high-affinity ROR1 binding) compared to bispecific TCEs 82H02 TCE #3 and 82H02 TCE #4 (low-affinity ROR1 binding), was approximately 2-fold. The decreased potencies of bispecific TCEs 82H02 TCE #1 and 82H02 TCE #3 (high-affinity CD3 binding) compared to bispecific TCEs 82H02 TCE #2 and 82H02 TCE #4 (low-affinity CD3 binding), was less than 2-fold. These results suggested that the major difference in potency for inducing IFNγ release between bispecific TCEs of the present disclosure is primarily driven by the apparent affinity for ROR1 and not CD3. In addition, the maximum release of IFNγ was reduced by about 50% and 60 to 65% by sequenced optimized bispecific TCEs 82H02 TCE #1 and 82H02 TCE #2; and bispecific TCEs 82H02 TCE #3 and 82H02 TCE #4, respectively.

As shown in FIG. 14C, all of bispecific TCEs 82H02 TCE #1, 82H02 TCE #2, 82H02 TCE #3, and 82H02 TCE #4 were associated with decoupling ratios which were not appreciably different from one another (approximately 2 as normalized to reference T cell binder).

TABLE 23
Target-dependent Cytotoxicity of Human PBMCs

| | PBMCs: MDA-MB-231 |
|---|---|
| | EC₅₀ (nM) |
| Ref. T cell-binder | 10.1 |
| 82H02 TCE #1 | 24.58 |
| 82H02 TCE #2 | 43.9 |
| 82H02 TCE #3 | 148.4 |
| 82H02 TCE #4 | 168.4 |

TABLE 24
Target-dependent Cytokine Release of Human PBMCs

| | PBMCs: MDA-MB-231 | PBMCs: MDA-MB-231 |
|---|---|---|
| | EC₅₀ (nM) | Max IFNγ (pg/mL) |
| Ref. T cell-binder | 10.7 | 2136.1 |
| 82H02 TCE #1 | 204.7 | 1124.8 |
| 82H02 TCE #2 | 264.9 | 1001.1 |
| 82H02 TCE #3 | 424.9 | 880.7 |
| 82H02 TCE #4 | 509.4 | 764.3 |

The capacity for ROR1 density discrimination related to sequence optimized bispecific TCEs of the present disclosure was assessed by co-culturing PBMCs with target MDA-MB-231 tumor cells engineered to express different cell surface densities of ROR1. MDA-MB-231 cells express approximately 40,000 ROR1 molecules/cell. To reduce surface expression of ROR1, MDA-MB-231 cells were transduced with lentiviruses encoding ROR1-specific shRNA. To increase surface expression of ROR1, MDA-MB-231 cells were transduced with lentiviruses encoding full-length ROR1. ROR1 knockout MDA-MB-231 cells were made using Cas9/CRISPR technology. In total, 6 cell lines were generated having various surface expression levels of ROR1: 0.2×10⁵ molecules/cell (MDA-MB-231-0.2); 0.4×10⁵ molecules/cell (MDA-MB-231-0.4); 6×10⁵ molecules/cell (MDA-MB-231-0.6); 1.3×10⁶ molecules/cell (MDA-MB-231-1.3); 7×10⁶ molecules/cell (MDA-MB-231-7).

As shown in FIG. 16F (and summarized in TABLE 25) and FIG. 17F (and summarized in TABLE 26), PBMCs co-cultures with ROR1 knockout MDA-MB-231 target cells did not result in appreciable levels of killing or IFNγ release above background. As shown FIGS. 16A to 16E, maximal PBMC-mediated cytotoxicity induced by sequence-optimized bispecific 82H02 TCEs or reference T cell-binder was reduced as cell surface density of ROR1 was reduced. Similarly, as shown in FIGS. 17A to 17E, maximal IFNγ release induced by sequence-optimized bispecific 82H02 TCEs or reference T cell-binder was reduced as cell surface density of ROR1 was reduced.

The rank order of potency for both induction of cytotoxic activity and IFNγ release was reference T cell-binder>82H02 TCE #1>82H02 TCE #2>82H02 TCE #3>82H02 TCE #4.

For cytotoxic killing of target tumor cells, differences in potency of the test molecules increased as ROR1 density decreased. At the lowest ROR 1 cell density (MDA-MB-231-0.2), potencies of cytotoxic activities of reference T cell-binder, 82H02 TCE #1, 82H02 TCE #2, 82H02 TCE #3, and 82H02 TCE #4, were less than 3 fold.

For IFNγ release, lower EC₅₀ and Eₘₐₓ values relative to cytotoxic killing were consistent with decoupling of cytotoxicity from cytokine release and observed 82H02 TCE #1, 82H02 TCE #2, 82H02 TCE #3, and 82H02 TCE #4, but not the reference T cell-binder. The magnitude of decoupling was inversely correlated with the cell surface density of ROR1 and was not appreciably superior to induction by reference T cell binder at super-physiological densities of ROR1 (i.e. >1.3×10⁶ molecules/cell).

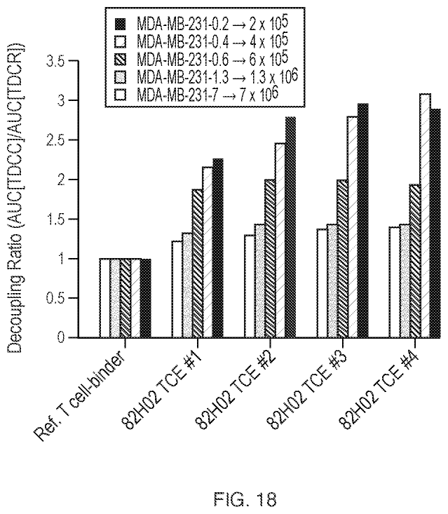

As shown in FIG. 18, the decoupling ratio (normalized to reference T cell-binder), increased with decreasing densities of cell surface ROR1 expression. The highest decoupling ratios were observed with 82H02 TCE #2, 82H02 TCE #3, and 82H02 TCE #4 and were not appreciably different from one another. The decoupling ratios associated with 82H02 TCE #1 at ROR1 densities between 0.2×10⁵ and 0.6×10⁵ molecules/cells were approximately 20% lower compared to 82H02 TCE #2, 82H02 TCE #3, and 82H02 TCE #4.

TABLE 25
Target-dependent Cytotoxicity of Human PBMCs Against Target Cells Having Various ROR1 Cell Surface Densities

| | PBMCs: MDA- | | | PBMCs: MDA- | | | PBMCs: MDA- | | | PBMCs: MDA- | | | PBMCs: MDA- | | |
|---|---|---|---|---|---|---|---|---|---|---|---|---|---|---|---|
| | MB-231-7 | | | MB-231-1.3 | | | MB-231-0.6 | | | MB-231-0.4 | | | MB-231-0.2 | | |
| | Eₘₐₓ | EC₅₀ | | Eₘₐₓ | EC₅₀ | | Eₘₐₓ | EC₅₀ | | Eₘₐₓ | EC₅₀ | | Eₘₐₓ | EC₅₀ | |
| | (%) | (pM) | AUC | (%) | (pM) | AUC | (%) | (pM) | AUC | (%) | (pM) | AUC | (%) | (pM) | AUC |
| Ref. T | 79.28 | 3.571 | 77740 | 70.3 | 5.676 | 68976 | 62.91 | 6.124 | 60564 | 55.53 | 7.295 | 54216 | 43.33 | 10.21 | 41534 |
| cell- | | | | | | | | | | | | | | | |
| binder | | | | | | | | | | | | | | | |
| 82H02 | 75.8 | 4.334 | 74224 | 66.55 | 7.03 | 64797 | 60.25 | 10.02 | 57087 | 52.22 | 10.32 | 50197 | 40.69 | 11.89 | 39032 |
| TCE #1 | | | | | | | | | | | | | | | |
| 82H02 | 74.13 | 4.705 | 72000 | 65.36 | 8.96 | 63659 | 56.84 | 10.52 | 54138 | 50.18 | 12.53 | 47643 | 37.54 | 13.72 | 35670 |
| TCE #2 | | | | | | | | | | | | | | | |
| 82H02 | 72.75 | 5.456 | 71422 | 63.22 | 10.15 | 61727 | 53.8 | 13.83 | 50978 | 47.79 | 15.27 | 44842 | 33.11 | 18.1 | 31334 |
| TCE #3 | | | | | | | | | | | | | | | |
| 82H02 | 70.26 | 6.529 | 68323 | 60.37 | 11.19 | 58897 | 51.45 | 15.24 | 46345 | 45 | 19.62 | 41916 | 27.41 | 22.33 | 25919 |
| TCE #4 | | | | | | | | | | | | | | | |

TABLE 26
Target-dependent Cytokine Release of Human PBMCs Against Target Cells Having Various RORI Cell Surface Densities

| | PBMCs: MDA- | | | PBMCs: MDA- | | | PBMCs: MDA- | | | PBMCs: MDA- | | | PBMCs: MDA- | | |
|---|---|---|---|---|---|---|---|---|---|---|---|---|---|---|---|
| | MB-231-7 | | | MB-231-1.3 | | | MB-231-0.6 | | | MB-231-0.4 | | | MB-231-0.2 | | |
| | Eₘₐₓ | EC₅₀ | | Eₘₐₓ | EC₅₀ | | Eₘₐₓ | EC₅₀ | | Eₘₐₓ | EC₅₀ | | Eₘₐₓ | EC₅₀ | |
| | (%) | (pM) | AUC | (%) | (pM) | AUC | (%) | (pM) | AUC | (%) | (pM) | AUC | (%) | (pM) | AUC |
| Ref. T | 6611 | 5.394 | 6575904 | 7059 | 7.189 | 6904992 | 6362 | 8.42 | 6246567 | 5859 | 13.4 | 5671198 | 5256 | 14.13 | 5044135 |
| cell- | | | | | | | | | | | | | | | |
| binder | | | | | | | | | | | | | | | |
| 82H02 | 5423 | 20.4 | 5157097 | 5258 | 24.28 | 4928042 | 3975 | 80.34 | 3151466 | 3051 | 123 | 2433954 | 2861 | 143.2 | 2094719 |
| TCE #1 | | | | | | | | | | | | | | | |
| 82H02 | 4972 | 26.47 | 4683147 | 4853 | 30.55 | 4494702 | 3668 | 97.65 | 9772797 | 2667 | 151 | 2034463 | 2267 | 191.6 | 1554079 |
| TCE #2 | | | | | | | | | | | | | | | |
| 82H02 | 4734 | 27.14 | 4430368 | 4677 | 30.79 | 4317548 | 3157 | 107.4 | 1622647 | 2247 | 167 | 1680388 | 1829 | 202 | 1286784 |
| TCE #3 | | | | | | | | | | | | | | | |
| 82H02 | 4478 | 30.87 | 4144898 | 4556 | 35.08 | 4108415 | 2784 | 121.1 | 8232477 | 1835 | 160.2 | 1424962 | 1729 | 279.5 | 1089083 |
| TCE #4 | | | | | | | | | | | | | | | |

### 3.4 TDCC and TDCR Assays Using CD3ε-Knock-In C57BL/6 Mouse Splenocytes

C571BL/6 mice in which mouse CD3ε is knocked out and replaced with a chimeric CD3ε consisting of the ectodomain of human CD3 and the transmembrane and cytosolic domains of mouse CD3 (human CD3 knock-in or hCD3-KI) were generated to see if such animals could be used as relevant models for assessing the preclinical in vivo activity of bispecific TCEs of the present disclosure. 4T1 cells stably expressing cell surface human ROR1 (hROR1⁺ 4T1) or mouse ROR1 (mROR1⁺ 4T1) using the PiggyBAC transposon based system.

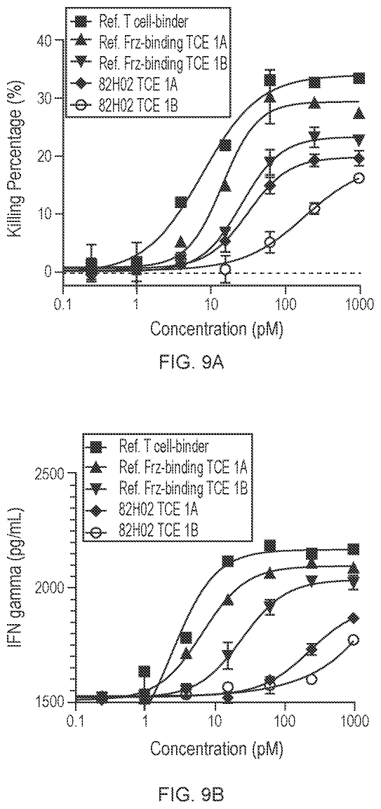

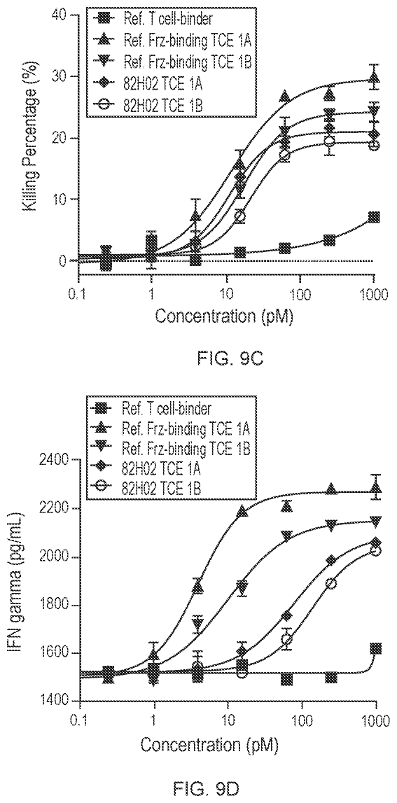

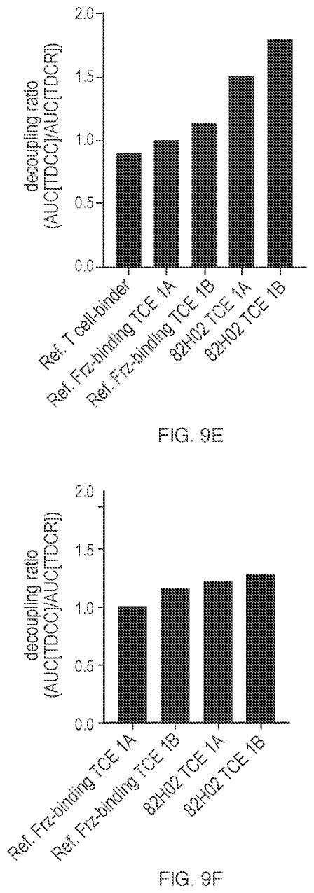

TDCC assays using CD3ε-knock-in C571BL/6 mouse splenocytes and target cells (4T1-hROR1 cells (FIG. 9A and FIG. 9B); or 4T1-mROR1 cells (FIG. 9C and FIG. 9D)), were performed using methods analogous to TDCC assays using human CD8⁺ T cells as described in EXAMPLE 3.1 above.

Specifically, mouse splenocytes were isolated from the spleens of human CD3ε-knock-in C571BL/6 mice by pressing the spleens through a 100-μm cell strainer into a 50 mL conical tube. The cell strainer was washed twice with 5 mL of PBS and the flow-through was collected to the 50-mL conical tube. The splenocytes were pelleted, resuspended in 5 mL of ACK lysis buffer (ThermoFisher, Cat No: A1049201), and incubated for 5 minutes at room temperature. Splenocytes were washed with PBS and resuspended in culture medium (RPMI1640 with 10% FBS and 1% penicillin/streptomycin) at a density of 3×10⁶ cells/mL.

TDCR assays using mouse CD8⁺ T cells were performed using a mouse IFNγ AlphaLISA Detection Kit (PerkinElmer, Cat No: AL501C) having a manufacturer's protocol analogous to that of the human IFNγ AlphaLISA Detection Kit as described in EXAMPLE 3.1 above.

To identify bispecific TCEs that induce robust TDCC of target tumor cells and low levels of TDCR from PBMCs, a decoupling ratio was calculated and defined as the quotient of the AUC of a TDCC assay killing percentage vs concentration plot and the AUC of a TDCR assay IFNγ release vs concentration plot.

As shown in FIG. 9A (and summarized in TABLE 27), the rank order of potency for induction of cytotoxic target cell killing against hROR1⁺ 4T1 cells was reference T cell-binder>reference ROR1 Frizzled domain-binding TCE in Format 1A>reference ROR1 Frizzled domain-binding TCE in Format 1B>bispecific TCE 82H02 in Format 1A>bispecific TCE 82H02 in Format 1B. As shown in FIG. 9C (and summarized in TABLE 27), the rank order of potency for induction of cytotoxic target cell killing against mROR1⁺ 4T1 cells was bispecific TCE 82H02 in Format 1A>reference ROR1 Frizzled domain-binding TCE in Format 1A>reference ROR1 Frizzled domain-binding TCE in Format 1B>bispecific TCE 82H02 in Format 1B. As expected, reference T cell-binder had no cytotoxic activity against mROR1⁺ 4T1 cells because it does not bind murine ROR1. For bispecific TCE 82H02, the difference in potencies against hROR1⁺ 4T1 and mROR1⁺ 4T1 was approximately 3-fold and 9-fold for Formats 1A and 1B, respectively.

As shown in FIG. 9B (and summarized in TABLE 28), the rank order of potency for induction of IFNγ release against hROR1⁺ 4T1 cells was reference T cell-binder>reference ROR1 Frizzled domain-binding TCE in Format 1A>reference ROR1 Frizzled domain-binding TCE in Format 1B>bispecific TCE 82H02 in Format 1A>bispecific TCE 82H02 in Format 1B. As shown in FIG. 9D (and summarized in TABLE 28), the rank order of potency for induction of IFNγ release against mROR1⁺ 4T1 cells was reference ROR1 Frizzled domain-binding TCE in Format 1A>reference ROR1 Frizzled domain-binding TCE in Format 1B>bispecific TCE 82H02 in Format 1A>bispecific TCE 82H02 in Format 1B. As expected, reference T cell-binder had no measurable IFNγ release against mROR1⁺ 4T1 cells because it does not bind murine ROR1. For bispecific TCE 82H02, the difference in potencies for IFNγ release against hROR1⁺ 4T1 and mROR1⁺ 4T1 was approximately 3-fold and greater than 3-fold for Formats 1A and 1B, respectively.

TABLE 27
Target-dependent Cytotoxicity of Human CD3-Knock-in Mouse
Splenocytes Against Human or Mouse ROR1⁺ 4T1 Cells

| | hCD3 KI | hCD3 KI |
|---|---|---|
| | splenocytes: | splenocytes: |
| | hROR1⁺ 4T1 | mROR1⁺ 4T1 |
| | cells | cells |
| | EC₅₀ (pM) | EC₅₀ (pM) |
| Ref. T cell- | 7.6 | No activity |
| binder | | |
| 5Ref. Frz- | 14.4 | 11.8 |
| binding TCE | | |
| 1A | | |
| Ref. Frz- | 25.9 | 17.7 |
| binding TCE | | |
| 1B | | |
| 82H02 TCE 1A | 31.6 | 11.2 |
| 82H02 TCE 1B | 185 | 21.5 |

TABLE 28
Target-dependent Cytokine Release of Human CD3-Knock-in
Mouse Splenocytes Against Human or Mouse ROR1⁺ 4T1 Cells

| | | hCD3 KI | | hCD3 KI |
|---|---|---|---|---|
| | hCD3 KI | splenocytes: | hCD3 KI | splenocytes: |
| | splenocytes: | hROR1⁺ 4T1 | splenocytes: | mROR1⁺ |
| | hROR1⁺ 4T1 | cells | mROR1⁺ | 4T1 cells |
| | cells | Max IFNγ | 4T1 cells | Max IFNγ |
| | EC₅₀ (pM) | (pg/mL) | EC₅₀ (pM) | (pg/mL) |
| Ref. T cell- | 2.4 | 2182 | No activity | Not Detected |
| binder | | | | |
| Ref. Frz- | 6.7 | 2100 | 4 | 2323 |
| binding TCE | | | | |
| 1A | | | | |
| Ref. Frz- | 24.9 | 2007 | 10 | 2127 |
| binding TCE | | | | |
| 1B | | | | |
| 82H02 TCE 1A | 235.2 | 1880 | 78.4 | 2050 |
| 82H02 TCE 1B | >300 | 1771 | 140.1 | 2012 |

In co-cultures of splenocytes hCD3-knock-in mouse splenocytes and hROR1⁺ 4T1 cells, decoupling of cytotoxicity from proinflammatory cytokine release was observed for bispecific TCE 82H02 in Formats 1A and 1B but not for either reference T cell binder or reference ROR1 Frizzled domain-binding TCE in Formats 1A or 1B. A decoupling ratio ≥1.5 was observed with bispecific TCE 82H02 in Formats 1A and 1B, with a delta reaching about 1.7 and 2-fold normalized to reference T cell-binder, respectively (FIG. 9E).

Because reference T cell-binder does not bind to mROR1, in co-culture experiments of hCD3-knock-in mouse splenocytes and mROR1⁺ 4T1 cells, the decoupling ratios were calculated normalized to reference ROR1 Frizzled domain-binding TCE in Format 1A. As shown in FIG. 9F, little to no decoupling of cytotoxicity from proinflammatory cytokine release was observed in co-cultures of splenocytes hCD3-knock-in mouse splenocytes and mROR1⁺ 4T1 cells,

Overall, the magnitude of ROR1-dependent tumor cell cytotoxicity was similar when mouse tumor targets expressing human or mouse ROR1 were co-cultured with splenocytes from hCD3-knockin mice. Decoupling of cytotoxicity from proinflammatory cytokine release was observed for co-cultures with hROR1⁺ 4T1 cells but appeared to be diminished in co-cultures with mROR1⁺ 4T1 cells. These observations support the use of human ROR1 engineered syngeneic tumor bearing hCD3-knock-in mice for non-clinical in vivo pharmacokinetics/pharmacodynamics and efficacy studies.

### Example 4: Serial Killing Assay by Tumor Infiltrating T Cells (TILs)

This example describes the induction of serial killing of target tumor cells by tumor infiltrating lymphocytes (TILs) in the presence or absence of bispecific TCEs of the present disclosure.

TILs were isolated from fresh triple negative breast cancer biopsies and expanded in culture. In brief, 1 mm/1 gram of biopsy tissue pieces were incubated with 5 mL of collagenase IA (Sigma Cat No: C9891, resuspend in RPMI1640 at 100 U/ml) and incubated for 30 minutes at 37° C., then another 30 minutes at room temperature. Samples were passed through a 40 μm cell strainer and collected into 50 ml tubes. Cells were washed with RPMI1640 media and mononuclear cells isolated using Ficoll-Paque plus media (Cytiva, Cat No: GE17-1440-02) according to the manufacturer's instructions. 1×10⁶ isolated mononuclear cells were cultured with 6000 U/mL of human IL2 in RPMI1640 media supplemented with 10% heat inactivated human AB Serum, 1% penicillin/streptomycin, 10 mM glutamine, and 10 mM HEPES for 24 hours at a cell density of 1×10⁶ cells per mL. Following 24 hour incubation, 1.3×10⁵ TILs were resuspended in 10 mL of RPMI1640 media supplemented with 10% heat inactivated human AB Serum, 1% penicillin/streptomycin, 10 mM glutamine, 10 mM HEPES, 3000 U/mL human IL-2 and 0.03 μg/mL CD3 antibody clone OKT3.

Peripheral blood mononuclear cells (PBMCs) collected from six different healthy donors were gamma irradiated at 5000 rad and 2.7×10⁶ irradiated PBMCs were resuspended in AIM-V medium and added to the 1.3×10⁵ TILs in a T-25 flask. PBMC-TIL co-cultures were incubated for 96 hours then 10 mL of the culture media was removed from each flask and replaced with 5 ml AIM-V media and 5 mL of RPMI1640 supplemented with 10% heat-inactivated human AB Serum, 1% penicillin/streptomycin, 10 mM glutamine, 10 mM HEPES, and 6000 U/mL human IL2. Cells were maintained at a culture density of less than 1×10⁶ cells/mL by adding AIM-V media supplemented with 3,000 U/mL IL2. After 14-days incubation, TIL cells were counted and analyzed by flow cytometry by staining with CD3, CD4 and CD8 expression (>99% of cells were CD3⁺ T cells, and >80% CD3⁺ T cells were CD8⁺ T cells). TILs were frozen in freezing medium (ThermoFisher, Cat No: 12648010) until use in serial killing assays.

To perform the serial killing assays, frozen TILs were thawed, washed, cultured in RPMI1640 medium supplemented with 10% fetal bovine serum (FBS), 1% penicillin/streptomycin, and 500 U/mL human IL2 for 48 hours at 37° C., 5% CO₂, then serially diluted in the same medium to make TIL cell suspensions having cell densities of 1×10⁶, 5×10⁵, 2.5×10⁵, 1.25×10⁵, 6.25×10⁴, 3.125×10⁴, 1.562×10⁴, 7.81×10³ cells/mL or media alone (i.e. 0 TILs).

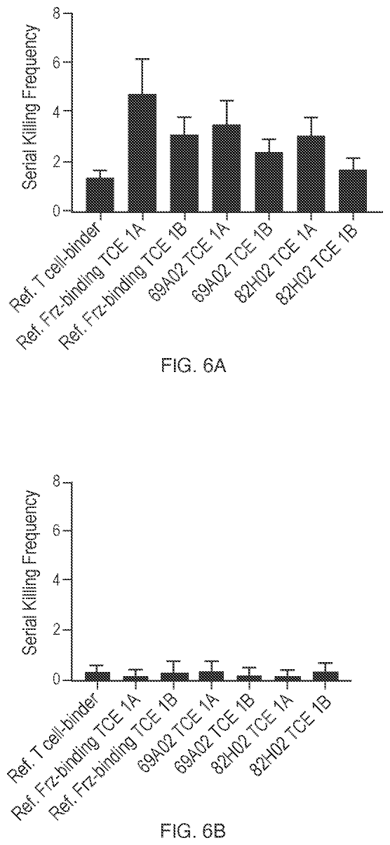

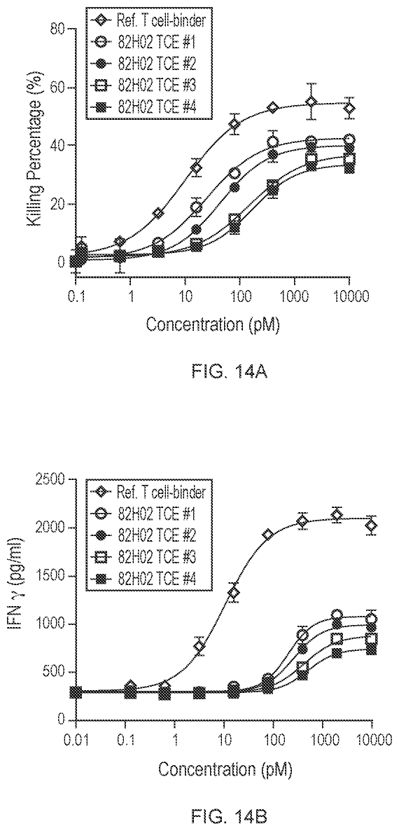

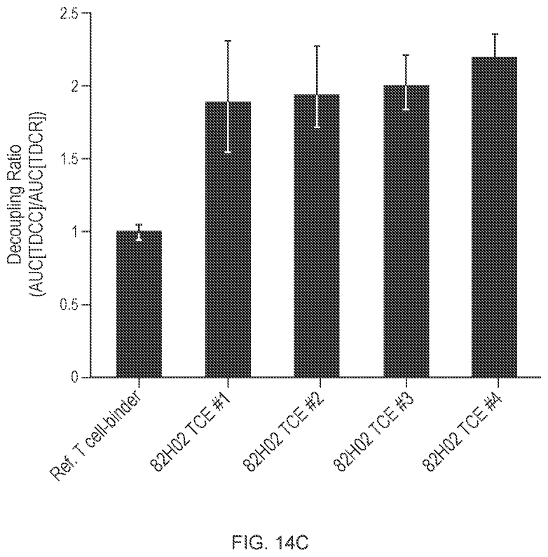

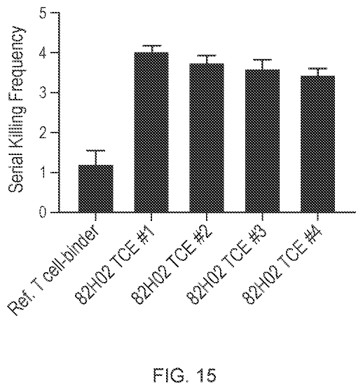

Target cells (NCI-H1975 cells (FIG. 6A and FIG. 15); and T-47D cells (FIG. 6B)) were cultured in the absence of antibiotics for 24 hours then resuspended at a cell density of 50,000 cells/mL in culture media. 100 μL of target cell suspension was carefully dispensed into to each well of a black walled, clear flat-bottom plate and incubated for 4 hours. Wells containing 20000, 10000, 5000, 2500, 1250, 625, 313, 156, 78 and 0 target cells in 100 μL of culture medium were also seeded in designated wells for use as standard references.

100 μL of each test bispecific TCE (100 pM), reference T cell-binder (100 pM), reference Frizzled-binding TCE dilution (100 pM), or media control was mixed with 100 μl of each TIL cell suspension dilution, or media control, then added to wells of the black walled, clear flat-bottom plate containing dilutions of target cells and co-cultures were incubated at 30° C., 5% CO₂ for 24 hours.

After 24 hours incubation, cells were centrifuged for 30 seconds at 1000 RPM and 100 μL of supernatant was removed from each well and transferred to a second plate for storage. 100 μL of One-Glo solution was added to each well containing the co-culture and remaining 100 μL of co-culture supernatant and incubated for 2 minutes at room temperature. The bioluminescence of the samples in the plates was measured using an Envision plate reader (or equivalent). A standard curve was plotted using the bioluminescence intensities of the target cell standard references. The number of living target cells in each co-culture sample was calculated using the standard curve and the number of killed target cells further calculated as the difference between the 5000 target cells seeded into each well and the calculated number of living target cells. The killing frequency was calculated by dividing the number of killed target cell by the number of TILs for each condition and averaged to provide killing frequencies at the E:T ratios of 1:8 and 1:16. Average killing frequencies were plotted using GraphPad Prism.

As shown in FIG. 6A, when TILs were co-cultured with NCI-H1975 cells in the presence of reference T cell-binder, little to no serial killing was observed. Bispecific TCE 82H02 in Format 1A induced between 30 to 40% greater serial killing than bispecific TCE 82H02 in Format 1A. Reference ROR1 Frizzled domain-binding TCE in Format 1A induced approximately 25% and 30% greater serial killing than bispecific TCE 69A02 in Format 1A and bispecific TCE 82H02 in Format 1A, respectively. Reference ROR1 Frizzled domain-binding TCE in Format 1B induced approximately 20% and 30% greater serial killing than bispecific TCE 69A02 in Format 1B and bispecific TCE 82H02 in Format 1B, respectively.

Overall, reference ROR1 Frizzled domain-binding TCE in Format 1A, bispecific TCE 69A02 in Format 1A and bispecific TCE 82H02 in Format 1A all induced a serial killing frequency of at least 3 tumor cells per T cell. The greater serial killing observed with molecules in Format 1A versus Format 1B was attributed to the higher apparent affinity of for CD3, likely due to reduced steric hindrance when engineered onto the N-terminus of the bridging moiety in Format 1A as compared to the C-terminus in Format 1B. As shown in FIG. 6B, no serial killing was observed when TILs were co-cultured with ROR1⁻ T47D cells, indicating that the observed serial killing was ROR1 dependent.

As shown in FIG. 15, reference T cell-binder induced little to no serial killing of NCI-H1975 tumor cells by TILs. Sequence optimized bispecific TCEs 82H02 #1, 82H02 #2, 82H02 #3, and 82H02 #4 had similar levels of serial killing (about 3 to 4 tumor cells/T cell)

### Example 5: Biosafety Assays

This example describes experiments performed to measure cytokine and chemokine release by freshly isolated PBMCs or PBMCs pre-cultured at high density, cultured in the presence or absence of bispecific TCEs of the present disclosure. Freshly isolated PBMCs from healthy donors is meant to mimic PBMCs in the circulation where cell to cell contacts would be sparse. High density precultured PBMCs (e.g., 1×10⁷ cells/mL for 48 hours) is designed to mimic PBMCs in secondary lymphoid structures (e.g., spleen and lymph nodes) where cell-to-cell contacts are high. Such cell-to-cell contacts are thought to result in weak tonic signaling of the TCR/CD3 complex on T cells through very low affinity interactions with MHC on all cells, thus mildly priming T cells. This “high density” pre-culture was shown to be necessary to recapitulate the non-specific T cell mediated proinflammatory cytokine release induced by the CD28 superagonist antibody, TGN1412 (Romer et al. (2011) Blood. 118(26):6772-82).

Human PBMCs were isolated from whole blood using Ficoll-Paque Plus medium (GE Healthcare, Cat No: 17144002) according to the manufacturer's instructions. PBMCs were then resuspended in culture medium (RPMI1640 supplemented with 10% FBS and 1% penicillin/streptomycin) at a cell density of 2×10⁶ cells/mL. 50 μL of PBMCs were added to each well of a flat-bottom 96-well plate (i.e. 100,000 cells/well). Test bispecific TCEs (82H02 TCE 1A or 82H02 TCE 1B), reference T cell-binder (Ref. T cell-binder), reference Frizzled-binding TCEs (Ref. Frz-binding TCE 1A or Ref. Frz-binding TCE 1A), isotype control antibody (hIgG) or mouse anti-human CD3 clone OKT3 (OKT3, positive control) were each prepared in RPMI1640 media supplemented with 10% FBS and 1% penicillin/streptomycin to a concentration of 24 pM. 50 μl of prepared test bispecific TCEs, reference T cell-binder, reference Frizzled-binding TCEs, hIgG, or OKT3 were added to the wells of the 96-well plate containing PBMCs to a final volume of 100 μL and incubated for 24 hours at 37° C., 5% CO₂. Following the 24 hour incubation, supernatants were collected for quantification of cytokine and chemokine release.

For high density co-culture experiments, freshly isolated PBMCs were pelleted and resuspended in RPMI1640 media supplemented with 10% FBS and 1% penicillin/streptomycin to a cell density of 1.0×10⁷ cells/mL and incubated for 48 hours at 37° C., 5% CO₂. Following 48 hour incubation, PBMCs were then resuspended in RPMI1640 media supplemented with 10% FBS and 1% penicillin/streptomycin to a cell density of 2.0×10⁶ cells/mL. 50 μL of PBMCs were added to each well of a flat-bottom 96-well plate (i.e. 100,000 cells/well). Test bispecific TCEs (82H02 TCE 1A or 82H02 TCE 1B), reference T cell-binder (Ref. T cell-binder), reference Frizzled-binding TCEs (Ref. Frz-binding TCE 1A or Ref. Frz-binding TCE 1A), isotype control antibody (hIgG) or mouse anti-human CD3 clone OKT3 (OKT3, positive control) were each prepared in RPMI1640 media supplemented with 10% FBS and 1% penicillin/streptomycin to a concentration of 24 pM. 50 μl of prepared test bispecific TCEs, reference T cell-binder, reference Frizzled-binding TCEs, hIgG, or OKT3 were added to the wells of the 96-well plate containing high density pre-cultured PBMCs to a final volume of 100 μL and incubated for 24 hours at 37° C., 5% CO₂. Following the 24 hour incubation, supernatants were collected for quantification of cytokine and chemokine release.

Cytokine and chemokine levels in cultured PBMC samples were measured using a MILLIPLEX® Human CD8⁺ T Cell Magnetic Bead Panel Premixed 17 Plex-Immunology Multiplex Assay kit (Sigma, Cat No: HCD8MAG15K17PMX) according to the manufacturer's instructions. In brief, 200 μL of assay buffer was added to the 96-well assay plate provided in the kit. The plate was then sealed and placed on a plate-shaker for 10 minutes at room temperature and the buffer was discarded. 25 μL of PBMC culture supernatants, standard or controls were added to designated wells and 25 μL of RPMI1640 culture medium was added to each well to bring the volume in each well to 50 μL. 25 μL of premixed beads were added to each well and the plate was sealed and placed on a plate-shaker for overnight incubation at 4° C. The plate was washed twice and 25 μL of prepared detection antibody was added to each well. The plate was sealed and incubated on a plate-shaker for 1 hour at room temperature. 25 μl of streptavidin-phycoerythrin (PE) was added to each well and the plate was sealed and incubated on a plate shaker for an additional 30 minutes at room temperature. After the incubation, the plate was washed twice and 150 μL of sheath fluid was added to each well. The plate was further incubated for 5 minutes on a plate-shaker and the samples were analyzed on a MAGPIX® instrument using xPONENT® software. Standard curves were generated using the readout of individual standards. The levels of cytokines or chemokines were calculated using the individual standard curve for each cytokine or chemokine. Results were plotted in GraphPad Prism (cytokine/chemokine release vs concentration of testing articles).

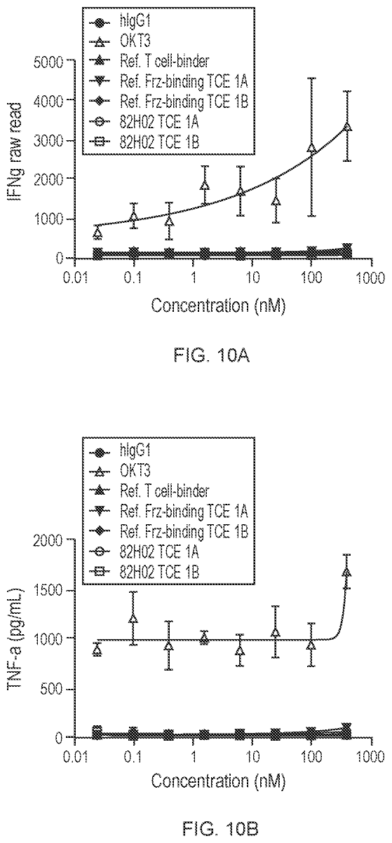

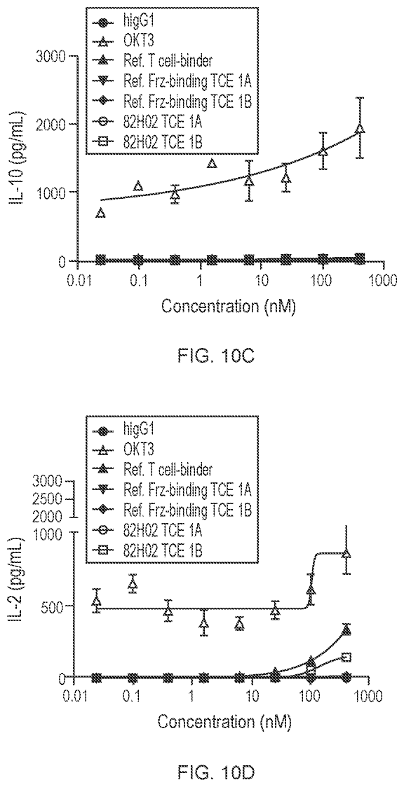

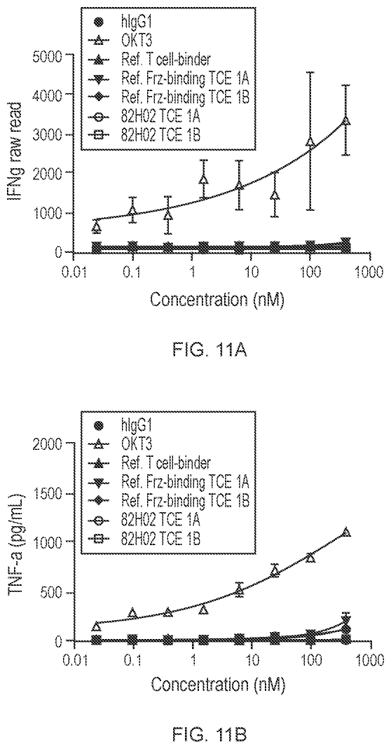

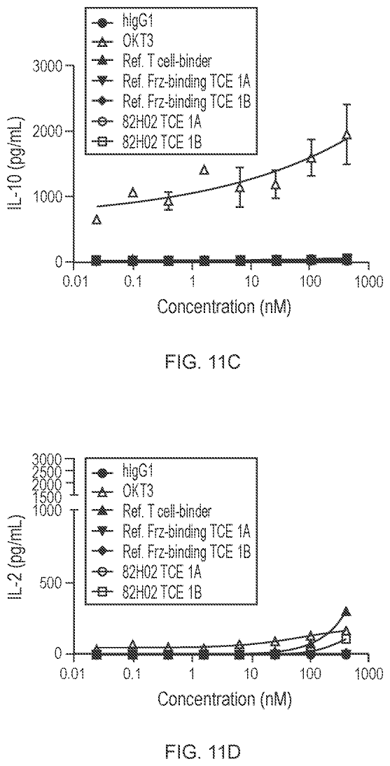

As shown in FIGS. 10A to 10D, and FIGS. 11A to 11D, anti-human CD3 clone OKT3 induced, significant cytokine release (IFNγ, TNFα, and IL10) in both freshly isolated PBMC cultures, and high-density pre-cultured PBMCs in a dose dependent manner.

Reference T cell-binder and reference Frizzled-binding TCE in Format 1B induced a small degree of non-specific IL2 release in freshly isolated PBMCs and TNFα in high-density pre-cultured PBMCs. Bispecific TCE 82H02 in Formats 1A and 1B did not induce release of significant levels of cytokines as compared to background or hIgG1 isotype control.

### Example 6: Competition Assays

This example describes experiments performed to determine whether ROR1-binding clones of the present disclosure (82H02 and 69A02), bispecific reference T cell-binders, or reference Frizzled-binding clones compete for binding to ROR1 on NCCIT cells.

NCCIT cell cultures were tested for viability and resuspended in culture media to a cell density of 1×10⁶ cells/ml. 200 μl of NCCIT cell suspension was added to each well of a U-bottom 96 well plate (i.e. 200,000 cells per well). Cells were washed twice with PBS and 100 μl of live/dead fixable staining solution (Thermo Fisher, Cat No: L34961) was added to each well. Cells were incubated at room temperature in the dark for 15 minutes, washed twice with PBS, then resuspended in 100 μL of serially-diluted unlabeled 82H02 scFv-Fc, 69A02 scFv-Fc, Ref. T cell-binder scFv-Fc, or Ref. Frz-binding scFv-Fc (three-fold dilutions from 1000 nM to 0.017 nM) in staining buffer (eBiosciences, Cat No: 00-4222-26). Cells were then incubated in the dark for 30 minutes on ice, washed with ice cold PBS, then resuspended in 100 μL of 146 nM 82H02 scFv-Fc labeled with Alexa Fluor 647-conjugated Zenon fragments (Thermo Fisher, Cat No: Z25408). Cells were incubated on ice for 30 minutes in the dark, washed three times with ice cold PBS, then fixed in 50 μl/well of fixing buffer (Biolegend, Cat No: 42080) for 10 minutes at room temperature. Cells were then resuspended in 200 μl of PBS and cells were analyzed by flow cytometry performed on a Fortessa X-20 instrument using FlowJo software.

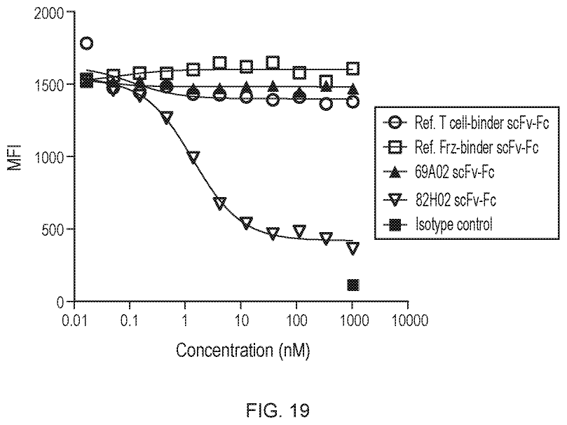

As shown in FIG. 19, Alexa Fluor 647-conjugated Zenon fragment-labeled 82H02 scFv-Fc did not bind ROR-1 on NCCIT cells that were occupied by its competing antibody (i.e. unlabelled 82H02 scFv-Fc) and gave weak Alexa Fluor 647 signal as measured by flow cytometry. In contrast, significant Alexa Fluor 647 signal was measured in NCCIT cell samples pre-treated with 69A02 scFv-Fc, Ref. T cell-binder scFv-Fc, or Ref. Frz-binding scFv-Fc suggesting that Alexa Fluor 647-conjugated Zenon fragment-labeled 82H02 scFv-Fc does not compete for binding of ROR1 with these molecules and that these molecules bind to different epitopes/regions of ROR1.

### Example 7: Binding Affinity Assays

This example describes biolayer interferometry experiments that were used to determine the binding affinities of bispecific TCEs and sequence optimized variants of the present disclosure.

TABLE 29 shows K_{D} values of immobilized bispecific TCEs against recombinant human ROR1 extracellular domain protein as measured by biolayer interferometry.

TABLE 29
Binding Affinities for ROR1-Binding Bispecific TCEs

| | K_{D} |
|---|---|
| | Human/Cyno |
| Construct ID | ROR1 (nM) |
| Reference Frizzled domain- | 1.1 |
| Binder | |
| 69A02 | 3.5 |
| 82H02 | 5.4 |
| 82H02 VL1.02 | 121.9 |
| 82H02 VL1.03 | 475.2 |
| 82H02 VL1.04 | 50.7 |
| 82H02 VL1.05 | 153.6 |
| 82H02 VL1.06 (low | 99.0 |
| 82H02 VL1.07 (high) | 4.2 |
| 82H02 VL1.08 | 166.4 |
| 82H02 VL1.09 | 160.5 |

TABLE 30 shows K_{D} values of immobilized bispecific TCEs against recombinant human CD3ε/δ heterodimer SEED Fc fusion protein as measured by biolayer interferometry.

TABLE 30
Binding Affinities for CD3-Binding Bispecific TCEs

| | K_{D} |
|---|---|
| | Human CD3ε |
| Construct ID | (nM) |
| Parental 1F3.5 | 2.7 |
| 1F3.5 VL1.00-VH1.01 | 2.9 |
| 1F3.5 VL1.00-VH1.02 | 5.5 |
| 1F3.5 VL1.00-VH1.03 (high) | 4.6 |
| 1F3.5 VL1.00-VH1.04 | 6.5 |
| 1F3.5 VL1.00-VH1.05 | 8.2 |
| 1F3.5 VL1.01-VH1.00 | 62.3 |
| 1F3.5 VL1.01-VH1.01 (low) | 42.0 |
| 1F3.5 VL1.01-VH1.02 | 47.1 |
| 1F3.5 VL1.01-VH1.03 | 84.8 |
| 1F3.5 VL1.01-VH1.04 | 74.2 |
| 1F3.5 VL1.01-VH1.05 | 59.2 |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1. A protein comprising:
a polypeptide or complex of two or more polypeptides that specifically binds ROR1 connected to an end of a bridging moiety; and
a polypeptide or complex of two or more polypeptides that specifically binds CD3 connected to an opposite end of the bridging moiety.

2-194. (canceled)

195. The protein of claim 1, wherein the polypeptide or complex of two or more polypeptides that specifically binds ROR1 is a first single-chain variable fragment (scFv) and the polypeptide or complex of two or more polypeptides that specifically binds CD3 is a Fab.

196. The protein of claim 195, wherein:
(i) the first scFv that specifically binds ROR1 is connected to the C-terminal end of the bridging moiety and the Fab that specifically binds CD3 is connected to the N-terminal end of the bridging moiety; or
(ii) the first scFv that specifically binds ROR1 is connected to the N-terminal end of the bridging moiety and the Fab that specifically binds CD3 is connected to the C-terminal end of the bridging moiety.

197. The protein of claim 196, further comprising a second scFv that specifically binds ROR1 connected to the N-terminal end of the bridging moiety.

198. The protein of claim 197, wherein the bridging moiety comprises:
(i) two polypeptide arms and wherein the first scFv that specifically binds ROR1 and the second scFv that specifically binds ROR1 are connected to the C-terminal end and the N-terminal end of the same polypeptide arm, respectively, and the Fab that specifically binds CD3 is connected to the N-terminal end of the second polypeptide arm; or
(ii) two polypeptide arms and wherein the first scFv that specifically binds ROR1 and the Fab that specifically binds CD3 are connected to the N-terminal end and the C-terminal end of the sample polypeptide arm, respectively, and the second scFv that specifically binds ROR1 is connected to the N-terminal end of the second polypeptide arm.

199. The protein of claim 197, wherein the first scFv and/or the second scFv that specifically binds ROR1 comprises:
(i) a heavy chain complementarity-determining region 1 (VHCDR1) comprising an amino acid sequence of GFTFTSYA (SEQ ID NO: 8) or an amino acid sequence of GFSITTSGVS (SEQ ID NO: 23); a heavy chain complementarity-determining region 2 (VHCDR2) comprising an amino acid sequence of ISGSGGGT (SEQ ID NO: 9) or an amino acid sequence of IYWDDDK (SEQ ID NO: 24); a heavy chain complementarity-determining region 3 (VHCDR3) comprising an amino acid sequence of AQGSSIFDY (SEQ ID NO: 10) or an amino acid sequence of AHAPRYTPGGYFDY (SEQ ID NO: 25); a light chain complementarity-determining region 1 (VLCDR1) comprising an amino acid sequence of QSVSSY (SEQ ID NO: 11) or an amino acid sequence of QSVSSN (SEQ ID NO: 26); a light chain complementarity-determining region 2 (VLCDR2) comprising an amino acid sequence of DAS or GAS; and a light chain complementarity-determining region 3 (VLCDR3) comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28);
(ii) (a) a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14);
(b) a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15);
(c) a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28); or
(d) according to the IMGT unique numbering scheme, a VHCDR1, a VHCDR2, a VHCDR3, a VLCDR1, a VLCDR2, and a VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 1, respectively;
(iii) (a) a sequence having at least 90% sequence identity to SEQ ID NO: 29 and a sequence having at least 90% sequence identity to SEQ ID NO: 30;
(b) a sequence having at least 90% sequence identity to SEQ ID NO: 29 and a sequence having at least 90% sequence identity to SEQ ID NO: 31;
(c) a sequence having at least 90% sequence identity to SEQ ID NO: 29 or SEQ ID NO: 39 and a sequence having at least 90% sequence identity to SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, or SEQ ID NO: 40;
(d) a sequence having at least 90% sequence identity to SEQ ID NO: 39 and a sequence having at least 90% sequence identity to SEQ ID NO: 40; or
(e) a heavy chain variable domain and a light chain variable domain each comprising an amino acid sequence corresponding to the heavy chain variable domain and light chain variable domain sequences of a heavy chain variable domain and light chain variable domain listed in TABLE 2, respectively;
(iv) a linker polypeptide comprising a (GGGGS)ₙ (SEQ ID NO: 1) sequence, wherein n is 1 to 12; and/or
(v) (a) a sequence having at least 90% sequence identity to SEQ ID NO: 41;
(b) a sequence having at least 90% sequence identity to SEQ ID NO: 42;
(c) a sequence having at least 90% sequence identity to SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, or SEQ ID NO: 59;
(d) a sequence having at least 90% sequence identity to SEQ ID NO: 59; or
(e) an scFv comprising an amino acid sequence corresponding to an scFv listed in TABLE 3.

200. The protein of claim 197, wherein the Fab that specifically binds CD3 comprises:
(i) a VHCDR1 comprising an amino acid sequence of GFTFNTYA (SEQ ID NO: 61); a VHCDR2 comprising an amino acid sequence of IRSKYNNYAT (SEQ ID NO: 62); a VHCDR3 comprising an amino acid sequence of VRHGNFGX₁X₂YVSWFAY (SEQ ID NO: 67), wherein X₁ is N, A, or E, and X₂ is S or A; a VLCDR1 comprising an amino acid sequence of TGAVTTSN (SEQ ID NO: 64); a VLCDR2 comprising an amino acid sequence of GT; and a VLCDR3 comprising an amino acid sequence of ALWYSNLWV (SEQ ID NO: 66);
(ii) (a) a VHCDR1 comprising an amino acid sequence of GFTFNTYA (SEQ ID NO: 61); a VHCDR2 comprising an amino acid sequence of IRSKYNNYAT (SEQ ID NO: 62); a VHCDR3 comprising an amino acid sequence of VRHGNFGNSYVSWFAY (SEQ ID NO: 63); a VLCDR1 comprising an amino acid sequence of TGAVTTSN (SEQ ID NO: 64); a VLCDR2 comprising an amino acid sequence of GT; and a VLCDR3 comprising an amino acid sequence of ALWYSNLWV (SEQ ID NO: 66);
(b) a VHCDR1 comprising an amino acid sequence of GFTFNTYA (SEQ ID NO: 61); a VHCDR2 comprising an amino acid sequence of IRSKYNNYAT (SEQ ID NO: 62); a VHCDR3 comprising an amino acid sequence of VRHGNFGASYVSWFAY (SEQ ID NO: 68); a VLCDR1 comprising an amino acid sequence of TGAVTTSN (SEQ ID NO: 64); a VLCDR2 comprising an amino acid sequence of GT; and a VLCDR3 comprising an amino acid sequence of ALWYSNLWV (SEQ ID NO: 66); or
(c) according to the IMGT unique numbering scheme, a VHCDR1, a VHCDR2, a VHCDR3, a VLCDR1, a VLCDR2, and a VLCDR3 each comprising an amino acid sequence corresponding to the VHCDR1, VHCDR2, VHCDR3, VLCDR1, VLCDR2, and VLCDR3 sequences of a heavy chain variable domain and a light chain variable domain listed in TABLE 4, respectively;
(iii) (a) a sequence having at least 90% sequence identity to SEQ ID NO: 71 and a sequence having at least 90% sequence identity to SEQ ID NO: 76;
(b) a sequence having at least 90% sequence identity to SEQ ID NO: 71, SEQ ID NO: 72, SEQ ID NO: 73, SEQ ID NO: 74, SEQ ID NO: 75, or SEQ ID NO: 80 and a sequence having at least 90% sequence identity to SEQ ID NO: 76, SEQ ID NO: 77, SEQ ID NO: 78, SEQ ID NO: 79, or SEQ ID NO: 81;
(c) a sequence having at least 90% sequence identity to SEQ ID NO: 72 and a sequence having at least 90% sequence identity to SEQ ID NO: 81; or
(d) according to the IMGT unique numbering scheme, a heavy chain variable domain and a light chain variable domain each comprising an amino acid sequence corresponding to the heavy chain variable domain and light chain variable domain sequences of a heavy chain variable domain and light chain variable domain listed in TABLE 5, respectively; and/or
(iv) (a) a sequence having at least 90% sequence identity to SEQ ID NO: 83 and a sequence having at least 90% sequence identity to SEQ ID NO: 85;
(b) a sequence having at least 90% sequence identity to SEQ ID NO: 82 or SEQ ID NO: 83, and a sequence having at least 90% sequence identity to SEQ ID NO: 84 or SEQ ID NO: 85;
(c) a sequence having at least 90% sequence identity to SEQ ID NO: 82 and a sequence having at least 90% sequence identity to SEQ ID NO: 84; or
(d) according to the IMGT unique numbering scheme, a heavy chain and a light chain each comprising an amino acid sequence corresponding to the heavy chain and light chain sequences of a heavy chain and light chain listed in TABLE 6, respectively.

201. The protein of claim 197, wherein:
(a) the bridging moiety is functional or non-functional; and/or
(b) the bridging moiety comprises a strand-exchange engineered domain (SEED) format Fc domain or functional fragment thereof, an immunoglobulin Fc domain or functional fragment thereof, a polypeptide linker, or a polypeptide hinge.

202. The protein of claim 201, wherein the bridging moiety comprises:
(i) a SEED format Fc domain and wherein the SEED format Fc domain comprises:
(a) one or more than one effector function silencing mutation;
(b) a polypeptide comprising a sequence having at least 90% sequence identity to SEQ ID NO: 86, SEQ ID NO: 117, or SEQ ID NO: 88, and a polypeptide comprising a sequence having at least 90% sequence identity to SEQ ID NO: 87, SEQ ID NO: 118, or SEQ ID NO: 89; and/or
(c) a polypeptide comprising or consisting of SEQ ID NO: 86 and a polypeptide comprising or consisting of SEQ ID NO: 87; or
(ii) an immunoglobulin Fc domain and wherein the immunoglobulin Fc domain comprises:
(a) two polypeptide arms each comprising one or more than one mutation promoting heterodimerization; and/or
(b) a first polypeptide arm and a second polypeptide arm each comprising an amino acid sequence corresponding to the first polypeptide arm and second polypeptide arm sequences of a first polypeptide arm and second polypeptide arm listed in TABLE 8, respectively.

203. The protein of claim 202, wherein the bridging moiety further comprises a hinge at its N-terminal end and wherein the hinge comprises a polypeptide arm comprising an amino acid sequence comprising of SEQ ID NO: 92 or an amino acid sequence listed in Table 9.

204. The protein of claim 203, wherein the hinge further comprises a second polypeptide arm comprising an amino acid sequence comprising of SEQ ID NO: 90, SEQ ID NO: 92, or an amino acid sequence listed in Table 9.

205. The protein of claim 197, wherein:
(a) the first scFv that specifically binds ROR1 is connected to the C-terminal end of the bridging moiety by a linker polypeptide; and/or
(b) the Fab that specifically binds CD3 is connected to the C-terminal end of the bridging moiety by a linker polypeptide

206. The protein of claim 205, wherein the linker polypeptide comprises a (GGGGS)ₙ (SEQ ID NO: 1) sequence, wherein n is 1 to 12.

207. A protein comprising:
(a) (i) a first single-chain variable fragment (scFv) that specifically binds ROR1 comprising a heavy chain complementarity-determining region 1 (VHCDR1) comprising an amino acid sequence of GFTFTSYA (SEQ ID NO: 8) or an amino acid sequence of GFSITTSGVS (SEQ ID NO: 23), a heavy chain complementarity-determining region 2 (VHCDR2) comprising an amino acid sequence of ISGSGGGT (SEQ ID NO: 9) or an amino acid sequence of IYWDDDK (SEQ ID NO: 24), a heavy chain complementarity-determining region 3 (VHCDR3) comprising an amino acid sequence of AQGSSIFDY (SEQ ID NO: 10) or an amino acid sequence of AHAPRYTPGGYFDY (SEQ ID NO: 25), a light chain complementarity-determining region 1 (VLCDR1) comprising an amino acid sequence of QSVSSY (SEQ ID NO: 11) or an amino acid sequence of QSVSSN (SEQ ID NO: 26), a light chain complementarity-determining region 2 (VLCDR2) comprising an amino acid sequence of DAS or GAS, and a light chain complementarity-determining region 3 (VLCDR3) comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28);
(ii) a second scFv that specifically binds ROR1 comprising a VHCDR1 comprising an amino acid sequence of GFTFTSYA (SEQ ID NO: 8) or an amino acid sequence of GFSITTSGVS (SEQ ID NO: 23), a VHCDR2 comprising an amino acid sequence of ISGSGGGT (SEQ ID NO: 9) or an amino acid sequence of IYWDDDK (SEQ ID NO: 24), a VHCDR3 comprising an amino acid sequence of AQGSSIFDY (SEQ ID NO: 10) or an amino acid sequence of AHAPRYTPGGYFDY (SEQ ID NO: 25), a VLCDR1 comprising an amino acid sequence of QSVSSY (SEQ ID NO: 11) or an amino acid sequence of QSVSSN (SEQ ID NO: 26), a VLCDR2 comprising an amino acid sequence of DAS or GAS, and a VLCDR3 comprising an amino acid sequence of QQRSNWPPXT (SEQ ID NO: 13), wherein X is F, S, C, R, Q, I, L, Y or V, or an amino acid sequence of QQYKNWPPA (SEQ ID NO: 28);
(iii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGX₁X₂YVSWFAY (SEQ ID NO: 67), wherein X₁ is N, A, or E, and X₂ is S or A, a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66); and
(iv) a bridging moiety connecting the scFv that specifically binds ROR1 to the Fab that specifically binds CD3, wherein the bridging moiety comprises (a) a polypeptide arm comprising amino acid sequences of SEQ ID NO: 90 and SEQ ID NO: 86 or SEQ ID NO: 92 and SEQ ID NO: 86, and (b) a second polypeptide arm comprising amino acid sequences of SEQ ID NO: 90 and SEQ ID NO: 87, or SEQ ID NO: 92 and SEQ ID NO: 87; or
(b) (i) a first scFv that specifically binds ROR1 comprising a heavy chain variable domain (VH) comprising an amino acid sequence of SEQ ID NO: 29 or SEQ ID NO: 39, and a light chain variable domain (VL) comprising an amino acid sequence of SEQ ID NO: 30, SEQ ID NO: 31, SEQ ID NO: 32, SEQ ID NO: 33; SEQ ID NO: 34, SEQ ID NO: 35, SEQ ID NO: 36, SEQ ID NO: 37, SEQ ID NO: 38, or SEQ ID NO: 40;
(ii) a second scFv that specifically binds ROR1 comprising a heavy chain variable domain (VH) comprising an amino acid sequence of SEQ ID NO: 29 or SEQ ID NO: 39, and a light chain variable domain (VL) comprising an amino acid sequence of SEQ ID NO: 30, SEQ ID NO: 31, SEQ ID NO: 32, SEQ ID NO: 33; SEQ ID NO: 34, SEQ ID NO: 35, SEQ ID NO: 36, SEQ ID NO: 37, SEQ ID NO: 38, or SEQ ID NO: 40;
(iii) a Fab that specifically binds CD3 comprising a VH comprising an amino acid sequence of SEQ ID NO: 71, SEQ ID NO: 72, SEQ ID NO: 73, SEQ ID NO: 74, SEQ ID NO: 75, or SEQ ID NO: 80, and a VL comprising an amino acid sequence of SEQ ID NO: 76, SEQ ID NO: 77, SEQ ID NO: 78, SEQ ID NO: 79, or SEQ ID NO: 81; and
(iv) a bridging moiety connecting the polypeptide or complex of two or more polypeptides that specifically binds ROR1 to the polypeptide or complex of two or more polypeptides that specifically binds CD3, wherein the bridging moiety comprises: (a) a polypeptide arm comprising amino acid sequences of SEQ ID NO: 90 and SEQ ID NO: 86, or SEQ ID NO: 92 and SEQ ID NO: 86, and (b) a second polypeptide arm comprising an amino acid sequence of SEQ ID NO: 90 and SEQ ID NO: 87, or SEQ ID NO: 92 and SEQ ID NO: 87; or
(c) (i) a first scFv that specifically binds ROR1 comprising an amino acid sequence of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or SEQ ID NO: 60;
(ii) a second scFv that specifically binds ROR1 comprising an amino acid sequence of SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or SEQ ID NO: 60;
(iii) a Fab that specifically binds CD3 comprising a heavy chain comprising an amino acid sequence of SEQ ID NO: 82 or SEQ ID NO: 83, and a light chain comprising an amino acid sequence of SEQ ID NO: 84 or SEQ ID NO: 85; and
(iv) a bridging moiety connecting the scFv that specifically binds ROR1 to the Fab that specifically binds CD3, wherein the bridging moiety comprises: (a) a polypeptide arm comprising amino acid sequences of SEQ ID NO: 90 and SEQ ID NO: 86, or SEQ ID NO: 92 and SEQ ID NO: 86, and (b) a second polypeptide arm comprising amino acid sequences of SEQ ID NO: 90 and SEQ ID NO: 87, or SEQ ID NO: 92 and SEQ ID NO: 87.

208. The protein of claim 207, comprising:
(a) (i) a first scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) a second scFv that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15); or
(b) (i) a first scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGASYVSWFAY (SEQ ID NO: 68), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) a second scFv that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15); or
(c) (i) a first scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) a second scFv that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14); or
(d) (i) a first scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGASYVSWFAY (SEQ ID NO: 68), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) a second scFv that specifically binds ROR1 connected to the N-terminal end of the bridging moiety comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPFT (SEQ ID NO: 14); or
(e) (i) a first scFv that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 90 and SEQ ID NO: 86 and (b) a second polypeptide arm comprising amino acid sequences of SEQ ID NO: 92 and SEQ ID NO: 87; and
(iv) a second scFv that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28); or
(f) (i) a first scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising amino acid sequences of SEQ ID NO: 92 and SEQ ID NO: 87; and
(iv) a second scFv that specifically binds ROR1 comprising a VHCDR1 comprising an amino acid sequence of GFTFTSYA (SEQ ID NO: 8), a VHCDR2 comprising an amino acid sequence of ISGSGGGT (SEQ ID NO: 9), a VHCDR3 comprising an amino acid sequence of AQGSSIFDY (SEQ ID NO: 10), a VLCDR1 comprising an amino acid sequence of QSVSSY (SEQ ID NO: 11), a VLCDR2 comprising an amino acid sequence of DAS, and a VLCDR3 comprising an amino acid sequence of QQRSNWPPST (SEQ ID NO: 15); or
(g) (i) a first scFv that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28);
(ii) a Fab that specifically binds CD3 comprising a VHCDR1 comprising an amino acid sequence of GFTFNTYA (SEQ ID NO: 61), a VHCDR2 comprising an amino acid sequence of IRSKYNNYAT (SEQ ID NO: 62), a VHCDR3 comprising an amino acid sequence of VRHGNFGNSYVSWFAY (SEQ ID NO: 63), a VLCDR1 comprising an amino acid sequence of TGAVTTSN (SEQ ID NO: 64), a VLCDR2 comprising an amino acid sequence of GT, and a VLCDR3 comprising an amino acid sequence of ALWYSNLWV (SEQ ID NO: 66);
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising amino acid sequences of SEQ ID NO: 92 and SEQ ID NO: 87; and
(iv) a second scFv that specifically binds ROR1 comprising a VHCDR1 comprising an amino acid sequence of GFSITTSGVS (SEQ ID NO: 23), a VHCDR2 comprising an amino acid sequence of IYWDDDK (SEQ ID NO: 24), a VHCDR3 comprising an amino acid sequence of AHAPRYTPGGYFDY (SEQ ID NO: 25), a VLCDR1 comprising an amino acid sequence of QSVSSN (SEQ ID NO: 26), a VLCDR2 comprising an amino acid sequence of GAS, and a VLCDR3 comprising an amino acid sequence of QQYKNWPPA (SEQ ID NO: 28).

209. The protein of claim 207, comprising:
(a) (i) a first scFv that specifically binds ROR1 comprising a VH comprising an amino acid sequence of SEQ ID NO: 29, and a VL comprising an amino acid sequence of SEQ ID NO: 31;
(ii) a fab that specifically binds CD3 comprising a VH comprising an amino acid sequence of SEQ ID NO: 72, and a VL comprising an amino acid sequence of SEQ ID NO: 81;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 29, and a VL comprising an amino acid sequence of SEQ ID NO: 31; or
(b) (i) a first scFv that specifically binds ROR1 comprising a VH comprising an amino acid sequence of SEQ ID NO: 29, and a VL comprising an amino acid sequence of SEQ ID NO: 31;
(ii) a Fab that specifically binds CD3 comprising a VH comprising an amino acid sequence of SEQ ID NO: 71, and a VL comprising an amino acid sequence of SEQ ID NO: 76;
(iii) a bridging moiety connecting the N-terminal end of the first scFv that specifically binds ROR1 to the C-terminal end of the Fab that specifically binds CD3, wherein the bridging moiety comprises: (a) two polypeptides at its N-terminal end comprising an amino acid sequence of SEQ ID NO: 92, and (b) a polypeptide comprising an amino acid sequence of SEQ ID NO: 86 and a polypeptide comprising an amino acid sequence of SEQ ID NO: 87 wherein the bridging moiety comprises: (1) a polypeptide arm comprising amino acid sequences of SEQ ID NO: 90 and SEQ ID NO: 86, and (2) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 29, and a VL comprising an amino acid sequence of SEQ ID NO: 31; or
(c) (i) a first scFv that specifically binds ROR1 comprising a VH comprising an amino acid sequence of SEQ ID NO: 29, and a VL comprising an amino acid sequence of SEQ ID NO: 30;
(ii) a Fab that specifically binds CD3 comprising a VH comprising an amino acid sequence of SEQ ID NO: 72, and a VL comprising an amino acid sequence of SEQ ID NO: 81;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising an amino acid sequence of SEQ ID NO: 92 and SEQ ID NO: 87; and
(iii) a second scFv that specifically binds ROR1 connected to the N-terminal end of the bridging moiety comprising a VH comprising an amino acid sequence of SEQ ID NO: 29, and a VL comprising an amino acid sequence of SEQ ID NO: 30; or
(d) (i) a first scFv that specifically binds ROR1 comprising a VH comprising an amino acid sequence of SEQ ID NO: 29, and a VL comprising an amino acid sequence of SEQ ID NO: 30;
(ii) a Fab that specifically binds CD3 comprising a VH comprising an amino acid sequence of SEQ ID NO: 71, and a VL comprising an amino acid sequence of SEQ ID NO: 76;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 29, and a VL comprising an amino acid sequence of SEQ ID NO: 30;
(e) (i) a first scFv that specifically binds ROR1 comprising a VH comprising an amino acid sequence of SEQ ID NO: 39, and a VL comprising an amino acid sequence of SEQ ID NO: 40;
(ii) a Fab that specifically binds CD3 comprising a VH comprising an amino acid sequence of SEQ ID NO: 80, and a VL comprising an amino acid sequence of SEQ ID NO: 81;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 39, and a VL comprising an amino acid sequence of SEQ ID NO: 40; or
(f) (i) a first scFv that specifically binds ROR1 comprising a VH comprising an amino acid sequence of SEQ ID NO: 29, and a VL comprising an amino acid sequence of SEQ ID NO: 31;
(ii) a Fab that specifically binds CD3 comprising a VH comprising an amino acid sequence of SEQ ID NO: 72, and a VL comprising an amino acid sequence of SEQ ID NO: 81;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 29, and a VL comprising an amino acid sequence of SEQ ID NO: 31; or
(g) (i) a first scFv that specifically binds ROR1 comprising a VH comprising an amino acid sequence of SEQ ID NO: 39, and a VL comprising an amino acid sequence of SEQ ID NO: 40;
(ii) a Fab that specifically binds CD3 comprising a VH comprising an amino acid sequence of SEQ ID NO: 72, and a VL comprising an amino acid sequence of SEQ ID NO: 81;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising an amino acid sequence of SEQ ID NO: 92 and SEQ ID NO: 87; and
(iv) a second scFv that specifically binds ROR1 connected to the N-terminal end of the bridging moiety comprising a VH comprising an amino acid sequence of SEQ ID NO: 39, and a VL comprising an amino acid sequence of SEQ ID NO: 40.

210. The protein of claim 207, wherein
(a) (i) the first scFv that specifically binds ROR1 comprises an amino acid sequence of SEQ ID NO: 42;
(ii) the Fab that specifically binds CD3 comprises a heavy chain comprising an amino acid sequence of SEQ ID NO: 82, and a light chain comprising an amino acid sequence of SEQ ID NO: 84;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 42; or
(b) (i) the first scFv that specifically binds ROR1 comprises an amino acid sequence of SEQ ID NO: 42;
(ii) a Fab that specifically binds CD3 comprises a heavy chain comprising an amino acid sequence of SEQ ID NO: 83, and a light chain comprising an amino acid sequence of SEQ ID NO: 85;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 42; or
(c) (i) the first scFv that specifically binds ROR1 comprises an amino acid sequence of SEQ ID NO: 41;
(ii) the Fab that specifically binds CD3 comprises a heavy chain comprising an amino acid sequence of SEQ ID NO: 82, and a light chain comprising an amino acid sequence of SEQ ID NO: 84;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 41;
(d) (i) the first scFv that specifically binds ROR1 comprises an amino acid sequence of SEQ ID NO: 41;
(ii) the Fab that specifically binds CD3 comprises a heavy chain comprising an amino acid sequence of SEQ ID NO: 83, and a light chain comprising an amino acid sequence of SEQ ID NO: 85;
(iii) a bridging moiety connecting the N-terminal end of the first scFv that specifically binds ROR1 to the C-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 41; or
(e) (i) the first scFv that specifically binds ROR1 comprises an amino acid sequence of SEQ ID NO: 59;
(ii) the Fab that specifically binds CD3 comprises a heavy chain comprising an amino acid sequence of SEQ ID NO: 93, and a light chain comprising an amino acid sequence of SEQ ID NO: 84;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 90 and SEQ ID NO: 86, and (b) a second polypeptide arm comprising amino acid sequences of SEQ ID NO: 92 and SEQ ID NO: 87; and
(iv) a second scFv that specifically binds ROR1 is connected to the N-terminal end of the bridging moiety comprising an amino acid sequence of SEQ ID NO: 59; or
(f) (i) the first scFv that specifically binds ROR1 comprises an amino acid sequence of SEQ ID NO: 42;
(ii) the Fab that specifically binds CD3 comprises a heavy chain comprising an amino acid sequence of SEQ ID NO: 93, and a light chain comprising an amino acid sequence of SEQ ID NO: 84;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 42; or
(g) (i) the first scFv that specifically binds ROR1 comprises an amino acid sequence of SEQ ID NO: 59;
(ii) the Fab that specifically binds CD3 comprises a heavy chain comprising an amino acid sequence of SEQ ID NO: 93, and a light chain comprising an amino acid sequence of SEQ ID NO: 84;
(iii) a bridging moiety connecting the C-terminal end of the first scFv that specifically binds ROR1 to the N-terminal end of the Fab that specifically binds CD3, wherein the bridging moiety comprises: (a) a polypeptide arm comprising amino acid sequences of SEQ ID NO: 92 and SEQ ID NO: 86, and (b) a second polypeptide arm comprising amino acid sequences of SEQ ID NO: 92 and SEQ ID NO: 87; and
(iv) the second scFv that specifically binds ROR1 is connected to the N-terminal end of the bridging moiety comprising an amino acid sequence of SEQ ID NO: 59.

211. The protein of claim 207, wherein:
(a) the first scFv that specifically binds ROR1 is connected to the C-terminal end of the bridging moiety, the second scFv that specifically binds ROR1 is connected to the N-terminal end of the bridging moiety, and the Fab that specifically binds CD3 is connected to the N-terminal end of the bridging moiety; or
(b) the first scFv that specifically binds ROR1 is connected to the N-terminal end of the bridging moiety, the second scFv that specifically binds ROR1 is connected to the N-terminal end of the bridging moiety, and the Fab that specifically binds CD3 is connected to the C-terminal end of the bridging moiety.

212. The protein of claim 211, wherein the first scFv that specifically binds ROR1, the second scFv that specifically binds ROR1, and/or the Fab that specifically binds CD3 are connected to the bridging moiety by a linker polypeptide.

213. The protein of claim 212, wherein the linker polypeptide comprises a (GGGGS)ₙ (SEQ ID NO: 1) sequence, wherein n is 1 to 12.

214. A protein comprising:
(i) a polypeptide having at least 90% sequence identity to an amino acid sequence selected from the group consisting of: SEQ ID NO: 95, SEQ ID NO: 98, SEQ ID NO: 101, SEQ ID NO: 104, SEQ ID NO: 219, SEQ ID NO: 109, and SEQ ID NO: 220;
(ii) polypeptides having at least 90% sequence identity to amino acid sequences selected from the group consisting of:
(a) SEQ ID NO: 97, SEQ ID NO: 98, and SEQ ID NO: 85;
(b) SEQ ID NO: 103, SEQ ID NO: 104, and SEQ ID NO: 85;
(c) SEQ ID NO: 94, SEQ ID NO: 95, and SEQ ID NO: 84;
(d) SEQ ID NO: 100, SEQ ID NO: 101, and SEQ ID NO: 84;
(e) SEQ ID NO: 218, SEQ ID NO: 219, and SEQ ID NO: 85;
(f) SEQ ID NO: 109, SEQ ID NO: 110, and SEQ ID NO: 84; and
(g) SEQ ID NO: 220, SEQ ID NO: 221, and SEQ ID NO: 84;
(iii) a polypeptide comprising an amino acid sequence of SEQ ID NO: 97, a polypeptide comprising an amino acid sequence of SEQ ID NO: 98, and a polypeptide comprising an amino acid sequence of SEQ ID NO: 85;
(iv) a polypeptide comprising an amino acid sequence of SEQ ID NO: 103, a polypeptide comprising an amino acid sequence of SEQ ID NO: 104, and a polypeptide comprising an amino acid sequence of SEQ ID NO: 85;
(v) a polypeptide comprising an amino acid sequence of SEQ ID NO: 100, a polypeptide comprising an amino acid sequence of SEQ ID NO: 101, and a polypeptide comprising an amino acid sequence of SEQ ID NO: 84;
(vi) a polypeptide comprising an amino acid sequence of SEQ ID NO: 94, a polypeptide comprising an amino acid sequence of SEQ ID NO: 95, and a polypeptide comprising an amino acid sequence of SEQ ID NO: 84;
(vii) a polypeptide comprising an amino acid sequence of SEQ ID NO: 218, a polypeptide comprising an amino acid sequence of SEQ ID NO: 219, and a polypeptide comprising an amino acid sequence of SEQ ID NO: 85;
(viii) a polypeptide comprising an amino acid sequence of SEQ ID NO: 109, a polypeptide comprising an amino acid sequence of SEQ ID NO: 110, and a polypeptide comprising an amino acid sequence of SEQ ID NO: 84;
(ix) a polypeptide comprising an amino acid sequence of SEQ ID NO: 220, a polypeptide comprising an amino acid sequence of SEQ ID NO: 221, and a polypeptide comprising an amino acid sequence of SEQ ID NO: 84; or
(x) first heavy chain, a second heavy chain, and a light chain, each comprising an amino acid sequence corresponding to a sequence of a first heavy chain, a sequence of a second heavy chain, and a sequence of a light chain listed in TABLE 11, respectively.

215. The protein of claim 213, comprising:
(a) a first heavy chain comprising an amino acid sequence corresponding to a sequence of SEQ ID NO: 97;
(b) a second heavy chain comprising an amino acid sequence corresponding to a sequence of SEQ ID NO: 98; and
(c) a light chain comprising an amino acid sequence of SEQ ID NO: 85.

216. The protein of claim 213, comprising:
(a) a first heavy chain comprising an amino acid sequence corresponding to a sequence of SEQ ID NO: 103;
(b) a second heavy chain comprising an amino acid sequence corresponding to a sequence of SEQ ID NO: 104; and
(c) a light chain comprising an amino acid sequence of SEQ ID NO: 85.

217. The protein of claim 194, wherein:
(a) the protein has:
(i) a K_{D} for CD3 binding of 10 nM to 50 nM as measured in a biolayer interferometry assay;
(ii) a K_{D} for CD3 binding of 15 nM to 20 nM as measured in a biolayer interferometry assay;
(iii) a K_{D} for ROR1 binding of 80 nM to 120 nM as measured in a biolayer interferometry assay;
(iv) a K_{D} for ROR1 binding of 95 nM to 105 nM as measured in a biolayer interferometry assay;
(v) a K_{D} for ROR1 binding of 1 nM to 10 nM as measured in a biolayer interferometry assay; and/or
(vi) a K_{D} for ROR1 binding of 3 nM to 5 nM as measured in a biolayer interferometry assay;
(b) the protein has:
(i) an EC₅₀ of 80 nM to 120 nM in a ROR1 binding assay using cells expressing about 3.5×10⁴ ROR1 molecules/cell;
(ii) an EC₅₀ of 90 nM to 110 nM in a ROR1 binding assay using cells expressing about 3.5×10⁴ ROR1 molecules/cell;
(iii) an EC₅₀ of 1 nM to 10 nM in a ROR1 binding assay using cells expressing about 3.5×10⁴ ROR1 molecules/cell;
(iv) an EC₅₀ of 2 nM to 5 nM in a ROR1 binding assay using cells expressing about 3.5×10⁴ ROR1 molecules/cell;
(v) an EC₅₀ of 20 nM to 50 nM in a CD3 binding assay using activated T cells;
(vi) an EC₅₀ of 25 nM to 35 nM in a CD3 binding assay using activated T cells;
(vii) an EC₅₀ of 160 pM to 200 pM for T cell mediated cytotoxicity of ROR1-expressing tumor cells as measured in a T cell/tumor cell co-culture killing assay;
(viii) an EC₅₀ of 165 pM to 190 pM for T cell mediated cytotoxicity of ROR1-expressing tumor cells as measured in a T cell/tumor cell co-culture killing assay;
(ix) an EC₅₀ of 25 pM to 50 pM for T cell mediated cytotoxicity of ROR1-expressing tumor cells as measured in a T cell/tumor cell co-culture killing assay;
(x) an EC₅₀ of 30 pM to 40 pM for T cell mediated cytotoxicity of ROR1-expressing tumor cells as measured in a T cell/tumor cell co-culture killing assay;
(xi) an EC₅₀ of 1000 pM to 2000 pM as measured in a T cell/tumor cell co-culture IFNγ production assay;
(xii) an EC₅₀ of 1400 pM to 1800 pM as measured in a T cell/tumor cell co-culture IFNγ production assay;
(xiii) an EC₅₀ of 200 pM to 600 pM as measured in a T cell/tumor cell co-culture IFNγ production assay; and/or
(ix) an EC₅₀ of 300 pM to 500 pM as measured in a T cell/tumor cell co-culture IFNγ production assay;
(b) the protein binds to the Ig-like domain of ROR1;
(c) the protein does not bind to the Frizzled and/or Kringle domain of ROR1;
(d) the protein induces T cell lytic granule degranulation;
(e) the protein does not induce a significant increase in the production and/or release of pro inflammatory cytokines by T cells when administered to cultures at concentrations sufficient to induce T cell mediated cytotoxicity;
(f) the protein has:
(i) a Cₘₐₓ of 20% to 60% compared to a reference T cell-binder as measured in a T cell/tumor cell co-culture IFNγ production assay;
(ii) a Cₘₐₓ of 35% to 45% compared to a reference T cell-binder as measured in a T cell/tumor cell co-culture IFNγ production assay; or
(iii) a Cₘₐₓ of 45% to 55% compared to a reference T cell-binder as measured in a T cell/tumor cell co-culture IFNγ production assay;
(g) the protein does not induce significant levels of TNFα release when administered to freshly isolated peripheral mononuclear cell (PBMC) cultures;
(h) the protein does not induce significant levels of TNFα release when administered to PBMCs pre-cultured for 48 hours at high density;
(i) the protein does not induce significant levels of IL2 release when administered to freshly isolated PBMC cultures;
(j) the protein does not induce significant levels of IL2 release when administered to PBMCs pre-cultured for 48 hours at high density;
(k) the protein has a cytokine release to killing decoupling ratio of 1:2 to 1:4 normalized to a reference T cell-binder as measured in a T cell/tumor cell co-culture killing assay;
(l) the protein has a cytokine release to killing decoupling ratio of 1:2.2 normalized to a reference T cell-binder as measured in a T cell/tumor cell co-culture killing assay;
(m) the protein has a cytokine release to killing decoupling ratio of 1:2.9 normalized to a reference T cell-binder as measured in a T cell/tumor cell co-culture killing assay;
(n) the protein induces a tumor serial killing index of 3 to 5 as measured in a T cell/tumor cell co-culture killing assay;
(o) the protein induces a tumor serial killing index of 3.4 to 3.6 as measured in a T cell/tumor cell co-culture killing assay;
(p) the protein induces a tumor serial killing index of 3.8 to 4.2 as measured in a T cell/tumor cell co-culture killing assay;
(q) the protein is cross-reactive with cynomolgus CD3 but not mouse CD3; and/or
(r) the protein is cross-reactive with cynomolgus ROR1 and mouse ROR1.

218. A formulation comprising a protein of claim 194, and a pharmaceutically acceptable carrier.

219. One or more nucleic acids encoding a protein of claim 194.

220. A cell comprising one or more nucleic acids encoding a protein of claim 194.

221. A method of treating a cancer in a patient, the method comprising administering to the patient a protein of claim 194.

222. The method of claim 221, wherein:
(a) the cancer is a hematological tumor cancer or a solid tumor cancer; and/or
(b) the cancer is selected from the group consisting of chronic lymphocytic leukemia, mantle cell lymphoma, non-Hodgkin lymphoma, ovarian cancer, lung cancer, bronchial cancer, colon cancer, rectal cancer, melanoma, renal cancer, gastric cancer, pancreatic cancer, prostate cancer, uterine cancer, breast cancer, oral cancer, pharyngeal cancer, hairy cell leukemia, liver cancer, intrahepatic bile duct cancer, and thyroid cancer.

223. An antibody or functional fragment thereof comprising:
(i) according to the IMGT unique numbering scheme, a heavy chain variable complementarity-determining region 1 (VHCDR1) comprising an amino acid sequence of GFTFTSYA (SEQ ID NO: 8), a heavy chain variable complementarity-determining region 2 (VHCDR2) comprising an amino acid sequence of ISGSGGGT (SEQ ID NO: 9), a heavy chain variable complementarity-determining region 3 (VHCDR3) comprising an amino acid sequence of AQGSSIFDY (SEQ ID NO: 10), a light chain variable complementarity-determining region 1 (VLCDR1) comprising an amino acid sequence of QSVSSY (SEQ ID NO: 11), a light chain variable complementarity-determining region 2 (VLCDR2) comprising an amino acid sequence of DAS, and a light chain variable complementarity-determining region 3 (VLCDR3) comprising an amino acid sequence of QQRSNWPPFT (SEQ ID NO: 14);
(ii) according to the IMGT unique numbering scheme, a heavy chain variable complementarity-determining region 1 (VHCDR1) comprising an amino acid sequence of GFTFTSYA (SEQ ID NO: 8), a heavy chain variable complementarity-determining region 2 (VHCDR2) comprising an amino acid sequence of ISGSGGGT (SEQ ID NO: 9), a heavy chain variable complementarity-determining region 3 (VHCDR3) comprising an amino acid sequence of AQGSSIFDY (SEQ ID NO: 10), a light chain variable complementarity-determining region 1 (VLCDR1) comprising an amino acid sequence of QSVSSY (SEQ ID NO: 11), a light chain variable complementarity-determining region 2 (VLCDR2) comprising an amino acid sequence of DAS, and a light chain variable complementarity-determining region 3 (VLCDR3) comprising an amino acid sequence of QQRSNWPPST (SEQ ID NO: 15); or
(iii) according to the IMGT unique numbering scheme, a heavy chain variable complementarity-determining region 1 (VHCDR1) comprising an amino acid sequence of GFSITTSGVS (SEQ ID NO: 23), a heavy chain variable complementarity-determining region 2 (VHCDR2) comprising an amino acid sequence of IYWDDDK (SEQ ID NO: 24), a heavy chain variable complementarity-determining region 3 (VHCDR3) comprising an amino acid sequence of AHAPRYTPGGYFDY (SEQ ID NO: 25), a light chain variable complementarity-determining region 1 (VLCDR1) comprising an amino acid sequence of QSVSSN (SEQ ID NO: 26), a light chain variable complementarity-determining region 2 (VLCDR2) comprising an amino acid sequence of GAS, and a light chain variable complementarity-determining region 3 (VLCDR3) comprising an amino acid sequence of QQYKNWPPA (SEQ ID NO: 28).

224. The antibody of claim 223, wherein:
(i) the heavy chain variable domain comprises an amino acid sequence at least 90% identical to SEQ ID NO: 29, and the light chain comprises an amino acid sequence at least 90% identical to SEQ ID NO: 30;
(ii) the heavy chain variable domain comprises an amino acid sequence at least 90% identical to SEQ ID NO: 29, and the light chain variable domain comprises an amino acid sequence at least 90% identical to SEQ ID NO: 31; or
(iii) the heavy chain variable domain comprises an amino acid sequence at least 90% identical to SEQ ID NO: 39, and the light chain variable domain comprises an amino acid sequence at least 90% identical to SEQ ID NO: 40.

225. The antibody or functional fragment thereof of claim 223, wherein the antibody is a human IgG1 antibody.


SEQUENCE LISTING (307 sequences)

SEQ ID NO: 1 - protein, 60 aa, synthetic construct
  SITE 1..60: note = This sequence may encompass 1-12 GGGGS repeats
GGGGSGGGGSGGGGSGGGGSGGGGSGGGGSGGGGSGGGGSGGGGSGGGGSGGGGSGGGGS

SEQ ID NO: 2 - protein, 20 aa, synthetic construct
GSGSGSGSGSGSGSGSGSGS

SEQ ID NO: 3 - protein, 30 aa, synthetic construct
GGSGGSGGSGGSGGSGGSGGSGGSGGSGGS

SEQ ID NO: 4 - protein, 40 aa, synthetic construct
GGGSGGGSGGGSGGGSGGGSGGGSGGGSGGGSGGGSGGGS

SEQ ID NO: 5 - protein, 40 aa, synthetic construct
GGSGGGSGGGSGGGSGGGSGGGSGGGSGGGSGGGSGGGSG

SEQ ID NO: 6 - protein, 50 aa, synthetic construct
GGSGGGGSGGGGSGGGGSGGGGSGGGGSGGGGSGGGGSGGGGSGGGGSGG

SEQ ID NO: 7 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 8 - protein, 8 aa, synthetic construct
GFTFTSYA

SEQ ID NO: 9 - protein, 8 aa, synthetic construct
ISGSGGGT

SEQ ID NO: 10 - protein, 9 aa, synthetic construct
AQGSSIFDY

SEQ ID NO: 11 - protein, 6 aa, synthetic construct
QSVSSY

SEQ ID NO: 12 - skipped

SEQ ID NO: 13 - protein, 10 aa, synthetic construct
  VARIANT 9: note = F, S, C, R, Q, I, L, Y, or V
QQRSNWPPXT

SEQ ID NO: 14 - protein, 10 aa, synthetic construct
QQRSNWPPFT

SEQ ID NO: 15 - protein, 10 aa, synthetic construct
QQRSNWPPST

SEQ ID NO: 16 - protein, 10 aa, synthetic construct
QQRSNWPPCT

SEQ ID NO: 17 - protein, 10 aa, synthetic construct
QQRSNWPPRT

SEQ ID NO: 18 - protein, 10 aa, synthetic construct
QQRSNWPPQT

SEQ ID NO: 19 - protein, 10 aa, synthetic construct
QQRSNWPPIT

SEQ ID NO: 20 - protein, 10 aa, synthetic construct
QQRSNWPPLT

SEQ ID NO: 21 - protein, 10 aa, synthetic construct
QQRSNWPPYT

SEQ ID NO: 22 - protein, 10 aa, synthetic construct
QQRSNWPPVT

SEQ ID NO: 23 - protein, 10 aa, synthetic construct
GFSITTSGVS

SEQ ID NO: 24 - protein, 7 aa, synthetic construct
IYWDDDK

SEQ ID NO: 25 - protein, 14 aa, synthetic construct
AHAPRYTPGGYFDY

SEQ ID NO: 26 - protein, 6 aa, synthetic construct
QSVSSN

SEQ ID NO: 27 - skipped

SEQ ID NO: 28 - protein, 9 aa, synthetic construct
QQYKNWPPA

SEQ ID NO: 29 - protein, 116 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 30 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIK

SEQ ID NO: 31 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK

SEQ ID NO: 32 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPCTFGQGTRLEIK

SEQ ID NO: 33 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRTFGQGTRLEIK

SEQ ID NO: 34 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPQTFGQGTRLEIK

SEQ ID NO: 35 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIK

SEQ ID NO: 36 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGQGTRLEIK

SEQ ID NO: 37 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTRLEIK

SEQ ID NO: 38 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 39 - protein, 122 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSS

SEQ ID NO: 40 - protein, 107 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIK

SEQ ID NO: 41 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 42 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 43 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPCTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 44 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 45 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPQTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 46 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 47 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 48 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 49 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 50 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIK

SEQ ID NO: 51 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK

SEQ ID NO: 52 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPCTFGQGTRLEIK

SEQ ID NO: 53 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPRTFGQGTRLEIK

SEQ ID NO: 54 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPQTFGQGTRLEIK

SEQ ID NO: 55 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPITFGQGTRLEIK

SEQ ID NO: 56 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGQGTRLEIK

SEQ ID NO: 57 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTRLEIK

SEQ ID NO: 58 - protein, 244 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPVTFGQGTRLEIK

SEQ ID NO: 59 - protein, 249 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSS

SEQ ID NO: 60 - protein, 249 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIK

SEQ ID NO: 61 - protein, 8 aa, synthetic construct
GFTFNTYA

SEQ ID NO: 62 - protein, 10 aa, synthetic construct
IRSKYNNYAT

SEQ ID NO: 63 - protein, 16 aa, synthetic construct
VRHGNFGNSYVSWFAY

SEQ ID NO: 64 - protein, 8 aa, synthetic construct
TGAVTTSN

SEQ ID NO: 65 - skipped

SEQ ID NO: 66 - protein, 9 aa, synthetic construct
ALWYSNLWV

SEQ ID NO: 67 - protein, 16 aa, synthetic construct
  VARIANT 8: note = N, A, or E
  VARIANT 9: note = S or A
VRHGNFGXXYVSWFAY

SEQ ID NO: 68 - protein, 16 aa, synthetic construct
VRHGNFGASYVSWFAY

SEQ ID NO: 69 - protein, 16 aa, synthetic construct
VRHGNFGESYVSWFAY

SEQ ID NO: 70 - protein, 15 aa, synthetic construct
VRHGNFGNAYVSFAY

SEQ ID NO: 71 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGNSYVSWFAYWGQGTMVTVSS

SEQ ID NO: 72 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGASYVSWFAYWGQGTMVTVSS

SEQ ID NO: 73 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTMVTVSS

SEQ ID NO: 74 - protein, 125 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGESYVSWFAYWGQGTMVTVSS

SEQ ID NO: 75 - protein, 124 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNAYVSFAYWGQGTMVTVSS

SEQ ID NO: 76 - protein, 109 aa, synthetic construct
QTVVTQEPSLTVSPGGTVTLTCASSTGAVTTSNYANWVQQKPGQAPRALIGGTNKRAPGTPARFSGSLLGGKAALTLSGVQPEDEAEYYCALWYSNLWVFGGGTKLTVL

SEQ ID NO: 77 - protein, 109 aa, synthetic construct
QTVVTQEPSLTVSPGGTVTLTCASSTGAVTTSNYANWFQQKPGQAPRALIGGTNKRAPGTPARFSGSLLGGKAALTLSGVQPEDEAEYYCALWYSNLWVFGGGTKLTVL

SEQ ID NO: 78 - protein, 109 aa, synthetic construct
QTVVTQEPSLTVSPGGTVTLTCASSTGAVTTSNYANWVQQKPGQAPRALIYGTNKRAPGTPARFSGSLLGGKAALTLSGVQPEDEAEYYCALWYSNLWVFGGGTKLTVL

SEQ ID NO: 79 - protein, 109 aa, synthetic construct
QTVVTQEPSLTVSPGGTVTLTCASSTGAVTTSNYANWFQQKPGQAPRALIYGTNKRAPGTPARFSGSLLGGKAALTLSGVQPEDEAEYYCALWYSNLWVFGGGTKLTVL

SEQ ID NO: 80 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSS

SEQ ID NO: 81 - protein, 109 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 82 - protein, 223 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGASYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 83 - protein, 223 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 84 - protein, 215 aa, synthetic construct
QAVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - protein, 215 aa, synthetic construct
QTVVTQEPSLTVSPGGTVTLTCASSTGAVTTSNYANWVQQKPGQAPRALIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 86 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 87 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 88 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 89 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 90 - protein, 22 aa, synthetic construct
EPKSCDKTHTCPPCPAPEAAGG

SEQ ID NO: 91 - protein, 22 aa, Homo sapiens
EPKSCDKTHTCPPCPAPELLGG

SEQ ID NO: 92 - protein, 22 aa, synthetic construct
EPKSSDKTHTCPPCPAPEAAGG

SEQ ID NO: 93 - protein, 223 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 94 - protein, 458 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGASYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 95 - protein, 743 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 96 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - protein, 458 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 98 - protein, 743 aa, synthetic construct; identical to SEQ ID NO: 95

SEQ ID NO: 99 - protein, 106 aa, Homo sapiens
PTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLF

SEQ ID NO: 100 - protein, 458 aa, synthetic construct; identical to SEQ ID NO: 94

SEQ ID NO: 101 - protein, 743 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 102 - protein, 135 aa, Homo sapiens
EEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRI

SEQ ID NO: 103 - protein, 458 aa, synthetic construct; identical to SEQ ID NO: 97

SEQ ID NO: 104 - protein, 743 aa, synthetic construct; identical to SEQ ID NO: 101

SEQ ID NO: 105 - protein, 80 aa, Homo sapiens
KCYNSTGVDYRGTVSVTKSGRQCQPWNSQYPHTHTFTALRFPELNGGHSYCRNPGNQKEAPWCFTLDENFKSDLCDIPAC

SEQ ID NO: 106 - protein, 457 aa, synthetic construct
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 107 - protein, 752 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGKGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSS

SEQ ID NO: 108 - protein, 223 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 109 - protein, 727 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 110 - protein, 479 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 111 - protein, 223 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGESYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 112 - protein, 731 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 113 - protein, 483 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGK

SEQ ID NO: 114 - protein, 222 aa, synthetic construct
EVQLVESGGGLVQPGGSLKLSCAASGFTFNTYAMNWVRQASGKGLEWVGRIRSKYNNYATYYADSVKGRFTISRDDSKNTAYLQMNSLKTEDTAVYYCVRHGNFGNAYVSFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 115 - protein, 21 aa, synthetic construct
EPKSCDKTHTCPPCPAPPVAG

SEQ ID NO: 116 - protein, 21 aa, synthetic construct
EPKSSDKTHTCPPCPAPPVAG

SEQ ID NO: 117 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 118 - protein, 213 aa, synthetic construct
PSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGK

SEQ ID NO: 119 - protein, 215 aa, synthetic construct
QTVVTQEPSLTVSPGGTVTLTCASSTGAVTTSNYANWVQQKPGQAPRALIYGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 120 - protein, 215 aa, synthetic construct
QTVVTQEPSLTVSPGGTVTLTCASSTGAVTTSNYANWFQQKPGQAPRALIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 121 - protein, 215 aa, synthetic construct
QTVVTQEPSLTVSPGGTVTLTCASSTGAVTTSNYANWFQQKPGQAPRALIYGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 122 - protein, 215 aa, synthetic construct; identical to SEQ ID NO: 85

SEQ ID NO: 123 - protein, 114 aa, synthetic construct
EVKLQQSGPELVKPGASVKISCKTSGYTFTEYTMHWVKQSHGKSLEWIGGINPNNGGTSYNQKFKGKATLTVDKSSSTAYMELRSLTSEDSAVYYCALQGFAYWGQGTPLTVSS

SEQ ID NO: 124 - protein, 106 aa, synthetic construct
EIEITQTPALMSASPGEKVTMTCSASSSVSYMYWYQQKPRSSPKPWIYLTSNLASGVPARFSGSGSGTSYSLTISSMEAEDAATYYCQQWSSNPYTFGGGTRLELK

SEQ ID NO: 125 - protein, 117 aa, synthetic construct
EVKLVESGGGLVKPGGSLKLSCAASGFTFSSYAMSWVRQIPEKRLEWVASISRGGTTYYPDSVKGRFTISRDNVRNILYLQMSSLRSEDTAMYYCGRYDYDGYYAMDYWGQGTSVSS

SEQ ID NO: 126 - protein, 107 aa, synthetic construct
DIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMK

SEQ ID NO: 127 - protein, 8 aa, synthetic construct
GFTFSSYA

SEQ ID NO: 128 - protein, 7 aa, synthetic construct
ISRGGTT

SEQ ID NO: 129 - protein, 11 aa, synthetic construct
YDYDGYYAMDY

SEQ ID NO: 130 - protein, 6 aa, synthetic construct
PDINSY

SEQ ID NO: 131 - skipped

SEQ ID NO: 132 - protein, 9 aa, synthetic construct
LQYDEFPYT

SEQ ID NO: 133 - protein, 5 aa, synthetic construct
EPKSC

SEQ ID NO: 134 - protein, 386 aa, Homo sapiens
MKLPVRLLVLMFWIPASLSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEY

SEQ ID NO: 135 - protein, 240 aa, synthetic construct
EIEITQTPALMSASPGEKVTMTCSASSSVSYMYWYQQKPRSSPKPWIYLTSNLASGVPARFSGSGSGTSYSLTISSMEAEDAATYYCQQWSSNPYTFGGGTRLELKGGGGSGGGGSGGGGSGGGGSEVKLQQSGPELVKPGASVKISCKTSGYTFTEYTMHWVKQSHGKSLEWIGGINPNNGGTSYNQKFKGKATLTVDKSSSTAYMELRSLTSEDSAVYYCALQGFAYWGQGTPLTVSS

SEQ ID NO: 136 - protein, 241 aa, synthetic construct
DIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMKGGGGSGGGGSGGGGSGGGGSEVKLQQSGPELVKPGASVKISCKTSGYTFTEYTMHWVKQSHGKSLEWIGGINPNNGGTSYNQKFKGKATLTVDKSSSTAYMELRSLTSEDSAVYYCALQGFAYWGQGTPLTVSS

SEQ ID NO: 137 - protein, 533 aa, Homo sapiens
MKLPVRLLVLMFWIPASLSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNH

SEQ ID NO: 138 - protein, 510 aa, Homo sapiens
MKLPVRLLVLMFWIPASLSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 139 - protein, 240 aa, synthetic construct
EVKLQQSGPELVKPGASVKISCKTSGYTFTEYTMHWVKQSHGKSLEWIGGINPNNGGTSYNQKFKGKATLTVDKSSSTAYMELRSLTSEDSAVYYCALQGFAYWGQGTPLTVSSGGGGSGGGGSGGGGSGGGGSEIEITQTPALMSASPGEKVTMTCSASSSVSYMYWYQQKPRSSPKPWIYLTSNLASGVPARFSGSGSGTSYSLTISSMEAEDAATYYCQQWSSNPYTFGGGTRLELK

SEQ ID NO: 140 - protein, 241 aa, synthetic construct
EVKLQQSGPELVKPGASVKISCKTSGYTFTEYTMHWVKQSHGKSLEWIGGINPNNGGTSYNQKFKGKATLTVDKSSSTAYMELRSLTSEDSAVYYCALQGFAYWGQGTPLTVSSGGGGSGGGGSGGGGSGGGGSDIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMK

SEQ ID NO: 141 - protein, 118 aa, synthetic construct
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 142 - protein, 107 aa, synthetic construct
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 143 - protein, 8 aa, synthetic construct
GYTFSDYE

SEQ ID NO: 144 - protein, 8 aa, synthetic construct
IDPETGGT

SEQ ID NO: 145 - protein, 11 aa, synthetic construct
TGYYDYDSFTY

SEQ ID NO: 146 - protein, 6 aa, synthetic construct
QNVDAA

SEQ ID NO: 147 - skipped

SEQ ID NO: 148 - protein, 9 aa, synthetic construct
QQYDIYPYT

SEQ ID NO: 149 - protein, 117 aa, synthetic construct
QVQLKESGPGLVAPSQTLSITCTVSGFSLTSYGVHWVROPPGKGLEWLGVIWAGGFTNYNSALKSRLSISKDNSKSQVLLKMTSLQTDDTAMYYCARRGSSYSMDYWGQGTSVTVSS

SEQ ID NO: 150 - protein, 106 aa, synthetic construct
EIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQ

SEQ ID NO: 151 - protein, 8 aa, synthetic construct
GFSLTSYG

SEQ ID NO: 152 - protein, 7 aa, synthetic construct
IWAGGFT

SEQ ID NO: 153 - protein, 11 aa, synthetic construct
ARRGSSYSMDY

SEQ ID NO: 154 - protein, 5 aa, synthetic construct
SNVSY

SEQ ID NO: 155 - skipped

SEQ ID NO: 156 - protein, 9 aa, synthetic construct
QQWNYPLIT

SEQ ID NO: 157 - protein, 118 aa, synthetic construct
QVQLQQPGAELVKPGTSVKLSCKASGYNFTNYWINWVKLRPGQGLEWIGEIYPGSGSTNYNEKFKSKATLTADTSSSTAYMQLSSLASEDSALYYCARDGNYYAMDYWGQGTSVTVSS

SEQ ID NO: 158 - protein, 108 aa, synthetic construct
DIQMTQTTSSLSASLGDRVTITCRASQDINNYLNWYQQKPDGTVKLLIYYTSALHSGVPSRFSGSGSGTDYSLTISNLEQEDIATYFCQQGNTLPPYTFGGGTKLEIK

SEQ ID NO: 159 - protein, 8 aa, synthetic construct
GYNFTNYW

SEQ ID NO: 160 - protein, 8 aa, synthetic construct
IYPGSGST

SEQ ID NO: 161 - protein, 11 aa, synthetic construct
ARDGNYYAMDY

SEQ ID NO: 162 - protein, 6 aa, synthetic construct
QDINNY

SEQ ID NO: 163 - skipped

SEQ ID NO: 164 - protein, 10 aa, synthetic construct
QQGNTLPPYT

SEQ ID NO: 165 - protein, 118 aa, synthetic construct
EVKLVESGGGLVKPGGSLKLSCAASGFTFSSYAMSWVRQTPEKRLEWVASISTGASAYFPDSVKGRFTISRDNARNILYLQMSSLRSEDTAMYYCARITTSTWYFDVWGAGTTVTVSS

SEQ ID NO: 166 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIK

SEQ ID NO: 167 - protein, 119 aa, synthetic construct
EVQLQQSGPELEKPGASVKISCKASGFAFTGYNMNWVKQTNGKSLEWIGSIDPYYGGSTYNQKFKDKATLTVDKSSSTAYMQLKSLTSDDSAVYYCARSPGGDYAMDYWGQGTSVTVSS

SEQ ID NO: 168 - protein, 7 aa, synthetic construct
ISTGASA

SEQ ID NO: 169 - protein, 12 aa, synthetic construct
ARITTSTWYFDV

SEQ ID NO: 170 - protein, 6 aa, synthetic construct
QDINSY

SEQ ID NO: 171 - protein, 107 aa, synthetic construct
DIKMTQSPSSMYASVGERVTITCKASQGINSYSGWFQQKPGKSPKTLIYRGNRLVDGVPSRFSGSGSGQDYSLTISSLEYEDMGIYYCLQYDEFPYTFGGGTKLEIK

SEQ ID NO: 172 - protein, 8 aa, synthetic construct
GFAFTGYN

SEQ ID NO: 173 - protein, 8 aa, synthetic construct
GYTFTEYT

SEQ ID NO: 174 - protein, 8 aa, synthetic construct
INPNNGGT

SEQ ID NO: 175 - protein, 7 aa, synthetic construct
ALQGFAY

SEQ ID NO: 176 - protein, 5 aa, synthetic construct
SSVSY

SEQ ID NO: 177 - skipped

SEQ ID NO: 178 - protein, 9 aa, synthetic construct
QQWSSNPYT

SEQ ID NO: 179 - protein, 8 aa, synthetic construct
GYTGTSYT

SEQ ID NO: 180 - protein, 8 aa, synthetic construct
INPSSGYT

SEQ ID NO: 181 - protein, 12 aa, synthetic construct
ARRVLWLRRGDY

SEQ ID NO: 182 - protein, 6 aa, synthetic construct
QDIGSS

SEQ ID NO: 183 - skipped

SEQ ID NO: 184 - protein, 7 aa, synthetic construct
LQYASSP

SEQ ID NO: 185 - protein, 245 aa, synthetic construct
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKGGGGSGGGGSGGGGSGGGGSQVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 186 - protein, 243 aa, synthetic construct
EIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQGGGGSGGGGSGGGGSGGGGSQVQLKESGPGLVAPSQTLSITCTVSGFSLTSYGVHWVROPPGKGLEWLGVIWAGGFTNYNSALKSRLSISKDNSKSQVLLKMTSLQTDDTAMYYCARRGSSYSMDYWGQGTSVTVSS

SEQ ID NO: 187 - protein, 246 aa, synthetic construct
DIQMTQTTSSLSASLGDRVTITCRASQDINNYLNWYQQKPDGTVKLLIYYTSALHSGVPSRFSGSGSGTDYSLTISNLEQEDIATYFCQQGNTLPPYTFGGGTKLEIKGGGGSGGGGSGGGGSGGGGSQVQLQQPGAELVKPGTSVKLSCKASGYNFTNYWINWVKLRPGQGLEWIGEIYPGSGSTNYNEKFKSKATLTADTSSSTAYMQLSSLASEDSALYYCARDGNYYAMDYWGQGTSVTVSS

SEQ ID NO: 188 - protein, 245 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKGGGGSGGGGSGGGGSGGGGSEVKLVESGGGLVKPGGSLKLSCAASGFTFSSYAMSWVRQTPEKRLEWVASISTGASAYFPDSVKGRFTISRDNARNILYLQMSSLRSEDTAMYYCARITTSTWYFDVWGAGTTVTVSS

SEQ ID NO: 189 - protein, 245 aa, synthetic construct
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGGSGGGGSGGGGSGGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 190 - protein, 243 aa, synthetic construct
QVQLKESGPGLVAPSQTLSITCTVSGFSLTSYGVHWVROPPGKGLEWLGVIWAGGFTNYNSALKSRLSISKDNSKSQVLLKMTSLQTDDTAMYYCARRGSSYSMDYWGQGTSVTVSSGGGGSGGGGSGGGGSGGGGSEIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQ

SEQ ID NO: 191 - protein, 246 aa, synthetic construct
QVQLQQPGAELVKPGTSVKLSCKASGYNFTNYWINWVKLRPGQGLEWIGEIYPGSGSTNYNEKFKSKATLTADTSSSTAYMQLSSLASEDSALYYCARDGNYYAMDYWGQGTSVTVSSGGGGSGGGGSGGGGSGGGGSDIQMTQTTSSLSASLGDRVTITCRASQDINNYLNWYQQKPDGTVKLLIYYTSALHSGVPSRFSGSGSGTDYSLTISNLEQEDIATYFCQQGNTLPPYTFGGGTKLEIK

SEQ ID NO: 192 - protein, 245 aa, synthetic construct
EVKLVESGGGLVKPGGSLKLSCAASGFTFSSYAMSWVRQTPEKRLEWVASISTGASAYFPDSVKGRFTISRDNARNILYLQMSSLRSEDTAMYYCARITTSTWYFDVWGAGTTVTVSSGGGGSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIK

SEQ ID NO: 193 - protein, 22 aa, synthetic construct
EPKSSDKTHTCPPCPAPELLGG

SEQ ID NO: 194 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 196 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 198 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVHLTCLVKGFYPSDIAVEWESNGQPENNYKTTPPVLDSDGSFALYSKLTVDKSRWQQGNVFSCSVMHEALHNHYTQKSLSLSPGK

SEQ ID NO: 199 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSRDELTKNQVSLTCLVKGFYPSDIAVEWESNGQPENNYKTFPPVLDSDGSFFLYSKLTVDKSRWQQGNVFSCSVMHEALHNHYTQKSLSLSPGK

SEQ ID NO: 200 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCDVSGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 203 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 204 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 205 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSRDELTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 206 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSNLTVDKSRWQQGNVFSCSVMHEALHNHYTQKSLSLSPGK

SEQ ID NO: 207 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 208 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 209 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 210 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 211 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 212 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 213 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 214 - protein, 210 aa, Homo sapiens; identical to SEQ ID NO: 210

SEQ ID NO: 215 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDNLTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 216 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 217 - protein, 210 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 218 - protein, 458 aa, synthetic construct; identical to SEQ ID NO: 97

SEQ ID NO: 219 - protein, 753 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSS

SEQ ID NO: 220 - protein, 732 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 221 - protein, 484 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 222 - protein, 743 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK

SEQ ID NO: 223 - protein, 743 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK

SEQ ID NO: 224 - protein, 743 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIK

SEQ ID NO: 225 - protein, 743 aa, synthetic construct; identical to SEQ ID NO: 224

SEQ ID NO: 226 - protein, 752 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGKQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIK

SEQ ID NO: 227 - protein, 753 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIK

SEQ ID NO: 228 - protein, 727 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 229 - protein, 479 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 230 - protein, 731 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 231 - protein, 483 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALAAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGK

SEQ ID NO: 232 - protein, 732 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 233 - protein, 484 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 234 - protein, 458 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGASYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 235 - protein, 743 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 236 - protein, 458 aa, synthetic construct
EVQLVESGGGLVQPGGSLKLSCAASGFTFNTYAMNWVRQASGKGLEWVGRIRSKYNNYATYYADSVKGRFTISRDDSKNTLYLQMNSLKTEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 237 - protein, 743 aa, synthetic construct; identical to SEQ ID NO: 235

SEQ ID NO: 238 - protein, 458 aa, synthetic construct; identical to SEQ ID NO: 234

SEQ ID NO: 239 - protein, 743 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSS

SEQ ID NO: 240 - protein, 458 aa, synthetic construct; identical to SEQ ID NO: 236

SEQ ID NO: 241 - protein, 743 aa, synthetic construct; identical to SEQ ID NO: 239

SEQ ID NO: 242 - protein, 458 aa, synthetic construct; identical to SEQ ID NO: 236

SEQ ID NO: 243 - protein, 753 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSS

SEQ ID NO: 244 - protein, 727 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 245 - protein, 479 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 246 - protein, 732 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 247 - protein, 484 aa, synthetic construct
ETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 248 - protein, 7 aa, synthetic construct
GFTSSYA

SEQ ID NO: 249 - protein, 20 aa, synthetic construct
GGGGSGGGGSGGGGSGGGGS

SEQ ID NO: 250 - protein, 458 aa, synthetic construct; identical to SEQ ID NO: 236

SEQ ID NO: 251 - protein, 743 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK

SEQ ID NO: 252 - protein, 458 aa, synthetic construct; identical to SEQ ID NO: 234

SEQ ID NO: 253 - protein, 743 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGQGTRLEIK

SEQ ID NO: 254 - protein, 458 aa, synthetic construct; identical to SEQ ID NO: 236

SEQ ID NO: 255 - protein, 743 aa, synthetic construct; identical to SEQ ID NO: 253

SEQ ID NO: 256 - protein, 458 aa, synthetic construct; identical to SEQ ID NO: 236

SEQ ID NO: 257 - protein, 753 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSQVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIK

SEQ ID NO: 258 - protein, 727 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 259 - protein, 479 aa, synthetic construct
EVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 260 - protein, 732 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKGGGGSGGGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 261 - protein, 484 aa, synthetic construct
QVTLKESGPTLVKPTQTLTLTCSFSGFSITTSGVSVGWIRQPPGKALEWLALIYWDDDKRYSPSLKSRLTITRDTSKNQVVLTMTNMDPVDTATYYCAHAPRYTPGGYFDYWGQGTLVTVSSGGGGSGGGGSGGGGSGGGGSETTLTQSPATLSVSPGETATLSCRASQSVSSNLAWYQQKPGQAPRLLIYGASTRATGIPATFSGSGSGTEFTLTISSLQSEDFAIYYCQQYKNWPPAFGGGTELEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 262 - protein, 8 aa, synthetic construct
IDPYYGGS

SEQ ID NO: 263 - protein, 12 aa, synthetic construct
ARSPGGDYAMDY

SEQ ID NO: 264 - protein, 6 aa, synthetic construct
QGINSY

SEQ ID NO: 265 - skipped

SEQ ID NO: 266 - protein, 246 aa, synthetic construct
DIKMTQSPSSMYASVGERVTITCKASQGINSYSGWFQQKPGKSPKTLIYRGNRLVDGVPSRFSGSGSGQDYSLTISSLEYEDMGIYYCLQYDEFPYTFGGGTKLEIKGGGGSGGGGSGGGGSGGGGSEVQLQQSGPELEKPGASVKISCKASGFAFTGYNMNWVKQTNGKSLEWIGSIDPYYGGSTYNQKFKDKATLTVDKSSSTAYMQLKSLTSDDSAVYYCARSPGGDYAMDYWGQGTSVTVSS

SEQ ID NO: 267 - protein, 246 aa, synthetic construct
EVQLQQSGPELEKPGASVKISCKASGFAFTGYNMNWVKQTNGKSLEWIGSIDPYYGGSTYNQKFKDKATLTVDKSSSTAYMQLKSLTSDDSAVYYCARSPGGDYAMDYWGQGTSVTVSSGGGGSGGGGSGGGGSGGGGSDIKMTQSPSSMYASVGERVTITCKASQGINSYSGWFQQKPGKSPKTLIYRGNRLVDGVPSRFSGSGSGQDYSLTISSLEYEDMGIYYCLQYDEFPYTFGGGTKLEIK

SEQ ID NO: 268 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 269 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 270 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 271 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 272 - protein, 107 aa, Homo sapiens
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 107 aa, Homo sapiens
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 274 - protein, 107 aa, Homo sapiens
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 107 aa, Homo sapiens
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 276 - protein, 107 aa, Homo sapiens
GQPREPQVYTLPPSRDELTKNQVHLTCLVKGFYPSDIAVEWESNGQPENNYKTTPPVLDSDGSFALYSKLTVDKSRWQQGNVFSCSVMHEALHNHYTQKSLSLSPGK

SEQ ID NO: 277 - protein, 107 aa, Homo sapiens
GQPREPQVTTLPPSRDELTKNQVSLTCLVKGFYPSDIAVEWESNGQPENNYKTFPPVLDSDGSFFLYSKLTVDKSRWQQGNVFSCSVMHEALHNHYTQKSLSLSPGK

SEQ ID NO: 278 - protein, 107 aa, Homo sapiens
GQPREPQVYTLPPSRDELTKNQVSLTCDVSGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 107 aa, Homo sapiens
GQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 107 aa, Homo sapiens
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 281 - protein, 107 aa, Homo sapiens
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 282 - protein, 107 aa, Homo sapiens
GQPREPQVYTLPPSRDELTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 283 - protein, 107 aa, Homo sapiens
GQPRRPRVYTLPPSRDELTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 284 - protein, 107 aa, Homo sapiens
GQPREPQVYTLPPSRDELTKNQVSLTCLVKGFYPSDIAVEWESNGQPENNYKTTPPVLDSDGSFFLYSNLTVDKSRWQQGNVFSCSVMHEALHNHYTQKSLSLSPGK

SEQ ID NO: 285 - protein, 107 aa, Homo sapiens
GQPREPQVYTLPPSRDELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 286 - protein, 107 aa, Homo sapiens
GQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 287 - protein, 107 aa, Homo sapiens
GQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 288 - protein, 107 aa, Homo sapiens
GQPREPQVYTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 289 - protein, 107 aa, Homo sapiens
GQPREPRVYTLPPSRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 290 - protein, 107 aa, Homo sapiens
GQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPGK

SEQ ID NO: 291 - protein, 107 aa, Homo sapiens
GQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 292 - protein, 107 aa, Homo sapiens; identical to SEQ ID NO: 288

SEQ ID NO: 293 - protein, 107 aa, Homo sapiens
GQPREPQVYTLPPSRDNLTKNQVSLTCLVKGFYPSDIAVEWESNGQPENNYKTTPPVLVSDGSFTLYSKLTVDKSRWQQGNVFSCSVMHEALHNHYTQKSLSLSPGK

SEQ ID NO: 294 - protein, 107 aa, Homo sapiens
GQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 295 - protein, 107 aa, Homo sapiens
GQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 296 - protein, 110 aa, synthetic construct
GQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 297 - protein, 110 aa, synthetic construct
GQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 298 - protein, 110 aa, synthetic construct; identical to SEQ ID NO: 296

SEQ ID NO: 299 - protein, 110 aa, synthetic construct; identical to SEQ ID NO: 297

SEQ ID NO: 300 - protein, 110 aa, synthetic construct; identical to SEQ ID NO: 296

SEQ ID NO: 301 - protein, 110 aa, synthetic construct
GQPREPQVYTLPPPSEELALNELVTLTCLVKGFYPSDIAVEWLQGSQELPREKYLTWAPVLDSDGSFFLYSILRVAAEDWKKGDTFSCSVMHEALHNRFTQKSLDRSPGK

SEQ ID NO: 302 - protein, 236 aa, synthetic construct
MGWSCIILFLVATATGVHSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 303 - protein, 694 aa, synthetic construct
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 451 aa, synthetic construct
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 305 - protein, 214 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 306 - protein, 458 aa, synthetic construct; identical to SEQ ID NO: 234

SEQ ID NO: 307 - protein, 607 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKGGGGSGGGGSGGGGSGGGGSEVQLLESGGGLVQPGGSLRLSCAASGFTFTSYAMSWVRQAPGKGLEWVSTISGSGGGTYYTDSVKGRFTISRDNSKNTLYLQMNSLRAEDTAVYYCAQGSSIFDYWGQGTLVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPSTFGQGTRLEIK